(19) United States
(12) Patent Application Publication
(10) Pub. No.: US 2010/0256342 A1
(43) Pub. Date: Oct. 7, 2010

(54) PROTEIN NODES FOR CONTROLLED NANOSCALE ASSEMBLY

(76) Inventors: Francis Raymond Salemme, Yardley, PA (US); Patricia C. Weber, Yardley, PA (US); Mark A. Rould, South Burlington, VT (US)

Correspondence Address: VENABLE LLP, P.O. BOX 34385, WASHINGTON, DC, 20043-9998, US

(21) Appl. No.: 12/589,529

(22) Filed: Apr. 27, 2009

Related U.S. Application Data
(60) Provisional application No. 61/136,097, filed on Aug. 12, 2008.

(51) Int. Cl. C07K 14/00; C07K 19/00

(52) U.S. Cl. 530/391.1; 530/350; 530/408

(57) ABSTRACT

Engineered proteins for the assembly of two-dimensional and three-dimensional nanostructure assemblies. Methods for the systematic design and production of protein node structures that can be interconnected with streptavidin or streptavidin-incorporating struts to produce structures with defined dimensions and geometry. Nanostructure assemblies having utility as functional devices or as resists for the patterning of substrates. Nanostructure architectures including polygons, polyhedra, two-dimensional lattices, and three-dimensional lattices.

This application claims the benefit of U.S. Provisional Application No. 61/136,097, filed Aug. 12, 2008. This application incorporates the specifications of U.S. Provisional Application No. 60/996,089, filed Oct. 26, 2007, International Application No. PCT/US2008/012174, filed Oct. 27, 2008, U.S. Provisional Application No. 61/136,097, filed Aug. 12, 2008, and U.S. Provisional Application No. 61/173,114, filed Apr. 27, 2009, in their entirety by reference. All documents cited herein or cited in any one of the specifications incorporated by reference are hereby incorporated by reference.

Aspects of the work presented in this application have been made with U.S. Government support under a Small Business Innovation Research (SBIR) grant 1 R43 GM080805-01 A1 entitled “Engineered Macromolecule for Controlled Synthesis in Nanotechnology” funded under the NIH RFP PA-06-009: Bioengineering Nanotechnology Initiative and an SBIR grant 1 R43 GM077743-01A1 entitled “Engineering Proteins for Nanotechnology Applications” funded under the NIH RFP PA06-013: Manufacturing Processes of Medical, Dental, and Biological Technologies. The Government has certain rights in the invention.

### BACKGROUND OF THE INVENTION

Streptavidin:biotin complex formation has high affinity (Kd˜10⁻¹⁴) and specificity, which can lead to an essentially irreversible interaction. Ringler and Schultz (2003) used streptavidin together with a biotin-functionalized protein tetramer with C4 symmetry to create engineered 2D nanolattices on self-assembling monolayers (SAMs). They applied site-specific mutagenesis to a bacterial aldolase with C4 symmetry to introduce pairs of cysteine residues as biotinylation sites with geometry that was complementary to pairs of biotin binding sites on streptavidin. The aldolase molecules were also modified at their termini to incorporate histidine tags to facilitate isolation and allow the oriented binding and free two-dimensional diffusion necessary for self-assembly of the tetrameric aldolase with streptavidin on a monolayer surface. Ringler and Schulz indicated that they observed the construction of 2D lattices with defined nanometer dimensions using streptavidin molecules as well as other molecular linkers as connectors between 4-fold symmetric nodes (FIG. 3.1). However, Ringler and Schulz were only able to assemble very small lattices, and these generally incorporated many defects. This demonstrated limitations to their approach that would prevent the construction of practically useful materials and devices. The basic limitations of the Ringler-Schulz approach also manifest themselves as important sources of nonuniformity in more conventional applications like biosensors or diagnostics that rely on biotin-streptavidin interactions.

Miniaturization is required for the improvement of existing technologies and the enablement of new ones. For example, increases in the speed and processing power of computing machinery are dependent on further miniaturization. Silicon semiconductor devices, are presently fabricated by a “top down” sequential patterning technology using photolithography, far-ultraviolet lithography, or, more recently, electron beam lithography. Although progress with this technology has been made to produce ever smaller devices, it is generally recognized that the reliable production of structures with consistent sub-10 nanometer features probably lies beyond the capabilities of top-down silicon fabrication technology.

Self-assembling nanosystems might create complex and higher density novel device architectures. Such devices could potentially have applications as biosensors, actuators, biomaterials, or nanoelectronic devices for a wide variety of applications in fields as diverse as medicine and material science.

“Bottom up” techniques of self-assembly are common to biological systems (Padilla et al. 2001; Whitesides et al. 2002; Liu & Amro 2002; Lee et al. 2002; Ringler & Schulz 2003). Several companies are developing nanotechnology based on carbon or silicon-based nanostructures, functionalized carbon nanotubes, or buckyballs. An alternative approach to the development of self-assembled nanostructures makes use of biomolecules like nucleic acids and proteins. Several 2-dimensional and 3-dimensional nanostructures formed of DNA have been generated. (Rothemund 2006; Seeman, 2005ab; Shih 2004).

Whole viruses have been used as substrates for nanostructures, as described in Blum et al. (2004), Blum et al. 2005, Chatterji et al. (2004), Chatterji et al. 2005, and Falkner et al. (2005). Cambrios uses virus structures for material sciences applications (www.cambrios.com).

Several examples of 1-dimensional (e.g. Medalsy et. al., 2008), 2-dimensional (e.g. Sleytr et. al. 2007) and 3-dimensional protein arrays (e.g. protein crystals) have been reported, with several suggestions for the use of such arrays as nanostructural templates.

Padilla et. al (2001) and Yeates et. al (2004) proposed the use of engineered fusion proteins, produced by using recombinant DNA technology to link the genes coding for subunits of protein multimers of different symmetry, as a means of producing both 2-dimensional and 3-dimensional protein lattices and polyhedra. In their 2001 work, Padilla et. al. described the spontaneous assembly in solution of both tetrahedral complexes and a linear helical filament using the fused protein domain approach.

An alternative approach to the formation of 2-dimensional self-assembling lattices of biomolecules involves diffusional organization on self-assembled monolayers (SAMs). Several examples have been cited (e.g. Liu rt. al. 1996, Liu & Amro 2002, Lee et. al. 2002, Sleytr et. al. 2007) with potential applications to nanostructure assembly.

With the exception of some DNA-based nanostructures, the protein-based assemblies cited above primarily result from the spontaneous association of molecules and so only allow limited control over nanostructure assembly.

Two-dimensional structures with controlled and definable lattice dimensions organized on SAMs were suggested as concepts by Sligar and Salemme (1992) and were based on composite assemblies incorporating the tetrameric binding protein streptavidin used in combination with DNA. In 2003, Ringler & Schulz described the geometrically controlled formation of a structure that incorporated a modified form of the tetrameric aldolase RhoA from E. coli and streptavidin. They used site-directed mutagenesis to incorporate cysteine residues on the RhoA protein surface as attachment points for biotin, so that each monomer of the RhoA tetramer could make pairwise interactions with streptavidin. The RhoA protein was also modified at its termini to incorporate histidine tags to facilitate isolation and allow oriented binding and free 2-dimensional diffusion necessary for self-assembly of the tetrameric aldolase with streptavidin on a self-assembling monolayer surface. The researchers assembled a 2-dimensional lattice formed of the RhoA tetramers and streptavidin through interaction of the proteins with the self-assembled monolayer.

There are several limitations in the 2003 work by Ringler & Schulz, as well as the work of Padilla et. al. (2001) using fused proteins. Specifically, in both cases the assembly process resulted in the formation of many non-uniform or defective structures. There were several factors that contributed to the poor quality of the structural assemblies. In the case of both the Padilla et. al. and Ringler & Schulz approaches, the assembly occurred spontaneously so that there was no control on the steps of assembly, resulting in partial structures and aggregated complexes. Partly this was caused by forming assemblies from proteins that were not particularly stable. Typical proteins from animals or microorganisms that live close to ambient temperatures (20 deg C. to 40 deg C.) are relatively difficult to manufacture and purify, and also undergo structural fluctuations that produce alternative conformational states that frustrate accurate self-assembly of molecular components. In addition, in the case of structures that are assembled through a combination of components linked chemically, such as the Ringler & Schulz 2-dimensional RhoA-streptavidin lattices, it is necessary to insure precise control of the linking geometry between the structural components. Without intending to be limited by theory, the inventors believe that it is likely that imperfections in the interaction geometry engineered by Ringler & Schulz produced cumulative twist in their 2-dimensional lattices that ultimately limited the size of the lattice that could self-assemble. A major general difference between the Padilla et. al. approach and the Ringer & Schultz approach is that in the former approach, interacting domains of the fused molecules forming the structures are essentially flexible, whereas in the latter approach they are essentially rigid.

### SUMMARY OF THE INVENTION

### Method of Using Proteins for Nanostructure Assemblies

1. A method of using a template multimeric protein with Cn, Dn, or higher symmetry, that incorporates specific attachment sites for nanostructure struts with predefined stoichiometry and orientation, and is derived from a thermophilic microorganism, as a nanostructure node.

2. A method of using a list of sequences of multimeric proteins with Cn, Dn or higher symmetries derived from template multimeric proteins (having a template number of polypeptide chains) of thermostable organisms with utility as node templates for the generation of nanostructure nodes including nanostructure node multimeric proteins incorporating specific binding sites for the symmetric attachment of nanostructure struts with defined stoichiometry and orientation.

3. A method of using a set of sequences with greater than 80 percent sequence identity with a list of multimeric proteins with Cn, Dn or higher symmetries derived from thermostable organisms with utility as node templates for the generation of nanostructure nodes incorporating specific binding sites for the symmetric attachment of nanostructure struts with defined stoichiometry and orientation.

4. A method of using a list of sequences of multimeric proteins with Cn symmetry derived from template multimeric proteins (having a template number of polypeptide chains) of thermostable organisms with utility as node templates for the generation of nanostructure nodes including nanostructure node multimeric proteins incorporating specific binding sites for the symmetric attachment of nanostructure struts with defined stoichiometry and orientation.

5. A method of using a set of sequences with greater than 80 percent sequence identity with a list of multimeric proteins with Cn symmetry derived from thermostable organisms with utility as node templates for the generation of nanostructure nodes incorporating specific binding sites for the symmetric attachment of nanostructure struts with defined stoichiometry and orientation.

6. A method of using a protein node incorporating multiple subunit polypeptide chains related by Cn symmetry, with each subunit incorporating two specific amino acid reactive sites (specific amino acid reactive residues) permitting the covalent attachment of biotin groups, subsequently allowing interconnection with streptavidin tetramers with defined stoichiometry and orientation.

### Method of Making Nanostructure Assemblies

7. A method of making a nanostructure node by operating on the 3-dimensional structure of a member of a list of multimeric node template proteins derived from thermostable organisms, to define the amino sequence of nodes that can form nanoassemblies incorporating multimeric nodes and streptavidin or streptavidin-incorporating struts attached with defined stoichiometry and orientation.

8. A method of making a nanostructure node by operating on the 3-dimensional structure of a member of a list of multimeric node template proteins with Cn symmetry derived from thermostable organisms, to define the amino sequence of nodes that can form planar nanoassemblies incorporating Cn planar nodes and streptavidin or streptavidin-incorporating struts attached with defined stoichiometry and orientation.

9. A method of making a nanostructure node by operating on the 3-dimensional structure of a member of a list of multimeric node template proteins with Cn symmetry derived from thermostable organisms, using an aligned search procedure with a relative rotational increment of between 0.001 and 5 degrees to define the amino sequence of nodes that can form planar nanoassemblies incorporating Cn planar nodes and streptavidin or streptavidin-incorporating struts attached with defined stoichiometry and orientation.

10. A method of making an optimal nanostructure node by operating on the 3-dimensional structure of a member of a list of multimeric node template proteins with Cn symmetry derived from thermostable organisms to define the amino sequence of nodes that can form planar nanoassemblies incorporating Cn planar nodes and streptavidin or streptavidin-incorporating struts attached with defined stoichiometry and orientation.

11. A method of making an optimal nanostructure node that is produced through expression in an E. coli bacterium or another heterologous protein expression system.

12. A method of making an optimal planar nanostructure node by using computer graphics, mathematical, or experimental methods of improving the interface interactions between a Cn polyhedral node and streptavidin resulting in modified node protein amino acid sequences.

13. A method of making a planar protein node based on a template node sequence from a thermophilic organism that incorporates multiple subunit polypeptide chains related by C3, C4, C5, C6, and C7 symmetry, and that has been modified according to a computer graphical or mathematical method to define and incorporate two reactive amino acid groups permitting the covalent attachment of biotin groups, subsequently allowing Cn-symmetric interconnection between the node and n streptavidin tetramers in a planar orientation.

14. A method of making a nanostructure node by operating on the 3-dimensional structure of a member of a list of multimeric proteins with Cn symmetry derived from thermostable organisms, to define the amino sequence of nodes that can form polyhedral nanoassemblies incorporating streptavidin or streptavidin-incorporating struts connected to nodes with geometry and stoichiometry corresponding to the apex of a regular polyhedron.

15. A method of making an optimal nanostructure node by operating on the 3-dimensional structure of a member of a list of multimeric proteins with Cn symmetry derived from thermostable organisms, to define the amino sequence of nodes that can form polyhedral nanoassemblies incorporating streptavidin or streptavidin-incorporating struts connected to nodes with geometry and stoichiometry corresponding to the apex of a regular polyhedron.

16. A method of making an optimal polyhedral nanostructure node by using computer graphics, mathematical, or experimental methods of improving the interface interactions between a Cn polyhedral node and streptavidin resulting in modified node protein amino acid sequences.

17. A method of making nanostructure nodes by operating on the 3-dimensional structure of a member of a list of multimeric proteins with Dn or higher symmetry derived from thermostable organisms, to define the amino sequence of nanostructure nodes that can form nanoassemblies incorporating streptavidin or streptavidin-incorporating struts connected to nodes with defined geometry and stoichiometry along node dyad symmetry axes.

18. A method of making optimal nanostructure nodes by operating on the 3-dimensional structure of a member of a list of multimeric proteins with Dn or higher symmetry derived from thermostable organisms, to define the optimal amino sequence of nodes that can form nanoassemblies incorporating streptavidin or streptavidin-incorporating struts connected to nodes with defined geometry and stoichiometry along node dyad symmetry axes.

19. A method of making optimal nanostructure nodes using computer graphics, mathematical methods, or experimental methods for defining amino acid sequences of nanostructure nodes with improved interface interactions between a Dn or higher symmetry node and streptavidin.

20. A method of making a protein node where at least one subunit polypeptide chain has been modified through reaction with a bifunctional reagent to incorporate additional binding or other functionality into the node polypeptide chain.

21. A method of making a protein node where at least one subunit polypeptide chains have been modified through covalent incorporation of a polypeptide chain sequence coding for protein binding or other functionality.

22. A method of making a protein node with subunit polypeptide chains related by Cn, Dn or higher symmetry, where some subunits have been covalently interconnected to form a protein multimer with a reduced number of polypeptide chains.

### Composition of Matter: Nanostructure Nodes

23. In an embodiment, a nanostructure node generated from a template multimeric protein with Cn, Dn, or higher symmetry, derived from a list of known three-dimensional protein structures with corresponding symmetry as determined from X-ray crystallography, that incorporates specific attachment sites for nanostructure struts with predefined stoichiometry and orientation, and is derived from a thermophilic microorganism, as a nanostructure node.

24. In an embodiment, a nanostructure node generated from a template multimeric protein with Cn, Dn, or higher symmetry, derived from a protein that is homologous to one derived from a list of known three-dimensional protein structures with corresponding symmetry as determined from X-ray crystallography, that incorporates specific attachment sites for nanostructure struts with predefined stoichiometry and orientation, and is derived from a thermophilic microorganism.

25. In an embodiment, a protein node where at least one subunit polypeptide chain has been modified through reaction with a bifunctional reagent to incorporate additional binding or other functionality into the node polypeptide chain.

26. In an embodiment, a protein node where at least one subunit polypeptide chain has been modified through covalent incorporation of a polypeptide chain sequence coding for protein binding or other functionality.

27. In an embodiment, a protein node with subunit polypeptide chains related by Cn, Dn or higher symmetry, where two of the subunits have been covalently interconnected to form a protein multimer with a reduced number of polypeptide chains, and modified to incorporate specific binding sites for chemical modification leading to the covalent attachment of biotin groups.

28. In an embodiment, a protein node with subunit polypeptide chains related by Cn, Dn or higher symmetry, where some subunits have been covalently interconnected to form a protein multimer with a reduced number of polypeptide chains, and modified to incorporate specific binding sites for chemical modification leading to the covalent attachment of biotin groups.

29. In an embodiment, a protein node with subunit polypeptide chains related by Cn, Dn or higher symmetry, where all of subunits have been covalently interconnected to form a protein multimer composed of a single polypeptide chain, and modified to incorporate specific binding sites for chemical modification leading to the covalent attachment of biotin groups.

30. In an embodiment, a protein node with subunit polypeptide chains related by Cn symmetry, where two of the subunits have been covalently interconnected to form a protein multimer with a reduced number of polypeptide chains, and modified to incorporate specific binding sites for chemical modification leading to the covalent attachment of biotin groups.

31. In an embodiment, a planar C3 node based on the pdb code: 1thj trimer whose subunits have been interconnected using a short polypeptide linker to form a single polypeptide chain or homologues thereof.

32. In an embodiment, a planar C3 node based on amino acid sequences that are homologous to the pdb code: 1thj trimer whose subunits have been interconnected using a short polypeptide linker to form a single polypeptide chain.

33. In an embodiment, a planar protein node based on the template protein pdb code: 1thj, incorporating three subunit polypeptide chains related by C3 symmetry, and incorporating cysteine amino acid residues as reactive sites for the covalent attachment of biotin groups, subsequently allowing C3 symmetric interconnection with 3 streptavidin tetramers in a planar orientation.

34. In an embodiment, a planar protein node based on the template protein pdb code: 1j5s, incorporating three subunit polypeptide chains related by C3 symmetry, and incorporating cysteine amino acid residues as reactive sites for the covalent attachment of biotin groups, subsequently allowing C3 symmetric interconnection with 3 streptavidin tetramers in a planar orientation.

35. In an embodiment, a planar protein node based on the template protein pdb code: 1vcg, incorporating four subunit polypeptide chains related by C4 symmetry, where each subunit incorporates cysteine amino acid residues as reactive sites for the covalent attachment of biotin groups, subsequently allowing C4 symmetric interconnection with 4 streptavidin tetramers in a planar orientation.

36. In an embodiment, a planar protein node based on the template protein pdb code: 2cu0, incorporating four subunit polypeptide chains related by C4 symmetry, where each subunit has been modified according to a computer graphical or mathematical method to define and incorporate two cysteine amino residues as reactive sites for the covalent attachment of biotin groups, subsequently allowing C4 symmetric interconnection with 4 streptavidin tetramers in a planar orientation.

37. In an embodiment, a planar protein node based on the template node protein pdb code: 1vdh that incorporates five subunit polypeptide chains related by C5 symmetry, and where each subunit incorporates two cysteine amino acid residues, as determined using a computer graphics or mathematical method, as reactive sites for the covalent attachment of biotin groups, subsequently allowing C5 symmetric interconnection with 5 streptavidin tetramers in a planar orientation.

38. In an embodiment, a planar protein node based on the template node sequence pdb code: 2ekd that incorporates six subunit polypeptide chains related by C6 symmetry, and where each subunit incorporates two cysteine amino acid residues, as determined using a computer graphics or mathematical method, as reactive sites for the covalent attachment of biotin groups, subsequently allowing C6 symmetric interconnection with 6 streptavidin tetramers in a planar orientation.

39. In an embodiment a planar protein node based on the template node sequence pdb code: 1i81 that incorporates seven subunit polypeptide chains related by C7 symmetry, and where each subunit incorporates two cysteine amino acid residues, as determined using a computer graphics or mathematical method, as reactive sites for the covalent attachment of biotin groups, subsequently allowing C7 symmetric interconnection with 7 streptavidin tetramers in a planar orientation.

40. In an embodiment, a polyhedral protein node incorporating three subunit polypeptide chains related by C3 symmetry, based on the template protein pdb code: 1v4n, and incorporating specific binding sites for chemical modification leading to the covalent attachment of biotin groups, subsequently allowing interconnection with 3 streptavidin tetramers in an orientation corresponding to the apex of a dodecahedron.

41. In an embodiment, a polyhedral protein node incorporating three subunit polypeptide chains related by C3 symmetry, based on the template protein pdb code: 1v4n, and incorporating specific binding sites for chemical modification leading to the covalent attachment of biotin groups, subsequently allowing interconnection with 3 streptavidin tetramers in an orientation corresponding to the apex of a truncated icosahedron or “bucky ball” structure.

42. In an embodiment, a polyhedral protein node incorporating five subunit polypeptide chains related by C5 symmetry, based on the template protein pdb code: 1vdh, and incorporating specific binding sites for chemical modification leading to the covalent attachment of biotin groups, subsequently allowing interconnection with 5 streptavidin tetramers in an orientation corresponding to the apex of an icosahedron.

43. In an embodiment, a protein node based on the tetrameric D2-symmetric node template pdb code: 1ma1, where positions on subunits related by D2 symmetry have been modified to incorporate specific cysteine residues allowing covalent attachment of biotin groups and subsequent interconnection with streptavidin tetramers with defined stoichiometry and orientation. According to whether cysteine modifications are introduced along one, two, or all three of the independent dyad axes of the tetramer, streptavidin linked structures with linear, 2-dimensional rectangular, or 3-dimensional orthorhombic lattice geometry may be formed.

44. In an embodiment, a protein node based on the tetrameric D2-symmetric node template pdb code: 1nto. According to whether cysteine modifications are introduced along one, two, or all three of the independent dyad axes of the tetramer, streptavidin linked structures with linear, 2-dimensional rectangular, or 3-dimensional orthorhombic lattice geometry may be formed.

45. In an embodiment, a protein node based on the tetrameric D2-symmetric node template pdb code: 1rtw. According to whether cysteine modifications are introduced along one, two, or all three of the independent dyad axes of the tetramer, streptavidin linked structures with linear, 2-dimensional rectangular, or 3-dimensional orthorhombic lattice geometry may be formed.

46. In an embodiment, a protein node based on the hexameric D3-symmetric node template pdb code: 1b4b. Such nodes have utility in the formation of 2-dimensional and 3-dimensional hexagonal lattices.

47. In an embodiment, a protein node based on the hexameric D3-symmetric node template pdb code: 1hyb. Such nodes have utility in the formation of 2-dimensional and 3-dimensional hexagonal lattices.

48. In an embodiment, a protein node based on the hexameric D3-symmetric node template pdb code: 2prd. Such nodes have utility in the formation of 2-dimensional and 3-dimensional hexagonal lattices.

49. In an embodiment, a protein node based on the octameric D4-symmetric node template pdb code: 1o4v. Such nodes have utility in the formation of 2-dimensional and 3-dimensional lattices with tetragonal node symmetry.

50. In an embodiment, a protein node based on the octameric D4-symmetric node template pdb code: 2h2i. Such nodes have utility in the formation of 2-dimensional and 3-dimensional lattices with tetragonal node symmetry.

51. In an embodiment, a protein node based on the octameric D4-symmetric node template pdb code: 2ie1. Such nodes have utility in the formation of 2-dimensional and 3-dimensional lattices with tetragonal node symmetry.

52. In an embodiment, a protein node based on the dodecameric tetrahedral T23-symmetric node template pdb code: 1pvv. Such nodes have utility in the formation of 3-dimensional lattices with cubic symmetry.

53. In an embodiment, modified forms of the D2-symmetric, tetrameric protein streptavidin (pdb code: 1stp), where cysteine residues have been introduced along tetramer dyad axes to protect biotin binding sites or allow subsequent in situ functionalization of nanostructures incorporating streptavidin struts.

### Composition of Matter: Extended Struts

54. In an embodiment, an extended strut composed of a protein node based on a tetrameric D2-symmetric node template pdb code: 1ma1 complexed with two streptavidin tetramers to form an extended nanostructure strut.

Composition of Matter: Assemblies with a Nanostructure Node

55. In an embodiment, a nanostructure assembly geometry incorporating Cn-symmetric or Dn symmetric nodes and streptavidin or streptavidin-incorporating struts.

56. In an embodiment, a nanostructure assembly incorporating streptavidin or streptavidin-incorporating struts together with Cn-symmetric or Dn symmetric nodes based on node templates derived from a list of known three-dimensional protein structures with corresponding symmetry as determined from X-ray crystallography, and that incorporate specific attachment sites for nanostructure struts with predefined stoichiometry and orientation, and are derived from thermophilic microorganisms.

57. In an embodiment, a nanostructure assembly incorporating streptavidin or streptavidin-incorporating struts together with Cn-symmetric or Dn symmetric nodes based on templates that are amino acid sequence homologs of structures derived from a list of known three-dimensional protein structures with corresponding symmetry as determined from X-ray crystallography, and that incorporate specific attachment sites for nanostructure struts with predefined stoichiometry and orientation, and are derived from thermophilic microorganisms.

58. In an embodiment, a nanostructure assembly incorporating streptavidin or streptavidin-incorporating struts together with D2 symmetric nodes that are based on a modified forms of streptavidin that incorporate specific attachment sites for nanostructure struts with predefined stoichiometry and orientation.

59. In an embodiment, a nanostructure assembly incorporating Cn-symmetric or Dn symmetric nodes and streptavidin or streptavidin-incorporating struts. The nanostructure may be functionalized through the incorporation of node constructs that have been modified either through reaction with a bifunctional reagent to incorporate additional binding or other functionality into the node polypeptide chain, or where node subunits have been modified through covalent incorporation of a polypeptide chain sequence coding for protein binding or other functionality.

60. In an embodiment, a nanostructure assembly incorporating Cn-symmetric or Dn symmetric nodes and streptavidin or streptavidin-incorporating struts taking the geometrical form of a radial planar array.

61. In an embodiment, a nanostructure with 2-dimensional polygonal geometry incorporating Cn-symmetric nodes and streptavidin or streptavidin-incorporating struts.

62. In an embodiment, a nanostructure with 2-dimensional polygonal geometry incorporating single-chain Cn-symmetric nodes and streptavidin or streptavidin-incorporating struts.

63. In an embodiment, a nanostructure with 2-dimensional hexagonal polygonal geometry incorporating single-chain C3-symmetric nodes and streptavidin or streptavidin-incorporating struts.

64. In an embodiment, a nanostructure with 2-dimensional hexagonal polygonal geometry incorporating single-chain C3-symmetric nodes based on node templates derived from a list of known three-dimensional protein structures with corresponding symmetry as determined from X-ray crystallography, and streptavidin or streptavidin-incorporating struts.

65. In an embodiment, a nanostructure with 2-dimensional hexagonal polygonal geometry incorporating single-chain C3-symmetric nodes based on the node templates pdb code: 1thj.

66. In an embodiment, a nanostructure with 2-dimensional square polygonal geometry incorporating single-chain C4-symmetric nodes and streptavidin or streptavidin-incorporating struts.

67. In an embodiment, a nanostructure with 2-dimensional square polygonal geometry incorporating single-chain C4-symmetric nodes based on node templates derived from a list of known three-dimensional protein structures with corresponding symmetry as determined from X-ray crystallography, and streptavidin or streptavidin-incorporating struts.

68. In an embodiment, a nanostructure with 2-dimensional square polygonal geometry incorporating single-chain C4-symmetric nodes based on the node template pdb code: 1vcg.

69. In an embodiment, a 2-dimensional lattice incorporating Cn-symmetric nodes and streptavidin or streptavidin-incorporating struts.

70. In an embodiment, a 2-dimensional lattice incorporating Dn-symmetric nodes and streptavidin or streptavidin-incorporating struts.

71. In an embodiment, a 2-dimensional hexagonal lattice incorporating C3-symmetric nodes and streptavidin or streptavidin-incorporating struts.

72. In an embodiment, a 2-dimensional hexagonal lattice incorporating C3-symmetric nodes based on node templates corresponding to the pdb code: 1thj protein trimer or the pdb code: 1j5s protein trimer and streptavidin or streptavidin-incorporating struts.

73. In an embodiment, a 2-dimensional square lattice incorporating C4-symmetric nodes and streptavidin or streptavidin-incorporating struts.

74. In an embodiment, a 2-dimensional square lattice incorporating C4-symmetric nodes homologous to node template sequences corresponding to the pdb code: 1vcg protein tetramer and streptavidin or streptavidin-incorporating struts.

75. In an embodiment, a 2-dimensional square lattice incorporating C4-symmetric nodes based on the node template sequence pdb code: 1vcg and streptavidin or streptavidin-incorporating struts.

76. In an embodiment, a 3-dimensional radial nanostructure incorporating a node derived from thermophilic node templates with Dn, tetrahedral (T23), cubeoctahedral (432), or with icosahedral/dodecahedral (532) symmetry derived from a thermophilic organism, and Dn symmetric nodes and streptavidin or streptavidin-incorporating struts.

77. In an embodiment, a 3-dimensional radial nanostructure incorporating a node that is homologous to thermophilic node templates with Dn, tetrahedral (T23), cubeoctahedral (432), or with icosahedral/dodecahedral (532) symmetry derived from a thermophilic organism, and Dn symmetric nodes and streptavidin or streptavidin-incorporating struts

78. In an embodiment, a 3-dimensional radial nanostructure incorporating a node with tetrahedral (T23) symmetry based on a dodecameric node and streptavidin or streptavidin-incorporating struts.

79. In an embodiment, a 3-dimensional radial nanostructure incorporating a node template with tetrahedral (T23) symmetry derived from a list of known three-dimensional protein structures with corresponding symmetry as determined from X-ray crystallography, and streptavidin or streptavidin-incorporating struts.

80. In an embodiment, a 3-dimensional radial nanostructure incorporating a node with tetrahedral (T23) symmetry based on the dodecameric node template pdb code: 1pvv and streptavidin or streptavidin-incorporating struts.

81. In an embodiment, a 3-dimensional radial nanostructure incorporating a node with cubeoctahedral symmetry based on the 24-subunit node template derived from a thermophilic organism and streptavidin or streptavidin-incorporating struts.

82. In an embodiment, a 3-dimensional radial nanostructure incorporating a node with cubeoctahedral symmetry based on a 24-subunit node template derived from a list of known three-dimensional protein structures with corresponding symmetry as determined from X-ray crystallography, and streptavidin or streptavidin-incorporating struts.

83. In an embodiment, a 3-dimensional radial nanostructure incorporating a node with icosahedral/dodecahedral 532 symmetry based on a 60-subunit node template derived from a thermophilic organism and Dn symmetric nodes and streptavidin or streptavidin-incorporating struts.

84. In an embodiment, a 3-dimensional radial nanostructure incorporating a node with icosahedral/dodecahedral 532 symmetry based on a 60-subunit node template derived from a list of known three-dimensional protein structures with corresponding symmetry as determined from X-ray crystallography, and streptavidin or streptavidin-incorporating struts.

85. In an embodiment, a 3-dimensional polyhedron formed of streptavidin or streptavidin-incorporating struts, and nodes with Cn symmetry incorporating binding interactions corresponding to the apex geometry of a polyhedron.

86. In an embodiment, a 3-dimensional polyhedron formed of streptavidin or streptavidin-incorporating struts, and nodes with Cn symmetry template derived from a list of known three-dimensional protein structures with corresponding symmetry as determined from X-ray crystallography, and incorporating binding interactions corresponding to the apex geometry of a polyhedron.

87. In an embodiment, a 3-dimensional dodecahedron formed of streptavidin or streptavidin-incorporating struts, and nodes with C3 symmetry, incorporating binding interactions corresponding to the apex geometry of a dodecahedron.

88. In an embodiment, a 3-dimensional dodecahedron formed of streptavidin or streptavidin-incorporating struts, and nodes with C3 symmetry, based on the pdb code: 1v4n node protein, incorporating binding interactions corresponding to the apex geometry of a dodecahedron.

89. In an embodiment, a 3-dimensional “bucky” polyhedron formed of streptavidin or streptavidin-incorporating struts, and nodes with C3 symmetry, incorporating binding interactions corresponding to the apex geometry of a truncated icosahedron.

90. In an embodiment, a 3-dimensional “bucky” polyhedron formed of streptavidin or streptavidin-incorporating struts, and nodes with C3 symmetry, based on the pdb code: 1v4n node protein, incorporating binding interactions corresponding to the apex geometry of a truncated icosahedron.

91. In an embodiment, a 3-dimensional icosahedron formed of streptavidin or streptavidin-incorporating struts, and nodes with C5 symmetry, incorporating binding interactions corresponding to the apex geometry of an icosahedron.

92. In an embodiment, a 3-dimensional icosahedron formed of streptavidin or streptavidin-incorporating struts, and nodes with C5 symmetry, based on the pdb code: 1vdh node protein, incorporating binding interactions corresponding to the apex geometry of an icosahedron.

93. In an embodiment, a 3-dimensional, three-connected hexagonal-pattern lattice formed of streptavidin or streptavidin-incorporating struts, and nodes with D3 symmetry, alternatively modified to allow binding to streptavidin in two orientations.

94. In an embodiment, a 3-dimensional, three-connected hexagonal-pattern lattice formed of streptavidin or streptavidin-incorporating struts, and different nodes with D3 symmetry derived from a list of known three-dimensional protein structures with corresponding symmetry as determined from X-ray crystallography, alternatively modified to allow binding to streptavidin in two orientations.

95. In an embodiment, a 3-dimensional, three-connected lattice formed of streptavidin or streptavidin-incorporating struts, and nodes with D3 symmetry derived from a list of known three-dimensional protein structures with corresponding symmetry as determined from X-ray crystallography, the same D3 templates being alternatively modified to allow binding to streptavidin in two orientations.

96. In an embodiment, a 3-dimensional, three-connected lattice formed of streptavidin or streptavidin-incorporating struts, and nodes with D3 symmetry based on the pdb code: 1hyb node protein template, alternatively modified to allow binding to streptavidin in two orientations.

97. In an embodiment, a nanostructure comprising a 3-dimensional, four-connected, cubic pattern lattice formed of nodes with D4 symmetry and streptavidin or streptavidin-incorporating struts.

98. In an embodiment, a nanostructure comprising a 3-dimensional, four-connected, cubic pattern lattice formed of streptavidin or streptavidin-incorporating struts, and nodes with D4 symmetry derived from a list of known three-dimensional protein structures with corresponding symmetry as determined from X-ray crystallography, alternatively modified to allow binding to streptavidin in two orientations.

99. In an embodiment, a 3-dimensional, four-connected lattice formed of streptavidin or streptavidin-incorporating struts, and nodes with D4 symmetry based on the pdb code: 2h21 node protein template, alternatively modified to allow binding to streptavidin in two orientations.

100. In an embodiment, a 3-dimensional, six-connected cubic lattice formed of streptavidin or streptavidin-incorporating struts.

101. In an embodiment, a 3-dimensional, six-connected cubic lattice formed of streptavidin or streptavidin-incorporating struts, and nodes with tetrahedral symmetry.

102. In an embodiment, a 3-dimensional, six-connected cubic lattice formed of streptavidin or streptavidin-incorporating struts, and nodes with T23 symmetry derived from a list of known three-dimensional protein structures with corresponding symmetry as determined from X-ray crystallography.

103. In an embodiment, a 3-dimensional, six-connected cubic lattice formed of streptavidin or streptavidin-incorporating struts, and nodes with tetrahedral symmetry based on the pdb code: 1pvv node protein template.

### Composition of Matter: Multimeric Node Protein Architectures

104. In an embodiment, a nanostructure node protein based on a template sequence derived from a thermostable microorganism and comprising multiple polypeptide chain subunits and specific binding functionality.

105. In an embodiment, a nanostructure node incorporating 3, 5, or 6 subunits.

106. In an embodiment, a nanostructure node incorporating 3, 5, or 6 subunits, where the subunits are related by rotational symmetry.

107. In an embodiment, a nanostructure node multimeric protein incorporating multiple polypeptide subunits related by tetrahedral, octahedral, or icosahedral symmetry.

108. In an embodiment, a nanostructure node protein based on a template sequence derived from a thermostable microorganism and comprising multiple polypeptide chain subunits, each with a specific binding functionality.

109. In an embodiment, a nanostructure node protein based on a template sequence derived from a thermostable microorganism and comprising multiple polypeptide chain subunits, where at least one subunit lacks a specific binding functionality.

110. In an embodiment, a nanostructure node protein based on a template sequence derived from a thermostable microorganism and comprising 4 polypeptide chain subunits, each with a specific binding functionality and related by 4-fold symmetry.

111. In an embodiment, a nanostructure node protein based on a template sequence derived from a thermostable microorganism and comprising 3, 4, or 6 polypeptide chain subunits incorporating specific binding functionality that lie in a plane.

112. In an embodiment, a nanostructure node protein based on a template sequence derived from a thermostable microorganism and comprising 3, 4, or 6 polypeptide chain subunits, each with a specific binding functionality and related by rotational symmetry.

113. In an embodiment, a nanostructure node protein based on a template sequence derived from a thermostable microorganism and comprising 4 polypeptide chain subunits, each with a specific binding functionality, and where at least one specific binding site does not lie within the same plane as the other specific binding sites.

114. In an embodiment, a nanostructure node protein based on a template sequence derived from a thermostable microorganism and comprising multiple polypeptide chain subunits and wherein a first subunit is covalently bonded to a second subunit.

115. In an embodiment, a nanostructure node protein based on a template sequence derived from a thermostable microorganism and comprising at least 3 polypeptide chain subunits and wherein at least three subunits are covalently bonded to form a single polypeptide chain.

116. In an embodiment, a thermostable nanostructure node protein based on a template sequence derived from a thermostable microorganism and comprising multiple polypeptide chain subunits and specific binding functionality.

117. In an embodiment, a nanostructure node protein based on a template sequence derived from a thermostable microorganism and comprising multiple polypeptide chain subunits and specific binding functionality and where the amino acid sequence of at least one subunit is different from the amino acid sequence of another subunit.

118. In an embodiment, a nanostructure node protein with at least 80% sequence homology with a template sequence derived from a thermostable microorganism and comprising multiple polypeptide chain subunits and specific binding functionality.

119. In an embodiment, a trimeric C3-symmetric nanostructure node multimeric protein where the amino acid sequence of each polypeptide subunit has at least 80% sequence identity with an amino acid sequence of the uronate isomerase TM0064 from Thermotoga maritime (pdb code: 1j5s).

120. In an embodiment, a tetrameric C4-symmetric nanostructure where the amino acid sequence of each polypeptide subunit has at least 80% amino acid sequence identity with an amino acid sequence of the isopentenyl-diphosphate delta-isomerase (pdb code: 1vcg) from Thermus thermophilus.

121. In an embodiment, a nanostructure node protein based on a template sequence derived from a thermostable microorganism and comprising multiple polypeptide chain subunits and specific binding functionality, wherein each specific binding site incorporates a specific amino acid residue separated from the other specific amino acid residue by a distance of about 20 Angstroms.

122. In an embodiment, a nanostructure node protein based on a template sequence derived from a thermostable microorganism and comprising multiple polypeptide chain subunits and specific binding functionality, wherein each specific binding site incorporating a specific amino acid residue is separated from the other specific amino acid residue by a distance such that with biotin groups bound to the specific amino acid residues, the biotin groups are positioned to bind with a pair of binding sites on streptavidin.

123. In an embodiment, a nanostructure node protein based on a template sequence derived from a thermostable microorganism and comprising multiple polypeptide chain subunits and specific binding functionality, where at least one subunit incorporates a polypeptide extension of from 5 to 1000 amino acid residues linked with a peptide bond to the designated amino and/or carboxy terminus.

124. In an embodiment, a nanostructure node protein based on a template sequence derived from a thermostable microorganism and comprising multiple polypeptide chain subunits and specific binding functionality, where at least one subunit incorporates a polypeptide extension of from 5 to 1000 amino acid residues linked with a peptide bond to the designated amino and/or carboxy terminus that comprises a binding function for a protein or a metallic or other solid surface.

125. In an embodiment, a nanostructure node protein based on a template sequence derived from a thermostable microorganism and comprising multiple polypeptide chain subunits and specific binding functionality, where at least one subunit incorporates a polypeptide extension of from 5 to 1000 amino acid residues linked with a peptide bond to the designated amino and/or carboxy terminus that comprises an amino acid subsequence that is a substrate for an enzyme.

126. In an embodiment, a nanostructure node protein based on a template sequence derived from a thermostable microorganism and comprising multiple polypeptide chain subunits and specific binding functionality, where at least one subunit incorporates a polypeptide extension of from 5 to 1000 amino acid residues linked with a peptide bond to the designated amino and/or carboxy terminus that comprises a polypeptide subsequence selected from the group consisting of an immunoglobulin polypeptide, a polyhistidine, a streptavidin binding polypeptide, Streptag, an antibody binding polypeptide, staphylococcus Protein A, staphylococcus Protein G, an antigenic polypeptide, and a hapten-binding polypeptide.

127. In an embodiment, a nanostructure node protein based on a template sequence derived from a thermostable microorganism and comprising multiple polypeptide chain subunits and specific binding functionality, where at least one subunit incorporates a polypeptide extension of from 5 to 1000 amino acid residues linked with a peptide bond to the designated amino and/or carboxy terminus that comprises an antibody binding polypeptide subsequence together with a bound antibody.

128. In an embodiment, a nanostructure assembly incorporating a multimeric nanostructure node protein together with a specifically bound nanostructure strut.

129. In an embodiment, a nanostructure node comprising three subunits where two subunits incorporate specific binding sites and one subunit does not. In its C3 symmetric form, the nanostructure node functions as a 120 degree linker between two nanostructure struts.

130. In an embodiment, a nanostructure node comprising three subunits where one subunit incorporates a specific binding site and two subunits do not. The nanostructure node functions as a cap or terminator for a nanostructure struts.

131. In an embodiment, a nanostructure node comprising four subunits where three subunits incorporate specific binding sites and one subunit does not. In its C4 symmetric form, the nanostructure node functions as a “T” linker between three nanostructure struts.

132. In an embodiment, a nanostructure node comprising four subunits where two subunits incorporate specific binding sites and two subunits do not. In its C4 symmetric form, and where two subunits are related by a 180 degree rotation about the C4 axis, the nanostructure node functions as a linear linker between two nanostructure struts.

133. In an embodiment, a nanostructure node comprising four subunits where two subunits incorporate specific binding sites and two subunits do not. In its C4 symmetric form, and where two subunits are related by a 90 degree rotation about the C4 axis, the nanostructure node functions as a right angle “L” linker between two nanostructure struts.

134. In an embodiment, a nanostructure node comprising four subunits where one subunit incorporates a specific binding site and three subunits do not. The nanostructure node functions as a cap or terminator for a nanostructure struts.

135. In an embodiment, a protein superstructure, comprising a multisubunit nanostructure node with specifically bound strut components.

136. In an embodiment, a protein superstructure, comprising a multisubunit nanostructure node with specifically bound strut components, where the struts are comprised of streptavidin and are bound to the node via biotin groups covalently bound to the specific amino acid residues on the node.

137. In an embodiment, a protein superstructure, comprising a multisubunit nanostructure node, specifically bound to a surface-immobilized strut component, where the strut is comprised of streptavidin and is bound to the node via biotin groups covalently coupled to the specific amino acid residues on the node.

138. In an embodiment, a protein superstructure, comprising a multisubunit nanostructure node with specifically bound strut components, where the struts are comprised of streptavidin together with an adaptor protein that is linked to streptavidin through a bifunctional biotin-ATP crosslinking agent.

139. In an embodiment, a protein superstructure, comprising a multisubunit nanostructure node with specifically bound strut components, where the strut component is an adaptor protein that is linked to the node via ATP derivative groups covalently coupled to specific amino acid residues on the node.

140. In an embodiment, a protein superstructure, comprising a multisubunit nanostructure node with specifically bound strut components, where the strut component is comprised of a complex of streptavidin and an adaptor protein, all associated through specific linkers.

141. In an embodiment, a kit, comprising a nanostructure multisubunit node and a monostructure strut.

142. In an embodiment, a kit, comprising a nanostructure multisubunit node and a monostructure strut comprised of streptavidin.

143. A method of making a thermostable nanostructure node multimeric protein that takes advantage of the thermostability in performing separation from the producing cells.

144. A method of making a thermostable nanostructure node multimeric protein that takes advantage of the thermostability in performing separation from the producing cells and uses recombinant DNA technology or site-specific modification techniques to modify a nucleotide sequence of a thermophilic organism for directing the expression of the nanostructure node multimeric protein.

145. A method of making a thermostable nanostructure node multimeric protein that takes advantage of the thermostability in performing separation from the producing cells and uses a gene fusion technique to modify a nucleotide sequence of a thermophilic organism for directing the expression of the nanostructure node multimeric protein to have at least two subunits covalently interconnected with a polypeptide linker.

146. A method of making a thermostable nanostructure node multimeric protein that takes advantage of the thermostability in performing separation from the producing cells and involves inserting the nucleotide sequence of a thermophilic organism or a modified nucleotide sequence of a thermophilic organism in the cell host to direct expression of the nanostructure node multimeric protein by the cell host.

147. The method of Paragraph 143, further comprising isolating the thermostable nanostructure node multimeric protein in substantially pure form from the lysate.

148. A method of making a nanostructure node multimeric protein by combining subunits, some of which have a linker binding site and others of which do not have linker binding sites.

149. A chromatographic or electrophoretic method of purifying nanostructure node multimeric proteins prepared by mixing combined subunits, some of which have a linker binding site and others of which do not have linker binding sites.

150. A chromatographic or electrophoretic method of purifying trimeric nanostructure node multimeric proteins prepared by mixing combined subunits, some of which have a linker binding site and others of which do not have linker binding sites.

151. A chromatographic or electrophoretic method of purifying tetrameric nanostructure node multimeric proteins prepared by mixing combined subunits, some of which have a linker binding site and others of which do not have linker binding sites.

152. A chromatographic or electrophoretic method of purifying 4-fold symmetric tetrameric nanostructure node multimeric proteins prepared by mixing combined subunits, some of which have a linker binding site and others of which do not have linker binding sites.

153. A chromatographic or electrophoretic method of purifying 4-fold symmetric tetrameric nanostructure node multimeric proteins prepared by mixing combined subunits, by separation into subfractions incorporating a variable number of subunits with linker binding sites

154. A chromatographic or electrophoretic method of purifying D2 or tetrahedrally symmetric tetrameric nanostructure node multimeric proteins prepared by mixing combined subunits, by separation into subfractions incorporating a variable number of subunits with linker binding sites

### Method of Making a Nanostructure Assembly (Chemical Synthesis)

155. A method of making a protein nanostructure that includes a nanostructure node multimeric protein binding to a nanostructure strut.

156. A method of making a protein nanostructure that includes a nanostructure node multimeric protein binding to a nanostructure strut, that allows mixing and reaction of the binding components.

157. A method of making a protein nanostructure that includes a nanostructure node multimeric protein and nanostructure struts comprising streptavidin.

158. A method of making a protein nanostructure that includes a nanostructure node multimeric protein incorporating covalently bound iminobiotin groups and nanostructure struts comprising streptavidin.

159. A method of making a protein nanostructure that includes a nanostructure node multimeric protein incorporating covalently bound photo-ATP groups and nanostructure struts comprising adaptor molecules with ATP binding sites.

### Method of Using a Proteinaceous Nanostructure Assembly as a Pattern or Resist

160. A method of using a proteinaceous nanostructure assembly as a pattern or resist masking material for the fabrication of devices with sub-100 nanometer features.

161. A method of using a 2-dimensional proteinaceous nanostructure assembly as a pattern for the fabrication of devices with sub-100 nanometer features.

162. A method of using a 2-dimensional proteinaceous nanostructure assembly as a mask for a resist material for the fabrication of devices with sub-100 nanometer features.

163. A method of using a 3-dimensional proteinaceous nanostructure assembly as a negative patterning material for the fabrication of devices with sub-100 nanometer features.

164. A method of using a 3-dimensional proteinaceous nanostructure assembly as a patterning material for the fabrication of devices with sub-100 nanometer features.

165. A method of using a proteinaceous nanostructure assembly as a pattern or resist material for the fabrication of a soft lithography stamp for nanolithography.

166. A method of using a proteinaceous nanostructure assembly as a pattern or resist material for the fabrication of a semiconductor device.

167. A method of using a proteinaceous nanostructure assembly as a pattern or resist material for the fabrication of a zero-mode waveguide.

168. A method of using a proteinaceous nanostructure assembly as a pattern or resist material for the fabrication of a microelectromechanical system (MEMS) device.

169. A method of using a proteinaceous nanostructure assembly as a pattern or resist material for the fabrication of a nanofluidics device.

Method of Making a Device Using a Proteinaceous Nanostructure Assembly as a Pattern or Resist Mask

170. A method of making devices with sub-100 nanometer features using a proteinaceous nanostructure assembly as a pattern or resist masking material.

### Method of Making a Device Using a 2-Dimensional Proteinaceous Nanostructure Assembly as a Pattern

171. A method of making devices with sub-100 nanometer features using a 2-dimensional proteinaceous nanostructure assembly as a patterning material.

172. A method of making devices with sub-100 nanometer features using a 2-dimensional proteinaceous nanostructure assembly as a patterning material on substrates composed of metal, glass, a self-assembling monolayer, plastic, ceramic, or a semiconductor material.

173. A method of making devices with sub-100 nanometer features using a 2-dimensional proteinaceous nanostructure assembly as a pattern that is assembled from engineered nodes derived from a list of thermostable multimers with known structure and optionally, streptavidin or streptavidin-incorporating struts.

### Method of Making a Device Using a 2-Dimensional Proteinaceous Nanostructure Assembly as a Resist Mask

174. A method of making devices with sub-100 nanometer features using a 2-dimensional proteinaceous nanostructure assembly as a method of patterning a resist material.

175. A method of making devices with sub-100 nanometer features using a 2-dimensional proteinaceous nanostructure assembly as a method of patterning a resist material and binding a node protein or the node protein assembly to the resist layer surface at specific attachment sites through a chemical linkage.

176. A method of making devices with sub-100 nanometer features using a 2-dimensional proteinaceous nanostructure assembly as a method of patterning a resist material for patterning a substrate composed of metal, glass, a self-assembling monolayer, plastic, ceramic, or a semiconductor material.

177. A method of making devices with sub-100 nanometer features using a 2-dimensional proteinaceous nanostructure assembly as a pattern for a resist material where the proteinaceous pattern is assembled from engineered nodes derived from a list of thermostable multimers with known structure and optionally, streptavidin or streptavidin-incorporating struts.

### Method of Making a Device Using a 3-Dimensional Proteinaceous Nanostructure Assembly as a Negative Pattern

178. A method of making devices with sub-100 nanometer, 3-dimensional channel features, wherein the features form a negative image of a 3-dimensional proteinaceous nanostructure assembly.

179. A method of making devices with sub-100 nanometer, 3-dimensional channel features, wherein the features form a negative image of a 3-dimensional proteinaceous nanostructure assembly, and binding a node protein or the node protein assembly to the resist layer surface at specific attachment sites through a chemical linkage.

180. A method of making devices with sub-100 nanometer features with 3-dimensional channel features, wherein the features form a negative image of a 3-dimensional proteinaceous nanostructure assembly, and a substrate is composed of a metal (such as iron, gold, platinum, or silver), a noble metal (such as gold, platinum, or silver), a glass (such as silicon dioxide), a self-assembling monolayer, plastic, a polymer, an organic polymer (such as polytetrafluoroethylene), a ceramic, an organic material, or a semiconductor material (such as silicon or germanium).

181. A method of making devices with sub-100 nanometer features with 3-dimensional channel features, wherein the features form a negative image of a 3-dimensional proteinaceous nanostructure assembly, and a matrix material comprises a metal (such as iron, gold, platinum, or silver), a noble metal (such as gold, platinum, or silver), a glass (such as silicon dioxide), a self-assembling monolayer, plastic, a polymer, an organic polymer (such as polytetrafluoroethylene) a ceramic, an organic material, or a semiconductor material (such as silicon or germanium).

182. A method of making devices with sub-100 nanometer, 3-dimensional channel features, wherein the 3-dimensional proteinaceous nanostructure assembly is assembled from engineered nodes derived from a list of thermostable multimers with known structure and optionally, streptavidin or streptavidin-incorporating struts.

### Method of Making a Device Using a 3-Dimensional Proteinaceous Nanostructure Assembly as a Pattern

183. A method of making devices with sub-100 nanometer, 3-dimensional features, wherein the features form a replica image of a 3-dimensional proteinaceous nanostructure assembly.

184. A method of making devices with sub-100 nanometer, 3-dimensional features, wherein the node protein or the node protein assembly is bound to the resist layer surface at specific attachment sites through a chemical linkage.

185. A method of making devices with sub-100 nanometer, 3-dimensional features, the substrate composed of a metal (such as iron, gold, platinum, or silver), a noble metal (such as gold, platinum, or silver), a glass (such as silicon dioxide), a self-assembling monolayer, plastic, a polymer, an organic polymer (such as polytetrafluoroethylene), an organic material, a ceramic, or a semiconductor material (such as silicon or germanium).

186. A method of making devices with sub-100 nanometer, 3-dimensional features, wherein the features form a replica image of a 3-dimensional proteinaceous nanostructure assembly, optionally embedded in a matrix material composed of metal, glass, plastic, ceramic, or a semiconductor material.

187. A method of making devices with sub-100 nanometer, 3-dimensional features, wherein the features form a replica image of a 3-dimensional proteinaceous nanostructure assembly, wherein the replica image is composed of metal, glass, plastic, ceramic, or a semiconductor material.

188. A method of making devices with sub-100 nanometer, 3-dimensional features, wherein the 3-dimensional proteinaceous nanostructure assembly forming the pattern to be replicated is assembled from engineered nodes derived from a list of thermostable multimers with known structure and optionally, streptavidin or streptavidin-incorporating struts.

### Devices

189. A method of making a proteinaceous nanostructure assembly as a pattern or resist material for the fabrication of a soft lithography stamp for nanolithography.

190. A method of making a proteinaceous nanostructure assembly as a pattern or resist material for the fabrication of a semiconductor device.

191. A method of making a proteinaceous nanostructure assembly as a pattern or resist material for the fabrication of a zero-mode waveguide.

192. A method of making a proteinaceous nanostructure assembly as a pattern or resist material for the fabrication of a microelectromechanical system (MEMS) device.

193. A method of making a proteinaceous nanostructure assembly as a pattern or resist material for the fabrication of a nanofluidics device.

194. A device that includes a substrate having a surface, a nucleation site on the substrate surface, and a nanostructure node coupled to the nucleation site.

195. A device that includes a substrate having a surface, a nucleation site on the substrate surface, and a nanostructure node coupled to the nucleation site, with more than one nucleation site on the substrate surface and with the nucleation sites arranged in a periodic, quasiperiodic, or nonperiodic pattern.

196. A device that includes a substrate having a surface, a nucleation site on the substrate surface, and a nanostructure node coupled to the nucleation site, the substrate comprising, for example, a metal (such as iron, gold, platinum, or silver), a noble metal (such as gold, platinum, or silver), a glass (such as silicon dioxide), a ceramic, a semiconductor (such as silicon or germanium), a carbon allotrope (such as diamond or graphite), a polymer, an organic polymer (such as tetrafluoroethylene), and/or an organic material and the nucleation site comprising, for example, a metal atom (such as iron, gold, platinum, or silver), a noble metal atom (such as a gold, platinum, silver, or copper), a metal and/or noble metal cluster, a chemically reactive molecule, and/or a patch of chemically reactive molecules.

197-199. A device that includes a substrate having a surface, a nucleation site on the substrate surface, and a nanostructure node coupled to the nucleation site, the nanostructure node comprising a nanostructure node multimeric protein comprising at least one polypeptide chain. The nanostructure node multimeric protein can have a known 3-dimensional structure, the nanostructure node multimeric protein can essentially have Cn, Dn, or higher symmetry with a number of subunits, the nanostructure node multimeric protein can be stable at a temperature of 70° C. or greater, the nanostructure node multimeric protein can have an amino acid sequence not found in nature, the nanostructure node multimeric protein can include a specific binding site for the attachment of a nanostructure strut with predefined stoichiometry and orientation, the specific binding site can include at least two specific amino acid reactive residues, and each specific amino acid reactive residue can have a covalently attached biotin group. The subunit can include an amino acid sequence having a designated amino and/or carboxy terminus and can include an amino acid (polypeptide) extension of from 5 to 1000 amino acid residues linked with a peptide bond to the designated amino and/or carboxy terminus, and the amino acid extension can include a binding function coupled to the nucleation site. A nanostructure strut can be attached to the specific binding site.

200-201. A device includes a substrate having a surface with a node-occupied area and a node-unoccupied area. A nanostructure node can be on the node-occupied area of the surface. A coating can cover the nanostructure node and can cover the surface node-unoccupied area of the surface. The coating can include a metal (such as iron, gold, platinum, or silver), a noble metal (such as gold, platinum, or silver), a glass (such as silicon dioxide), a ceramic, a semiconductor (such as silicon or germanium), a carbon allotrope (such as diamond or graphite), a polymer, an organic polymer (such as tetrafluoroethylene), and/or an organic material.

202-204. A device can include a substrate having a surface with a node-occupied area and a node-unoccupied area. The surface can be coated with a resist layer. A nanostructure node can be on the resist layer. The node-occupied area of the surface of the substrate can be coated with the resist layer. The node-unoccupied area of the surface of the substrate can be not coated with the resist layer. The node-unoccupied area of the surface of the substrate can be lower than (recessed with respect to) the node-occupied area of the surface of the substrate.

205-209. A device can include a proteinaceous nanostructure assembly comprising a nanostructure node. The device can include a substrate having a surface, and the proteinaceous nanostructure assembly can be coupled to the surface of the substrate. The device can include a first matrix, and the first matrix can interpenetrate the proteinaceous nanostructure assembly. The proteinaceous nanostructure assembly can have the form of a cubic lattice, and the first matrix can have the form of a cubic lattice. The first matrix can include a metal (such as iron, gold, platinum, or silver), a noble metal (such as gold, platinum, or silver), a glass (such as silicon dioxide), a ceramic, a semiconductor (such as silicon or germanium), a polymer, an organic polymer (such as tetrafluoroethylene), and/or an organic material.

210-211. A device can include a second matrix material having the same or similar form as a proteinaceous nanostructure assembly. The device can include a second matrix that includes a metal (such as iron, gold, platinum, or silver), a noble metal (such as gold, platinum, or silver), a glass (such as silicon dioxide), a ceramic, a semiconductor (such as silicon or germanium), a polymer, an organic polymer (such as tetrafluoroethylene), and/or an organic material.

### BRIEF DESCRIPTION OF THE DRAWINGS

Table 1 lists template structures useful for the construction of nanostructure struts and nodes with different symmetry.

Table 2 lists specifications and amino acid sequences for node embodiments with various symmetries.

FIG. 1 shows schematic backbone and surface representations of the streptavidin strut molecule, a tetrameric protein with D2 symmetry, indicating geometry of biotin ligand binding sites and interaction geometry of node attachment sites.

FIG. 2 shows the reaction of protein cysteine sulfhydryl groups with biotinylation reagents.

FIG. 3 presents schematic illustrations of nodes with three-fold (C3) rotational symmetry and examples of corresponding protein multimers from thermostable microorganisms useful as node templates.

FIG. 4 presents schematic illustrations of single-chain nodes based on protein multimers with three-fold rotational (C3) symmetry.

FIG. 5 presents schematic illustrations of multiple and single-chain nodes based protein multimers with three-fold rotational (C3) symmetry, incorporating functional binding sites and fused protein domains.

FIG. 6 shows the reaction of protein cysteine sulfhydryl groups with bifunctional crosslinking reagents.

FIG. 7 presents schematic illustrations of nodes with four-fold (C4) rotational symmetry and examples of corresponding protein multimers from thermostable microorganisms useful as node templates.

FIG. 8 presents schematic illustrations of single-chain nodes based on a protein multimers with four-fold rotational (C4) symmetry.

FIG. 9 presents schematic illustrations of multiple and single-chain nodes with four-fold rotational (C4) symmetry, incorporating functional binding sites and fused protein domains.

FIG. 10 presents schematic illustrations of nodes with C5, C6, and C7 rotational symmetry and representative examples of corresponding protein multimers from thermostable microorganisms useful as node templates.

FIG. 11 presents schematic illustrations of 3-dimensional polyhedra incorporating nodes with C3 and C5 symmetry.

FIG. 12 presents schematic illustrations of D2 symmetric nodes used as strut extenders.

FIG. 13 presents illustrations of a D2 node used as a strut extender that introduces an axial rotation along the strut axis.

FIG. 14 presents illustrations of D2 symmetric protein multimers from thermostable microorganisms useful as node templates.

FIG. 15 presents schematic illustrations of a hexameric node with D3 symmetry and an octameric node with D4 symmetry.

FIG. 16 presents schematic illustrations of a doubly-modified hexameric node with D3 symmetry and an doubly-modified octameric node with D4 symmetry.

FIG. 17 presents illustrations of hexameric protein multimers with D3 symmetry from thermostable microorganisms useful as node templates.

FIG. 18 presents illustrations of octameric protein multimers with D4 symmetry from thermostable microorganisms useful as node templates.

FIG. 19 presents illustrations of regular polyhedra with dyad axes of symmetry.

FIG. 20 presents illustrations of protein multimers from thermostable microorganisms having the symmetry properties of regular polyhedra and utility as templates for nanostructure node proteins.

FIG. 21 requirements for complementary binding geometry between streptavidin and node surfaces.

FIG. 22 illustrates methods used to determine the sites of surface amino acid substitution to transform multimeric node templates into nodes able to bind streptavidin with defined relative geometry.

FIG. 23 presents a stereoscopic image of a D2 symmetric protein showing bounding boxes used to determine the sites of surface amino acid substitution to transform multimeric node templates into nodes able to bind streptavidin with defined relative geometry.

FIG. 24 presents schematic illustrations and computer models of C3 and C4 symmetric nodes together with streptavidin tetramers oriented to allow linkages through biotin linkages.

FIG. 25 presents a stereoscopic representation of an engineered single-chain C3 symmetric node.

FIG. 26 presents computer models of a C3 symmetric node complexed with three streptavidin tetramers with geometry suitable for the apex formation of a dodecahedron.

FIG. 27 presents computer models of a C5 symmetric node complexed with five streptavidin tetramers with geometry suitable for the apex formation of an icosahedron.

FIG. 28 presents schematic illustrations of D2 symmetric nodes engineered from streptavidin.

FIG. 29 presents schematic illustrations and computer models of a D2 symmetric node oriented to allow linkages to streptavidin tetramers through biotin linkages along 3 dyad axes.

FIG. 30 presents schematic illustrations and computer models of D2 symmetric nodes useful as a strut extender together with streptavidin tetramers oriented to allow biotin linkages along one dyad axis.

FIG. 31 presents schematic illustrations and computer models of hexameric nodes with D3 symmetry with streptavidin tetramers oriented to allow linkages to streptavidin through biotin linkages along a dyad axis.

FIG. 32 presents schematic illustrations and computer models of octameric nodes with D4 symmetry with streptavidin tetramers oriented to allow linkages to streptavidin through biotin linkages along dyad axes.

FIG. 33 presents schematic illustrations and computer models of dodacameric nodes with tetrahedral symmetry with streptavidin tetramers oriented to allow linkages through biotin linkages along dyad axes.

FIG. 34 presents schematic illustrations of complexes of streptavidin with linear strut connectors having D2 symmetry to produce struts of various lengths.

FIG. 35 presents schematic illustrations of streptavidin-linked two-dimensional radial structures formed using variants of nodes with three-fold (C3) and four-fold (C4) and seven-fold (C7) rotational symmetry.

FIG. 36 presents schematic illustrations of streptavidin-linked two-dimensional lattices formed using nodes with three-fold (C3) and four-fold (C4) rotational symmetry.

FIG. 37 presents schematic illustrations of streptavidin-linked two-dimensional polygonal structures formed using single-chain variants of nodes with three-fold and four-fold rotational symmetry.

FIG. 38 presents a molecular illustration of two hexameric D3 nodes interconnected by streptavidin enabling formation of a three-connected three-dimensional lattice.

FIG. 39 presents a molecular illustration of two octameric D4 nodes interconnected by streptavidin enabling formation of a four-connected three-dimensional lattice.

FIG. 40 presents schematic illustrations of various three-dimensional lattices with different node connectivity.

FIG. 41 presents a method of making a proteinaceous nanostructure pattern on a substrate surface.

FIG. 42 presents a method of making a repetitively patterned proteinaceous nanostructure on a substrate surface.

FIG. 43 presents a method of making a coated patterned nanostructure on a substrate surface.

FIG. 44 presents a method of making a patterned structure using a proteinaceous nanostructure as a mask for a photoresist material.

FIG. 45 presents a method of making a 3-dimensionally patterned structure in a solid matrix material or, in additional steps, a replica of a 3-dimensional proteinaceous nanostructure assembly.

FIG. 46 illustrates the use of a SAMA in the construction of an extended linear strut immobilized on a surface. Part (a) shows a surface that has been functionalized with an azido-ATP reagent. Part (b) shows a dimeric SAMA molecule that has 2 ATP binding sites and has also been functionalized with biotin groups that are complementary to biotin binding sites on streptavidin. Part (c) shows the SAMA immobilized on the surface so that it can bind to 2 sites of a streptavidin tetramer (d). The immobilized complex (e) can then bind a biotin-azido-ATP crosslinking reagent (f) to form the modified complex (g). The resulting complex (h) can react with additional SAMAs (i) to form extended structures (j,k). The linear structure can be terminated with a wide variety of commercially available biotinylated proteins (e.g. Biotinylated Protein A that binds immunoglobulin Fc domains) to create functionalized assemblies on solid surfaces for devices such as biosensors. We have developed the SAMA based on an engineered, dimeric ATP binding protein that incorporates 2 surface cysteine residues whose geometry is complementary with streptavidin biotin binding sites. The cysteine residues can be biotinylated so that the SAMA forms interactions with 2 of the biotin binding groups on streptavidin. The molecule also incorporates 2 ATP binding sites that are geometrically complementary to 2 biotin binding sites on streptavidin. Because the reactions with the two different pairs of sites on SAMA can be formed independently, the SAMA can be used for sequential multi-step assembly processes.

FIG. 47 presents representative NanoArchitectures, which can be assembled using engineered protein nodes interlinked with streptavidin. Such structures can be additionally functionalized through binding or covalent attachment of other proteins or chemical groupings to impart diverse chemical, physical, or biological functions.

FIG. 48 presents schematics of C3-symmetric and C4-symmetric nodes designed and expressed.

FIG. 49A shows the structure of the C3 symmetric uronate isomerase from Thermotoga maritima. Each chain of the trimer comprises 450 amino acid residues, has no disulfide bonds, and only 3 surface cysteine residues. Uronate isomerase is an amidohydrolase that catalyzes the isomerization of D-glucuronate and D-fructuronate. This enzyme appears to be the only member of the amidohydrolase superfamily that does not require divalent cation for activity (Williams et al. 2006).

FIG. 49B shows the structure of the C4 symmetric type 2 isopentenyl diphosphate isomerase from Thermus thermophilus. Each chain of the tetramer incorporates 332 amino acid residues, has no disulfides, and 2 surface cysteines. Type 2 IPP isomerases are flavoenzymes responsible for the interconversion of isopentenyl diphosphate and dimethylallyl diphosphate that are both building blocks in isoprenoid biosynthesis. Because the interconversion occurs without net change in redox state, the FMN (shown in violet) is thought to act as a general acid or base, an hypothesis supported by recent crystallographic studies on the related Sulfolobus shibatae IPP isomerase (Unno et al. 2009).

FIG. 49C shows the structure of the C3 symmetric g-carbonic anhydrase from the thermophilic archeon Methanosarcina thermophila. Each chain incorporates 212 amino acids, has no disulfides and only one cysteine. This protein has no sequence homology with other classes of carbonic anhydrase. The crystal structure revealed a b-helix fold, unique among carbonic anhydrases, that contains rarely observed left-handed crossover connections between parallel b-strands (Kisker et al. 1996). Active site zinc atoms (green spheres) are located at the monomer interfaces. The active site geometry (Humphrey et al. 2000) shares structural similarities with the vertebrate enzymes suggesting convergent evolution based on catalytic requirements for carbon dioxide hydration to form bicarbonate plus a proton. Recently other g-carbonic anhydrases have been reported (Fu et al. 2008; Jeyakanthan et al. 2008).

FIG. 50 illustrates the relative location of biotin binding sites on streptavidin. Part a shows the symmetric arrangement of subunits in the streptavidin tetramer. Subunits are related by three perpendicular 2-fold axes, labeled x, y and z. Positions of the bound biotin valeric acids are shown as open circles for sites on the “front” of the tetramer and as shaded circles for sites on the back. Linkers to biotin are coupled via the biotin valeric acids (Table 3) that in the 3D structure are situated on opposite sides of the tetramer as two pairs about 20.5 A apart. Filled circles in parts b and c show locations of node cysteine sidechains geometrically complementary to the biotin valeric acid sidechains of streptavidin.

FIG. 51 illustrates the selection of cysteine sites by sampling node and streptavidin protein:protein interfaces. FIG. 51A shows a 3-fold node and streptavidin aligned along the x-axis with their z-axes perpendicular to the page. The node was rotated about the z-axis and for each orientation streptavidin was translated along x to form a complex. FIG. 51B shows superposition of the 5 representative docking solutions of the C3 g-carbonic anhydrase node with streptavidin.

FIG. 52 illustrates geometric selection of cysteine sites on Dn node templates using bounding boxes. It shows a stereographic representation of a template node protein (pdb code: 1 rtw) aligned with boxes defined by the geometry of biotin binding sites on streptavidin. Computationally the boxes are aligned along the node dyad symmetry axes. The boxes that cross along each symmetry axis correspond to the two possible orientations of streptavidin (FIGS. 50b, 50c). Sites for cysteine incorporation are defined by the intersection of the bounding box with the node protein surface.

FIG. 53 illustrates node:streptavidin complexes. FIGS. 53a and 53b show the M. thermophila g-carbonic anhydrase trimer symmetrically complexed with three streptavidin tetramers, in ribbon and space filling representations respectively. Similarly, T. thermophilus IPP isomerase complexed with 4 streptavidin tetramers is shown in FIGS. 53c and 53d.

FIG. 54 illustrates the surface orientation of a 4-fold node on a 2D surface. Panel shows IPP isomerase as it would be oriented on a 2D surface by immobilization via His-tags inserted before the natural amino terminus. View is along the plane of the 2D surface. In each subunit, residue 9 (the first residue visible in the electron density maps) is highlighted with a blue sphere. For reference the C-termini are highlighted as red spheres and FMN prosthetic groups are shown in violet. Backbone chains of the identical IPP isomerase subunits are shown in different colors.

FIG. 55 illustrates the surface orientation of a 3-fold node on a 2D surface and single chain g-carbonic anhydrase. FIG. 55A shows g-carbonic anhydrase as it would be oriented on a 2D surface by immobilization via His-tags. View is along the plane of the 2D surface. N- and C-terminal residues in the native structure appear as blue and red spheres, respectively. Catalytic zinc atoms are shown as violet spheres. Backbone chains of the subunits are colored differently. FIG. 55B shows a stereoview of the single chain g-carbonic anhydrase where the three subunits are incorporated as domains of a single polypeptide chain.

FIG. 56 illustrates representative E. coli expression vectors. FIGS. 56A and 56B, respectively, show the expression constructs for IPP isomerase and the single-chain g-carbonic anhydrase.

FIG. 57 presents an Anti-His Western blot showing expression of soluble full-length single chain g-carbonic anhydrase. Molecular weight standards are in Lanes 1 & 12 with 110 kDa and 60 kDa markers highlighted to verify the 68 kDa molecular weight expected for single chain g-carbonic anhydrase. Lanes 2-5 and 6-9 show protein production during 4 and 20 hour fermentations in LB broth. Lanes 10 & 11 show soluble lysates from fermentation controls for cells without g-carbonic anhydrase genes.

FIG. 58 illustrates the purification of g-carbonic anhydrase by Ni-agarose chromatography. Molecular weight standards are at the left. Whole cell lysates is shown in the middle, and lanes to the right show successive washes and elutions. Band at the top of the right most pair of lanes corresponds to g-carbonic anhydrase 68 kDa.

FIG. 59 presents PAGE analysis of SAV:IPP isomerase complexes. Complexes of IPP isomerase and streptavidin were analyzed on 4-12% TRIS-Glycine gels under native (left) and denaturing (right) conditions. On the left panel, lanes 1, 3 & 4 show the aggregate forms of streptavidin (lane 1) and the streptavidin:biotin complex (lanes 3 & 4). The streptavidin:IPP isomerase complexes are shown in lanes 6&7 and 9&10 with the latter samples containing higher relative concentrations of streptavidin. The band near 242 kDa corresponds to the 2SAV:IPPisomerase complex and the broader peak above it likely corresponds to the 4SAV:IPPisomerase complexes. Higher molecular weight complexes also formed and did not migrate into the gel and appear as dark bands at the tops of the wells in lanes 6, 7, 9, and 10. Molecular weight markers are in lane 2. The gel on the right was prepared under conditions where the streptavidin:biotin complex is stable (Gonzalez et al. 1997), but other protein oligomers including IPP isomerase and unliganded streptavidin are not. Prior to loading, samples were heated at 80° C. for 10 min with SDS. Under these conditions streptavidin is denatured (Lane 8) but the streptavidin:biotin tetramer remains folded (lane 7). Brackets and schematics in the middle indicate probable electrophoretic mobilities of SAV:IPP isomerase complexes. We envision defining the complexes by MS methods, so that gels can be used as a laboratory screen of complex formation. Bands for complexes are somewhat broadened, because while the streptavidin:biotin is not denatured under these conditions, IPP isomerase is likely denatured during sample preparation and electrophoresis. On comparison of lanes 1 and 3 with lane 2 it is clear that when excess biotin is added to the reaction mixture to saturate all biotin-binding sites on streptavidin after the complexes are formed and before the sample is prepared for electrophoresis, bands corresponding to the IPP isomerase:streptavidin complexes are sharpened, as is the band for the streptavidin:biotin tetramer. Higher molecular weight SAV:IPP isomerase complexes, presumably formed by streptavidin tetramers bridging IPP isomerase tetramers are evident at the tops of Lanes 1-4. Lower molecular weight bands are a mixture of the derivatized and underivatized IPP isomerase monomers, the streptavidin dimer, and streptavidin monomers whose molecular weights range from about 12 000 to 13 500 kDa due to proteolysis of full-length streptavidin during production. MW standards are in Lane 5.

FIG. 60 presents schematics of first-generation C3 and C4-symmetric nodes.

FIG. 61 presents schematic illustrations of single chain C3 nodes with a fused IgG binding domain.

FIG. 62 presents molecular models of single-chain C3 node with fused IgG binding sequence. FIG. 62A shows backbone trace of the single-chain g-carbonic anyhdrase incorporating a Streptococcal Protein G domain. The arrangement allows Protein G insertion at a surface loop near the carbonic anhydrase carboxy terminus. In the left panel, Protein G (yellow spheres with amino and carboxy terminal residues shown as dark blue and red spheres, respectively) is inserted between loop residues shown as violet and green spheres, respectively. The N-terminal portion of the single-chain construct is shown in cyan, and the C-terminal helix is shown in orange. FIG. 62B shows a surface representation of the construct in complex with an intact IgG. IgG coordinates were taken from Padlan 1994.

FIG. 63 presents schematics of single-chain C4 Nodes. Panels from left to right schematically illustrate single-chain C4 nodes engineered to bind 4, 3, 2, 2 and 1 streptavidin tetramers with defined geometry.

FIG. 64 illustrates the design of a single-chain C4 node using the IPP isomerase template. FIG. 64A shows native IPP isomerase with the subunit chains in different colors. The FMN prosthetic groups are colored violet. The N- and C-termini are designated with blue and red spheres, respectively. The linear distance between C- and N-terminus of adjacent subunits is ˜35 A. FIG. 64B shows engineering of the IPP isomerase subunit to allow more efficient interdomain connections in a single-chain molecule. In the engineered subunit, residues 229 (highlighted with a blue sphere) thru 332 constitute the N-terminal portion, residues 30 thru 228 the central portion, and residues 9 thru 29 (highlighted with a red sphere) the C-terminal portion. Completion of the chain involves design of short polypeptide fragments interconnecting residues 332 and 30 (green spheres) and 228 and 9 (yellow spheres). FIG. 64C shows the IPP isomerase tetramer with the engineered N- and C-termini shown as blue and red spheres, respectively.

FIG. 65 presents examples of nanostructure assemblies constructed using some of the C3 and C4 nodes developed. FIGS. 65a and 65b show structures potentially accessible through solution reaction schemes. FIGS. 65c and 65d show structures potentially accessible through assembly on a C3- or C4-symmetric node initially immobilized on a Ni resin or other substrate.

FIG. 66 presents examples of 2D nanostructures assembled on SAMs using nodes described herein. FIG. 66a shows a section of a continuous 2D hexagonal lattice. FIG. 66b shows a section of a continuous 2D square lattice.

FIG. 67 presents schematics of C3 and C4 protein nodes for nanoassembly. Nodes marked with a “1” have been developed.

FIG. 68 presents schematic diagrams of additional reagents and protein components for nanoassembly fabrication. FIG. 68a shows a schematic illustration of streptavidin with 4 biotin binding sites. FIG. 68b shows an engineered streptavidin (Streptavipol) incorporating 4 surface cysteine sites aligned along a molecular dyad axis so that they are geometrically complementary with 2 biotin binding sites on streptavidin. FIG. 68c shows show a streptavidin macromolecular adaptor (SAMA) based on an engineered form of a dimeric ATP binding protein. FIG. 68d shows an SH-reactive biotinylation reagent. FIG. 68e shows an SH-reactive azido-ATP linking reagent. FIG. 68f shows a biotin-azido-ATP bifunctional crosslinking reagent. FIG. 68g shows a dimeric ATP binding protein incorporating a protein-G IgG binding domain. FIG. 68h shows biotinylated Protein G. FIGS. 68i and 68j show 2 different IgG molecules. Molecules shown in FIGS. 68a, 68d, 68e, 68f, 68i, and 68j are commercially available. For example, molecules in FIGS. 68b, 68c, and 68h are being developed by Imiplex.

FIG. 69 provides an illustrative example of how functionalized nanostructures can be assembled using a convergent assembly strategy. Part a shows a Streptavipol (FIG. 68b) tetramer reacting with a thiol-reactive azido-ATP reagent to produce an azido-ATP modified Streptavipol tetramer. FIGS. 69c and 69d show the interaction between a given IgG and a dimeric ATP-binding protein incorporating fused Protein-G domains, to form the complex FIG. 69e. Two moles of FIG. 69e can associate with the streptavipol and subsequently be photo-crosslinked to form the element of FIG. 69f. FIG. 69f can then bind two C3-single chain nodes with fused IgG binding domains and attached IgG molecules to form the structure in FIG. 69h, incorporating two sets of two different antibodies in close proximity to each other. In fact, the structures of Streptavipol and the trimeric single-chain fusion (FIG. 62) orient the IgGs in a direction normal to the plane of the page in which the schematic is drawn as illustrated in the inset FIG. 69i and conventionally used in FIG. 70.

FIG. 70 illustrates nanostructures functionalized with bound antibodies. FIGS. 70a through 70e show schematic representations of various nanoarray assemblies built using the parts shown in FIGS. 67 and 68 incorporating immobilized antibodies in close proximity. (Alternative representations shown in inset FIG. 70f.)

### DETAILED DESCRIPTION

Embodiments of the invention are discussed in detail below. In describing embodiments, specific terminology is employed for the sake of clarity. However, the invention is not intended to be limited to the specific terminology so selected. A person skilled in the relevant art will recognize that other equivalent parts can be employed and other methods developed without parting from the spirit and scope of the invention. All references cited herein are incorporated by reference as if each had been individually incorporated.

In this document, an amino acid may be indicated by its standard one-letter abbreviation, as understood by one of skill in the art. For example, a polypeptide sequence may be represented by a string of letters.

In this document, indication of a protein having “80 percent or greater sequence identity” with the sequence of another protein is to be understood as including, as alternatives, proteins that are required to have a higher percentage of sequence identity with the other protein. For example, alternatives include proteins that have 90, 95, 98, 99, 99.5, or 99.9 percent or greater sequence identity with the sequence of the other protein.

### Overview of Components and Approach

An objective of the work leading to the present invention, of which several embodiments are presented in this text, is the development of biomolecular components allowing for the systematic and precise fabrication of complex nanodevices with two and 3-dimensional architectures. Proteins, typically having (subunit) dimensions in the range of 3 to 20 nm (or the equivalent, 30 to 200 Angstrom units), and other organic molecules serve as the biomolecular components, and allow for unprecedented miniaturization of devices. By providing proteins with two or more points of controllable attachment, a limited set of a small number of biomolecular components allows for construction of an unlimited number of structures, over the design of which a user has full control. Thus, the biomolecular components will advance research and development into nanodevice applications. The control over assembly and reproducible precision of structures formed by these biomolecular components allows for the fabrication of nanodevices of unprecedented complexity, extent, and diversity.

Described embodiments according to the present invention include molecular components that are extremely stable, easily manufactured and purified, and designed with high precision to enable the controlled assembly of a wide range of one-, two- and 3-dimensional protein-based nanostructure assemblies. Described embodiments according to the present invention include the design and manufacture of such molecular components.

In an embodiment, the protein components of the nanostructure assembly are functional, as appropriate for the development of biological sensors, filters, materials, or bioelectronic devices where charge, spin, or optical properties are intrinsic properties of the protein or prosthetic groups that are bound to the protein structure.

In an embodiment, the protein nanostructure assembly provides a means of high-resolution patterning of a silicon, glass, metal, or other substrate, either by using the protein nanostructure assembly directly as a means of patterning a substrate, or alternatively as a mask for a radiation-sensitive resist. This approach can allow manufacture of microelectronic devices, devices incorporating zero-mode waveguides (Levene et. al, 2003) or microelectromechanical systems (MEMS) using conventional semiconductor fabrication (Widman et. al, 2000) and/or MEMS fabrication technology (Judy, 2001). Additional patterning applications include the generation of soft lithography stamps and molds (Xia & Whitesides 1998, Rogers & Nuzzo 2005) for MEMS and nanofluidic applications.

### “Parts Box” Philosophy

The biomolecular components can include molecular-scale “struts” and “nodes”. Struts are components that basically function as linear structural elements or linear connectors, and typically have attachment points to nodes oriented in a linear arrangement. Different struts or arrays of strut extenders or adaptors can be used to establish predetermined distances in a structure. Nodes are connectors that can have either two attachment points with defined, for example, nonlinear, geometry, or more generally, multiple attachment points with defined geometry. Nodes can be linked together, for example, by struts, to establish the topology of a structure. Thus, with the struts and nodes, structures with 2-dimensional and 3-dimensional geometry can be constructed. Structures organized in two dimensions can be finite to allow the formation of locally structured patterns of molecules arrayed on a surface, or alternatively form infinitely extensible 2-dimensional lattices. The symmetry properties required of nodes suitable to build structures with the regular 2-dimensional geometry are well known from mathematics and crystallography (Williams 1979, Pearce 1979, Vainshtein, 1994). Two-dimensional structures can have utility themselves and/or can be further functionalized through chemical modification or the incorporation of additional specific binding proteins.

Structures organized in three dimensions can also be usefully classified as finite or infinite. Common examples of finite structures potentially constructed using molecular strut and node architecture include dendritic structures as well as the Platonic and Archimedian polyhedra and their many variations (Pugh 1976, Pearce 1979). The strut and node architecture also potentially allows the assembly of infinite 3-dimensional lattices. The symmetry requirements for nodes that can form infinite 3-dimensional lattices have been described comprehensively by Wells and others (Wells 1977, Wells 1979, Williams 1979). Three-dimensional structures can have utility themselves as materials and filters and/or can be further functionalized through chemical modification or the incorporation of additional specific binding proteins.

Assembly of biomolecular components such as struts and nodes can proceed in stages that provide the user with the efficiency and parallel nature characteristic of “bottom-up” self-assembly and the control and ability to form asymmetric and complex structures characteristic of “top-down” manufacturing. Because a limited number of biomolecular components can be combined to produce any one of an unlimited number of structures, attention can be focused on developing a small number of these biomolecular components that serve as a “parts box”. Because only a limited number of biomolecular components and associated assembly techniques need be designed, produced, and tested, economies of scale can be achieved, so that inexpensive development and production of nanodevices can be realized. That is, the compositions and methods discussed herein apply the philosophies of interchangeable parts and mass production, which drove unprecedented economic expansion in the last two centuries, to the nanoscale. Providing such a “parts box” of biomolecular components will allow users to experiment with a range of structures and thereby facilitate the development of a new generation of functional nanodevices, biosensors, and biomaterials, potentially finding broad application in areas as diverse as biomedical devices and nanoelectronic applications.

### Use of Proteins

Proteins have a number of advantages for use as components and templates for biomolecular components, including, but not limited to the following. Proteins already exist in nature as functional polypeptide units with well-defined 3-dimensional structures, so that effort can focus on tailoring them as building blocks for specific applications, rather than having to develop building blocks from scratch. A very large number of proteins exist, and the detailed atomic structure of many are known, so that there is an excellent chance of finding a protein that, with minimal tailoring, can perform as a desired building block.

Naturally occurring proteins have diverse and sophisticated functionality. They can show high interaction specificity and manifest catalytic properties. They can exhibit interesting and useful optical, magnetic, and redox properties, for example, by incorporating metal centers and a wide variety of prosthetic groups. Such metal centers and prosthetic groups can, as well as the polypeptide sequence itself, be tailored to produce a protein having a desired functionality.

In nature, DNA encodes a polypeptide sequence that spontaneously and reproducibly folds to form a predetermined 3-dimensional protein of thousands of atoms of which each atom is precisely placed. Because proteins as building blocks are reproducible and have precise configuration, they can be relied upon as components in the construction of extensive and complex structures. Naturally occurring proteins frequently form cooperative hierarchical assemblies of great structural and functional complexity. These natural assemblies can be studied to derive assembly techniques and simplify the development of analogous artificial structures having an intended purpose.

Naturally occurring proteins often form highly stable multimeric structures that are symmetric and contain multiple copies of the individual polypeptide chains. Symmetric multimeric structures are geometrically precise. If modification sites are introduced into a component polypeptide chain, then these are symmetrically arrayed in the multimeric structure with great geometrical precision; typically within errors of less than 1-2 Angstrom units (0.1 to 0.2 nM) from structure to structure. Symmetric protein multimers are excellent template structures for the generation of macromolecular protein nodes.

The techniques for modifying proteins by the techniques of molecular biology and synthetic organic chemistry are well established. For example, a selected amino acid unit of a natural protein can be substituted with a different natural amino acid, or with an artificial amino acid. Reliable production of large numbers of proteins is a well-established biotechnical procedure. Thus proteins are excellent candidates for a “parts box” with which the philosophies of interchangeable parts and mass production can be applied at the nanoscale.

### Applications

The diverse and sophisticated functionality of naturally occurring proteins allows them to perform a wide range of processing and signal transduction functions in nature, including catalysis, chemomechanical, electromechanical, optomechanical, and optoelectronic transduction for sensing and actuation purposes. This anticipates a diverse range of man-made devices that can be produced with a “parts box” of proteins as biomolecular components.

Structures, for example, node: strut nanostructure assemblies, can be assembled from the struts and nodes described herein.

A “parts box” of proteins may initially be applied to make devices that are analogous to or in some way emulate natural systems. For example, two- and 3-dimensional structures formed from struts and nodes, as described herein, may be applied in the fields of biosensors and diagnostics. The specific immobilization and precise geometric control facilitated by strut-node technology presented herein, along with the functionality inherent in proteins, can enable the development of new kinds of sensors incorporating, for example, multiple antibodies specifically immobilized in patterned arrays.

Other applications may not have direct natural analogs, but are intended to interact with natural biological systems. For example, the strut-node technology presented herein can be used in devices that couple directly to living systems, for example, that provide an interface between semiconductor substrates and living organisms and nanostructures. Such devices could, for example, be used as biocompatible materials for prostheses.

Applications of a “parts box” of proteins as biomolecular components are not limited to devices analogous to or for interacting with natural biological systems. For example, structures can be assembled that emulate the architecture and functions of silicon-based microprocessor architecture and computer memory or possess novel material properties. Many materials science and computer applications depend upon the miniaturization of structural features in two or three dimensions to allow the separation and storage of charge, control of electrical conductance or optical properties, or the addressable storage of data in electrical or magnetic form. As such, the technology described is applicable to the development of new electronic devices including improved batteries, capacitors, computer memory, microprocessors, nonlinear optical devices and materials. Additional applications include ultrafilters that provide protection from pathogens like viruses, or have utility in liquid separations or the desalination of salt water.

The protein components of the nanostructure assembly can be functional, as appropriate for the development of biological sensors, filters, materials, or bioelectronic devices where charge, spin, or optical properties are intrinsic properties of the protein or prosthetic groups that are bound to the protein structure.

Alternatively, the protein nanostructure assembly can provide a means of high-resolution patterning of a silicon, glass, metal, or other substrate, so providing high resolution templates or resists that allow production of microelectronic devices, devices incorporating zero-mode waveguides (Levene et al., 2003), or microelectromechanical systems (MEMS) using conventional semiconductor fabrication (Widman et al., 2000) and/or MEMS fabrication technology (Judy, 2001). Thus, the “parts box” strategy can be fundamentally exploited as a way of creating self-assembling or sequentially assembled structures where the nanometer size and designed-in precision of the interaction geometry between the protein molecular components can be used to create complex and highly precise structures in two and three dimensions. These patterns can then be used as optical resists, molds, metallization substrates, or negatives for the fabrication of semiconductor, MEMS, soft lithography molds (Xia & Whitesides 1998, Rogers & Nuzzo 2005), or other devices where miniaturization at the sub-100 nanometer scale is useful.

### Biomolecular Components

### Protein Stability, Selection, and Engineering

The 3-dimensional atomic structures of over 25,000 proteins are known (see, http://www.rcsb.org, accessed Oct. 2, 2007), providing an extensive set from which biomolecular components having desired structural and functional characteristics can be selected for a “parts box” (see, http://scop.mrc-lmb.cam.ac.uk/scop/, accessed Oct. 2, 2007). Moreover, the tools of recombinant DNA technology enable the synthesis of virtually any polypeptide sequence or functional domain fusion, providing the basis for rapidly designing and optimizing novel assemblies from engineered biological macromolecules.

Although not widely recognized, numerous studies show that the structural and functional properties of proteins that normally function in aqueous solution are preserved intact when the protein is dehydrated to the level of a few water molecules per protein molecule (Rupley & Careri 1991; Zaks & Klibanov 1988; Fitzpatrick et. al. 1993; Castro & Knubovets 2003; Gupta & Roy 2004). Many examples exist of structural proteins, for example spider silk, that form essentially solid-state structural materials and have thermal stabilities in excess of 100° C. In addition, many proteins that form unusually stable complexes (Weber et al. 1992), or that carry out the biological functions of thermophilic organisms that live in hot environments also have thermal stabilities in excess of 70° C., an environment not very dissimilar from the maximum operating temperatures for conventional semiconductor devices.

Evolutionary forces have allowed living organisms to exploit a wide range of habitats including environments that represent extremes of temperature, salinity, pH, specific mineral content, and/or pressure. The organisms adapted to the most extreme environment like hot springs, thermal vents at the ocean bottom, high salt environments like the Dead Sea, etc. are termed extremeophiles and are generally microorganisms such as bacteria or algae. A subclass of extremeophiles are thermophilic organisms (again, usually microorganisms such as bacteria or algae), which live at substantially higher temperatures (typically above 60 deg C.) than the vast majority of plants and animals populating the terrestrial ecosystem (usually termed mesophilic organisms or mesophiles). Most plants and animals could not survive at such elevated temperatures because the basic molecules responsible for most of the biological functions of the organism, i.e. the polypeptide proteins encoded by the organism's genetic material or DNA, would become denatured. Proteins are poly-amino acid polymers (or polypeptides) of defined sequence that fold to form highly organized 3-dimensional structures. Maintenance of the biological function of a protein as a chemical catalyst, receptor, channel, etc. is completely dependent on the preservation of its properly folded, 3-dimensional structure. The vast majority of proteins of mesophilic organisms become thermally denatured when subjected to temperatures above about 50 deg C. In contrast, and although they are generally composed of exactly the same chemical components (amino-acids) as mesophilic proteins, all of the proteins in thermophilic organisms have evolved their amino acid sequences so that they are especially stable and can maintain their properly folded 3-dimensional structures and biological functions at high temperatures. Although experimental approaches have been developed to improve the thermal stability of mesophilic proteins, these are laborious, costly and often ineffective, so that it is highly advantageous to use proteins from thermophilic organisms in situations where high protein stability is desired. Typically, these applications have included industrial processes that use enzymes to carry out chemical reactions. There have been no reports of using thermostable proteins for nanotechnology applications. The use of engineered thermostable proteins for nanotechnology applications has many advantages.

One advantage is the ease of production of thermostable proteins for nanotechnology applications. Thermostable proteins are much more stable than proteins in found in most bacteria (e.g. E. coli, B. subtilis, etc.), insect (e.g. sf9, etc.), or mammalian (e.g. CHO, HELA, etc.) cell lines typically used for recombinant expression of proteins. This greatly facilitates the isolation of these protein since once the thermostable protein has been expressed in the host cell line, it is often possible to gain a significant initial purification simply by treating the cells containing the thermostable protein to denaturing conditions (e.g. by heating or urea treatment) that cause most all of the mesophilic cell components to denature and become insoluble, leaving the thermophilic protein intact and in solution where it can be easily separated from the insoluble cell components by centrifugation, filtration, or a number of other methods. This substantially reduces the time and cost required to produce the materials required for nanotechnology applications.

A second advantage is the ease of production of engineered and chemically modified variants of thermostable proteins for nanotechnology applications. In many cases thermostable proteins that will be used for nanotechnology applications will not be used in their native form as they are found in nature, but in some modified form. However, owing to the very high initial stability of the native forms of thermostable proteins, such modifications are expected to have a relatively small effect on the functional stability of a thermostable protein relative to a protein derived from a mesophilic organism.

Useful modifications of the native thermostable protein can be achieved in two general ways. The first approach involves the modification of the “native” protein amino acid sequence as it occurs in nature through manipulation of the DNA sequence that encodes the protein. The manipulated DNA sequence can then be expressed in an expression system, for example, a bacterium, such as E. coli, to produce the desired modified amino acid sequence. This process is generally termed protein engineering and is broadly used in the biotechnology industry. The second general method involves reacting a protein composed of naturally occurring amino acids with chemical reagents or enzymes that post-process the protein to make a chemical derivative of the product encoded by the DNA sequence.

Introduction of modifications in the sequence of proteins using recombinant DNA technology is broadly used in biomedical research and is the basis of many pharmaceutical products. However, with the exception of Salemme & Weber (2007), no reports exist for using protein engineering for structural nanotechnology applications using thermostable proteins. Structural modifications of thermostable proteins intended for nanotechnology applications can be introduced using recombinant DNA technology to modify the DNA sequence that encodes the corresponding protein polypepetide sequence. Useful modifications could include, for example:

a. The introduction of one or more individual substitutions of one amino acid for another at defined positions in the native sequence (commonly termed a site-specific modification). Examples of the utility of such modifications include the substitution of an amino acid like cysteine with a chemically reactive side chain for a non-reactive amino acid like alanine to provide a specific chemical linkage site on the surface of a protein.

b. The addition or deletion of one or more contiguously-bonded amino acids (a polypeptide extension) from either the amino or carboxy terminus of the native protein polypeptide chain. Examples of the utility of such modifications include the addition or removal of sequences or protein domains that may confer additional binding or catalytic functionality to the native protein or that may be structurally disordered.

c. The insertion or deletion of one or more amino acids into the sequence of the native or modified protein sequence. Examples of the utility of such modifications include the insertion of sequences or protein domains that may confer additional binding or catalytic functionality to the native protein.

d. The reconnection of the protein polypeptide chain of the native or native-like sequence, so as to allow the preservation of essentially the same 3-dimensional folded structure of the native protein, but folded from a sequence where the positions of the amino and carboxy termini have been altered or permuted. Examples of the utility of such modifications include the covalent connection of multiple polypeptide chains that normally form an associated complex into a single contiguous polypeptide sequence.

e. The interconnection of multiple copies or types of protein sequences that naturally form multimeric structures in nature composed of multiple polypeptide chains, into a structure made up of a smaller number of continuous polypeptide chains.

In actual application, any or all of the types of the modifications of the native protein sequence described in a. through e. above can be used in combination to produce a modified protein sequence.

The second type of modification, which may often be combined with the gene modification strategies outlined above that alter the native protein sequence, involves the reaction of the modified protein with a chemical reagent or enzyme to produce a “chemically modified” protein. Examples of the utility of such chemical modifications include the formation of a covalent connection between the polypeptide structure and chemical groups with specific protein binding activity. For example, chemical reagents are known that can react covalently with the cysteine groups on the surface of proteins to covalently attach biotin. Biotin is a vitamin that has very high and specific binding affinity for several proteins of the avidin family including streptavidin from Streptomyces avidinii and bird avidins. Consequently, proteins that are chemically modified through covalent attachment of biotin groups can form tight and specific interactions with streptavidin and avidin, and as a result have found wide application in biotechnology and diagnostic applications. Because all chemical reactions, including those that tend to spontaneously modify proteins (e.g. oxidation of sulfur containing amino acids and side chain deamidation of asparagine and glutamine residues) tend to occur more rapidly at high temperatures, proteins that are adapted to be stable at high temperature are also unusually stable to changes in chemical environment. This does not mean that modifications like the biotinylation reaction outlined above will not occur with thermostable proteins, but that there is less likelihood that undesirable side reactions will take place that could give rise to defective molecular structures with reduced assembly fidelity for self-assembling nanostructures.

A third advantage afforded to the use of thermostable proteins is the ease of processing during the production and assembly of nanostructures. The production of components for assembly of nanostructures incorporating thermostable proteins will often involve separation steps using chromatography, electrophoresis or other methods used to isolate biological macromolecules and complexes. The enhanced stability of thermostable proteins relative to mesophilic proteins is an advantage that allows better separations of intermediate reaction products and/or molecular subassemblies using a wider range of separation conditions (e.g. solution pH, ionic strength, range of allowable solvents, presence of detergents, etc.). Similarly, the production of nanodevices that are assembled on self-assembling monolayers or semiconductor substrates like silicon wafers will often involve solution conditions and/or the use of reactive or photo-chemistries where the improved stability of thermostable proteins relative to mesophilic proteins will result in better yields of the desired products and more reliable devices.

A fourth advantage afforded to the use of thermostable proteins in nanodevices relates to the allowable range of practical operating conditions for devices incorporating engineered nanostructures. Many important applications for functional nanodevices will be in temperature environments that are not too much different from those normally tolerated by human beings—nominally 0 deg C. to 50 deg C. In particular, nanodevices designed for medical applications will have to operate at about 37 deg C., the temperature of the human body. Even current semiconductor-based electronics typically do not operate reliably above ˜70 deg C. and typically require active cooling in applications like computers. Many proteins from thermophilic organisms, as well as a small number of unusually stable proteins from mesophilic organisms like streptavidin from the microorganism Streptomyces avidinii, remain stable above 70 deg C., whereas most proteins from mesophilic organisms denature in the range of 40 to 50 deg C. making them less suitable for nanodevice applications.

Most of the biomolecular components that we describe here are based on proteins of thermostable microorganisms of known 3-dimensional crystal structure. As outlined above, the use of thermostable proteins provides us with several advantages in economical node production, handling and purification.

The enzymatic binding sites of proteins used as nodes can provide additional sites for functionalization of the nanostructure through covalent binding of inhibitors linked to other chemical moieties or proteins.

### Struts

Two fundamental nanoscale biomolecular components of a “parts box” from which a structure, for example, a device, can be assembled are “struts” and “nodes”. Struts are molecular components that function as linear connectors. Nodes connect struts and orient them with defined geometries.

Throughout the following descriptions we use standard scientific nomenclature to discuss the symmetry properties of node templates and nodes (Vainstein 1994). For a complete description of point group symmetry and symmetry operation nomenclature see: http://www.phys.ncl.ac.uk/staff/njpg/symmetry/index.html and <http://csi.chemie.tu-darmstadt.de/ak/immel/script/redirect.cgi?filename=http://csi.chemie.tu-darmstadt.de/ak/immel/tutorials/symmetry/index.html>

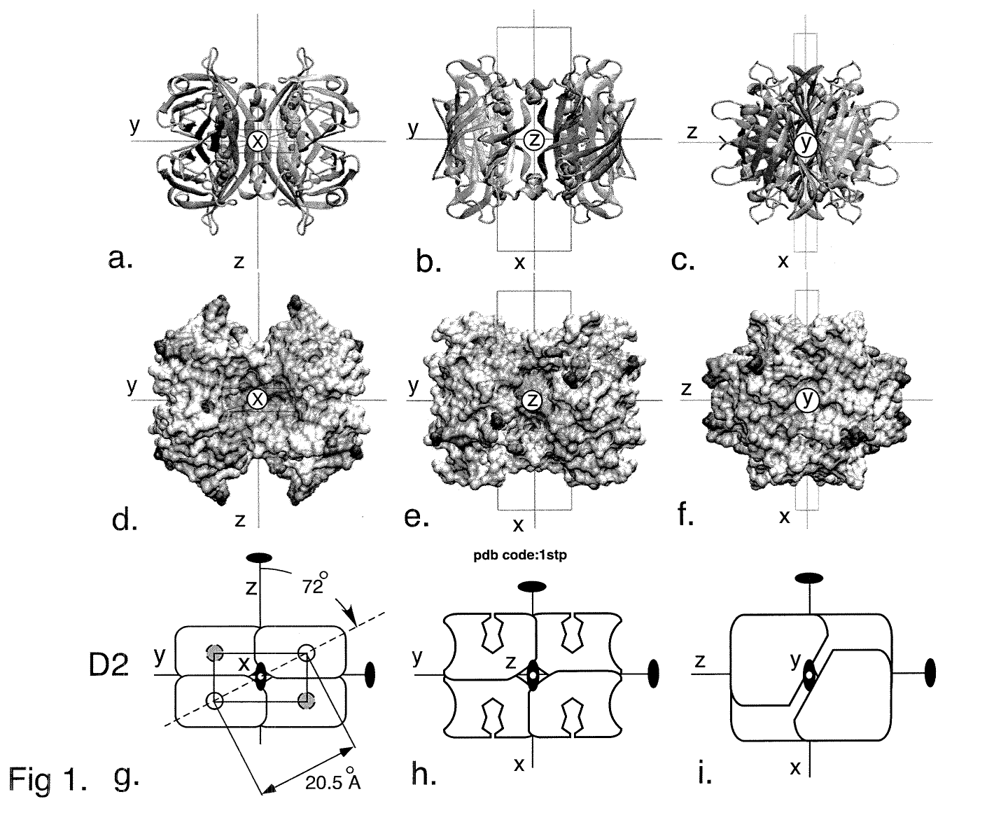

A strut can be formed from streptavidin, a tetrameric protein of 60 kiloDalton molecular weight secreted by the bacterium Streptomyces avidinii. FIG. 1 shows molecular models and schematic illustrations of the streptavidin tetramer showing biotin ligand binding sites. The streptavidin tetramer has D2 symmetry with 3 mutually perpendicular two-fold or dyad axes of symmetry relating the 4 subunits of the tetramer. Dyad axes are labeled x, y, and z in FIG. 1. FIGS. 1a,b, and c show schematic backbone representations of the streptavidin tetramer viewed down the x, z, and y dyad axes of symmetry, respectively. The bound biotin ligands are shown in space filling representation. Also shown in FIGS. 1 a through f is a “bounding box” aligned along the x dyad axis that defines the positions of the biotin ligands along the direction that they make bonded interactions with nodes. FIGS. 1d,e, and f show surface representations of the streptavidin tetramer viewed down the x, z, and y dyad axes of symmetry respectively. FIGS. 1g,h, and i show schematic representations used elsewhere in this document for illustrative purposes. In FIG. 1f, a schematic view down the x-axis dyad of the streptavidin tetramer, the 2 facing biotin binding sites (shown schematically as open circles) are spaced approximately 20.5 Angstroms apart and aligned along a line that is inclined at a 72 degree angle relative to the z dyad axis of the streptavidin tetramer. The streptavidin tetramer has dimensions of approximately 45 Angstroms (4.5 nanometers) along the x-axis, by 60 Angstroms (6 nanometers) on the y-axis, by 55 Angstroms (5.5 nanometers) on the z-axis.

Weber et al. (1989) determined the X-ray structure of streptavidin and described the origins of its ability to bind the vitamin biotin. Although the biotin:streptavidin interaction is non-covalent, the biotin dissociation constant is about 10⁻¹⁴M, so that the biotin:streptavidin bond is essentially irreversible. The strength of the biotin:streptavidin bond has led to the broad application of streptavidin in research and diagnostics applications where interaction specificity is required in a complex biological milieu.

In streptavidin, the biotin-binding sites are arranged as pairs where the surface accessible valeric acid side chains of the biotin moieties are oriented along the verticals of an “H” in an orientation that facilitates specific pairwise binding. The biotin binding sites are arranged with D2 symmetry. When bound to the streptavidin biotin-binding sites, the biotin molecules have their terminal valeric acid chains (which are the usual chemical modification sites for generating biotin conjugated reagents) in extended conformation and oriented approximately parallel to the x diad axis of the streptavidin tetramer. The distance between the two closest and approximately parallel pair of bound biotin chain termini is about 20.5 Angstroms, which are aligned along a line that is inclined at a 72 degree angle relative to the z-dyad axis of the streptavidin tetramer (FIG. 1). Thus, when serving as a strut, a streptavidin tetramer can form be linked to other biomolecular components, such as nodes, at two sites through biotin molecules.

Although the present descriptions refer specifically to streptavidin, several related proteins are known (e.g. egg white avidin) that have similar amino acid sequence, structure, and biotin binding properties as streptavidin. These proteins could be substituted for streptavidin in the applications described here.

In addition to streptavidin and its homologues, many other stable protein tetramers with D2 symmetry, such as those derived from thermostable microorganisms, could function as struts either in their native state or through suitable modification of their amino acid sequence, ligand binding functionality, or chemical modification state. Examples of alternative thermostable strut templates with D2 symmetry are given in Table 1.

### Nodes

A node can connect two or more struts with predefined orientation of each strut with respect to the other connected struts.

For example, a node can be a symmetric protein multimer. For example, a node can be an enzyme that has catalytic binding sites with high binding specificity for certain substrates and cofactors. A naturally occurring protein can be used in its native state, or can be engineered, for example, using site-specific modification techniques, to render it suitable or optimal for an intended function as a node. Selection of a naturally occurring protein for use as a node can be made from the large number of X-ray crystal structures of stable protein multimers having different symmetries available. Alternatively, selection can be made from protein sequences that have over 70% sequence homology with sequences with known X-ray structures, since it is known that homologous protein sequences also have similar 3-dimensional structures, and the multimeric state of a protein can be determined by physical methods like light scattering, electrophoresis, ultracentrifugation, gel exclusion chromatography, or other methods.

In general, such multimers serving as nodes can be interconnected by biomolecular components serving as struts (such as streptavidin) to create nano-scale structures with defined two- and 3-dimensional geometry.

As outlined in Table 1, suitable multimeric proteins with utility as node templates are known having 3-fold (C3), 4-fold (C4), 5-fold (C5), 6-fold (C6), 7-fold (C7), and other rotational symmetries. In addition, multimeric proteins with utility as node templates are available with higher symmetry, including D2, D3, D4, tetrahedral, cubeoctahedral, icosahedral, and other symmetries. While nodes or node variants having Cn rotational symmetry are primarily suited to the assembly of 2-dimensional planar structures, nodes with higher fold symmetry more naturally lend themselves to the assembly of 3-dimensional structures and lattices. The structures referenced in Table 1 of these and additional proteins that can serve as templates for nodes can be viewed at the Protein Data Bank (PDB) website http://www.rcsb.org/pdb/home/home.do (accessed Oct. 2, 2007) by entering the appropriate PDB Code as listed in Table 1. The Protein Data Bank is a Federally supported, archival database that includes complete 3-dimensional structure coordinate data, amino acid sequence data, and links to relevant scientific literature. The structures in the Protein Data Bank are hereby incorporated by reference. Proteins are labeled with their 4-letter protein Protein Data Bank identification code (pdb code) throughout this document.

For example, site-specific modification techniques can be used to introduce surface cysteine residues at pairs of points on the surface of a multimer to function as a node. Biotinylating reagents, for example, a thiol-reactive biotinylating reagent, can be covalently bonded to such surface cysteine residues to introduce biotin groups at defined, for example, at symmetric points on multimeric node. Thus, a node of defined geometry can be formed. The pairs of biotin groups on the multimer functioning as a node can then be bound to the binding sites on streptavidin tetramers, which can act as struts, to form a two- or 3-dimensional nanostructure.

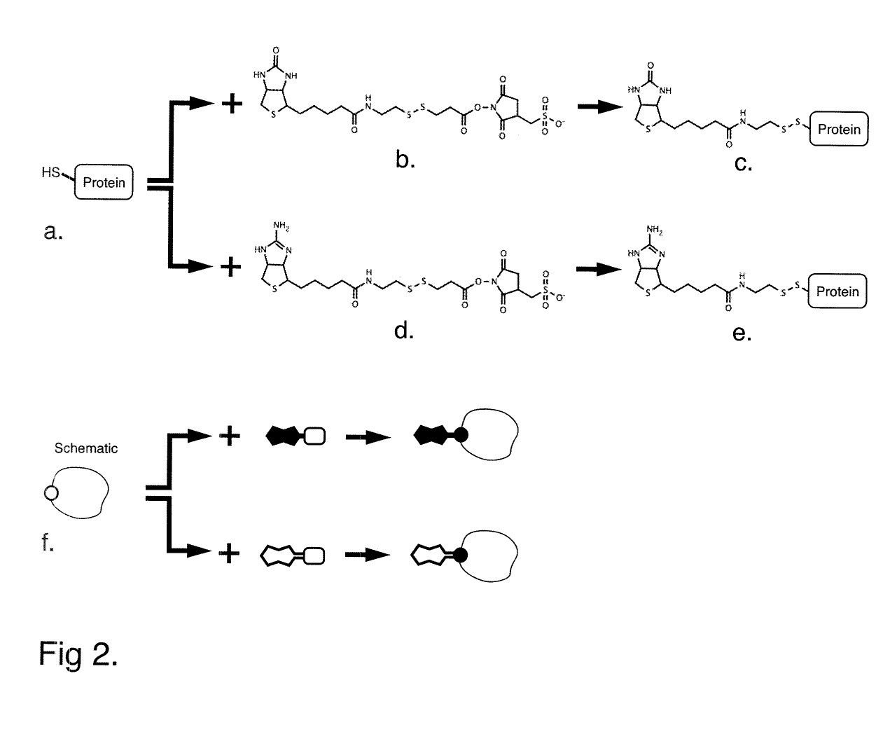

Reactions of biotinylating reagents that can modify protein cysteine sulfhydryl groups are presented in FIG. 2. FIG. 2a shows a free sulfhydryl group on a protein. FIG. 2b shows the biotinylation reagent Sulfosuccinimidyl 2-(biotinamido)-ethyl-1,3-dithiopropionate (EZ-Link Sulfo-NHS-SS-Biotin: Pierce). FIG. 2c shows the reaction product after biotinylation. FIG. 2d shows an analogous reagent for the introduction of 2-imino biotin groups. The binding of imino-biotin to streptavidin is pH dependent. At low pH (˜pH4) the imino group becomes charged, causing imino-biotin displacement from the streptavidin biotin binding site. Iminobiotin linking is useful for the formation of reversible interactions between streptavidin struts and node proteins. FIG. 2e shows the imino-biotin reaction product. FIG. 2f shows the above reaction sequences schematically as used in schematic illustrations elsewhere in this application. Although the reagents in FIG. 2 show a specific linker length, biotinylation reagents are readily available with various linker lengths and custom ones are readily synthesized through incorporation of amino-alkyl-thiol coupling groups with variable alkyl or glycol chain lengths.

### General Descriptions of Node Geometry

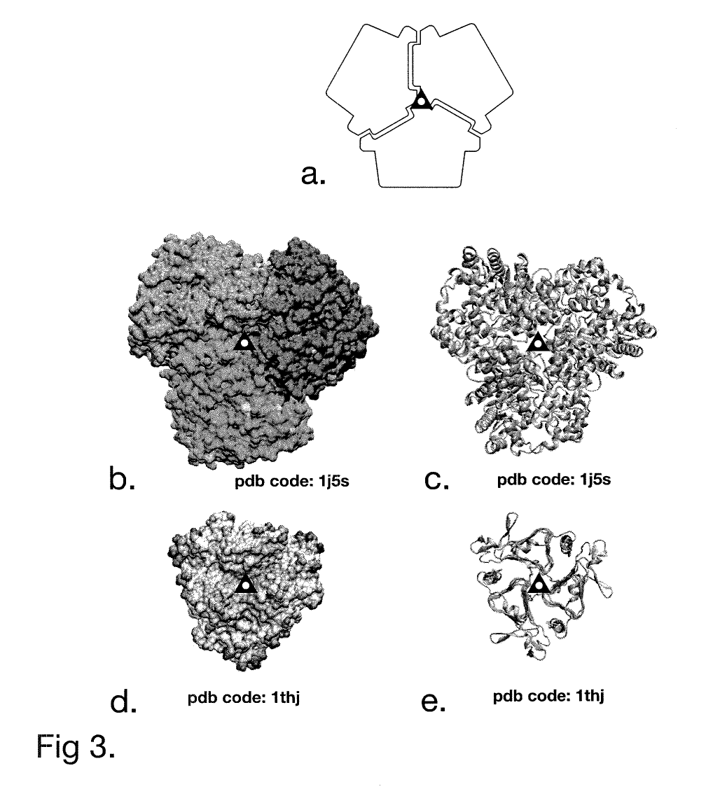

Nodes with Cn Symmetry: The simplest symmetry that a multimeric note can have is Cn rotational symmetry. Since proteins are polymers composed of L-amino acids they are intrinsically asymmetric, and consequently nodes with Cn symmetry have polarity. As such nodes with Cn symmetry are well-suited to the assembly of 2-dimensional structures on surfaces where, for example, structural features on one polar face of the multimer (which is generally normal to the Cn symmetry axis), can be functionalized to provide the ability to bind to a planar substrate that can be a surface or self-assembling monolayer. FIG. 3a shows a schematic view of a three-fold symmetric multimer, while FIGS. 3b and 3c shows representations of the uronate isomerase protein (TM0064) from Thermotoga maritima (Schwarzenbacher et al. 2003, pdb code: 1j5s) in space filling and schematic backbone representations respectively. Each chain of the trimer comprises 450 amino acid residues. FIGS. 3d and 3e shows a representations of a carbonic anhydrase protein from Methanosarcina thermophila (Kicker, et al 1996, pdb codes: 1thj & 1qrf) in space filling representation and schematic backbone views respectively. Each chain of the trimer comprises 213 amino acid residues. Additional C3 symmetric node templates are presented in Table 1.

Single chain constructs of a node protein can be formed. For example, these fused protein multimers can be constructed by incorporating a DNA sequence coding for a polypeptide linker connecting the C-terminus of a first multimer gene to the N-terminus of a second multimer, and so on, to create a single contiguous gene coding for the complete multimer. This approach can allow for the subunits of a multimeric protein to be non-identical. For example, surface cysteine residues for biotinylation can be included in some subunits, but not in other subunits, so that struts can be attached at certain faces of the multimeric protein, but not at others. In addition to the controlling strut-binding geometry, other features of the individual multimer subunits may be individually varied to introduce asymmetry into the node. For example, if the individual multimer subunits have enzyme or cofactor binding sites that can serve as attachment points of additional inorganic, organic or biomolecules that can additionally functionalize the structure, these may be selectively eliminated using recombinant DNA technology to produce nodes where the only some of the binding sites remain intact. Conversely, methods of protein engineering may be used to introduce new binding functionality into the individual multimer subunits to produce single-chain multimeric nodes with asymmetric binding geometry.

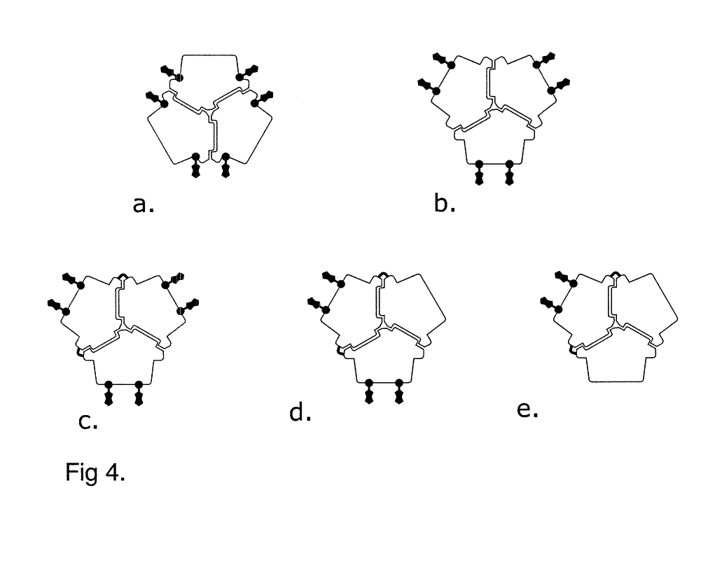

Some variations of the structure of C3 multimeric nodes are illustrated in FIG. 4 Each node is composed of a trimeric protein where the subunits have been modified through site-specific mutagenesis to introduce surface amino acid residues that can be chemically modified to introduce pairs of biotin groups with geometry that is complementary to two of the binding sites on the streptavidin tetramer. FIG. 4a shows a node that is a trimer formed from three independent, identical chains that are not covalently connected. Two biotins are bound to each chain, so that a streptavidin strut can bind to each subunit. In this construct, the pairs of sites of surface biotinylation that are geometrically complementary to streptavidin are on different subunits. FIG. 4b shows a node that is a trimer as formed from three independent, identical chains that are not covalently connected. Two biotins are bound to each chain, so that a streptavidin strut can bind to each subunit. In this construct, the pairs of sites of surface biotinylation that are geometrically complementary to streptavidin are on the same subunits. FIG. 4c shows a node based on a protein trimer formed from a single chain construct, that is, with each subunit linked to another by a polypeptide linker. That is, the individual chains of the non-covalently associated trimer have been covalently connected together in a single continuous polypeptide chain. Two biotins are bound to each chain, so that a streptavidin strut can bind to each subunit. FIG. 4d shows a node based on a protein trimer formed from a single chain construct. Two of the subunits of the trimer have bound biotin pairs, but the third does not. Thus, only two streptavidin struts can be linked to the trimer. As such, the trimer can serve as a connector between struts, but does not allow branching from one strut to two other struts. FIG. 4e shows a node based on a protein trimer formed from a single chain construct. Only one of the subunits of the trimer has a bound biotin pair; the other two do not. Thus, only one streptavidin strut can be linked to the trimer. As such, the trimer can serve as a terminator of a strut, and cannot serve as a connector or branch point between struts.

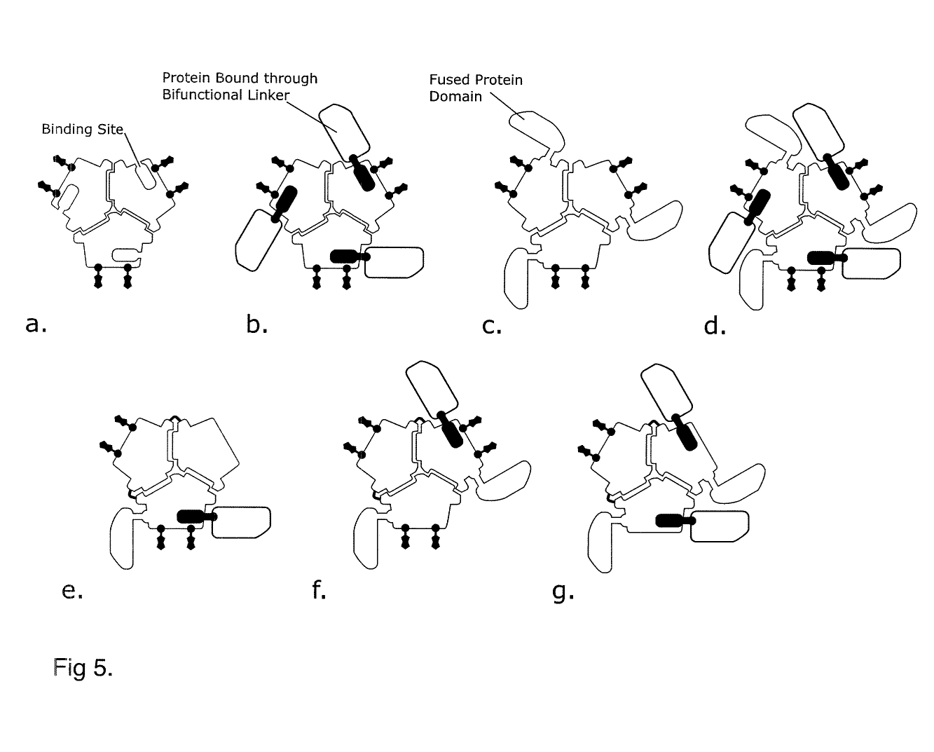

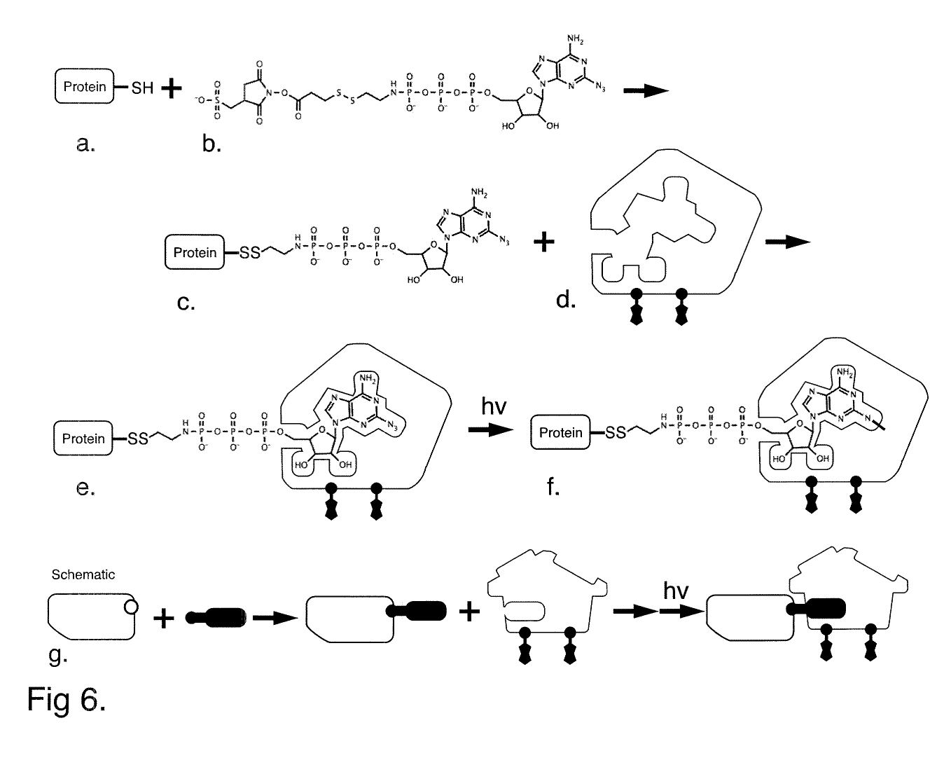

Nodes can be functionalized in at least two ways. Nodes may be selected that are enzymes that are characterized by the presence of specific substrate and cofactor binding sites. An approach to functionalizing nodes uses bifunctional crosslinking reagents that specifically bind to binding sites on enzymes for substrates or cofactors (FIG. 5a,b). Bifunctional crosslinking reagents can incorporate an enzyme-specific reactive agent on one end and specific protein-reacting group (for example, a group able to react with cysteine side chain thiol group or a polypeptide chain terminal amine group) on the other end of the linker. For example, many enzymes use ATP as a specific cofactor. FIG. 6 shows reactions of a bifunctional crosslining reagent incorporating an azido-ATP group on one end (which forms a covalent bond between the reagent and the protein upon ultraviolet light irradiation) and a thiol reactive reagent on the other end that will specifically react with a protein cysteine side chain. Specifically, FIG. 6a shows a protein with a surface cysteine sulfhydryl group that can react with the sulfhydryl reactive reagent (FIG. 6b) incorporating a 2-azidoadenosine 5′-triphosphate group to produce the reaction product in FIG. 6c. The 2-azido ATP modified protein (FIG. 6c) can then bind to an ATP cofactor binding site on a node protein (FIG. 6e). Upon irradiation with UV light, the azido-ATP reacts with amino acid side chains of the node protein in the ATP binding site to form a covalent bond (FIG. 6f). FIG. 6g presents the reaction sequence schematically using symbols used elsewhere in this application. Although the bifunctional reagents in FIG. 6 show a specific linker length, reagents with various linker lengths are readily synthesized through incorporation of amino-alkyl-thiol coupling groups with variable alkyl chain lengths. The preceding and related linkers can be generated using commercially available reagents (Affinity Labeling Technologies, Lexington Ky.; Pierce, Rockford Ill.) or are compounds readily synthesized by one with skill in the art.

The aforementioned azido-ATP analog represents one example, but many additional examples can be envisioned where other biochemical cofactors such as flavins, vitamins, and other biochemical cofactors that bind specifically to proteins can be chemically modified so that they can be photo-crosslinked to protein molecules functioning as either struts or nodes in assembled nanostructures.

Many proteins and enzymes naturally incorporate binding sites that are specific for binding substrates and cofactors. In many cases, this binding specificity can be modified, eliminated, or new binding specificity created de novo from site-specific modification of the template protein sequence.

Since di- or multimeric strut or node proteins can potentially be modified forms of enzymes that carry out specific catalytic processes on biochemical substrates, many such nodes built on enzyme templates will incorporate active sites that bind substrates and catalyze reactions with great specificity. For many classes of enzymes, covalent inhibitors or suicide substrates are known that irreversibly inhibit the enzyme activity by forming a highly specific covalent bond with the catalytic amino acid side chain groups in the enzyme's active site. These agents are generally termed suicide substrates or covalent inhibitors of enzyme activity. These agents, when connected to one end of a bifunctional crosslinking reagent as described above, can provide a means of specific immobilization of a protein to an underlying strut-node architecture. For example, immunoglobulins, lectin, or other specific binding molecules could be linked to nanostructures constructed of struts and nodes using this means, as outlined below. FIG. 5b shows a schematic of a C3 symmetric node that is a trimer formed from three independent, identical subunits, where each subunit possesses additional specific binding functionality, and where proteins have been specifically linked to the node using a bifunctional crosslinking reagent. Such functionalization can be used in nanostructures intended to serve in filters, diagnostics or biological sensing applications.

In addition to the use of chemical crosslinking agents as a way to couple proteins to the underlying strut-node structure, it is possible to engineer either nodes or strut components where the nucleotide sequence coding for the node or strut component is modified by a sequence insertion or extended (e.g., in the form of a polypeptide extension) at either the amino or carboxy terminus with nucleotide sequences coding for additional binding function. When these “fused” genes incorporating the binding domain sequences are expressed, the result will be a single continuous polypeptide chain incorporating the encoded linked protein domain. FIG. 5c shows a schematic of a C3 node composed of 3 independent chains, where each chain incorporates a covalently linked or fused protein domain. The fused domains can have utility in both protein isolation and in creating protein assemblies. Examples of such fused domain binding sequences (for example, a polypeptide extension) include immunoglobulin domains, polyhistidine sequences, polypeptide sequences that bind to streptavidin (Streptag), Staphylococcus Protein-A, Staphylococcus Protein-G, an antibody binding polypeptide sequence to which an antibody can bind, an antigenic polypeptide sequence, a hapten polypeptide binding sequence, a binding function for a protein or a metallic surface, a polypeptide sequence that is a substrate for an enzyme, and others together with sequences designed to be linkers with greater or lesser conformational flexibility. FIG. 5e shows a single-chain construct of a C3 node where multimer subunits with different functionalities have been interconnected with polypeptide linkers creating an asymmetric multimeric node. Starting from upper right, and going counter-clockwise, the first node subunit has no binding capability (e.g. enzyme active site groups removed through site-specific mutagenesis) or incorporated biotinylation sites, the second node subunit has also had binding capability removed but has incorporated biotinylation sites, and the third subunit incorporates both a fused domain and a protein bound through a bifunctional crosslinking reagent. FIGS. 5f and 5g show additional possibilities that generally illustrate the modularity and combinatorial flexibility of the approach in generating a wide variety of geometries and functionalized structures.

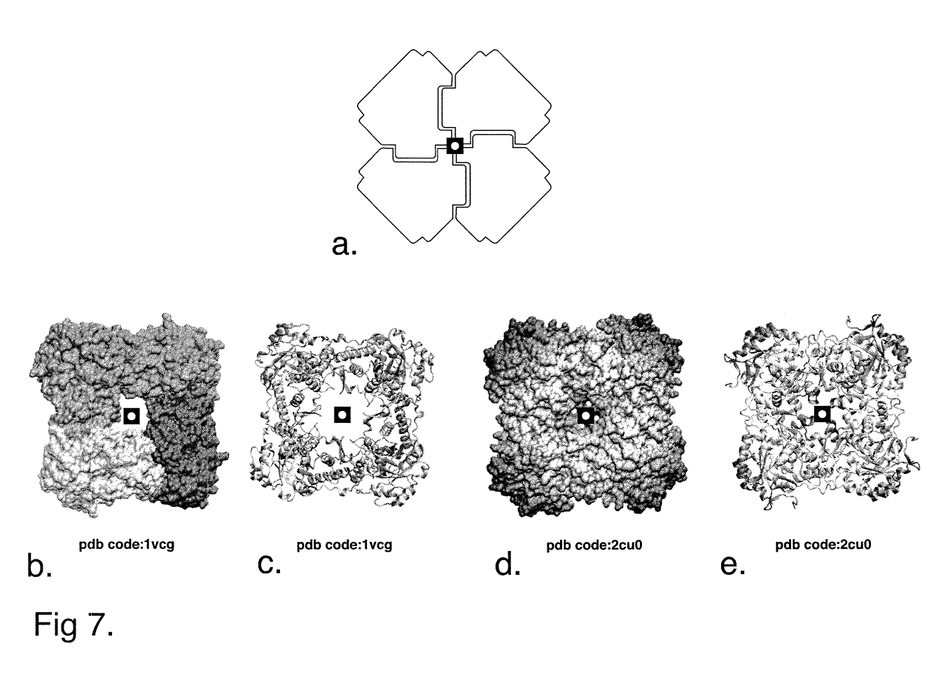

FIG. 7a shows a schematic view of a four-fold (C4) symmetric multimer, while FIGS. 7b and 7c show representations of the isopentenyl-diphosphate delta-isomerase from Thermus thermophilus (Wada et al. 2006, pdb code: 1vcg) protein in space filling and schematic backbone representation respectively. Each chain of the tetramer incorporates 332 amino acid residues, and a non-covalently bound flavin mononucleotide cofactor. FIGS. 7d and 7e show representations of the inosine-5′-monophosphate dehydrogenase protein from Pyrococcus horikoshii (Asada & Kunishima 2006, pdb code: 2cu0) in space filling and schematic backbone representation respectively. Each chain of the tetramer incorporates 486 amino acid residues, and a non-covalently bound xanthosine-5′-monophosphate substrate analog. Additional C4 symmetric node templates are presented in Table 1.

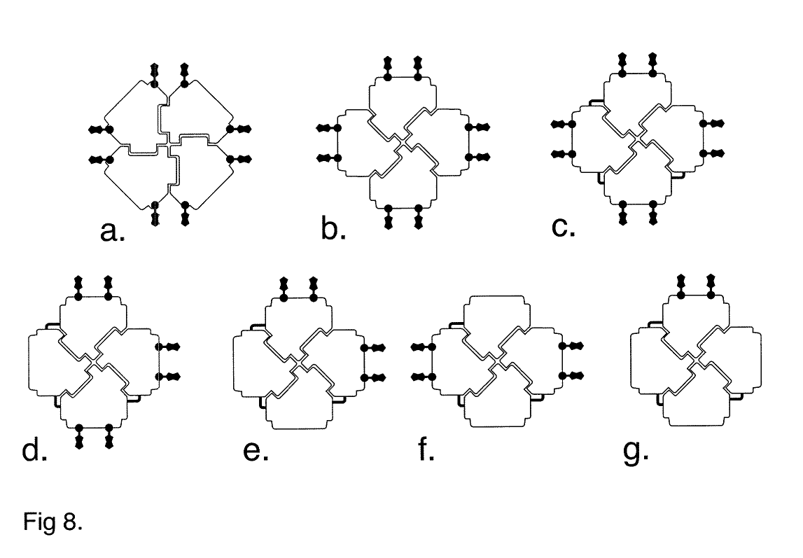

FIGS. 8a through 8g show schematic views of nodes based on a protein tetramer having four-fold (C4) rotational symmetry. Each node is composed of a tetrameric protein where the subunits have been modified through site-specific mutagenesis to introduce surface amino acid residues that can be chemically modified to introduce pairs of biotin groups with geometry that is complementary to two of the binding sites on the streptavidin tetramer. FIG. 8a shows a node that is a tetramer as formed from four independent, identical chains that are not covalently connected. All of the subunits of the tetramer are symmetrically equivalent. Two biotins are bound to each chain, so that a streptavidin strut can bind to each subunit. In this construct, the pairs of sites of surface biotinylation that are geometrically complementary to streptavidin are on different subunits. FIG. 8b shows a node that is a tetramer as formed from four independent, identical chains that are not covalently connected. All of the subunits of the tetramer are symmetrically equivalent. Two biotins are bound to each chain, so that a streptavidin strut can bind to each subunit. In this construct, the pairs of sites of surface biotinylation that are geometrically complementary to streptavidin are on the same subunits. FIG. 8c shows a node based on a protein tetramer formed from a single chain construct, that is, with each subunit linked to another by a polypeptide linker. That is, in the structure shown in FIG. 8c the individual chains of the non-covalently associated tetramer are covalently connected together in a single continuous polypeptide chain. Two biotins are bound to each chain, so that a streptavidin strut can bind to each subunit. FIG. 8d shows a node based on a protein tetramer formed from a single chain construct. Three of the subunits of the tetramer have bound biotin pairs, but the fourth does not. Thus, only three streptavidin struts can be linked to the tetramer. As such, the tetramer can serve as a branch point for three struts. FIG. 8e shows a node based on a protein tetramer formed from a single chain construct. Two adjacent subunits of the trimer have bound biotin pairs, but the third and fourth subunits do not. Thus, only two streptavidin struts can be linked to the tetramer. As such, the tetramer can serve as a connector between struts, but does not allow branching from one strut to two or more other struts. The tetramer can serve, for example, to form a corner of a rectangular assembly. FIG. 8f shows a node based on a protein tetramer formed from a single chain construct. Two opposed subunits of the tetramer have bound biotin pairs; the first and third subunits do not. Because only two streptavidin struts can be linked to the tetramer, the tetramer can serve as a connector between struts, but does not allow branching from one strut to two or more other struts. The tetramer can serve, for example, to form a connector between two struts oriented along the same axis. FIG. 8g shows a node based on a protein tetramer formed from a single chain construct. Only one of the subunits of the tetramer has a bound biotin pair; the other three do not. Thus, only one streptavidin strut can be linked to the tetramer. As such, the tetramer can serve as a terminator of a strut, and cannot serve as a connector or branch point between struts. Thus, FIGS. 8d through 8g show covalently connected tetramers of which the surface binding sites on some subunits have been deleted, creating nodes with various streptavidin binding geometry and valency.

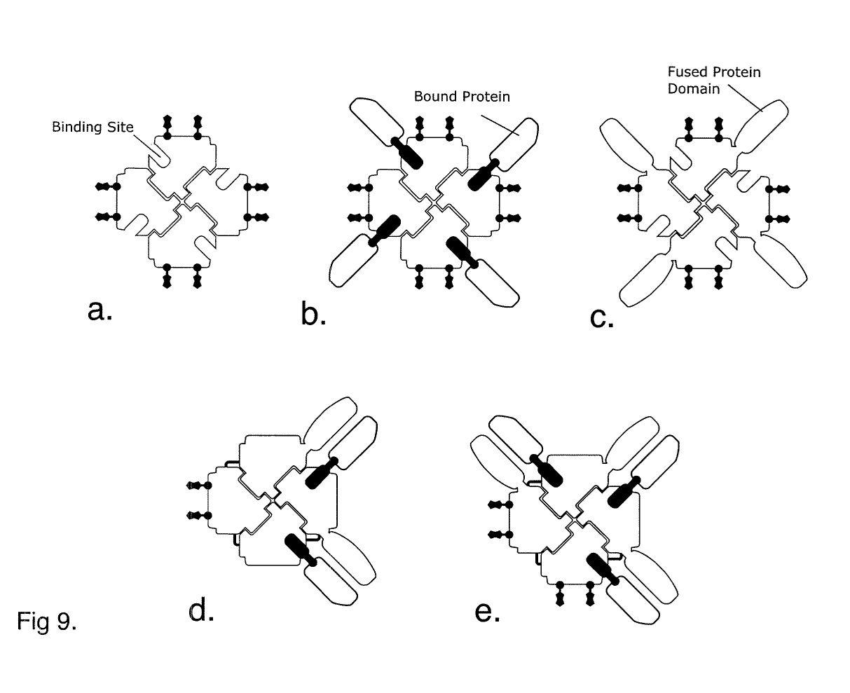

FIG. 9 shows variations of C4 symmetric nodes that have been functionalized and have various geometrical properties. As noted above for C3 nodes, C4 nodes can be functionalized in at least two ways. Nodes may be selected that are enzymes that can be reacted with bifunctional crosslinking reagents that specifically bind to enzyme binding sites for substrates or cofactors. FIG. 9a shows a schematic of a C4 symmetric node that is a tetramer formed from four independent, identical subunits, where each subunit possesses additional specific binding functionality corresponding to an enzyme substrate and/or cofactor binding site. FIG. 9b shows a schematic of a C4 symmetric node formed from four independent, identical subunits, where proteins have been specifically linked to the node using a bifunctional crosslinking reagent.

As in the case of C3 symmetric nodes, multimer subunits of C4 nodes my also be modified by a sequence insertion or extended at either the amino or carboxy with nucleotide sequences coding for additional binding function. FIG. 9c shows a schematic of a C4 node composed of 4 independent chains, where each chain incorporates a covalently linked or “fused” protein domain. FIG. 9d shows a single-chain construct of a C4 node where multimer subunits with different functionalities have been interconnected with polypeptide linkers creating an asymmetric multimeric node. Starting from the top, and going counter-clockwise, the first node subunit incorporates a fused binding domain (any substrate or cofactor binding ability of the native template node having been removed through site-specific mutagenesis), the second subunit incorporates streptavidin binding capability, the third subunit incorporates a binding protein linked through a bifunctional linker, and the fourth subunit incorporates both a binding protein linked through a bifunctional linker and a fused binding domain. As outlined above, fused domains could include immunoglobin binding domains such as Staphylococcus Protein-A, Staphylococcus Protein-G, nucleotide binding domains, or others while bound proteins could include immunoglobulins or other proteins. The node show in FIG. 9d can function as a strut terminator in nanostructures. As outlined below, the ability to precisely position two different kinds of proteins or antibodies in nanostructures with close apposition could have many applications in functional or diagnostic applications. FIG. 9e shows an analogous construct that can form a 90 degree corner in a 2-dimensional planar array. Many additional constructs based on C4 symmetric templates are possible through combinations of the features outlined above, retaining all of the properties of modularity and combinatorial flexibility of the approach in generating a wide variety of geometries and functionalized structures.

The following descriptions of nodes with higher symmetry do not generally include explicit descriptions of nodes functionalized through incorporation of fused domains or bound proteins, although it can be recognized that these approaches are equally applicable to node subunits forming complexes of higher symmetry. Similarly, nodes of higher symmetry may be formed using polypeptide chains where two or more of the polypeptide sequences comprising a multimer subunit in the node template structure, have been interconnected to form a single continuous polypeptide chain by interconnection through a polypeptide linker. Thus, design of nodes of higher symmetry can incorporate all of the properties of modularity and combinatorial flexibility of the approach defined above in generating a wide variety of geometries and functionalized structures.

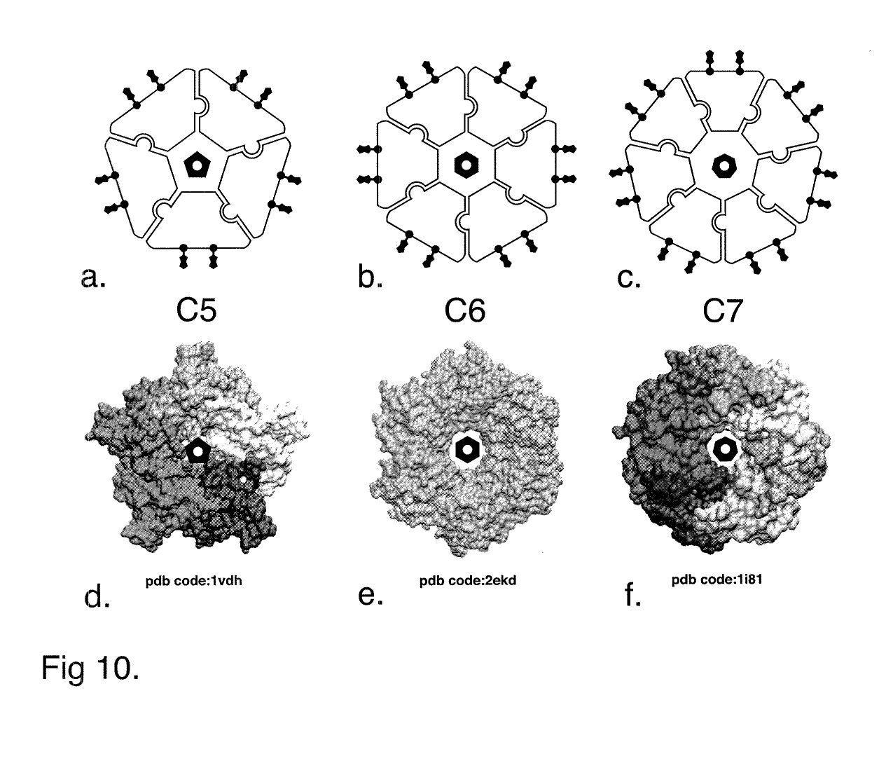

In addition to nodes with C2, C3, and C4 symmetry, natural protein multimers from thermophilic organisms occur with higher Cn rotational symmetry. FIG. 10 presents schematic illustrations of biotinylated nodes with C5 (FIG. 10a), C6 (FIG. 10b), C7 (FIG. 10c) symmetry together will illustrations of thermophile-derived proteins with corresponding symmetry. The C5 symmetric protein shown in surface representation in FIG. 10e is a pentameric heme-binding protein from Thermus thermophilus HB8 (Ebihara et. al. 2005, pdb code: 1vdh). Each polypeptide chain of the pentamer has 249 amino acid residues. FIG. 10e shows a surface representation of the PH0250 protein from Pyrococcus horikoshii OT3 (Asada and Kunishima 2007, pdb code: 2ekd) with C6 symmetry. Each polypeptide chain of the hexamer has 207 amino acid residues. FIG. 10f shows a surface representation of an heptameric RNA binding protein from Methanobacterium thermoautotrophicum (Collins et. al. 2001, pdb code: 1i81) with C7 symmetry. Each polypeptide chain of the heptamer has 83 amino acid residues. Additional Cn symmetric node templates are presented in Table 1.

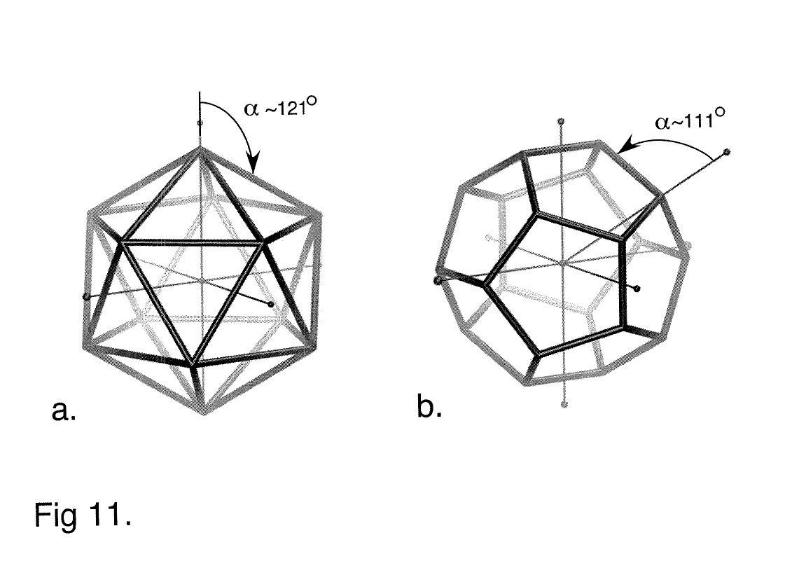

Non-planar Cn Nodes: In addition to Cn nodes with radial planar symmetry (e.g. with biotinylation sites introduced to orient bound streptavidin tetramers in a plane normal to the Cn axis of the multimeric node), Cn multimers with suitable geometrical features can be site-specifically modified to orient streptavidin tetramers at an angle α to the Cn multimer axis. As shown in FIG. 11, such nodes have utility in the assembly of closed polyhedra for specific values of n and α consistent with polyhedron formation (Pugh 1976, Williams 1979, Pearce, 1979). For example, for the generation of an icosahedron, n=5, and the approximate apex angle α=121.72 degrees (FIG. 11a). For the generation of a dodecahedron, n=3, and the approximate apex angle α=z 110.73 degrees (FIG. 11b). Similar considerations apply to the generation of other regular and irregular polyhedra, such as buckyballs (truncated icosahedron) and buckytubes (Weber 1999) where n=3 and the approximate apex angle α=z 104.15 degrees.

Nodes with Dn Symmetry: Many multimeric structures with Dn symmetry are known from x-ray crystallography studies of proteins from thermophilic organisms (Table 1). Dn-symmetric structures arise through the combination of dyad symmetry and other rotational symmetry operations (Table 1). Nodes with Dn symmetry are particularly useful in the assembly of extended nanostructures since biotinylation sites can be introduced symmetrically across multimer dyad symmetry axes to precisely complement dyad-related biotin binding sites on streptavidin (FIG. 1).

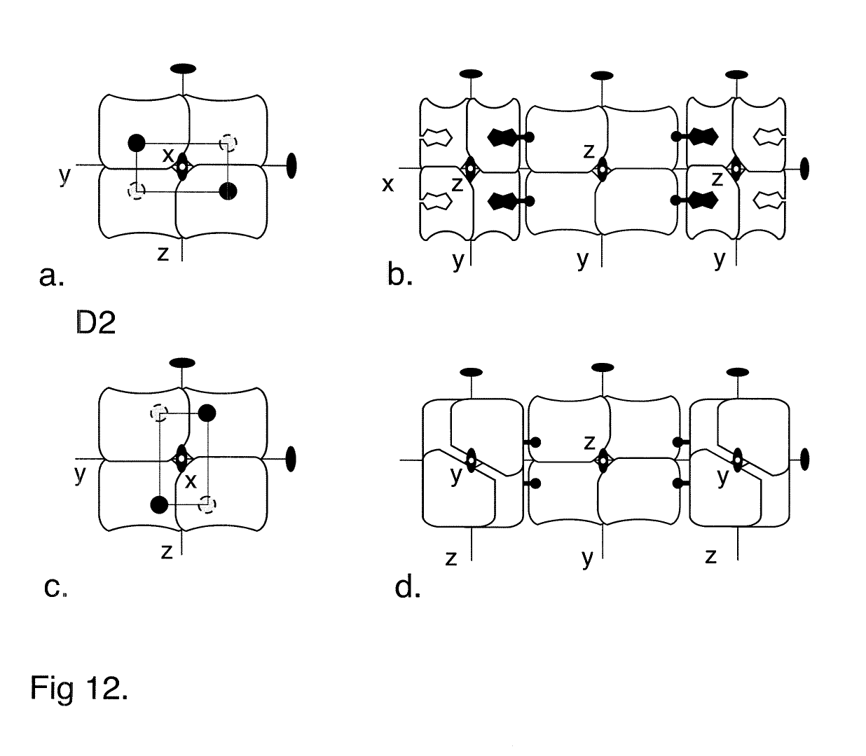

The simplest Dn symmetry is D2, a symmetric tetramer where the multimer subunits are related by 3 mutually perpendicular dyad axes. As noted in FIG. 1, the streptavidin molecule is itself a tetramer with D2 symmetry. Although tetrameric D2 symmetric nodes can potentially function as 3-dimensional nodes in orthorhombic lattices, they are more practically utilized as strut extenders and/or to provide attachment points for additional functionalization. Many tetrameric multimers with D2-symmetry that exhibit a wide range of geometrical features are known from thermophilic microorganisms (Table 1). Some are relatively flat and rectangular in shape, while others approximate tetrahedral geometry. As outlined in FIG. 12 and the specific embodiments described below, D2 nodes with suitable structural features can be used to control the relative geometrical orientation and rotational geometry of connected streptavidin struts. FIGS. 12a and 12c schematically show projection views of a D2-symmetric node able to connect to two streptavidin tetramers through surface biotinylation sites that are introduced at the D2 node surface through site-specific modification of the template protein sequence, followed by a chemical biotinylation reaction (FIG. 2). In FIGS. 12a and 12c, the biotinylation sites are schematically indicated by black circles (defining the corners of a rectangle) on the front surface of the D2 tetramer and as shaded circles on the rear surface of the tetramer. These biotinylation sites are geometrically complementary to the biotin binding sites on streptavidin (FIG. 1g). As illustrated in FIGS. 12 a and c, there are generally two possible orientations for pairs of biotinylation sites that are symmetrically disposed about the D2 (or any other multimer) dyad axis; one where the orientation of the bounding box defining the biotinylation sites is horizontal and which aligns the z-axis dyad of the node with the z-axis dyad of streptavidin (FIG. 12a), and the second where the orientation of the bounding box defining the biotinylation sites is vertical and which aligns the z-axis dyad of the node with the y-axis dyad of streptavidin (FIGS. 1g and 12c). FIGS. 12b and 12d schematically illustrate 2 streptavidin:node:streptavidin complexes, one incorporating a node of the sort shown in FIG. 12a (FIG. 12b), and a second (FIG. 12d) incorporating the node of FIG. 12c. As illustrated, the difference between the complexes is a relative rotation of the streptavidin and node proteins by 90 degrees about the common x-axes of the complexes. In either case, the relative orientations of the terminal free biotin binding sites in the complex are preserved as if the complex were essentially a single streptavidin molecule elongated along the streptavidin x-axis (FIG. 1). Such extended struts are useful for the construction of nanostructures with defined dimensions between nodes as outlined below. The ability to control node orientation in such struts is also a useful property allowing controlled of orientation of additional node functionalizing groups. As noted above for Cn symmetric nodes, many D2 symmetric node structures will incorporate substrate or cofactor binding sites that can be utilized as linkage sites for the introduction of additional protein domains with binding or functional properties. These binding sites provide a means for introducing functional features into the strut components of the nanostructure.

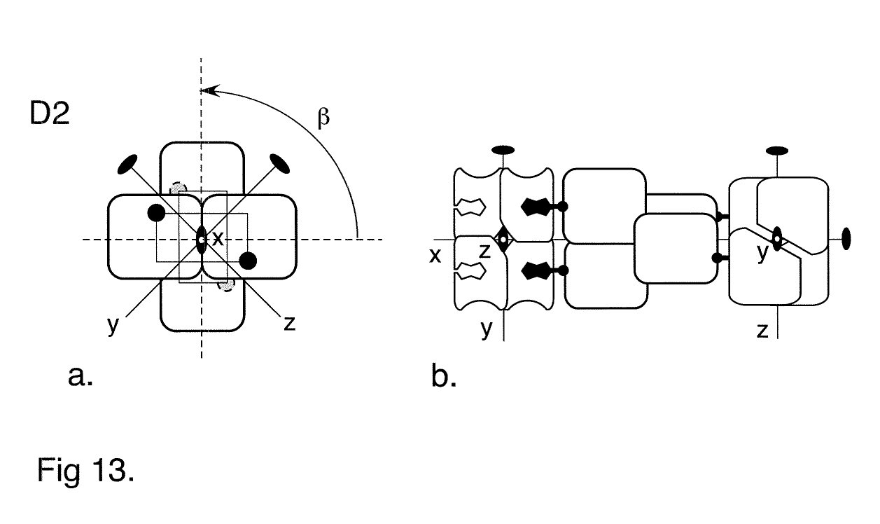

In addition to forming struts that maintain terminal biotin binding site geometry it is possible to construct extended struts where the terminal streptavidin binding sites are oriented at angles other than 180 degrees relative to each other around the common complex x-axis. FIG. 13a schematically shows a projection view of a nearly tetrahedral D2 node where the geometry allows symmetrically equivalent introduction of biotinylation sites in two bounding boxes that are oriented at an angle β to each other along the multimer node x-axis. This feature can introduce a twist in orientation of bound streptavidin tetramers around the common axis of a multimeric complex. FIG. 13b schematically illustrates a streptavidin:node:streptavidin complex where β=90 degrees, so that the relative orientations of the free biotin binding sites on the complex are rotated by 90 degrees along the corresponding streptavidin x-axes (FIG. 1). Extended struts that incorporate some degree of axis rotation of terminal streptavidin binding sites are useful for the geometrical placement of components in nanostructures as well as for construction of 3-dimensional nanostructures with defined dimensions between nodes as outlined below.

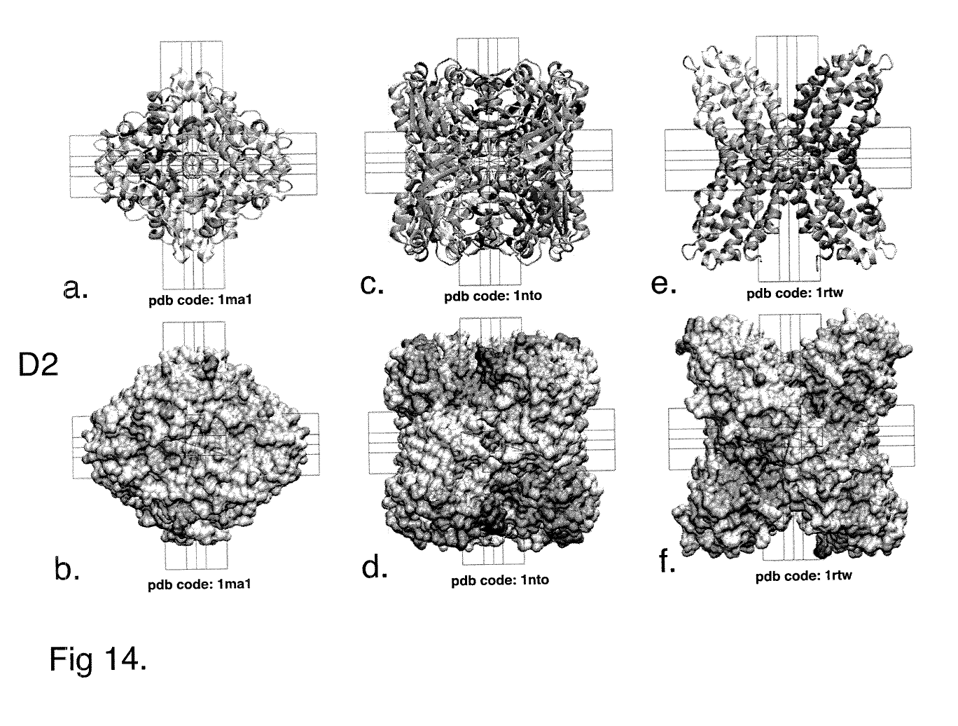

FIG. 14 presents illustrations of some D2 symmetric protein multimers useful as node templates. FIG. 14a shows the iron superoxide dismutase protein from Methanobacterium thermoautotrophicum (Adams et. al. 2002, pdb code: 1ma1) in schematic backbone representation and in surface representation (FIG. 14b). FIG. 14c shows the alcohol dehydrogenase protein from Sulfolobus solfataricus (Esposito, et. al. 2003, pdb code: 1nto) in schematic backbone representation and in surface representation (FIG. 14d). FIG. 14e shows the TenA homolog protein from Pyrococcus furiosus (Benach, et. al. 2005, pdb code: 1rtw) in schematic backbone representation and in surface representation (FIG. 14f). Also shown in FIG. 14 a through f are “bounding boxes” that are aligned along the dyad symmetry axes of the respective D2 symmetric tetramers. The intersections between the molecular surface and the diagonal edges of the box define sites that are symmetric and complementary to the biotin binding in streptavidin, as described in more detail below. Additional structures with D2 symmetry are listed in Table 1.

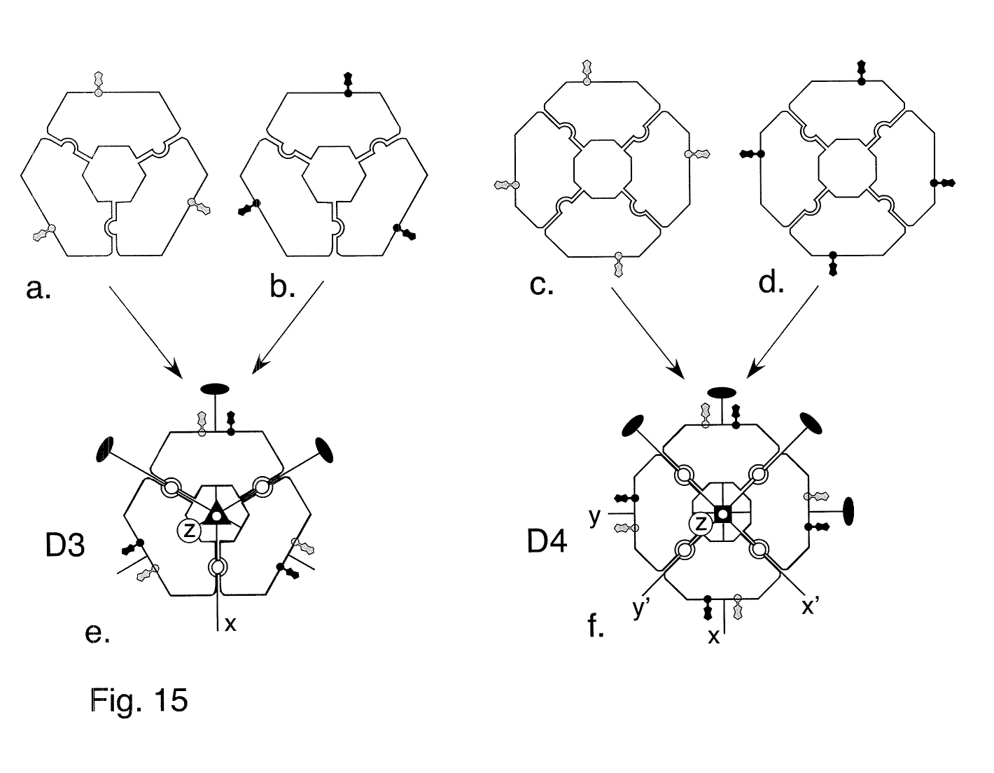

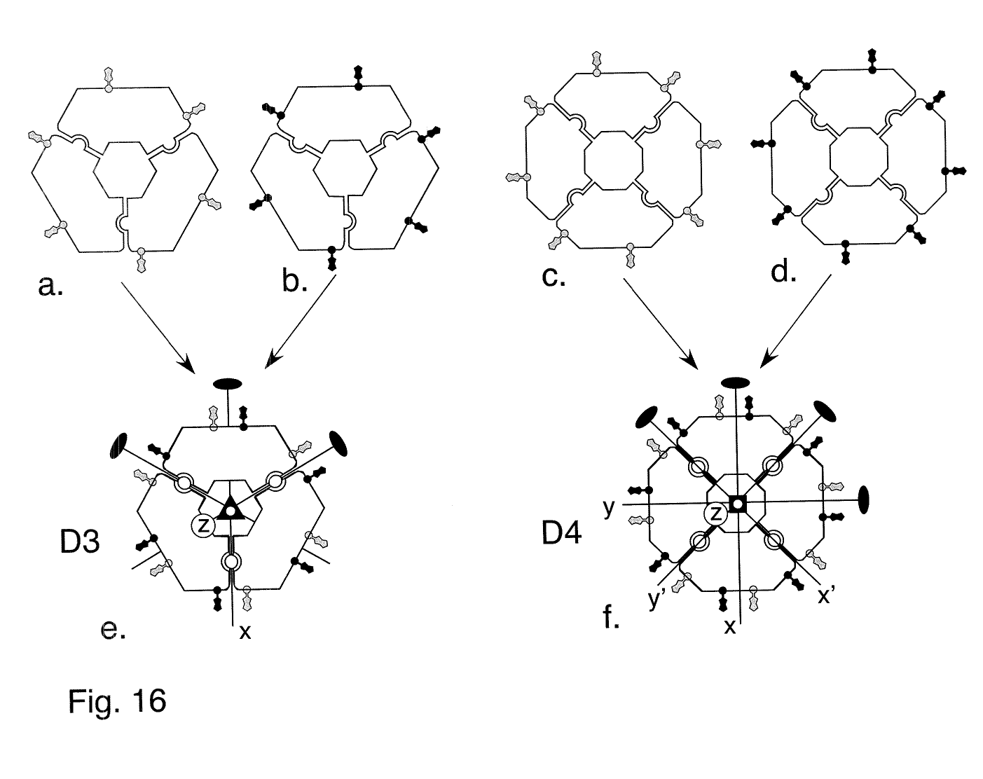

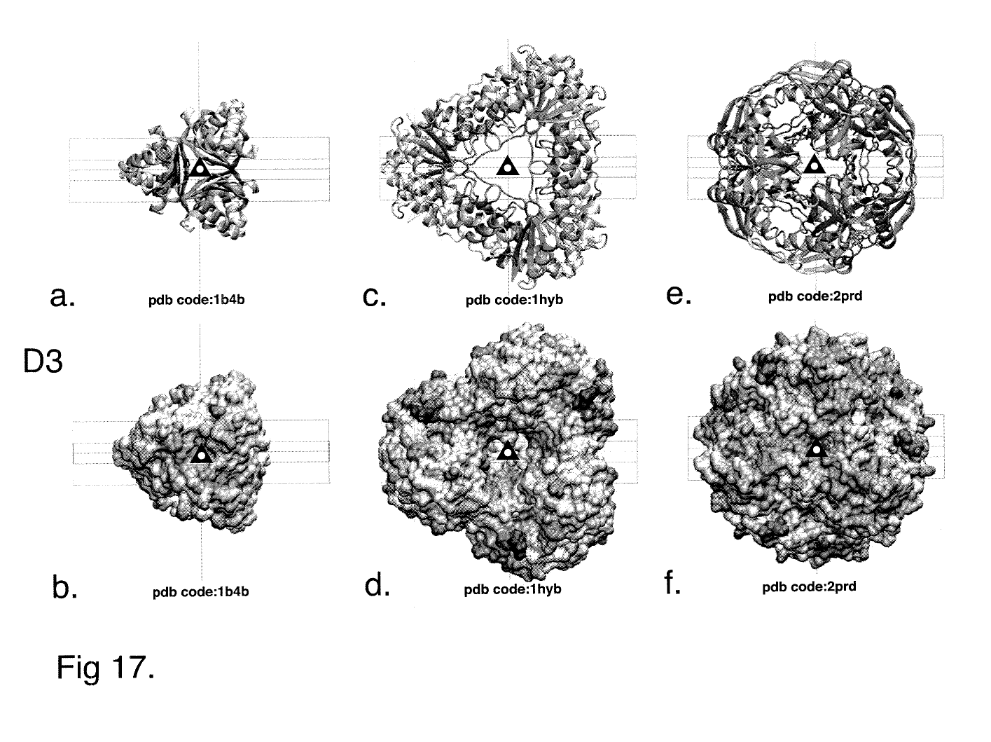

FIG. 15 presents schematic illustrations of a hexameric node with D3 symmetry and an octameric node with D4 symmetry. As noted above, nodes with Dn symmetry are particularly useful in the assembly of extended nanostructures since biotinylation sites can be introduced symmetrically across multimer dyad symmetry axes to precisely complement dyad-related biotin binding sites on streptavidin (FIG. 1). As schematically illustrated in FIG. 15a,b,c,d, this will generally involve introduction of a single site-specific modification in each polypeptide chain of the multimer to introduce a suitable biotinylation site. Note that for multimers with D3 and higher symmetry, there may be several different dyad-related symmetrical sites on the individual multimer subunits that are potentially complementary to the streptavidin dyad-symmetric binding sites. For example, the subunits of the D3 node shown in FIG. 15e are shown with biotinylation modification sites on their “faces”, but alternative symmetric sites occur that “bridge” between subunits. The situation is similar for the D4 node shown in FIG. 15f, except that the 4-fold symmetry creates an additional, symmetrically non-equivalent, set of dyad axes (labeled x′ and y′ in FIG. 15f) in the structure. If the Dn multimer structures are sufficiently large, it may be possible to introduce 2 biotinylation sites into each polypeptide chain of a Dn multimer (FIG. 16 a,b and FIG. 16 c,d) that are related by multimer dyad symmetry elements and complementary to the dyad symmetry of the biotin binding sites on streptavidin. The resulting structures shown in FIG. 16e (D3) and FIG. 16f (D4) could bind 6 and 8 streptavidin strut elements, respectively. FIG. 17 presents illustrations of hexameric protein multimers with D3 symmetry and octameric proteins with D4 symmetry useful as node templates.

FIG. 17 presents illustrations of some D3 symmetric protein hexamers useful as node templates. FIG. 17a shows the arginine repressor protein from Bacillus stearothermophilus. (Ni et. al. 1999, pdb code: 1b4b) in schematic backbone representation and in surface representation (FIG. 17b). FIG. 17c shows the adenylyltransferase protein from Methanobacterium thermoautotrophicum (Saridakis et. al 2001, pdb code: 1hyb) in schematic backbone representation and in surface representation (FIG. 17d). FIG. 17e shows the inorganic pyrophosphatase protein from Thermus thermophilus (Teplyakov et. al. 1994, pdb code: 2prd) in schematic backbone representation and in surface representation (FIG. 17f). Also shown in FIG. 17 a through f are “bounding boxes” that are aligned along the dyad symmetry axes of the respective D3 symmetric hexamers. The intersections between the molecular surface and the diagonal edges of the box define sites that are symmetric and complementary to the biotin binding in streptavidin, as described in more detail below. Additional structures with D3 symmetry are listed in Table 1.

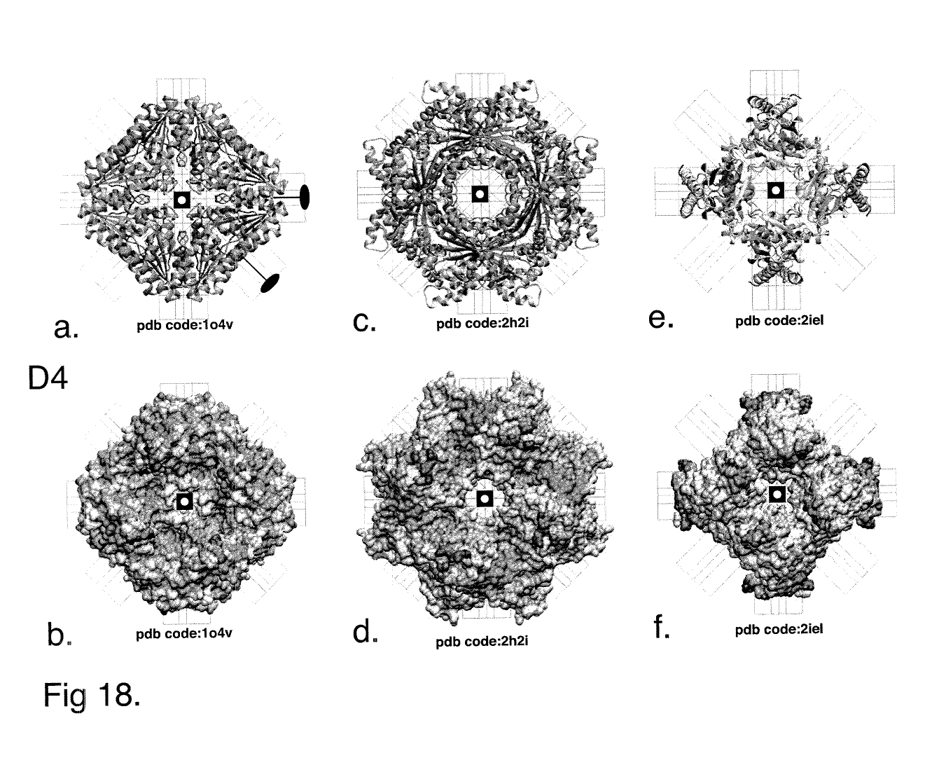

FIG. 18 presents illustrations of some D4 symmetric protein octamers useful as node templates. FIG. 18a shows the PurE protein from Thermotoga maritima (Schwarzenbacher et. al. 2004, pdb code: 1o4v) in schematic backbone representation and in surface representation (FIG. 18b). FIG. 18c shows the sirtuin protein from Thermotoga maritima (Cosgrove et. al. 2006, pdb code: 2h2i) in schematic backbone representation and in surface representation (FIG. 18d). FIG. 18e shows the TT0030 protein from Thermus Thermophilus (Zhu et. al. 2006, pdb code: 2iel) in schematic backbone representation and in surface representation (FIG. 18f). Also shown in FIG. 18 a through f are “bounding boxes” that are aligned along the dyad symmetry axes of the respective D4 symmetric octamers. The intersections between the molecular surface and the diagonal edges of the box define sites that are symmetric and complementary to the biotin binding in streptavidin, as described in more detail below. Additional structures with D4 symmetry are listed in Table 1.

In addition to multimers with D3 and D4 symmetry, multimers with higher Dn symmetry are also found in thermophilic organisms (Table 1). These protein multimers have utility as node templates in applications where nanostructures with certain geometrical properties and higher node connectivity is desired than is possible using nodes with D3 and D4 symmetry

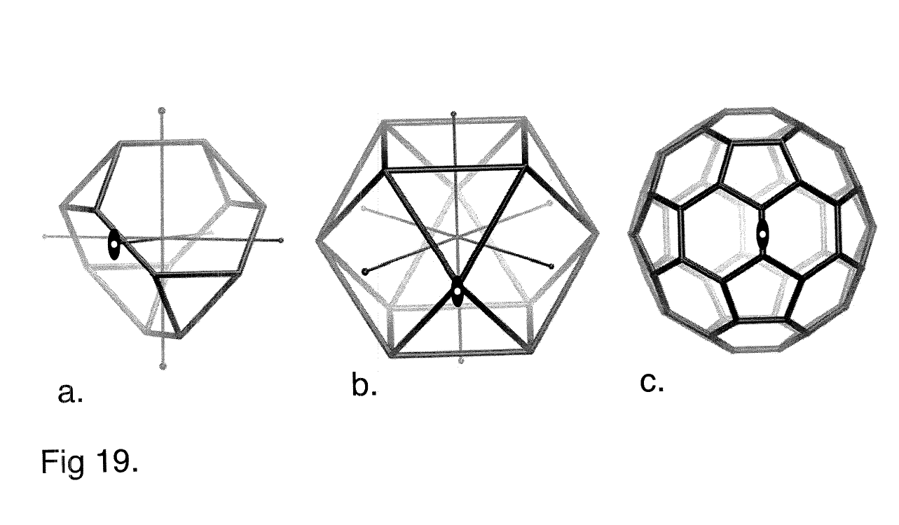

Nodes with Polygonal Symmetry: In addition to nodes with Dn symmetry, several occurrences exist of symmetric multimeric protein complexes with tetrahedral (usually incorporating 12 protein subunits), cubeoctahedral symmetry (usually incorporating 24 protein subunits), or icosahedral symmetry (usually incorporating 20n subunits). The surfaces of these multimers, which usually form hollow shell structures, range from nearly spherical, to shapes that approximate truncated tetrahedra. As shown schematically in FIG. 19, all of these polyhedra incorporate dyad symmetry elements. For example, FIG. 19a shows a truncated tetrahedron, FIG. 19b shows a cubeoctahedron, and FIG. 19c shows an icosahedron, together with their dyad symmetry axes. Connections made along the dyad axes of these polyhedra can be used to generate structures with features that radiate in three dimensions from a central node. Such dendritic structures may find application in new materials. Some modified polyhedra may serve as nodes in regular 3-dimensional lattices.

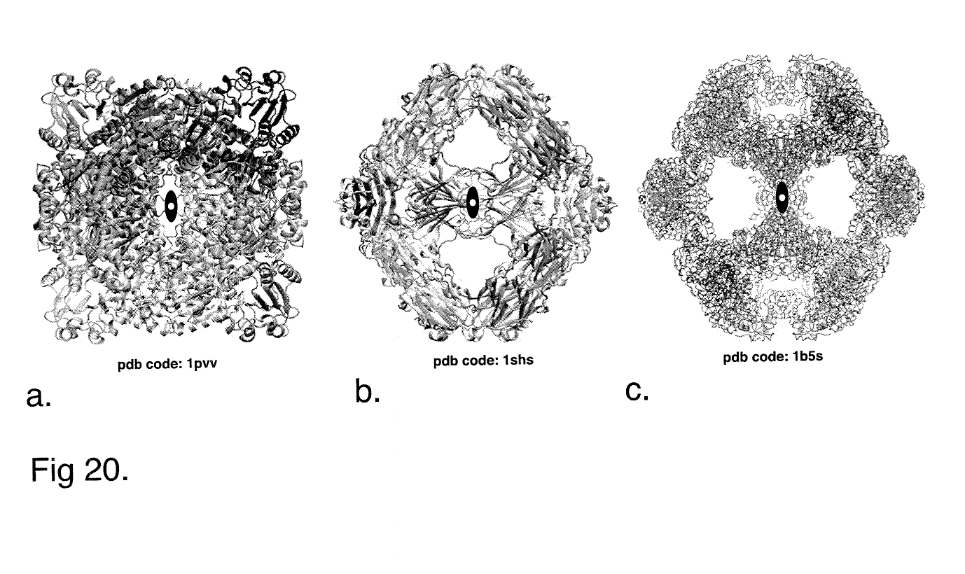

FIG. 20 presents illustrations of protein multimers having the symmetry properties of regular polyhedra and utility as templates for nanostructure node proteins. FIG. 20a shows the ornithine carbamoyltransferase dodecameric tetrahedral protein complex protein from Pyrococcus furiosus (Massant et. al. 2003, pdb code: 1pvv) in schematic backbone representation. FIG. 20b shows the 24-subunit cubeoctahedral heat shock protein complex from Methanococcus jannaschii, (Kim et. al. 1998 pdb code: 1shs) in schematic backbone representation. FIG. 20c shows the 60-subunit dodecahedral protein complex of the dihydrolipoyl transacetylase catalytic domain (residues 184-425) from Bacillus stearothermophilus (Izard, et. al. 1999, pdb code: 1b5s) in schematic backbone representation. Additional structures with polyhedral symmetry are listed in Table 1.

Method of Determining Sites of Site-Specific Modifications on Proteins Suitable for Production of Multimeric Node Proteins with Geometrically Defined Attachment Points for Binding Streptavidin

In general, protein multimers suitable for use as node templates can be composed of two or more protein subunits related by symmetry. Node proteins are created by using site-specific mutagenesis to introduce reactive amino acids at specific sites on the template node protein surface that can be subsequently functionalized to allow the geometrically defined attachment of a linear strut through chemical linkages or non-covalent interactions between specific sites on the node and strut. In the current application, the envisioned nanostructures will incorporate streptavidin as a strut, or streptavidin in complex with other proteins that can preserve certain binding and geometrical features of the streptavidin tetramer as outlined above (FIG. 1, FIG. 12) and described elsewhere (Salemme & Weber, 2007). As shown in FIG. 1, streptavidin is a protein tetramer with D2 symmetry that incorporates 4 binding sites for the vitamin biotin. Node proteins suitable for binding streptavidin are template proteins that have been modified through site-specific modification to allow covalent reaction of specific amino acid side chains to covalently attach biotin groups to the node protein.

Many amino acids can potentially be introduced as sites for specific chemical modification on the template node protein surface, including cysteine, methionine, lysine, histidine, tyrosine and arginine. Any other occurrences of an amino acid of a type that is to be introduced through site-specific modification on the node template surface must also be modified through site-specific mutagenesis by substituting a structurally similar amino acid, so that the final node protein subunit sequence incorporates reactive amino acids only at those sites that facilitate the predefined node-strut geometry.

In the present embodiments, the node structures are modified to incorporate cysteine residues, which can be modified with suitable reagents to incorporate covalently bound biotin groups able to bind streptavidin with defined geometry and high affinity (FIG. 2). Cysteine residues occurring on the surface in the naturally occurring sequence of the node template protein are substituted with serine, alanine or another amino acid depending upon the local structural environment.

FIG. 1 illustrates the structure of streptavidin, a D2 tetramer whose subunits are related by three mutually perpendicular two-fold rotational (dyad) axes of symmetry. As illustrated, the biotin binding sites on streptavidin (or more specifically the coordinates of the protein-bound biotin carboxyl oxygen atoms that are the sites of bifunctional chemical reagent attachment) are separated by 20.5 Angstroms and oriented along a line at an angle of 20 degrees relative to the “y” dyad axis of the streptavidin tetramer (as defined in FIG. 1) and at an angle of 72 degrees relative to the “z” dyad axis of the streptavidin tetramer. In general, maximum precision and flexibility in the assembly of streptavidin-linked structures is achieved when the bound streptavidin is positioned with defined geometry relative to the node to which it is bound. For nodes with Cn symmetry, the bound streptavidin should be aligned so that either the y or z-dyad axes of the streptavidin tetramer are aligned parallel with the Cn symmetry axes of Cn symmetric nodes. For nodes with D2 symmetry, the y or z-dyad axes of the streptavidin tetramer are aligned with one of the D2 dyad axes, and the streptavidin x dyad axis is coincident with a second dyad axis of the D2 node (Although there are some exceptions to this rule as shown in FIG. 13). For nodes with Dn symmetry, the y or z-dyad axes of the streptavidin tetramer is aligned with the Dn axis of the node and the streptavidin x dyad axis is coincident with a dyad axis of the Dn node. For polyhedral nodes with dyad axes, the y or z-dyad axes of the streptavidin tetramer are aligned with a major symmetry axis of the node (depending on the polyhedral node symmetry), and the streptavidin x-dyad axis is coincident with a dyad axis of the polyhedral node.

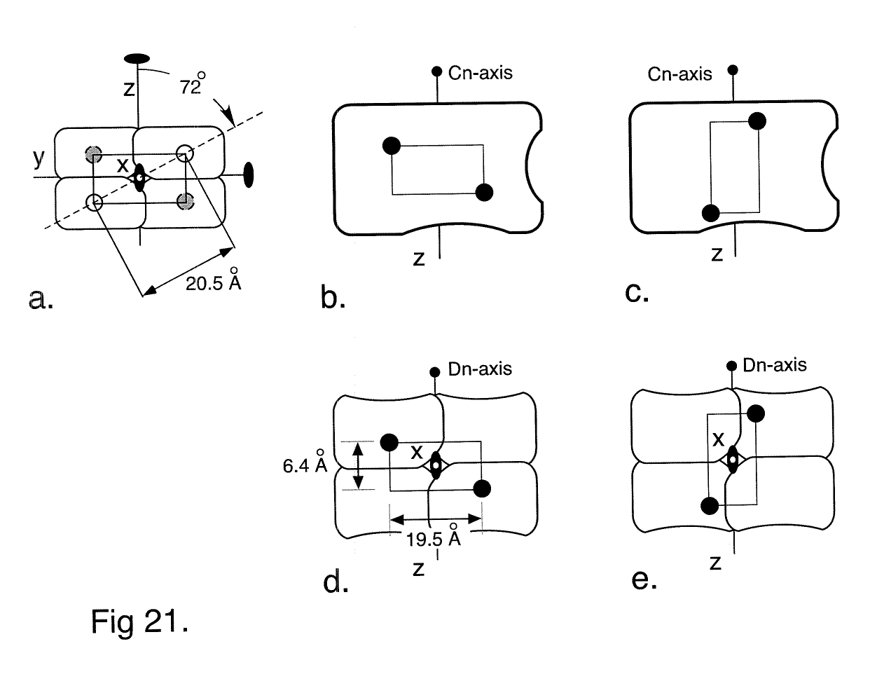

FIG. 21a reiterates the geometry of the biotin binding sites on streptavidin. FIGS. 21b and 21c show schematic views of a node with Cn rotational symmetry, where the Cn symmetry axis defines the z-axis of the structure. In order to assemble extended structures that do not twist when interconnected by streptavidin, the geometry of site-specific modifications on the node template (or more specifically the coordinates of the thiol sulfur atoms of the incorporated cysteine side chains on the node protein) must be complementary to the geometry of the biotin binding sites on streptavidin, and must align the streptavidin z-axis (FIG. 21a) or y-axis (FIG. 21b) with the node Cn or z-axis This requires that the modification sites on the nodes are oriented at an angle (e.g. −72 degrees) relative to the Cn rotational (z) axis of the node protein complex so that the modification sites are complementary to the biotin binding sites on streptavidin. There can be some variation (20.5 plus or minus 5 Angstroms) in the separation of the node modification sites, since this variation can be accommodated by adjusting the length of the chemical linking reagent that couples the biotin to the cysteine sulfhydryl groups. However, significant (>5 degrees) variations in the angle from −72 degrees will accumulate to cause extended structures to twist and potentially introduce strain into extended structures.

The above criteria represent general requirements for the assembly of any planar structure incorporating streptavidin struts and nodes with Cn rotational symmetry. It is notable that in the prototype 2-dimensional lattice structure assembled by Ringler and Schulz (2003), the two cysteine residues (Asn 133 to Cys, and Lys 261 to Cys) introduced through site-specific modification on their C4 node protein template, L-rhamnulose-1-phosphate aldolase from E. coli, are oriented at 52 degrees relative to the C4 axis of the tetramer, giving each bound streptavidin a slight “propeller” twist relative to the central node. It is consequently evident that extended structures must have been quite strained, and that this was an important contributing factor to their inability to build very extensive 2-dimensional lattices.

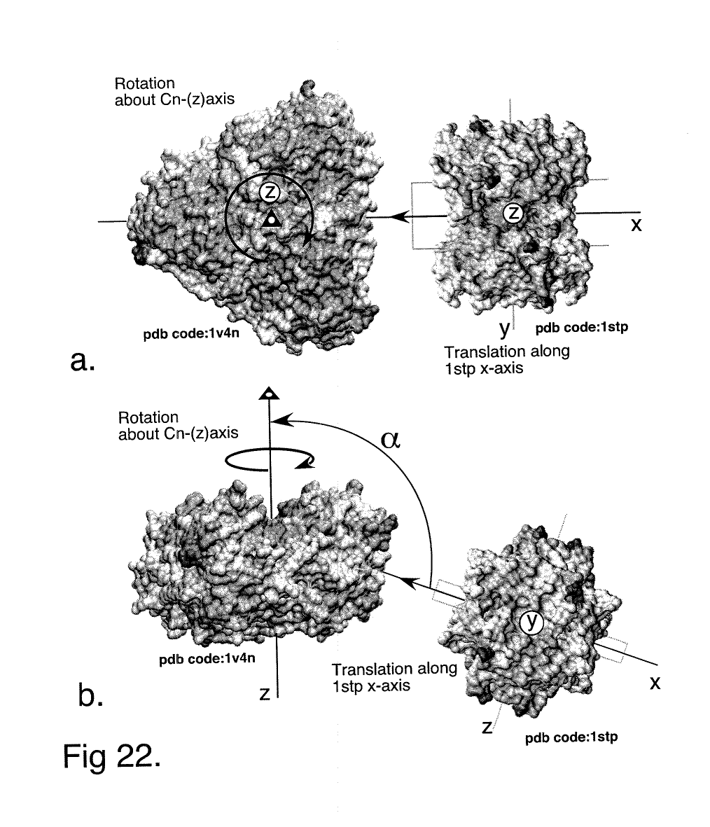

Cn Symmetric Node Specification: Definition of the sites for site-specific modification on Cn symmetric node templates can be determined using computer modeling, computational methods or a combination of these methods. Generally the methods involve a constrained geometrical search for favorable interaction complexes. FIG. 22 schematically illustrates the variable search parameters for Cn and Dn node structures. The Cn search parameters include a rotation of the Cn node about its z-axis, and a translation of streptavidin along its x-axis in the xy plane of the node (FIG. 22a). The method involves initially orienting the Cn template node and streptavidin so that they a) do not spatially overlap, b) are oriented with the Cn (z-axis) of the node parallel to either the y-axis or z-axis of streptavidin, and c) have similar z coordinate values for their respective centers of mass. The node is incrementally rotated about the Cn axis through an angular range somewhat greater than 360/n degrees. For each angular increment about the Cn axis, the streptavidin tetramer is translated along its dyad x-axis until van der Waals contact contact or near van der Waals contact is made between the atomic coordinates of the node template and atomic coordinates of streptavidin. Each of the resulting streptavidin-node complexes is then examined using computer graphics (Jones et. al. 1990, Humphry et. al. 1996), geometrical, energetic computational methods (Case et. al. 2005), or a combination of these methods to determine the quality of overall fit and feasibility and locations of site-specific modifications on the node template that can provide chemical attachment points for biotin, including the use of coupling reagents with different linker lengths. Parameters describing the complex structure (e.g 3-dimensional model coordinates, computed energy, pictures, etc.) may be then entered into a table. The process outlined above is repeated for relatively small incremental changes in rotation around the template node Cn symmetry axis (for example, about 0.1 to 2.0 degrees in rotation), so that interactions of the Cn node surface and streptavidin are extensively sampled, evaluated and compared. Complexes with the best features are then selected for manufacture using recombinant DNA technology.

Cn Polyhedral Node Specification: The method outlined above is suitable for nodes that are incorporated into essentially planar, 2-dimensional structures oriented on surfaces. Similar constrained searches can be developed to design nodes for the assembly of 3-dimensional structures. For example, nodes can be designed that can assemble into 3-dimensional polyhedra that such as a regular a regular dodecahedron incorporating C3 symmetric nodes or a regular icosahedron incorporating C5 symmetric nodes (FIG. 11). Additional polyhedral nodes are possible as well. The approach to defining sites for modification is similar to that outlined above for Cn planar nodes, except that the orientation of the approach axis between streptavidin and the Cn axis of the node complex is not 90 degrees, but is the angle α formed between the edge of the polyhedron and a vector from the center of the polyhedron to an apical node (FIG. 22b). As outlined above, the apex angle α for an icosahedron is approximately 121.92 degrees (FIG. 11a) and for a dodecahedron α is approximately 110.93 degrees (FIG. 11b). Similar considerations apply to the generation of other regular and irregular polyhedra, such as buckyballs (truncated icosahedron) and buckytubes (Weber 2199) where n=3 and the approximate apex angle α=z 104.15 degrees.

Dn Node Specification: Nodes based on node templates with Dn symmetry represent an extensive family with diverse structural geometry (Table 1). As noted above, structures with dyad symmetry axes such as Dn symmetric structures offer the possibility of symmetric placement of biotin linkage sites on node subunits that are complementary to the binding sites on streptavidin. The process generally produces node subunit proteins that incorporate only a single site-specific modification for the purposes of incorporating a reactive cysteine residue, so that the bound streptavidin tetramer in the complex forms a symmetric link between node subunits oriented by a dyad axis of symmetry.

As outlined in FIGS. 21d and 21e, there are generally two symmetric orientations that are best suited for the formation of extended structures composed of Dn symmetric nodes. These alternative orientations correspond to streptavidin alignments where either the streptavidin z-axis (FIG. 21d, “H” or horizontal) or y-axis (FIG. 21e “V” or vertical) is oriented parallel to the node Dn or z-axis. Definition of the sites for site-specific modification on Dn symmetric node templates can be determined using a constrained computer search (FIG. 21c) where a) the z-axis of the streptavidin tetramer and either the x, y, or z-dyad axes of D2 node are constrained to be parallel, and b) the approach x-axis of streptavidin (which is a dyad axis) is constrained to be coincident with a dyad axis relating subunits of the Dn-symmetric node template. Final complex configurations are those where the atoms of streptavidin and the node make Van der Waals contact or near Van der Waals contact.

Nodes based on node templates with D2 symmetry are appropriate for many applications including formation of 2D and 3D lattices, as well as for strut extenders that connect two streptavidin tetramers in a linear array (FIG. 12b,d). Definition of the sites for site-specific modification on D2 symmetric node templates can be determined using a constrained computer search as outlined above, noting however, that since the D2 node has three mutually perpendicular dyad axes, and that there are 2 alternative streptavidin orientations around each dyad, that there are potentially a total of six possible complex configurations where streptavidin can be symmetrically bonded to a D2 node so that its dyad symmetry axes are coincident and/or perpendicular to the symmetry axes of the node. As examples, FIG. 12a illustrates the case where the streptavidin z-dyad axis is parallel to the z-dyad axis of a D2 node, while FIG. 12c illustrates the example where the streptavidin y-dyad axis is parallel to the z-dyad axis of a D2 node.

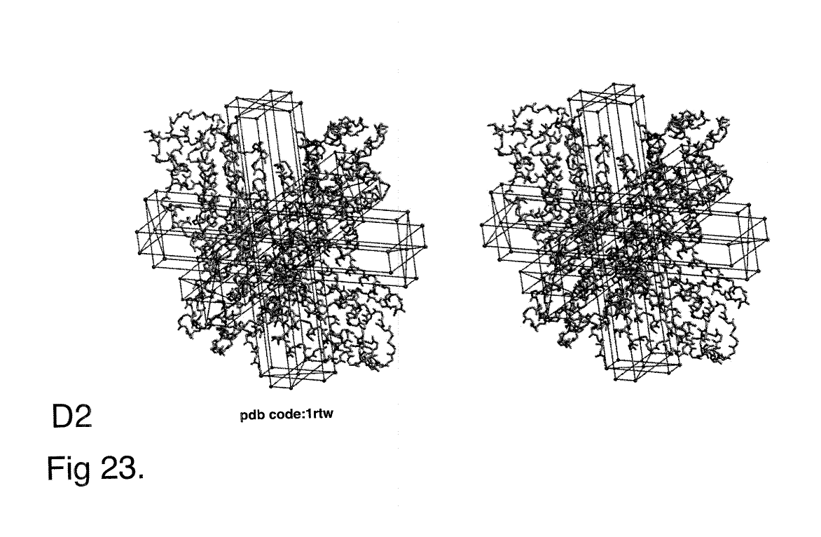

Locating the positions on a Dn node surface suitable for the introduction cysteine residues for biotinylation may also be performed through an alternative graphical or mathematical process. Basically this involves the superposition of “bounding boxes” (with dimensions of approximately 6.4 Angstroms by 19.5 Angstroms, FIG. 21d.) that represent the projected positions of the potential biotinylation sites (e.g. sites complementary to the biotin bonding sites in each of the 2 possible streptavidin binding orientations) around each dyad axis in a structure. For example, FIG. 23 shows a stereoscopic view of the D2 symmetric node template pdb code: 1 rtw with pairs of bounding boxes embedded along each of the three dyad axes. By examination of the 3-dimensional atomic coordinates of the node template protein using a computer method (Lee and Richards, 1971) it is possible to compile a list of the coordinates of the atoms and/or amino acid residue side chains that lie on the surface of a protein. Specific side chain atoms can be selected for reference in a list; e.g. Cα, back bone carbon atoms for Gly residues, and Cβ side chain atoms for all other amino acid residues. A computer program can then be used to find the shortest distances between selected amino acid side chain atoms in the exposed atom/residue list and the lines defining the bounding box that project the positions of the biotin binding sites. Alternatively, the Cβ atoms can be identified by inspection using computer graphics modeling programs. The atoms so identified will generally define the amino acid residues in the template sequence that can be mutated to Cys residues, and when functionalized by biotinylation, will form sites that are symmetric to streptavidin and align the Dn axis of the node to either the y-axis or z-axis of streptavidin.

Several of the multimeric nodes shown in this application are shown with embedded bounding boxes (in projection) along node dyad axes.

For D2 nodes with appropriate geometrical features, alternate linear couplers can be engineered that introduce twist between the streptavidin tetramers linked to the D2 node along the complex x-axis (FIG. 13). Identification of modifications sites on the node template involves a process that is slightly different from that described above, where the search (or alternatively, the rotational orientation of the bounding boxes around the complex x-axis) is performed with the z-axes of the streptavidin tetramer and the D2 node oriented at some predetermined angle β (FIG. 13a), depending on the total angular twist desired in the final linear coupler. FIG. 13ab shows a special case where a D2 node with nearly tetrahedral symmetry is modified to produce a D2 linear coupler that orients the terminal streptavidin molecules with β=90 degrees.

Additional nodes, appropriate for the formation of extended 3-dimensional lattices, can be based on node templates with D3 or D4 symmetry as detailed below. Definition of the sites for site-specific modification on Dn symmetric node templates can be determined using a constrained computer search process similar to that described above for Cn nodes, where the orientation of the approach axis between streptavidin and the Dn axis of the node complex is 90 degrees, but the search is additionally constrained so that the approach axis along which the streptavidin molecule advanced is coincident with a dyad axis relating subunits of the Dn-symmetric node template. Note that this process generally produces node subunit proteins that incorporate only a single site-specific modification, so that the streptavidin tetramer in the complex forms a symmetric link between node subunits oriented by a dyad axis of symmetry.

Polyhedral Node Specification: Additional nodes, appropriate for the formation of extended 3-dimensional radial structures or 3-dimensional lattices, can be based on node templates with higher symmetry that incorporate dyad symmetry elements. Observed node symmetries include tetrahedral, cubic, cuboctahedral, and truncated icosahedral (Table 1). Definition of the sites for site-specific modification on these higher symmetry node templates can be determined using a constrained computer search process similar to that described above for D2 nodes, where the orientation of the x approach axis of streptavidin is constrained to be coincident with a dyad axis relating subunits of the symmetric node template. Note that this process generally produces node subunit proteins that incorporate only a single site-specific modification per subunit, so that the streptavidin tetramers in the complex form symmetric links between node subunits oriented by a dyad axis of symmetry.

For any given modeled complex it may be possible, using computational and modeling methods (Jones 1990, Case et. al. 1995), to further improve the complex through the introduction of site-specific modifications in streptavidin or the template node to improve electrostatic complementarity, van der Waals interactions or other features that will improve the stability or functionality of the complex.

### Examples of Specific Node Embodiments

The sequence and symmetry specifications of the several embodiments described below are detailed in Table 2. Table 2 provides the Protein Data Bank code (pdb code) for the node template structure, the node symmetry, the amino acid sequence of the node template (as downloaded from the Protein Data Bank), and the modifications of the sequence that are required to create a node that can be functionalized by biotinylation so that it interacts with streptavidin or other proteins with binding sites disposed with the same geometry as the streptavidin binding sites (Salemme & Weber 2007). Sequence modifications are grouped as “general” and “specific biotinylation sites”. General sequence modifications usually represent modifications to replace potentially interfering cysteine residues occurring in a template sequence with structurally similar residues. Depending on the structural environment and role of the cysteine side chain in the template protein, the replacement amino acid may be Ala, Ser, H is, Asp, or potentially some other amino acid. Additional sequence modifications that “generally” alter the template protein sequence could include terminal modifications and/or the introduction of subunit linking polypeptide sequences to create single-chain structures. Note that many proteins expressed in E. coli are modified by addition of an N-terminal methionine residue, which is by often counted as residue “zero” of the polypeptide chain for structural purposes and so designated in Protein Data Bank (pdb) coordinate files. In any case, residues designated as sites of modification in Table 2 correspond to the sequence numbering provided in the designated pdb file containing the structural coordinates of the node template.

Specific biotinylation sites are sites for the introduction of Cys residues into the template sequence that will provide optimal geometry and, for Dn and tetrahedral nodes, symmetric placement of the biotinylation sites around the node dyad symmetry axes. The locations of these sites were determined by use of the computer graphical and computational methods defined above. As noted above in FIG. 21 there are generally two orthogonal orientations that streptavidin can take with respect to the major symmetry axes of complexes with Cn, Dn or higher symmetry. For Cn nodes, these are enumerated as “H” and “V” where the y-axis or z-axis of streptavidin is parallel to the node Cn axis, respectively (FIG. 21b,c). Since streptavidin tetramer makes an asymmetric interaction with Cn node, there are potentially a large number of possible complementary interactions that are feasible for a Cn-node streptavidin-strut interaction. Table 1 generally shows one “H” interaction that analysis suggests provides the best steric and charge complimentarity between streptavidin and the node surface.

As noted above, nodes with Dn or higher symmetry offer the possibility of aligning the dyad symmetry axes of streptavidin with dyad symmetry axes of the node. These are enumerated as “H” and “V” along diad axes (x, y, or z) of a Dn or higher symmetry node (FIG. 21d,e). D2 nodes have three dyad axes, so there are a total of 6 orientations by which streptavidin can be attached to a D2 node. Although it is not physically possible to bind streptavidin in both “H” and “V” orientations simultaneously, there nevertheless arise a large number of combinations for node streptavidin complexes that are possible as outlined in Table 2 G, H, I, J. D3 nodes are special, since interactions made at one end of the dyad axis are different from the other (FIG. 15e, FIG. 17), so that there are a total of 4 possible streptavidin node interaction geometries, producing a total of 8 strut-node interaction patterns (Table 2 KLM). For D4 symmetric nodes (FIG. 15f, FIG. 18) there are two independent dyad axes, giving a total 8 different streptavidin substitution patterns (Table 2 NOP). For tetrahedral nodes, all the dyad axes are symmetrically equivalent, so that there are only 2 possible node:streptavidin orientations possible (Table 2Q).

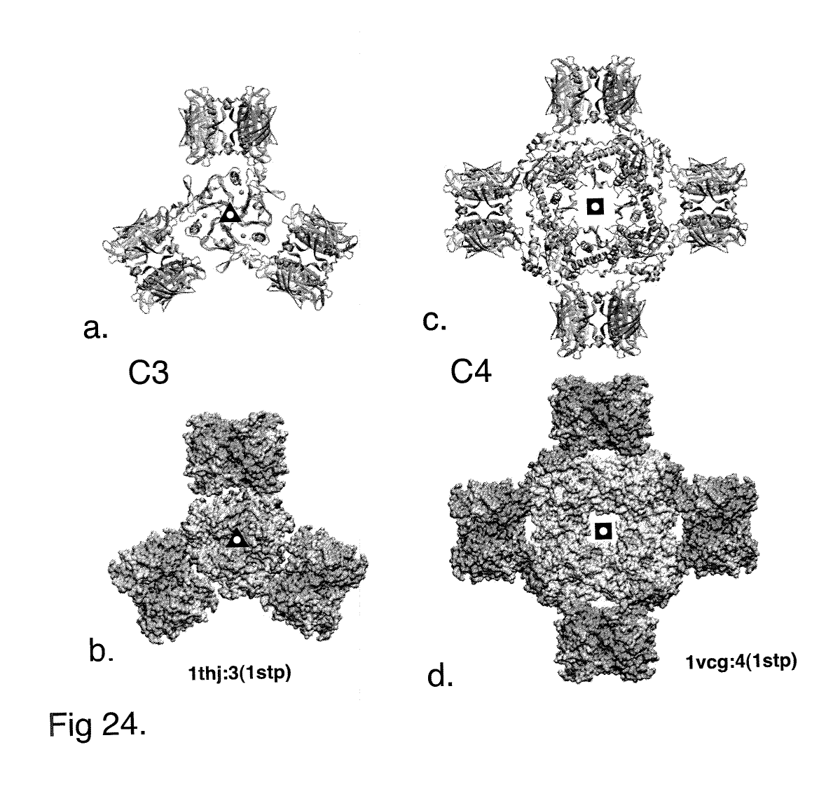

Three-Fold (C3) Symmetric Planar Node: FIGS. 24a and 24b respectively show a schematic view and space filling view of a node based on the previously described trimeric C3 symmetric protein 1thj, in covalent complex with 3 bound molecules of streptavidin. (In this an succeeding figures of such complexes, the biotins bound to streptavidin are shown in space filling representation in the schematic diagrams although atomic coordinates for linking atoms or amino acid side chains residues are not shown for simplicity.) Although there are several potential sites of interaction between the surface of 1 thj and streptavidin that can be generated using the methods described above, the one shown corresponds to a node construct where a Cys148 to Ala modification and specific biotinylation sites have been introduced at sequence positions 70 (Asp70 to Cys) and 200 (Tyr200 to Cys) in the 1thj polypeptide sequence (Table 2A).

Table 2C also provides a node specification of for C3 trimeric planar node based on the 1j5s protein described above.

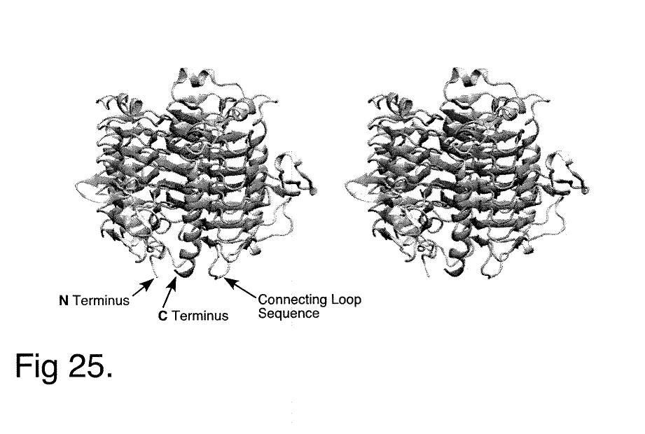

Single Chain Variants of Three-Fold (C3) Symmetric Planar Node: FIG. 25 shows a stereoscopic view of a single chain variant of the 1thj trimer. The sequence of the single-chain trimer incorporates three 207-residue amino acid sequences derived from the original 1thj sequence that are interconnected by two seven residue linkers. Table 2B gives sequence specifications for the trimer variants with both symmetric and asymmetric binding sites for streptavidin as schematically illustrated in FIG. 4c,d,e.

Four-Fold (C4) Symmetric Planar Node: FIGS. 24c and 24d respectively show a schematic view and space filling view of a node based on the previously described trimeric C4 symmetric protein 1vcg, in covalent complex with 4 bound molecules of streptavidin. Although there are several potential sites of interaction between the surface of 1vcg and streptavidin that can be generated using the methods described above, the illustration shown corresponds to a node construct where Cys 14 and Cys236 modifications have been made and specific biotinylation sites have been introduced at sequence positions 44 (Ser44 to Cys) and 49 (Thr49 to Cys) in the 1vcg polypeptide sequence (Table 2E).

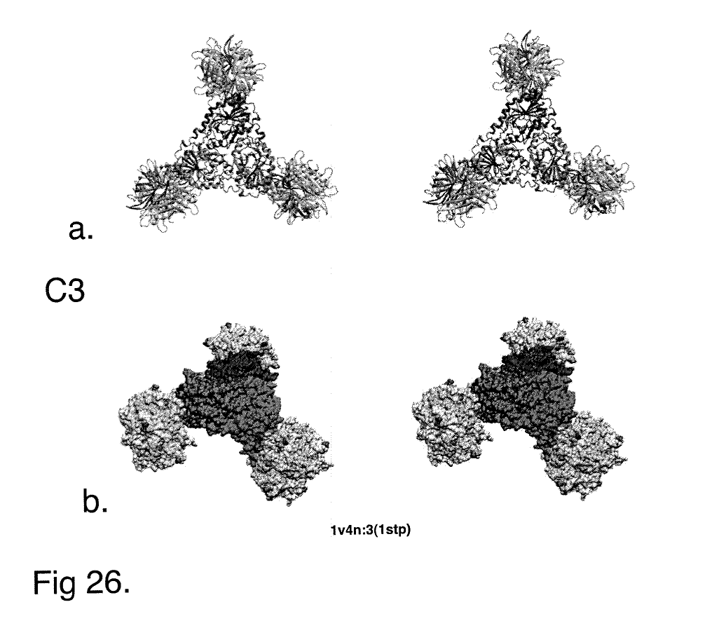

Three-Fold (C3) Symmetric Polyhedral Node: FIG. 26 shows schematic and surface stereoscopic views of a C3 symmetric node, with 3 streptavidin tetramers bound at angles corresponding to an dodecahedron apex (FIG. 11a). The dodecahedral polyhedral node is based upon the structure of a 5′-deoxy-5′-methylthioadenosine phosphorylase homologue from Sulfolobus tokodaii (Kitago et. al 2003) protein as the template node, and generated by the methods described above (pdb code: 1v4n). Specific sequence specifications are given in Table 2D. Table 2D also gives a specification for a “bucky” or truncated icosahedral apex node (See FIG. 19.c), based on 1v4n as the node template.

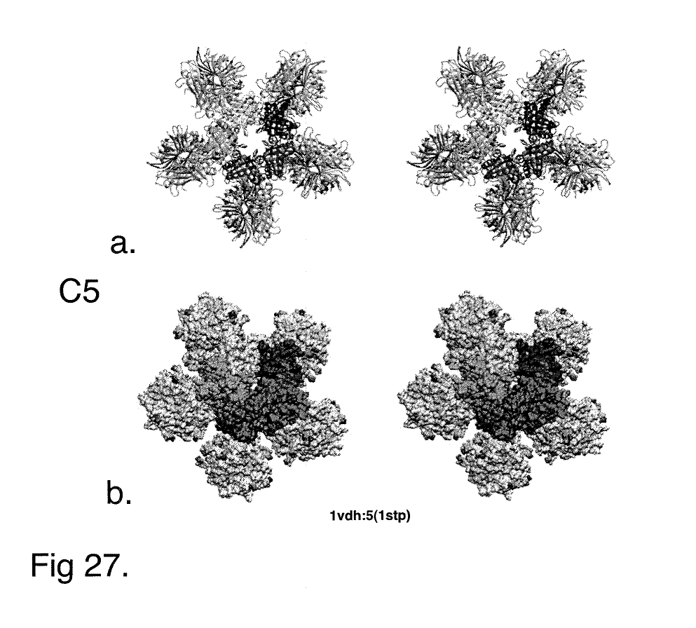

Five-Fold (C5) Symmetric Polyhedral Node: FIG. 27 shows schematic and surface stereoscopic views of a C5 symmetric node, with 5 streptavidin tetramers bound at angles corresponding to an icosahedron apex (FIG. 11a). The icosahedral polyhedral node is based upon the 1vdh protein (described above FIG. 10d) as the template node, and generated by the methods described above. Specific sequence specifications are given in Table 2F.

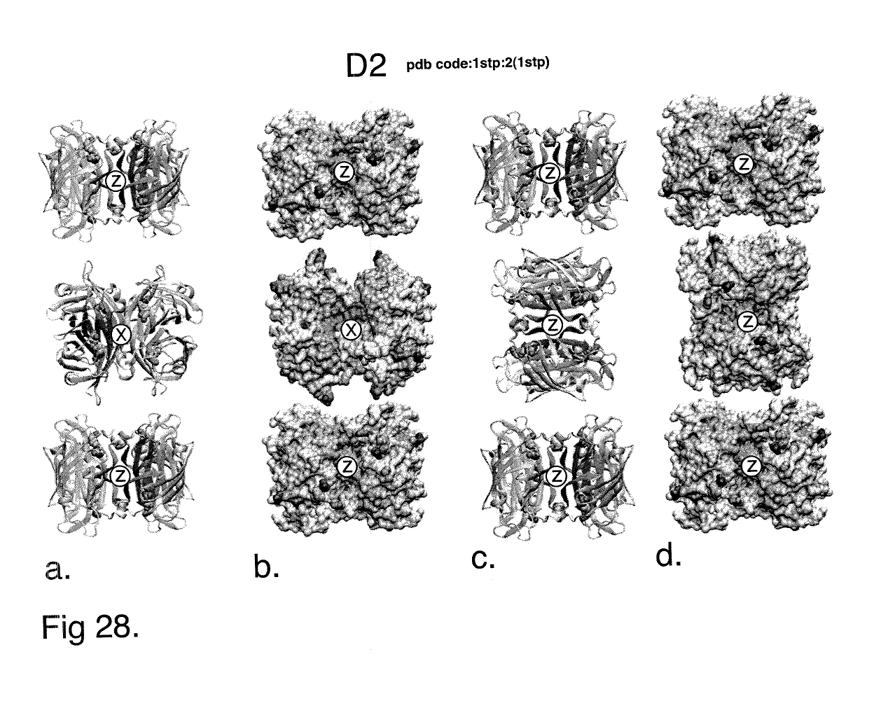

Streptavidin D2 Strut Coupler: As noted above (FIG. 1), streptavidin itself is a tetramer with D2 symmetry and can function as a node in the context of some assemblies. Although not specifically derived from a thermophilic bacterium, streptavidin is unusual for its thermostability both in its unliganded and biotin-bound forms (Weber et. al. 1989, 1992, 1994). FIG. 28 schematically shows streptavidin tetramers that have been modified through site-specific mutagenesis to incorporate four dyad symmetry-related biotinylation sites (e.g. surface cysteine residues), allowing in situ functionalization with biotin to allow the attachment of additional streptavidin tetramers. FIGS. 28a and b respectively show schematic and surface representations where the x-axis of the central “node” streptavidin tetramer is oriented parallel to the z-axes of 2 bound streptavidin tetramers. FIG. 28 c and d respectively show schematic and surface representations where the z-axis of the central “node” streptavidin tetramer is oriented parallel to the z-axes of 2 bound streptavidin tetramers. The specifications for the streptavidin “nodes” modified for binding streptavidin tetramers along streptavidin dyad are given in Table 2G. This method of attaching streptavidin-linked binding or other functional protein domains provides an additional means for creating functionalized struts in nanostructures. Table 2G also provides a specification for a streptavidin “node” with streptavidins bound along the “node” x-axis, so blocking access to the streptavidin “node” biotin binding sites. Such constructs may be useful when it is desirable to protect the “node” biotin binding sites during an intermediate stage of an assembly process.

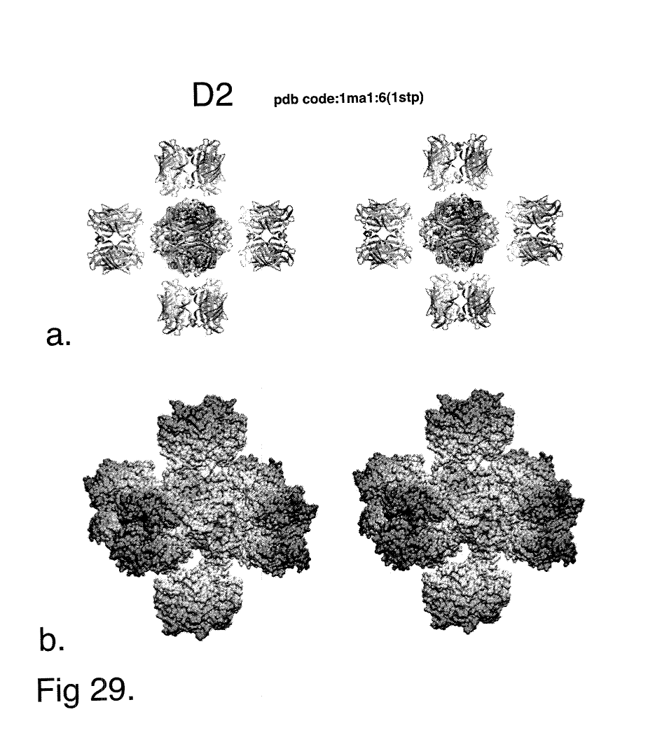

D2 Nodes: FIG. 29a,b show stereoscopic views of a tetrameric D2 node based on the 1ma1 node template in schematic and space filling representation respectively. There are 6 streptavidin tetramers bound to the node, two along each symmetrically independent dyad axis. Table 2H gives the specifications for variations in the 1m1a node based on different orientations of bound streptavidin tetramers (e.g. see FIG. 12,a,c) and combinations of biotinylation sites along each of the three independent node dyad axes. Variations in dyad axis site substitution patterns can produce nodes suitable for the formation of orthorhombic 3D lattices (e.g. the node shown in FIG. 29), 2D rectangular lattices, or linear strut extenders.

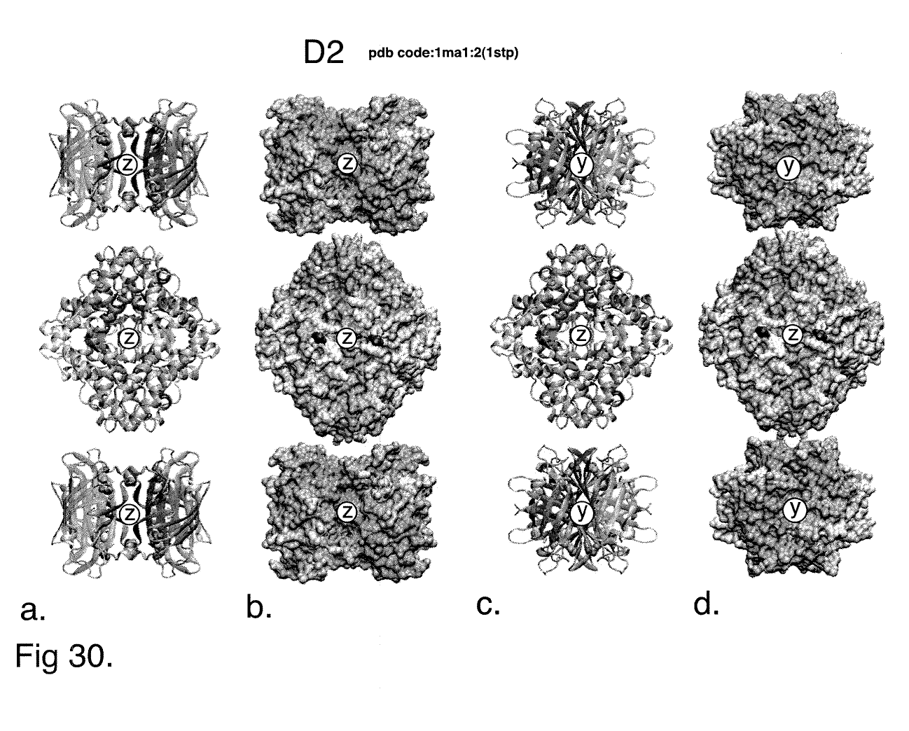

FIG. 30 shows illustrations in schematic and space filling representation of two examples of linear struts incorporating a D2 node based on 1ma1 and two streptavidin tetramers. In FIG. 30a,b streptavidin tetramers are oriented with their z-axes parallel to one of the D2 node dyad axes. In FIG. 30c,d streptavidin tetramers are oriented with their y-axes parallel to the same D2 node dyad axis. Since there are a total of three independent dyad axes, there are a total of six alternative linear strut complexes that can be formed with a D2 node and two streptavidin tetramers to form linear struts. The specifications for these nodes are included in Table 2H. Table 2I and 2J respectively provide additional specifications for D2 symmetric nodes based on the 1nto and 1rtw node templates.

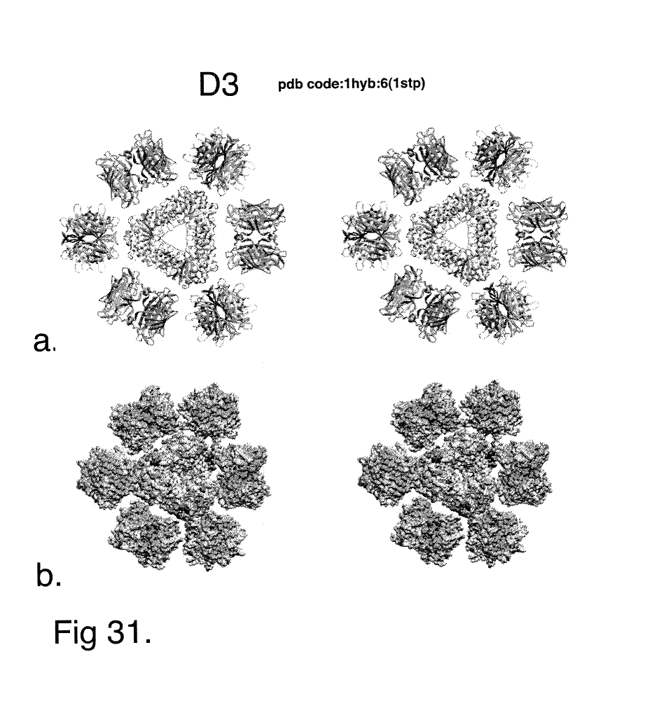

D3 Nodes: FIG. 31a,b show stereoscopic views of a hexameric D3 node based on the 1hyb node template in schematic and space filling representation respectively. There are 6 streptavidin tetramers bound to the node, including 3 tetramers with their y-axes oriented parallel to the D3 node symmetry axis and 3 tetramers with their z-axes oriented parallel to the D3 node symmetry axis. Note that the 2 “poles” of the D3 dyad axes (FIG. 15e) are symmetrically non-equivalent, and that variations can be produced with for example, with 3 bound streptavidin tetramers bound at either pole in either of two orientations (FIG. 12a,c). Table 2L gives the specifications for variations in the 1hyb node based on different orientations of bound streptavidin tetramers (e.g. see FIG. 12,a,c) and combinations of biotinylation sites at poles of the dyad axis. Tables 2K and 2M respectively provide additional specifications for D3 symmetric nodes based on the 1b4b and 2prd node templates.

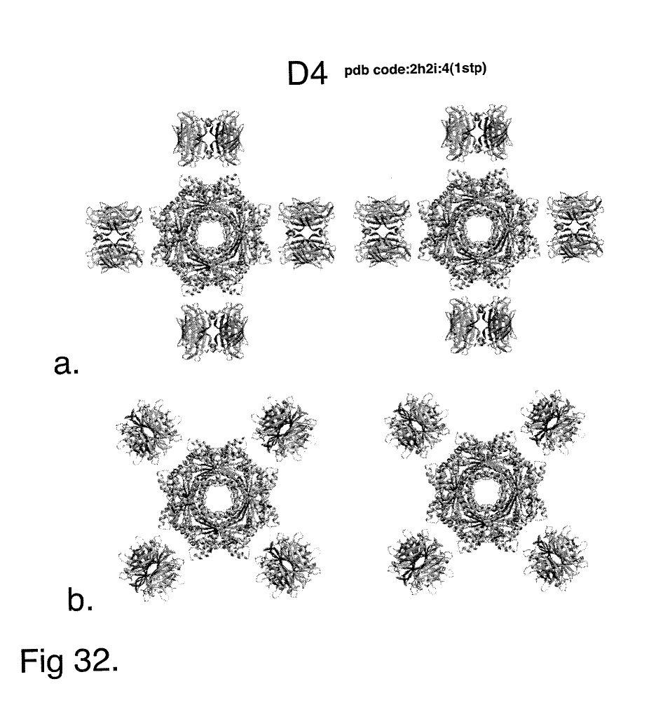

D4 Nodes: FIG. 32a,b show stereoscopic views of two octameric D4 node complexes based on the 2h21 node template in schematic representations. There are 4 streptavidin tetramers bound to each node, along the two symmetrically non-equivalent axes of the D4 node (FIG. 15f). The complex shown in FIG. 31a incorporates streptavidin tetramers with their z-axes oriented parallel to the D4 node symmetry axis, while the complex shown in FIG. 31b incorporates streptavidin tetramers with their y-axes oriented parallel to the D4 node symmetry axis. Table 20 gives the sequence specifications for variations the 2h21 node based on different orientations of bound streptavidin tetramers (e.g. see FIG. 12,a,c) and combinations of biotinylation sites along the symmetrically non-equivalent dyad axes. Tables 2N and 2P respectively provide additional sequence specifications for D4 symmetric nodes based on the 1o4v and 2iel node templates.

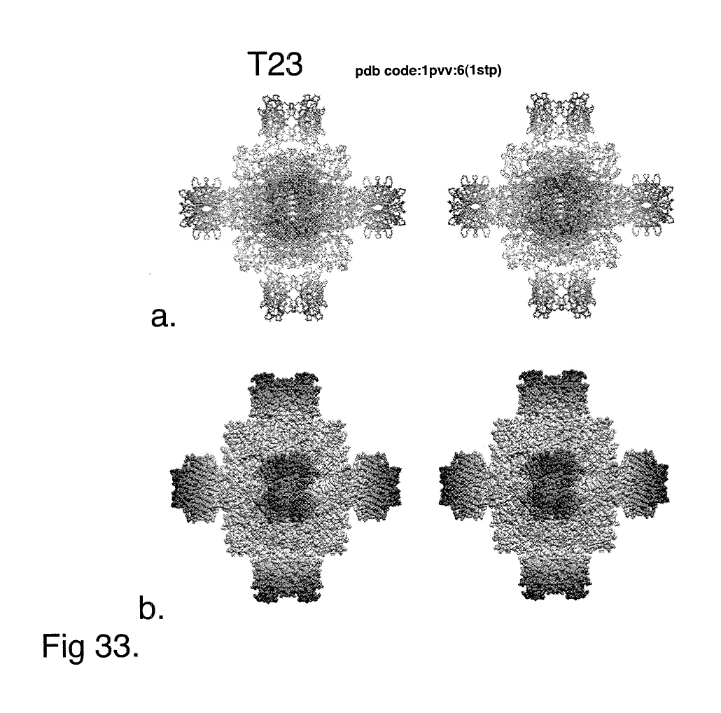

Tetrahedral (Cubic Lattice) Node: FIG. 33a,b show stereoscopic backbone and space-filling views of a dodecameric (T23) tetrahedral node based on the 1pvv node template in complex with 6 streptavidin complexes bound along the 3 symmetrically equivalent, mutually perpendicular dyad axes of the structure. Table 2Q gives the sequence specifications for the 2 possible binding orientations for streptavidin to the node along the dyad axis.

Examples of One-Dimensional, Two-Dimensional and Three Dimensional Assemblies Constructed with Streptavidin Struts and Nodes of Different Symmetry.

The following describes representative nanoassemblies that can be constructed using the node and strut components described above. Many more possibilities exist than are shown, although the structures outlined fall into several basic classifications.

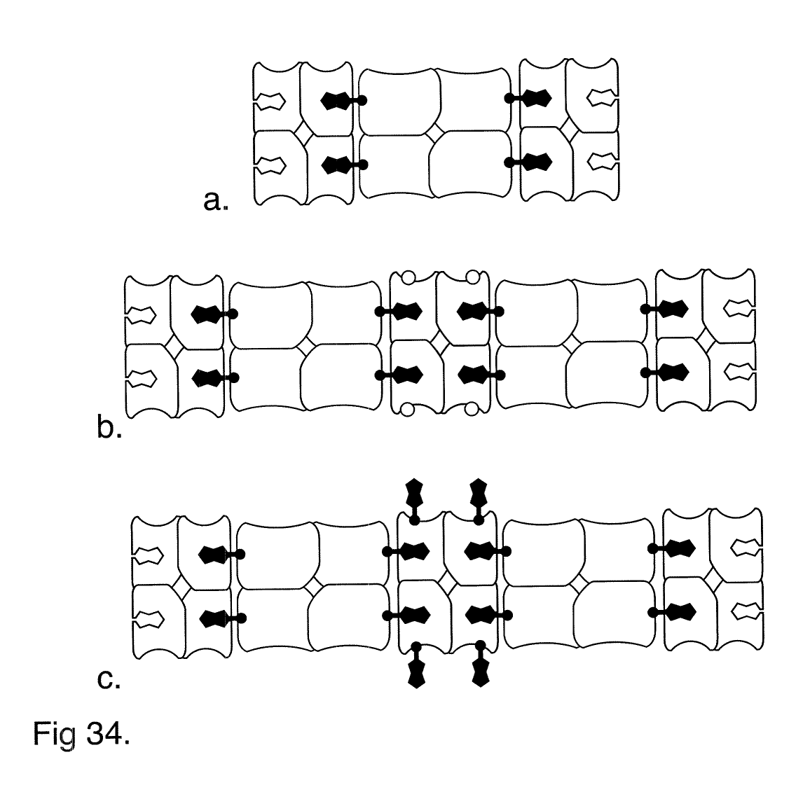

One Dimensional Structures: FIG. 34 shows schematic views of struts of different length consisting of combinations of streptavidin and nodes with D2 symmetry. Such constructs are useful in controlling the dimensions of assembled nanostructures. FIG. 34a shows an extended strut incorporating two streptavidin tetramers and a single D2 symmetric node (e.g see FIG. 30ab). FIG. 34b shows an extended strut incorporating three streptavidin tetramers and two D2 symmetric nodes. The central streptavidin has been modified (e.g. see FIG. 28) by the introduction of cysteine residues along one orthogonal dyad axis to allow the biotinylation of the strut after it is incorporated in a nanostructure (FIG. 34c).

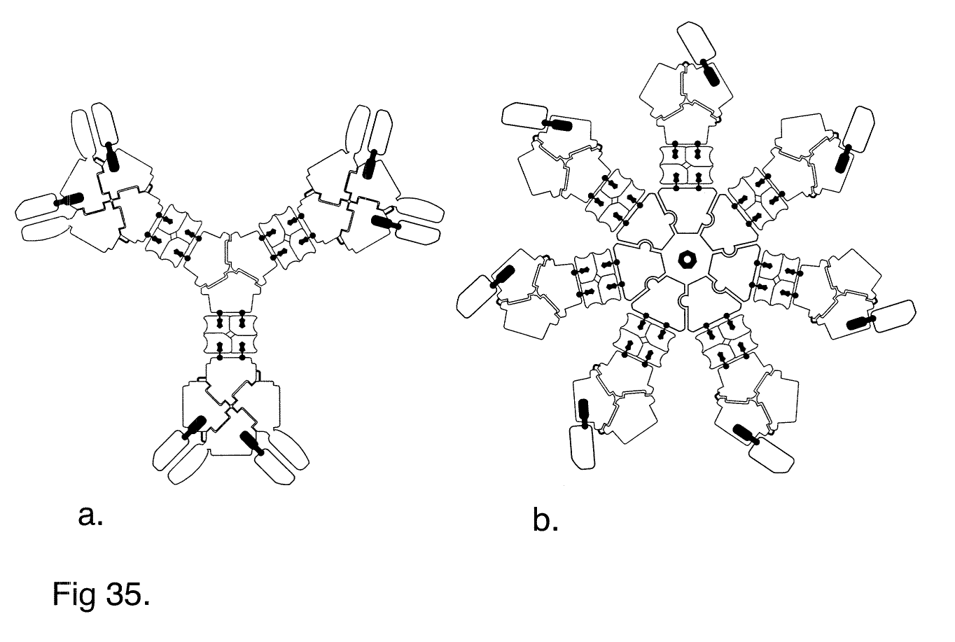

2-Dimensional Radial Structures: FIG. 35 schematically shows examples of radial structures as, for example, could be formed on self-assembling monolayers or anchored to discrete metal particles deposited on a silicon or other non-metallic substrate surface. FIG. 35a shows a C3 node which is linked through streptavidin struts to 3 single-chain C4 tetramers that have all been functionalized as described in FIG. 9. FIG. 35b shows a C7 node which is linked through streptavidin struts to 7 single-chain C3 trimers that are variations of the functionalized trimers described in FIG. 5. The structures can be also be functionalized through modifications introduced into the struts (e.g. see FIGS. 28 and 34). Two-dimensional lattices functionalized with specific binding molecules like immunoglobulin binding domains could find application in diagnostics, biological filters or other applications.

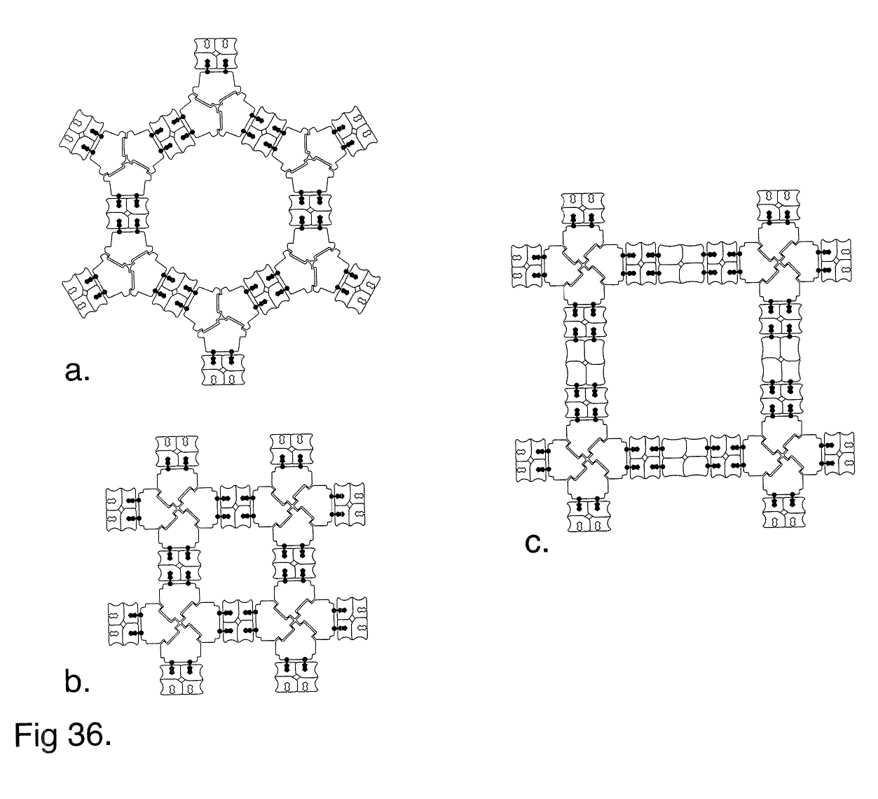

2-Dimensional Lattices: FIG. 36 schematically shows examples of 2-dimensional lattices, as, for example, could be formed on self-assembling monolayers. FIG. 36a shows a hexagonal lattice incorporating C3 nodes linked through streptavidin struts. FIGS. 36b,c show square lattices incorporating C4 nodes and struts of different lengths to control the lattice dimensions. The struts in FIG. 36c incorporate a D2 strut extender as outlined in FIG. 30. The structures can be functionalized either through modifications introduced into the nodes (e.g FIGS. 5 and 9) or struts (e.g FIGS. 28 and 34). Two-dimensional lattices functionalized with specific binding molecules like immunoglobulin binding domains could find application in diagnostics, biological filters or other applications.

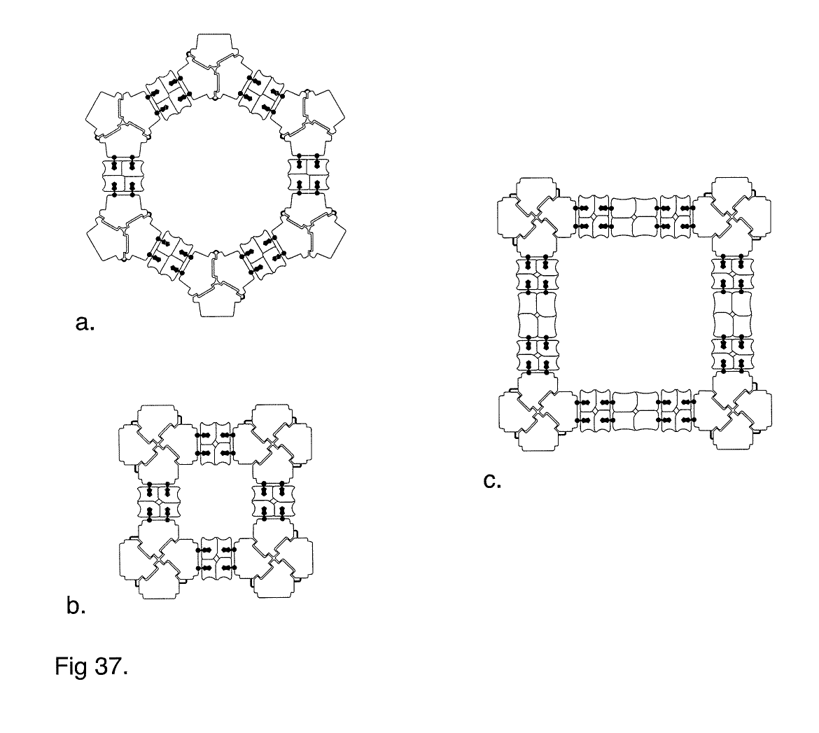

2-Dimensional Polygon Structures: FIG. 37 schematically shows examples of 2-dimensional polygonal structures, as, for example, could be formed on self-assembling monolayers. FIG. 37a shows a hexagon array incorporating single-chain C3 nodes linked through streptavidin struts. FIG. 37b,c show square arrays incorporating single-chain C4 nodes and struts of different lengths to control the lattice dimensions. The struts in FIG. 37c incorporate a D2 strut extender as outlined in FIG. 30. The structures can be functionalized either through modifications introduced into the nodes (e.g see FIGS. 5 and 9) or struts (e.g. see FIGS. 28 and 34). Two-dimensional polygonal structures functionalized with specific binding molecules like immunoglobulin binding domains could find application in diagnostics, biological filters or other applications.

3-Dimensional Radial Structures: Radial 3-dimensional structures can be produced by the attachment of struts incorporating streptavidin to the dyad axes of polyhedral nodes such as those shown in FIGS. 19 and 20. Struts or terminating nodes of struts can be functionalized either through modifications introduced into the nodes (e.g see FIGS. 5 and 9) or struts (e.g. see FIGS. 28 and 34). Radial structures functionalized with specific binding molecules like immunoglobulin binding domains could find application in diagnostics, biological filters or other applications.

3-Dimensional Polygon Structures: Three-dimensional polygonal structures with defined geometry and dimensions can be generated through the combination of struts incorporating streptavidin and nodes with the symmetry and geometry corresponding to a polygonal apex node. Representative structures of regular polyhedra are shown in FIG. 11a,b and FIG. 19c. Examples of apex node structures for regular dodecahedra and icosahedra are given in FIGS. 26 and 27 respectively. Sequence specifications for these nodes are given in Table 2D and 2F respectively. Table 2D also provides a specification for a “bucky” node. Given the great variety of known carbon-based “buckyball” geometries (Weber 1999), it is probable that a corresponding variety of protein-based nanostructures can be generated. Three-dimensional polygonal structures can be functionalized with specific binding molecules like immunoglobulin binding domains and could find application in diagnostics, biological filters or other applications. In addition, 3-dimensional polygonal structures, which are generally hollow inside, can be used to encapsulate or coat organic, inorganic, or biomaterials for imaging, diagnostic, drug delivery or other applications.

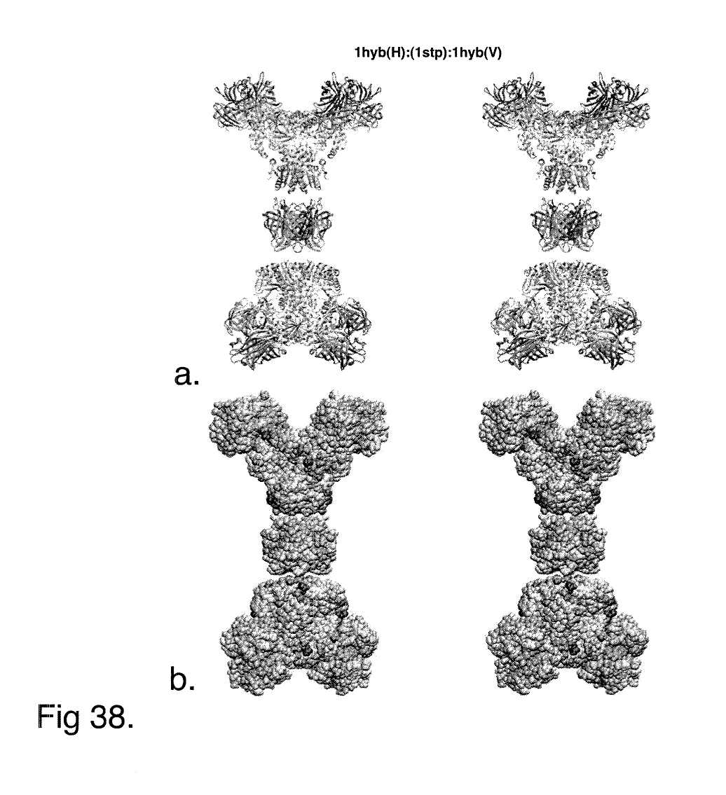

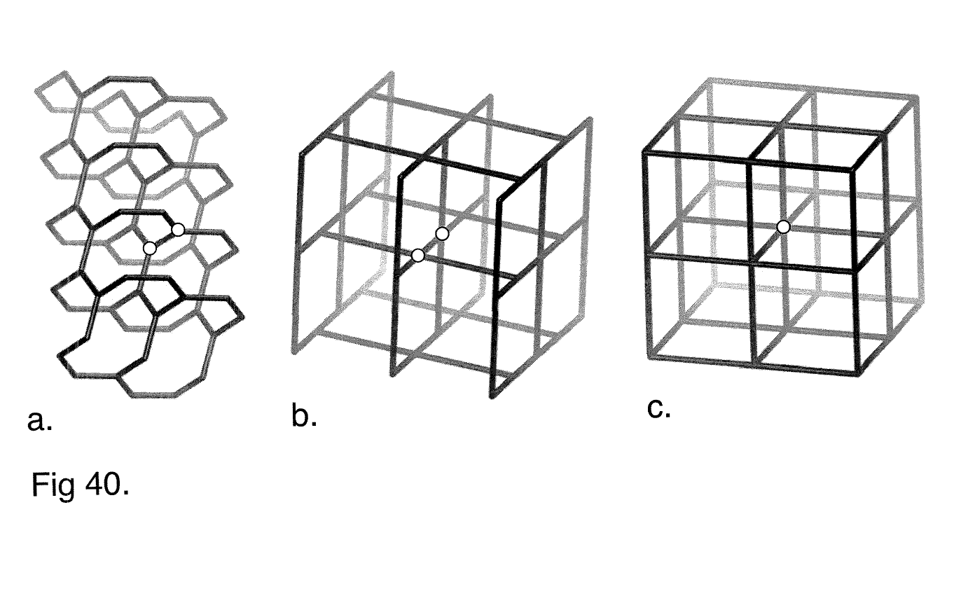

3-Dimensional Lattices: Three-dimensional lattices can be built up from molecular nodes and struts using a number of different strategies, allowing precise control of geometrical and symmetry properties of the resulting lattice. FIG. 38a,b presents stereoscopic views, in schematic and space filling representation, of a 3D lattice node incorporating two variations of a D3 node derived from the node template 1hyb (FIG. 17cd and Table 2L). The two node variations have biotinylation sites that orient bound streptavidin tetramers at 90 degrees to each other (e.g see FIG. 12a,c) along their equivalent dyad axes. Consequently, when a streptavidin tetramer bridges two such nodes, they are rotated 90 degrees relative to each other. FIG. 40a schematically illustrates the 3-connected 3D lattice that can be formed incorporating such linked nodes (shown as two white dots in the schematic lattice illustration).

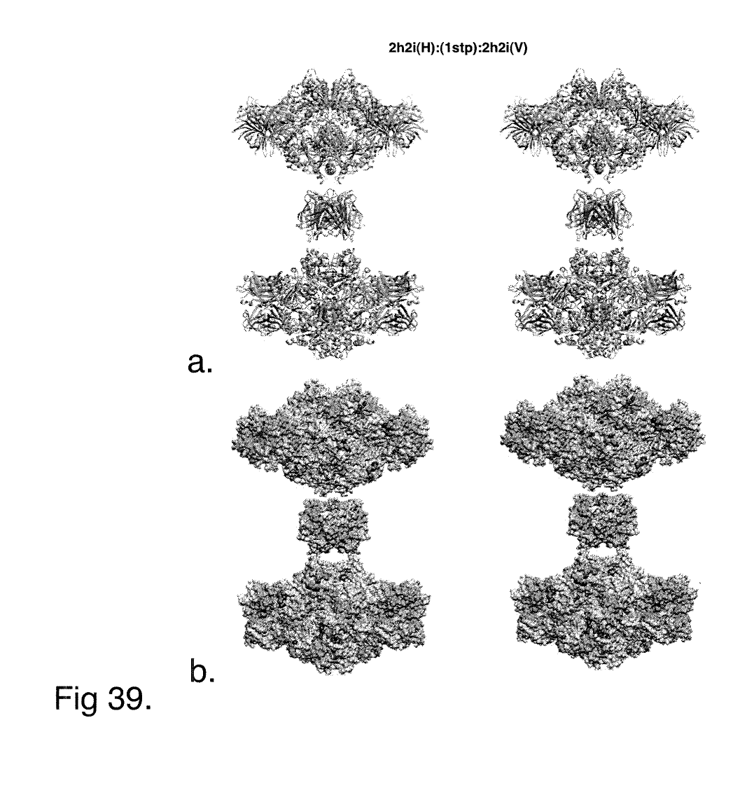

FIG. 39a,b present stereoscopic views, in schematic and space filling representation, of a 3D lattice node incorporating two variations of a D4 node derived from the node template 2h2i (FIG. 18cd and Table 20). The two node variations have biotinylation sites that orient bound streptavidin tetramers at 90 degrees to each other (e.g see FIG. 12a,c) along their equivalent dyad axes. Consequently, when a streptavidin tetramer bridges two such nodes, they are rotated 90 degrees relative to each other. FIG. 40b schematically illustrates the 4-connected 3D lattice that can be formed incorporating such linked nodes (shown as two white dots in the schematic lattice illustration).

FIG. 40a,b present stereoscopic views, in backbone and space filling representation, of a 3D lattice node derived from the dodecahedral node template 1pvv (Table 2Q). FIG. 40c schematically illustrates the 6-connected 3D cubic lattice that can be formed by linking such nodes with streptavidin or extended struts. In FIG. 40c, the central white dot represents the location of a node.

The nodes and struts of 3-dimensional lattices can be functionalized with specific binding molecules like immunoglobulin binding domains and could find application in diagnostics, biological filters or other applications. In addition, there are many applications where the ability to immobilize magnetic centers, charge, chromophoric groups, or other inorganic, organic, or biological groups at high density and with controlled geometry can lead to useful applications such as batteries, capacitors, non-linear optical materials, data storage, and other devices.

### Examples of Nanostructural Assemblies for Nanoscale Patterning and Resist Masks

In addition to applications where the protein components of nanoscale assemblies play a functional role, proteinaceous nanoscale assemblies can provide a means of high-resolution patterning of silicon, glass, metal, or other substrates, to allow production of microelectronic devices, devices incorporating zero-mode waveguides (Levene et. al, 2003) or microelectromechanical systems (MEMS) using conventional semiconductor fabrication (Widman et al., 2000) and/or MEMS fabrication technology (Judy, 2001). The proteinaceous nanoscale assembly can be used directly as a way of introducing a pattern on a substrate material. Alternatively, the proteinaceous nanoscale assembly is used as a way of masking a resist to transfer the pattern of the nanoscale assembly to an underlying substrate material. The approaches outlined below are applicable to both 2-dimensional and 3-dimensional assembly architectures.

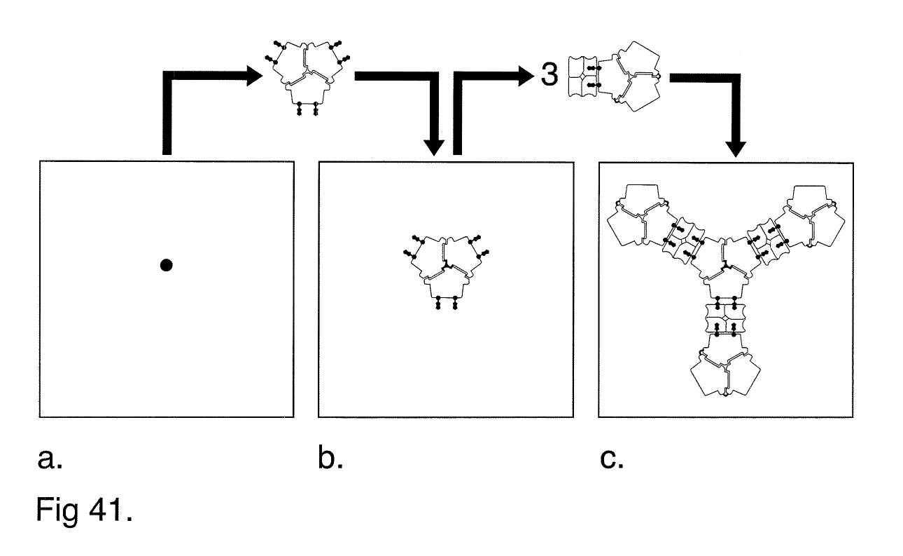

FIG. 41 schematically illustrates a method of making a nanostructure pattern on a surface. FIG. 41a shows, for example, a substrate that has a semiconductor material surface with a single gold atom or cluster (Haztor-di Picciotto, 2007) or, alternatively, a patch of chemically reactive molecules (e.g., Liu & Amro, 2002) located on the surface to nucleate the formation of the nanostructural assembly. Upon addition (e.g. by contacting the surface with a solution containing the nanostructure node trimer) of a trimeric node construct functionalized with bound biotin groups and modified at one terminus with a reactive moiety (binding function) that enables coupling to the nucleation site on the substrate, the node can be specifically immobilized on the surface (FIG. 41b). The immobilized node can be further reacted with nanostructural components incorporating streptavidin or streptavidin-incorporating struts to form immobilized nanostructures such as schematically illustrated in FIG. 41c.

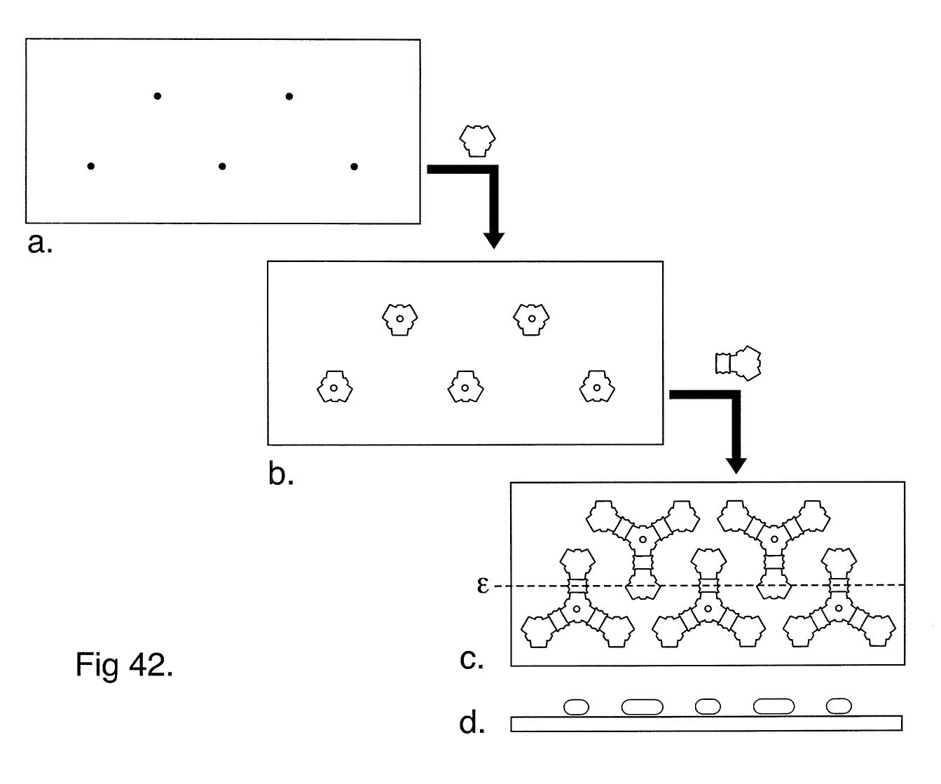

FIG. 42 schematically presents a method of making a repetitively patterned protein nanostructure on a metallic or non-metallic substrate following the steps exemplified in FIG. 41 using a simplified representation for the node and strut components. Here a substrate (FIG. 42a) is patterned with an array of nucleation sites. The nucleation sites can be arranged in a regular or periodic pattern, a quasiperiodic pattern (such as a Penrose tiling), or a non-periodic predetermined patter. Following the steps of addition of the node proteins to the surface (FIG. 42b) and addition of the streptavidin-incorporating struts, a patterned array (FIG. 42c) is produced. FIG. 42d shows a section of the patterned surface at the section line ε in FIG. 42c.

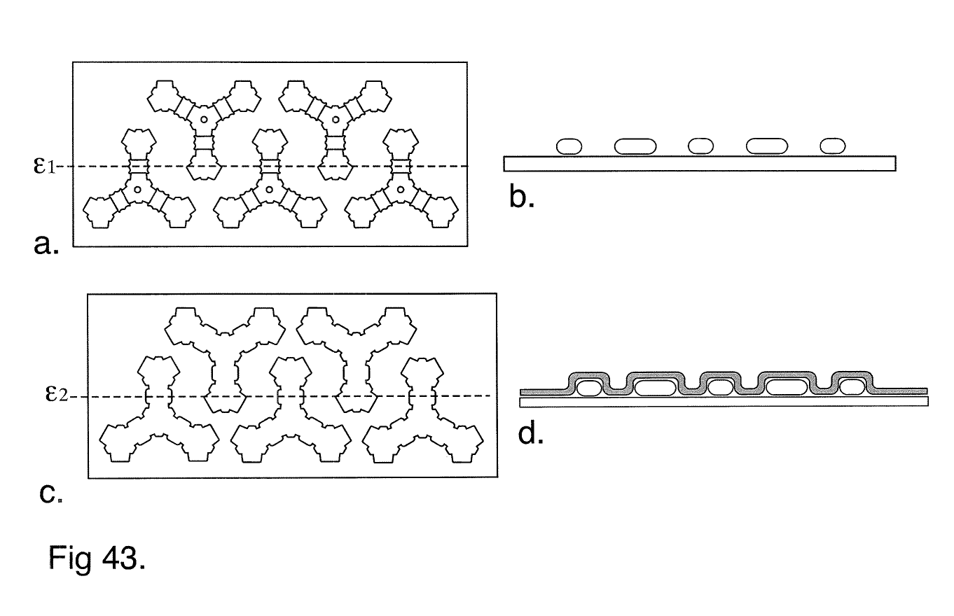

FIG. 43 presents a method of making a patterned nanostructure assembly with sub-100 nanometer features on a substrate surface. FIG. 43a reiterates the patterned surface of FIG. 42c and FIG. 43b shows the section of FIG. 43a at ε1. FIG. 43c shows the result of using any of several methods of semiconductor fabrication (e.g., using various forms of plasma and/or chemical vapor deposition technology, Widman, et al., 2000) to coat the substrate patterned with the protein nanostructure to produce the patterned surface shown in plan in FIG. 43c and in section in FIG. 42d (corresponding to the section line ε2 in FIG. 43c). For example, the patterned substrate can be coated with materials such as a metal (such as iron), a noble metal (such as gold, platinum, or silver), a glass (such as silicon dioxide), a ceramic, a semiconductor (such as silicon or germanium), a carbon allotrope (such as diamond or graphite), a polymer, and/or an organic polymer (such as tetrafluoroethylene). The resulting patterned surface (FIG. 43c) can be used as a template for soft lithography (Xia & Whitesides 1998, Rogers & Nuzzo 2005) or as a step in a multistep semiconductor fabrication process (Widman et al., 2000).

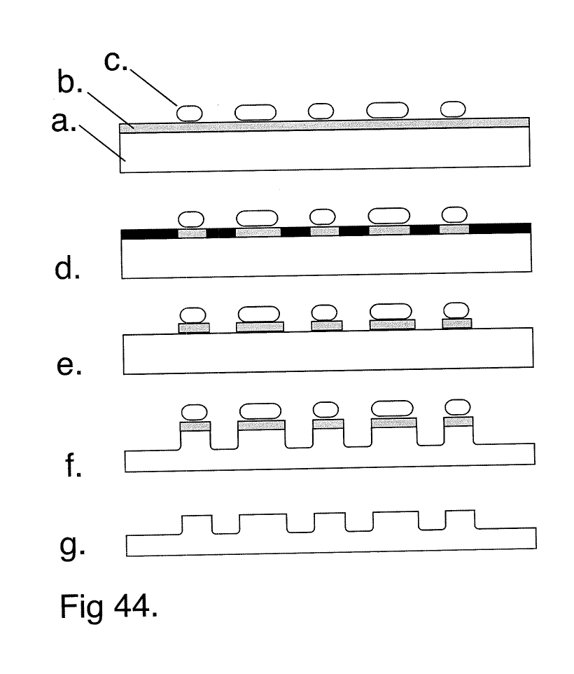

FIG. 44 presents a method of making a patterned structure with sub-100 nanometer features on a substrate surface using a proteinaceous nanostructure assembly as a patterned mask superimposed on a photoresist material. FIG. 44a,b,c shows a cross section of a protein nanostructure (FIG. 44c) superimposed on a layer of a resist material (FIG. 44b), that is in turn coated on a substrate to be patterned (FIG. 44a). Exposure of the assembly to, for example, irradiation of a suitable nature to modify the resist, produces the structure of FIG. 44d, where the superimposed nanostructure has prevented exposure of the resist to the incident radiation. FIG. 44e shows the structure where the exposed resist has been dissolved away, for example using chemical means. FIG. 44f shows the structure where the exposed substrate surfaces have been etched producing nanoscale features that are complementary to the structural features of the proteinaceous nanoscale assembly used to pattern the resist. FIG. 44g shows the structure after the proteinaceous nanoscale assembly and non-reacted resist have been removed, for example by using chemical means. The resulting patterned surface (FIG. 44g) can be used as a template for soft lithography (Xia & Whitesides 1998, Rogers & Nuzzo 2005) or as a step in a multistep semiconductor fabrication process (Widman et al., 2000).

Additional example of finite or periodic 2-dimensional proteinaceous nanostructural assemblies that can serve as patterning templates on surfaces are described above and schematically illustrated in FIGS. 35, 36, and 37.

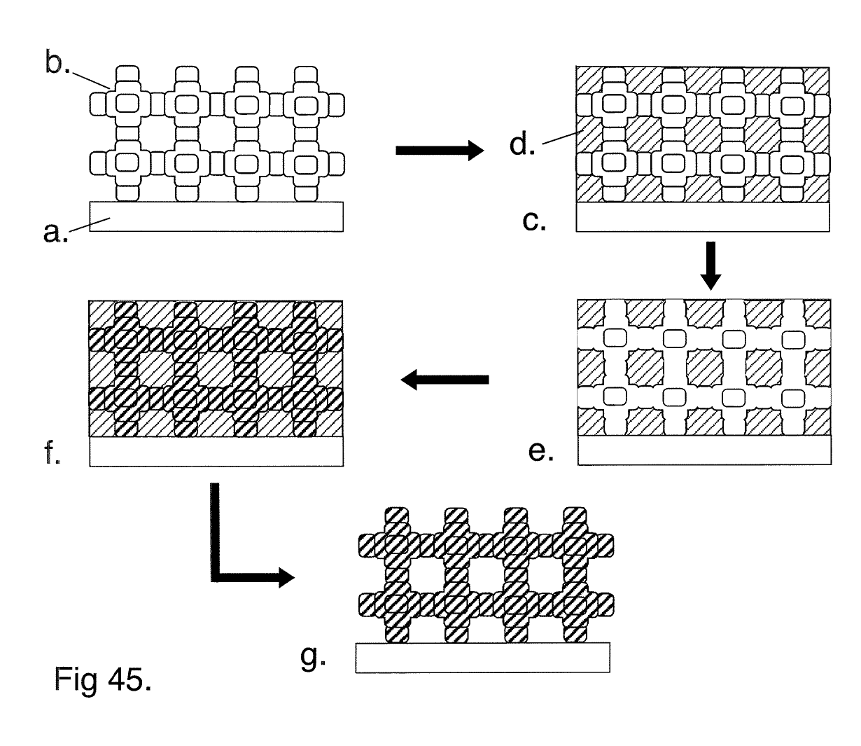

3-dimensional, as well as 2-dimensional, proteinaceous nanostructure assemblies can be used as nanoscale patterning elements. The structures can be coated as outlined in the process of FIG. 43 or, alternatively, serve as a 3-dimensional resist to form a negative of the proteinaceous nanostructural assembly. For example, FIG. 45ab schematically shows a cubic lattice structure composed of six-connected cubic nodes (for example, see FIG. 33) and streptavidin struts (FIG. 45b) assembled on a solid substrate (FIG. 45a). FIG. 45cd shows the structure (FIG. 45c) embedded in a matrix (FIG. 45d) that can polymerize and/or be transformed by chemical reaction, heat, and/or radiation to form a chemically and/or thermally stable matrix material. The matrix (FIG. 45d) can interpenetrate the structure (FIG. 45c). For example, the matrix (FIG. 45d) can itself have the form of a cubic lattice offset from the cubic lattice of the proteinaceous nanostructure assembly. The cubic lattice of the structure (FIG. 45c) and the cubic lattice of the matrix (FIG. 45d) can interpenetrate each other. FIG. 45e shows the structure after chemical, heat, and/or radiation treatment is applied to ablate the proteinaceous nanoscale structure, leaving a “negative” three-dimensional cubic channel structure in the matrix material. That is, the matrix material can occupy the space not occupied by the proteinaceous nanostructure assembly. The matrix material (first matrix material) can include a metal (such as iron), a noble metal (such as gold, platinum, or silver), a glass (such as silicon dioxide), a ceramic, a semiconductor (such as silicon or germanium), a polymer, and/or an organic polymer (such as tetrafluoroethylene). For example, such “negative” structures incorporating nanoscale channels have potential utility as components in nanofluidics systems. FIG. 45f shows the structure of FIG. 45e after further chemical treatment is applied to deposit a metallic or other second matrix material in the negative cavity originally occupied by the proteinaceous nanostructure assembly. The second matrix material can include a metal (such as iron), a noble metal (such as gold, platinum, or silver), a glass (such as silicon dioxide), a ceramic, a semiconductor (such as silicon or germanium), a polymer, and/or an organic polymer (such as tetrafluoroethylene). FIG. 45g shows the structure after chemical, heat, or radiation treatment is applied to remove the first matrix material, leaving a nanoscale structure composed of metal or other second matrix material that is a replica of, that is, has the same or similar form as the original proteinaceous nanostructure assembly. For example, three-dimensional nanoscale assemblies made of metal or semiconductor materials have potential utility as components in semiconductor or MEMS applications.

Additional examples of finite or periodic 3-dimensional proteinaceous nanostructural assemblies are described above and some are schematically illustrated in FIG. 40. Such 3-dimensional structures with nano-dimensional features can have utility as optical or physical waveguides or filters, nanofluidic devices, or in other semiconductor or MEMS applications.

### TERMS AND DEFINITIONS

A subunit can be a tertiary polypeptide structure. The amino acid residues in a subunit can be covalently linked through peptide bonds in a polypeptide sequence. A subunit can be formed of one or more polypeptide chains. The polypeptide subunit can, under certain conditions, e.g., certain pH conditions, aggregate with one or more other polypeptide subunits to form a multisubunit node polypeptide that is a quaternary polypeptide structure. For example, in a native streptavidin tetramer, 4 identical subunits, each formed of an identical but separate polypeptide chain, aggregate. A multimeric protein having a symmetry can be formed of several essentially identical subunits that are repeated with an orientation with respect to each other to achieve the symmetry. For example, a Cn symmetric multimeric protein can be formed of n subunits placed about a common axis. For example, a C3 symmetric multimeric protein can be formed of 3 subunits placed about a common axis. For example, a Dn symmetric multimeric protein can be formed of 2n subunits, where each subunit is related to another subunit to form a pair, and each pair of subunits is placed about a common axis. For example, a D4 symmetric multimeric protein can be formed of 8 subunits, where each of 4 pairs of subunits are placed about a common axis. For example, a multimeric protein having the symmetry of a Platonic or Archimedean solid can be formed of a number of subunits equal to the number of edges in each polygonal face of the solid, summed over the polygonal faces. For example, a multimeric protein with tetrahedral symmetry can be formed of a number of subunits equal to the number of edges in a face, 3, times the number of faces, 4, that is, 12 subunits. For example, a multimeric protein with dodecahedral symmetry can be formed of a number of subunits equal to the number of edges in a pentagonal face, 5, times the number of faces, 12, to yield a total of 60 subunits.

Polypeptide subunits (subunits) within the quaternary polypeptide structure can be held to each other by noncovalent bonds (e.g., ionic bonds, van der Waals bonds, and/or hydrophobic bonds) and/or by covalent bonds (e.g., disulfide bridges and/or peptide bonds). Thus, each subunit may be formed of one or more polypeptide chains that are not covalently bound to the polypeptide chains of any other subunit of a quaternary polypeptide structure, each subunit may be formed of a polypeptide chain that is covalently bound to a polypeptide chain of at least one other subunit (e.g., the quaternary polypeptide structure can formed of a number of polypeptide chains less than the number of subunits, for example, the quaternary polypeptide structure can be formed of a single polypeptide chain), or some subunits may be formed of a polypeptide chain not covalently bound to a polypeptide chain of another subunit whereas other subunits are formed of a polypeptide chain that is covalently bound to a polypeptide chain of at least one other subunit.

For example, the amino acid residues of a polypeptide subunit can be in a single polypeptide sequence.

Multimerization can refer to the process in which individual polypeptide subunits aggregate to form a multisubunit node polypeptide. Three individual polypeptide subunits, each formed of a polypeptide chain that is not covalently linked to another subunit, aggregating under the influence of non-covalent bonds to form a trimer is an example of multimerization. Alternatively, three individual polypeptide subunits can be formed of a polypeptide sequence that is covalently linked to the polypeptide sequence of another subunit, so that the three polypeptide subunits are formed from a single polypeptide chain. Even though the polypeptide subunits are covalently linked through the polypeptide chain, each individual polypeptide subunit can be folded into a separate tertiary structure without the individual polypeptide subunits being assembled into a quaternary trimer. When these polypeptide subunits undergo multimerization, the tertiary structures of the individual polypeptide subunits can come into close proximity, for example, under the influence of non-covalent bonds, to form a quaternary trimer in which a number of amino acid residues of each polypeptide subunit are in close proximity to a number of the amino acid residues of the other polypeptide subunits.

A rotational symmetry axis of an object can be an axis about which a less than full rotation of the object can result in a matching superposition of the object upon itself. An ordering of subunits about the rotational symmetry axis can refer to the subunits corresponding to the N-fold symmetry in a successive clockwise or counter-clockwise sequence when sighting along the rotational symmetry axis.

Features, such as polypeptide subunits of a multisubunit node polypeptide that are essentially related by a symmetry, might not be strictly identical. For example, two of the polypeptide subunits may differ from each other in that one, two, or a short oligomeric subsequence of the polypeptide sequences from which they are formed are different. However, this minor difference in the polypeptide sequence does not affect the overall form of the subunit. For example, if one subunit of a trimer has one amino acid in the polypeptide sequence from which it is formed that is different than the corresponding amino acid in the polypeptide sequences of the other two subunits, but the folding of all the subunits is similar, the trimer still has essential three-fold rotational symmetry.

A derivative of an initial molecule includes molecules resulting from the replacement of an atom, group of atoms, bond, or bonds of the initial molecule by a different atom, group of atoms, bond, or bonds and molecules resulting from the addition or deletion of an atom or a group of atoms to the initial molecule. For example, 2-iminobiotin is a derivative of biotin. The structure of 2-iminobiotin is the same as that of biotin, except that the oxygen double bonded to the imidazolidine is replaced with a single bonded primary amine and the single bond between the 2-carbon and the 3-nitrogen of the imidazolidine ring is replaced by a double bond. Similarly, if a residue of an initial polypeptide is replaced with a different residue, the resultant polypeptide is a derivative of the initial polypeptide. If a group of atoms is added to an initial polypeptide, for example, if a linker molecule having a thiol reactive group and a biotin covalently linked to each other is reacted with a cysteine of the initial polypeptide, so that the biotin becomes bonded through a disulfide to the cysteine, the resultant polypeptide is a derivative of the initial polypeptide. An analog of a molecule is included within the term derivative.

When a chemical or biochemical group is mentioned, derivatives and analogs of that chemical or biochemical group are also implied. For example, if biotin is recited, 2-iminobiotin is also implied.

A polypeptide extension of a polypeptide subunit can be a polypeptide sequence that is linked to an amino or carboxy terminus of a polypeptide sequence comprising the polypeptide subunit. The polypeptide extension may or may not be folded into the tertiary structure of the polypeptide subunit.

A binding function of a polypeptide sequence (such as a polypeptide extension) can be a subsequence of amino acids to which an atom, group of atoms, or molecule, such as a portion of a protein or a metallic surface, can form a covalent or non-covalent bond.

A polypeptide subsequence can be a continuous set of covalently bonded amino acid residues within a polypeptide sequence. The polypeptide subsequence may comprise all, less than all, or only one of the amino acid residues in the polypeptide sequence.

A nanostructure strut can bind covalently or non-covalently to a specific binding site of a nanostructure node multimeric protein.

A protein, such as a multimeric protein, can include a ligand binding pocket. Such a pocket can be a depression in or inward folding of the surface of the protein. The ligand binding pocket can include a specific binding site. For example, a nanostructure node multimeric protein can include a ligand binding pocket. A nanostructure strut can bind to the ligand binding pocket. For example, the nanostructure strut can include a region of an immunoglobulin that binds to the ligand binding pocket of the nanostructure node multimeric protein. For example, the nanostructure strut can include biotin, iminobiotin, a nucleotide, an enzyme inhibitor, an enzyme activator, an enzyme substrate, an enzyme cofactor, a coenzyme, and/or derivatives that bind to the ligand binding pocket of the nanostructure node multimeric protein.

A bridge molecule can serve to attach two other molecules, such as proteins. For example, a bridge molecule can include a biotin group covalently bound to an adenosine triphosphate (ATP) group. The biotin group can bind to a biotin binding site, such as present on streptavidin, and the adenosine triphosphate (ATP) group can bind to an ATP binding site, such as present on the MJ0577 protein.

A bindable polypeptide subunit, for example, of a multimeric protein, can be capable of binding, directly or through an intermediary molecule, such as a bridge molecule, to another molecule, such as a protein. For example, a bindable subunit can include a specific binding site to which a nanostructure strut, e.g., a streptavidin-containing nanostructure strut, can bind.

A non-bindable polypeptide subunit, for example, of a multimeric protein, can be incapable of binding to another molecule, such as a protein. For example, a non-bindable subunit may lack a specific binding site to which a nanostructure strut, e.g., a streptavidin-containing nanostructure strut, can bind.

The embodiments illustrated and discussed in this specification are intended only to teach those skilled in the art the best way known to the inventors to make and use the invention. Nothing in this specification should be considered as limiting the scope of the present invention. All examples presented are representative and non-limiting. The above-described embodiments of the invention may be modified or varied, without departing from the invention, as appreciated by those skilled in the art in light of the above teachings. It is therefore to be understood that, within the scope of the claims and their equivalents, the invention may be practiced otherwise than as specifically described.

### Examples of Embodiments and Methods

### Method of Using Proteins for Nanostructure Assemblies

Paragraph 1. A method of using a template multimeric protein as a nanostructure node, comprising connecting the template multimeric protein with a nanostructure strut, wherein the template multimeric protein has a known 3-dimensional structure, wherein the template multimeric protein is derived from a thermostable microorganism, wherein the template multimeric protein has Cn, Dn, or higher symmetry, and wherein the template multimeric protein incorporates a specific binding site for the attachment of at least one nanostructure strut with predefined stoichiometry and orientation.

Paragraph 2. The method of Paragraph 1, wherein the template multimeric protein is selected from the group of proteins provided in Table 1.

Paragraph 3. The method of Paragraph 1, wherein the template multimeric protein has a sequence having greater than 80 percent sequence identity with the sequence of a protein provided in Table 1.

Paragraph 4. The method of Paragraph 2, wherein the template multimeric protein has Cn subunit symmetry.

Paragraph 5. The method of Paragraph 3, wherein the template multimeric protein has Cn subunit symmetry.

Paragraph 6. The method of Paragraph 2, further comprising modifying the template multimeric protein as necessary so that the specific binding site comprises at least two specific amino acid reactive residues, wherein the template multimeric protein has Cn subunit symmetry, and wherein each specific amino acid reactive site is capable of covalently attaching a biotin group, so that at least one nanostructure strut can be attached to the template multimeric protein with predefined stoichiometry and orientation.

Method of Making Nanostructure Assemblies

Paragraph 7. A method comprising: generating a mathematical and/or computer graphic representation of the 3-dimensional molecular structure of a template multimeric protein and a streptavidin tetramer; replacing each surface cysteine residue of the template multimeric protein with an alternative amino acid in the representation; iterating through several spatial configurations of the streptavidin tetramer relative to the template multimeric protein in the representation, with the streptavidin tetramer in approximate Van der Waals contact with the template multimeric protein; for each spatial configuration, assigning cysteine to replace two amino acid side chains on the surface of the template multimeric protein that are geometrically complementary to positions in the streptavidin tetramer that correspond to the terminal chemical groups on biotin (e.g., the biotin valeric acid carbon atom) when bound to the streptavidin tetramer to generate a nanostructure node multimeric protein representation; assigning a measure of quality to each spatial configuration (e.g., root-mean-square (rms) error between the coordinates of the projected positions of valeric acid carbon atoms of a biotin group bound to the streptavidin tetramer and of the sulfur atoms of the nearest cysteine on the surface of the nanostructure node multimeric protein and/or the potential energy of electrostatic interaction between the nanostructure node multimeric protein and the streptavidin tetramer); storing each spatial configuration and associated nanostructure node multimeric protein; and selecting an optimal nanostructure node multimeric protein for production (for example, based on the measure of quality associated with a spatial configuration of the optimal nanostructure node multimeric protein).

Paragraph 8. A method according to Paragraph 7 of operating on a template sequence of a template multimeric protein with Cn subunit symmetry having a surface to define the amino acid sequence of a nanostructure node multimeric protein that can form planar nanoassemblies incorporating Cn planar nodes and streptavidin or streptavidin-incorporating struts attached with predefined stoichiometry and orientation, comprising: (8a.) generating a mathematical and/or computer graphic representation of the 3-dimensional molecular structure of the Cn symmetric template multimeric protein and a streptavidin tetramer; (8b.) using a computer graphics and/or mathematical method to identify surface cysteine residues on the surface of the template multimeric protein; (8c.) assigning an alternative amino acid(s) (e.g., Ala, Serine, Asp, etc.) to replace the identified surface cysteine residues in the template sequence; (8d.) using the mathematical and/or computer graphic representation to initially position the 3-dimensional coordinates of the template multimeric protein and streptavidin tetramer, so that the Cn symmetry (or z) axis of the template multimeric protein is parallel to the streptavidin tetramer z-dyad axis or y-dyad axis, the centers of mass of the template multimeric protein and streptavidin coordinates have the same or nearly the same z coordinate, and the molecules do not physically intersect; (8e.) using the mathematical and/or computer graphic representation to incrementally translate the 3-dimensional coordinates of the streptavidin tetramer along one of its dyad axes that is normal to and intersects the Cn axis of the template multimeric protein, until the template multimeric protein and streptavidin tetramer approximately reach Van der Waals contact; (8f.) using a computational and/or computer graphics method to identify as specific amino acid reactive sites two amino acid residues on the surface of the template multimeric protein that are geometrically complementary to positions in the streptavidin tetramer that correspond to the terminal chemical groups on biotin (e.g., the biotin valeric acid carbon atom) when bound to the streptavidin tetramer; (8g.) assigning a cysteine to replace each of two amino acid residues identified as specific amino acid reactive sites, wherein the assigned cysteine has an associated biotin group, to generate a nanostructure node multimeric protein; (8h.) using a computational and/or computer graphics method, creating a model of the complex formed between the nanostructure node multimeric protein, having the biotin groups associated with the assigned cysteines bound to the streptavidin tetramer, evaluating the overall quality of a potential linkage between the nanostructure node multimeric protein and the streptavidin tetramer, and assigning a measure of binding quality (e.g., root-mean-square (rms) error between the coordinates of the projected positions of valeric acid carbon atoms of the biotin group as bound to the streptavidin tetramer and of the sulfur atoms of the assigned cysteine with which the biotin group is associated); (81.) using a computational and/or computer graphics method to evaluate the overall quality of the complementarity of fit between the surface of the nanostructure node multimeric protein and the surface of the streptavidin tetramer and assigning a measure of complementarity of fit and/or energetic stability based on e.g., steric and electrostatic complementarity of amino acid residues at the interface, maintenance of preferred amino acid side chain rotomer conformations, low potential energy as estimated using a computational method such as molecular mechanics, quantum mechanics, or potential energy calculations, or through experimental methods of measuring complex stability, including affinity measurements, calorimetry, or other experimental methods; (8j.) storing the 3-dimensional coordinates of the nanostructure node multimeric protein: streptavidin complex along with quality measures in a database; (8k.) beginning with the initial orientation of 8d, incrementing a rotation of the template multimeric protein about the Cn axis; (8l.) iterating steps 8d. to 8k. over an angular increment of at least 360/n degrees, where n defines the foldedness of the multimeric protein symmetry axis; and (8m.) ranking quality measures of stored nanostructure node multimeric protein: streptavidin complexes and/or examining coordinates of stored nanostructure node multimeric protein: streptavidin complexes and selecting an optimal nanostructure node multimeric protein for production.

Paragraph 9. The method of Paragraph 8 wherein the angular increment is in a range of from about 0.001 degrees to about 5 degrees.

Paragraph 10. The method of Paragraph 8, further comprising producing the optimal nanostructure node multimeric protein.

Paragraph 11 The method of Paragraph 10, wherein the optimal nanostructure node multimeric protein is produced by expression in an E. coli bacterium or another heterologous protein expression system.

Paragraph 12. The method of Paragraph 8, further comprising modifying the template sequence of the template multimeric protein (e.g., by adding, removing, or replacing one or more amino acids at the surface of the template multimeric protein) to improve the complementarity of fit quality between the nanostructure node multimeric protein and the streptavidin tetramer.

Paragraph 13. The method of Paragraph 8, wherein the template multimeric protein has subunit symmetry selected from the group consisting of C3, C4, C5, C6, and C7 symmetry, and so as to permit the covalent attachment of biotin groups to the assigned cysteines of the specific amino acid reactive sites to allow for interconnection of the nanostructure node multimeric protein with streptavidin tetramers in a planar orientation.

Paragraph 14. A method according to Paragraph 7 of operating on a template sequence of a template multimeric protein with Cn subunit symmetry having a surface and a template number of polypeptide chains to define the amino acid sequence of a nanostructure node multimeric protein that can form polyhedral nanoassemblies incorporating Cn polyhedral nodes and streptavidin or streptavidin-incorporating struts attached with predefined stoichiometry and orientation, comprising: (14a.) generating a mathematical and/or computer graphic representation of the 3-dimensional molecular structure of the Cn symmetric template multimeric protein and a streptavidin tetramer; (14b.) using a computer graphics and/or mathematical method to identify surface cysteine residues on the surface of the template multimeric protein; (14c.) assigning an alternative amino acid(s) (e.g., Ala, Serine, Asp, etc.) to replace the identified surface cysteine residues in the template sequence; (14d.) using the mathematical and/or computer graphic representation to initially position the 3-dimensional coordinates of the template multimeric protein and streptavidin tetramer, so that the Cn symmetry (or z) axis of the template multimeric protein and streptavidin tetramer z-dyad axis or y-dyad axis are oriented at an angle corresponding to a polyhedral node apex angle, the centers of mass of the template multimeric protein and streptavidin coordinates are variably displaced along their z coordinates to facilitate the generation of polyhedron apex node geometry, and the molecules do not physically intersect; (14e.) using the mathematical and/or computer graphic representation to incrementally translate the 3-dimensional coordinates of the streptavidin tetramer along one of its dyad axes that intersects the Cn axis of the template multimeric protein, until the template multimeric protein and the streptavidin tetramer approximately reach Van der Waals contact; (14f.) using a computational and/or computer graphics method to identify as specific amino acid reactive sites two amino acid residues on the surface of the template multimeric protein that are geometrically complementary to positions in the streptavidin tetramer that correspond to the terminal chemical groups on biotin (e.g., the biotin valeric acid carbon atom) when bound to the streptavidin tetramer; (14g.) assigning a cysteine to replace each of two amino acid residues identified as specific amino acid reactive sites, wherein the assigned cysteine has an associated biotin group, to generate a nanostructure node multimeric protein; (14h.) using a computational and/or computer graphics method, creating a model of the complex formed between the nanostructure node multimeric protein and the streptavidin tetramer, having the biotin groups associated with the assigned cysteines bound to the streptavidin tetramer, evaluating the overall quality of a potential linkage between the node multimeric protein and the streptavidin tetramer, and assigning a measure of binding quality (e.g., root-mean-square (rms) error between the coordinates of the projected positions of valeric acid carbon atoms of the biotin group as bound to the streptavidin tetramer and of the sulfur atoms of the assigned cysteine with which the biotin group is associated); (14i.) using a computational and/or computer graphics method to evaluate the overall quality of the complementarity of fit between the surface of the nanostructure node multimeric protein and the surface of the streptavidin tetramer and assigning a measure of complementarity of fit and/or energetic stability based on e.g., steric and electrostatic complementarity of amino acid residues at the interface, maintenance of preferred amino acid side chain rotomer conformations, low potential energy as estimated using a computational method such as molecular mechanics, quantum mechanics, or potential energy calculations, or through experimental methods of measuring complex stability, including affinity measurement, calorimetry, or other experimental methods; (14j.) storing the 3-dimensional coordinates of the nanostructure node multimeric protein: streptavidin complex along with quality measures in a database; (14k.) beginning with each member of a set of initial orientations of 14d where the centers of mass of the template multimeric protein and streptavidin coordinates are variably displaced along their z coordinates by increments of from about 0.001 to about 5 angstroms, up to a total of 50 Angstroms, and incrementing a rotation of the template multimeric protein about the Cn axis by increments of from about 0.001 to about 5 degrees; (141.) iterating steps 14d. to 14k. over an angular increment of at least 360/n degrees, where n defines the foldedness of the template multimeric protein symmetry axis; and (14m.) ranking quality measures of stored nanostructure node multimeric protein: streptavidin complexes and/or examining coordinates of stored nanostructure node multimeric protein: streptavidin complexes and selecting an optimal nanostructure node multimeric protein for production.

Paragraph 15. The method of Paragraph 14, further comprising producing the optimal nanostructure multimeric protein.

Paragraph 16. The method of Paragraph 14, further comprising modifying the template sequence of the template multimeric protein (e.g., by adding, removing, or replacing an amino acid at the surface of the template multimeric protein) to improve the complementarity of fit quality between the nanostructure node multimeric protein and the streptavidin tetramer.

Paragraph 17: A method of operating on a template sequence of a Dn or higher symmetry template multimeric protein having a surface and a template number of polypeptide chains to define the amino acid sequence of a nanostructure node multimeric protein that can form nanoassemblies incorporating nodes of Dn or higher symmetry and streptavidin or streptavidin-incorporating struts attached with predefined stoichiometry and orientation, comprising: (17a.) generating a mathematical and/or computer graphic representation of the 3-dimensional molecular structure of the Dn or higher symmetry template multimeric protein; (17b.) using a mathematical and/or computer graphics method, identifying and listing amino acid residues with backbone or side chain atoms exposed at the protein surface; (17c.) assigning an alternative amino acid(s) (e.g., Ala, Serine, Asp, etc.) to replace cysteine residues in the list of amino acid residues with backbone or side chain atoms exposed at the protein surface; (17c.) generating a list of 3-dimensional coordinates of candidate amino acid carbons comprising Cβ amino acid atoms for all non-Gly and backbone Cα carbon atoms for all Gly amino acid residues with backbone or side chain atoms exposed at the protein surface; (17d.) generating a mathematical and/or computer graphic representation of a “bounding box” of which edge lines correspond to projected positions complementary to the biotin binding sites of streptavidin, wherein the bounding box has dimensions of about 6.4 Angstroms by about 19.5 Angstroms by a lengthwise dimension; (17e.) using a computer graphics and/or mathematical method to identify the location of the dyad axes in the template multimeric protein; (17f.) using a mathematical and/or computer graphic method to superimpose representations of the “bounding box” on the representation of the 3-dimensional molecular structure of the template multimeric protein, wherein a pair of bounding boxes are positioned with lengthwise dimension parallel to a dyad axis of the template multimeric protein and symmetrically about each dyad axis, wherein the longest dimension of each bounding box is greater than the largest dimension of the template multimeric protein along the dyad axis about which the bounding box is positioned, wherein for a pair of bounding boxes positioned about a dyad axis, a first member of the pair of bounding boxes is positioned with its 19.5 Angstrom dimension parallel to a Dn or higher symmetry axis of the template multimeric protein, and a second member of the pair of bounding boxes is positioned with its 19.5 Angstrom dimension perpendicular to the Dn or higher symmetry axis of the template multimeric protein; (17g.) using a mathematical and/or computer graphic method, identifying candidate amino acid carbons within 5 Angstroms of an edge line along the lengthwise dimension of each bounding box; (17h.) identifying amino acid residues that include a candidate amino acid carbon as specific amino acid reactive sites; (17i.) assigning a cysteine to replace an amino acid residue identified as specific amino acid reactive site, wherein the assigned cysteine has an associated biotin group, to generate a representation of the 3-dimensional molecular structure of a nanostructure node multimeric protein; (17j.) using a computational and/or computer graphics method, creating a model of the complex formed between the nanostructure node multimeric protein and the streptavidin tetramer, having the biotin groups associated with the assigned cysteines bound to the streptavidin tetramer, evaluating the overall quality of a potential linkage between the node multimeric protein and the streptavidin tetramer, and assigning a measure of binding quality (e.g., root-mean-square (rms) error between the coordinates of the projected positions of valeric acid carbon atoms of the biotin group as bound to the streptavidin tetramer and of the sulfur atoms of the assigned cysteine with which the biotin group is associated); (17k.) using a computational and/or computer graphics method to evaluate the overall quality of the complementarity of fit between the surface of the nanostructure node multimeric protein and the surface of the streptavidin tetramer and assigning a measure of complementarity of fit quality (e.g., potential energy of electrostatic interaction); (171.) storing the 3-dimensional coordinates of the nanostructure node multimeric protein: streptavidin complex along with quality measures in a database; (17m.) iterating steps 17i through 171 over the amino acid residues identified as specific amino acid reactive sites; and (17n.) ranking quality measures of stored nanostructure node multimeric protein: streptavidin complexes and/or examining coordinates of stored nanostructure node multimeric protein: streptavidin complexes and selecting an optimal nanostructure node multimeric protein for production.

Paragraph 18. The method of Paragraph 17, further comprising producing the optimal nanostructure multimeric protein.

Paragraph 19. The method of Paragraph 17, further comprising modifying the template sequence of the template multimeric protein (e.g., by adding, removing, or replacing an amino acid at the surface of the template multimeric protein) to improve the complementarity of fit quality between the template multimeric protein and the streptavidin tetramer.

Paragraph 20. The method of Paragraph 10, Paragraph 15, and/or Paragraph 18, further comprising modifying at least one polypeptide chain of the optimal nanostructure node multimeric protein through reaction with a bifunctional reagent to incorporate additional binding or other functionality into the at least one polypeptide chain.

Paragraph 21. The method of Paragraph 10, Paragraph 15, and/or Paragraph 18, further comprising modifying at least one polypeptide chain of the optimal nanostructure node multimeric protein through covalent incorporation of an amino acid sequence coding for protein binding or other functionality.

Paragraph 22. The method of Paragraph 10, Paragraph 15, and/or Paragraph 18, further comprising introducing at least one linking polypeptide sequence (e.g., through a genetic method) to covalently interconnect at least two subunits of the template multimeric protein, so that the optimal nanostructure node multimeric protein comprises a number of polypeptide chains reduced from the template number of polypeptide chains, wherein the linking polypeptide sequence is determined through application of a computer graphic, mathematical, or empirical method.

Composition of Matter: Nanostructure Node Paragraphs

Paragraph 23. A nanostructure node, comprising: a nanostructure node multimeric protein comprising at least one polypeptide chain, wherein the nanostructure node multimeric protein has a known 3-dimensional structure, wherein the nanostructure node multimeric protein essentially has Cn, Dn, or higher symmetry with a number of subunits, wherein the nanostructure node multimeric protein is stable at a temperature of 70° C. or greater, wherein the nanostructure node multimeric protein has an amino acid sequence not found in nature, wherein the nanostructure node multimeric protein comprises a specific binding site for the attachment of a nanostructure strut with predefined stoichiometry and orientation, wherein the specific binding site comprises at least two specific amino acid reactive residues, and wherein each specific amino acid reactive residue can have a covalently attached biotin group.

Paragraph 24. The nanostructure node of Paragraph 23, wherein the nanostructure node multimeric protein has a sequence having 80 percent or greater sequence identity with the sequence of a protein provided in Table 1.

Paragraph 25. The nanostructure node of Paragraph 23, wherein at least one polypeptide chain of the nanostructure node multimeric protein is bonded to a bifunctional reagent with additional binding or other functionality.

Paragraph 26. The nanostructure node of Paragraph 23, wherein at least one polypeptide chain of the nanostructure node multimeric protein comprises an amino acid sequence coding for protein binding or other functionality.

Paragraph 27. The nanostructure node of Paragraph 23, wherein at least two subunits are covalently interconnected with a polypeptide linker.

Paragraph 28. The nanostructure node of Paragraph 27, wherein the nanostructure node multimeric protein comprises a number of polypeptide chains less than the number of subunits.

Paragraph 29. The nanostructure node of Paragraph 27, wherein the nanostructure node multimeric protein comprises a single polypeptide chain.

Paragraph 30. The nanostructure node of Paragraph 27, wherein the nanostructure node multimeric protein has Cn symmetry.

Paragraph 31. The nanostructure node of Paragraph 27, wherein the nanostructure node is a planar node, wherein the nanostructure strut is a streptavidin strut, wherein the nanostructure node multimeric protein comprises one polypeptide chain, wherein the nanostructure node multimeric protein has an amino acid sequence with greater than 80 percent sequence identity with the amino acid sequence of a pdb code: 1thj protein trimer.

Paragraph 32. The nanostructure node of Paragraph 31, wherein the nanostructure node multimeric protein has an amino acid sequence given in Table 2B or has an amino acid sequence with greater than 80 percent sequence identity with a sequence provided in Table 2B.

Paragraph 33. The nanostructure node of Paragraph 23, wherein the nanostructure node is a C3-symmetric planar node, wherein the nanostructure strut is a streptavidin or streptavidin-incorporating strut, wherein the nanostructure node multimeric protein has an amino acid sequence with greater that 80 percent identity to an amino acid sequence provided in Table 2A (for example, the amino acid sequence of the pdb code: 1 thj protein trimer).

Paragraph 34. The nanostructure node of Paragraph 23, wherein the nanostructure node is a C3-symmetric planar node, wherein the nanostructure strut is a streptavidin or streptavidin-incorporating strut, wherein the nanostructure node multimeric protein has an amino acid sequence with greater than 80 percent identity to an amino acid sequence provided in Table 2C (for example, the amino acid sequence of the pdb code: 1j5s protein trimer).

Paragraph 35. The nanostructure node of Paragraph 23, wherein the nanostructure node is a C4-symmetric planar node, wherein the nanostructure strut is a streptavidin or streptavidin-incorporating strut, wherein the nanostructure node multimeric protein has an amino acid sequence with greater than 80 percent identity to an amino acid sequence provided in Table 2E (for example, the amino acid sequence of the pdb code: 1vcg protein tetramer).

Paragraph 36. The nanostructure node of Paragraph 23, wherein the nanostructure node is a C4-symmetric planar node, wherein the nanostructure strut is a streptavidin or streptavidin-incorporating strut, wherein the nanostructure node multimeric protein has an amino acid sequence with greater than 80 percent identity to the pdb code: 2cu0 protein tetramer of Table 1.

Paragraph 37. The nanostructure node of Paragraph 23, wherein the nanostructure node is a C5-symmetric planar node, wherein the nanostructure strut is a streptavidin strut, wherein the nanostructure node multimeric protein has an amino acid sequence with greater than 80 percent identity to the pdb code: 1vdh protein pentamer of Table 1.

Paragraph 38. The nanostructure node of Paragraph 23, wherein the nanostructure node is a C6-symmetric planar node, wherein the nanostructure strut is a streptavidin or streptavidin-incorporating strut, wherein the nanostructure node multimeric protein has an amino acid sequence with at least 80 percent identity to the pdb code: 2ekd protein hexamer of Table 1.

Paragraph 39. The nanostructure node of Paragraph 23, wherein the nanostructure node is a C7-symmetric planar node, wherein the nanostructure strut is a streptavidin or streptavidin-incorporating strut, wherein the nanostructure node multimeric protein is the pdb code: 1i81 protein heptamer of Table 1.

Paragraph 40. The nanostructure node of Paragraph 23, wherein the nanostructure node is a C3-symmetric apex node, wherein the nanostructure strut is a streptavidin or streptavidin-incorporating strut, wherein the nanostructure struts are attachable to the nanostructure node multimeric protein with dodecahedral apex geometry, wherein the nanostructure node multimeric protein has an amino acid sequence with at least 80 percent identity to an amino acid sequence provided in Table 2D (for example, the amino acid sequence of the pdb code: 1v4n protein trimer).

Paragraph 41. The nanostructure node of Paragraph 23, wherein the nanostructure node is a C3-symmetric apex node, wherein the nanostructure strut is a streptavidin or streptavidin-incorporating strut, wherein the nanostructure struts are attachable to the nanostructure node multimeric protein with “buckyball” or truncated icosahedral apex geometry, wherein the nanostructure node multimeric protein has an amino acid sequence with at least 80 percent identity to an amino acid sequence provided in Table 2D (for example, the amino acid sequence of the pdb code: 1v4n protein trimer).

Paragraph 42. The nanostructure node of Paragraph 23, wherein the nanostructure node is a C5-symmetric apex node, wherein the nanostructure strut is a streptavidin or streptavidin-incorporating strut, wherein the nanostructure struts are attachable to the nanostructure node multimeric protein with icosahedral apex geometry, wherein the nanostructure node multimeric protein has an amino acid sequence with at least 80 percent identity to an amino acid sequence provided in Table 2F (for example, the amino acid sequence of the pdb code: 1vdh protein pentamer).

Paragraph 43. The nanostructure node of Paragraph 23, wherein the nanostructure node is a D2 symmetric node, wherein the nanostructure strut is a streptavidin or streptavidin-incorporating strut, wherein the nanostructure struts are attachable to the nanostructure node multimeric protein with, for example, linear, 2-dimensional rectangular, or 3-dimensional orthorhombic lattice geometry, and wherein the nanostructure node multimeric protein has an amino acid sequence with at least 80 percent identity to an amino acid sequence provided in Table 1, Table 2, or Table 2H (for example, the amino acid sequence of the pdb code: 1ma1 protein tetramer).

Paragraph 44. The nanostructure node of Paragraph 23, wherein the nanostructure node is a D2 symmetric node, wherein the nanostructure strut is a streptavidin or streptavidin-incorporating strut, wherein the nanostructure struts are attachable to the nanostructure node multimeric protein with, for example, linear, 2-dimensional rectangular, or 3-dimensional orthorhombic lattice geometry, and wherein the nanostructure node multimeric protein has an amino acid sequence with at least 80 percent identity to an amino acid sequence provided in Table 21 (for example, the amino acid sequence of the pdb code: 1nto protein tetramer).

Paragraph 45. The nanostructure node of Paragraph 23, wherein the nanostructure node is a D2 symmetric node, wherein the nanostructure strut is a streptavidin or streptavidin-incorporating strut, wherein the nanostructure struts are attachable to the nanostructure node multimeric protein with, for example, linear, 2-dimensional rectangular, or 3-dimensional orthorhombic lattice geometry, and wherein the nanostructure node multimeric protein has an amino acid sequence with at least 80 percent identity to an amino acid sequence provided in Table 2J (for example, the amino acid sequence of the pdb code: 1rtw protein tetramer).

Paragraph 46. The nanostructure node of Paragraph 23, wherein the nanostructure node is a D3 symmetric node, wherein the nanostructure strut is a streptavidin or streptavidin-incorporating strut, wherein the nanostructure struts are attachable to the nanostructure node multimeric protein with, for example, hexagonal geometry, and wherein the nanostructure node multimeric protein has an amino acid sequence with at least 80 percent identity to an amino acid sequence provided in Table 2K (for example, the amino acid sequence of the pdb code: 1b4b protein hexamer).

Paragraph 47. The nanostructure node of Paragraph 23, wherein the nanostructure node is a D3 symmetric node, wherein the nanostructure strut is a streptavidin or streptavidin-incorporating strut, wherein the nanostructure struts are attachable to the nanostructure node multimeric protein with, for example, hexagonal geometry, and wherein the nanostructure node multimeric protein has an amino acid sequence with at least 80 percent identity to an amino acid sequence provided in Table 2L (for example, the amino acid sequence of the pdb code: 1hyb protein hexamer).

Paragraph 48. The nanostructure node of Paragraph 23, wherein the nanostructure node is a D3 symmetric node, wherein the nanostructure strut is a streptavidin or streptavidin-incorporating strut, wherein the nanostructure struts are attachable to the nanostructure node multimeric protein with, for example, hexagonal geometry, and wherein the nanostructure node multimeric protein has an amino acid sequence with at least 80 percent identity to an amino acid sequence provided in Table 2M (for example, the amino acid sequence of the pdb code: 2prd protein hexamer).

Paragraph 49. The nanostructure node of Paragraph 23, wherein the nanostructure node is a D4 symmetric node, wherein the nanostructure strut is a streptavidin or streptavidin-incorporating strut, wherein the nanostructure struts are attachable to the nanostructure node multimeric protein with, for example, rectangular geometry, and wherein the nanostructure node multimeric protein has an amino acid sequence with at least 80 percent identity to an amino acid sequence provided in Table 2N (for example, the amino acid sequence of the pdb code: 1o4v protein octamer).

Paragraph 50. The nanostructure node of Paragraph 23, wherein the nanostructure node is a D4 symmetric node, wherein the nanostructure strut is a streptavidin or streptavidin-incorporating strut, wherein the nanostructure struts are attachable to the nanostructure node multimeric protein with, for example, rectangular geometry, and wherein the nanostructure node multimeric protein has an amino acid sequence with at least 80 percent identity to an amino acid sequence provided in Table 2O (for example, the amino acid sequence of the pdb code: 2h21 protein octamer).

Paragraph 51. The nanostructure node of Paragraph 23, wherein the nanostructure node is a D4 symmetric node, wherein the nanostructure strut is a streptavidin or streptavidin-incorporating strut, wherein the nanostructure struts are attachable to the nanostructure node multimeric protein with, for example, rectangular geometry, and wherein the nanostructure node multimeric protein has an amino acid sequence with at least 80 percent identity to an amino acid sequence provided in Table 2P (for example, the amino acid sequence of the pdb code: 1iel protein octamer).

Paragraph 52. The nanostructure node of Paragraph 23, wherein the nanostructure node is a tetrahedral T23 symmetric node, wherein the nanostructure strut is a streptavidin or streptavidin-incorporating strut, wherein the nanostructure struts are attachable to the nanostructure node multimeric protein with, for example, cubic lattice geometry, and wherein the nanostructure node multimeric protein has an amino acid sequence with at least 80 percent identity to an amino acid sequence provided in Table 2Q (for example, the amino acid sequence of the pdb code: 1pvv protein dodecamer).

Paragraph 53. A nanostructure node, comprising a nanostructure node tetrameric protein having D2 symmetry, wherein the nanostructure node tetrameric protein comprises a specific binding site for the attachment of at least one nanostructure strut with predefined stoichiometry and orientation, wherein the specific binding site comprises at least two specific amino acid reactive residues, and wherein each specific amino acid reactive residue can have a covalently attached biotin group, wherein the nanostructure strut is a streptavidin or streptavidin-incorporating strut, wherein the nanostructure node tetrameric protein has an amino acid sequence with at least 80 percent identity to an amino acid sequence provided in Table 2G (for example, the amino acid sequence of the streptavidin protein tetramer (pdb code: 1stp)).

Composition of Matter: Extended Struts

Paragraph 54. An extended or streptavidin-incorporating nanostructure strut, comprising: the nanostructure node of Paragraph 43, and further comprising a first streptavidin strut and a second streptavidin strut, wherein the nanostructure node has at least two specific binding sites and wherein the first streptavidin strut is bound to a first specific binding site and the second streptavidin strut is bound to the second specific binding site.

Composition of Matter: Assemblies with a Nanostructure Node

Paragraph 55. A nanostructural assembly, comprising: at least one nanostructure node according to Paragraph 23; and a nanostructure strut bound to the specific binding site, wherein the nanostructure strut comprises or incorporates streptavidin.

Paragraph 56. The nanostructural assembly of Paragraph 55, wherein the at least one nanostructure node of Paragraph 23 is selected from the nodes provided in Table 2.

Paragraph 57. The nanostructural assembly of Paragraph 55, wherein the nanostructure node multimeric protein has an amino acid sequence with greater than 80 percent identity to a protein of Table 1.

Paragraph 58. The nanostructural assembly of Paragraph 55, wherein the nanostructure strut comprises the nanostructure node of Paragraph 53.

Paragraph 59. The nanostructural assembly of Paragraph 55, wherein at least one polypeptide chain of the nanostructure node multimeric protein is bonded to a bifunctional reagent with additional binding or other functionality and/or at least one polypeptide chain of the nanostructure node multimeric protein comprises an amino acid sequence coding for protein binding or other functionality.

Paragraph 60. The nanostructural assembly of Paragraph 55, wherein the nanostructural assembly has the form of a radial planar array.

Paragraph 61. The nanostructural assembly of Paragraph 55, wherein the nanostructural assembly has the form of a planar polygon and wherein the nanostructure node has Cn symmetry.

Paragraph 62. The nanostructural assembly with the form of a planar polygon of Paragraph 61, wherein the nanostructure node is formed of a single polypeptide chain.

Paragraph 63. The nanostructural assembly with the form of a planar polygon of Paragraph 61, wherein the nanostructural assembly has the form of a planar hexagon and wherein the nanostructure node has C3 symmetry.

Paragraph 64. The nanostructural assembly of Paragraph 63, wherein the nanostructure node multimeric protein has an amino acid sequence with greater than 80 percent identity to a C3 symmetry protein of Table 1 (for example, the amino acid sequence of the pdb code: 1thj protein trimer).

Paragraph 65. The nanostructural assembly of Paragraph 63, wherein the nanostructure node is specified in Table 2B.

Paragraph 66. The nanostructural assembly with the form of a planar polygon of Paragraph 61, wherein the nanostructural assembly has the form of a planar square and wherein the nanostructure node has C4 symmetry.

Paragraph 67. The nanostructural assembly of Paragraph 66, wherein the nanostructure node multimeric protein has an amino acid sequence with greater than 80 percent identity to a C4 symmetry protein of Table 1 (for example, the pdb code: 1vcg protein tetramer).

Paragraph 68. The nanostructural assembly of Paragraph 66, wherein the nanostructure node is specified in Table 2E.

Paragraph 69. The nanostructural assembly of Paragraph 55, wherein the nanostructural assembly has the form of a 2-dimensional lattice and wherein the nanostructure node has Cn symmetry.

Paragraph 70. The nanostructural assembly with the form of a 2-dimensional lattice of Paragraph 69, further comprising a nanostructure node with Dn symmetry.

Paragraph 71. The nanostructural assembly with the form of a 2-dimensional lattice of Paragraph 69, wherein the nanostructural assembly has the form of a 2-dimensional hexagonal lattice.

Paragraph 72. The nanostructural assembly with the form of a 2-dimensional hexagonal lattice of Paragraph 71, wherein the nanostructure node multimeric protein has an amino acid sequence with greater than 80 percent identity to the pdb code: 1thj protein trimer or the pdb code: 1j5s protein trimer and wherein the amino acid sequence of the nanostructure node multimeric protein is specified in Table 2A, Table 2B, or Table 2C.

Paragraph 73. The nanostructural assembly with the form of a 2-dimensional lattice of Paragraph 69, wherein the nanostructural assembly has the form of a 2-dimensional square lattice.

Paragraph 74. The nanostructural assembly with the form of a 2-dimensional square lattice of Paragraph 73, wherein the nanostructure node multimeric protein has an amino acid sequence with greater than 80 percent identity to a C4 symmetry protein of Table 1 (for example, the pdb code: 1vcg protein tetramer).

Paragraph 75. The nanostructural assembly with the form of a 2-dimensional square lattice of Paragraph 73, wherein the nanostructure node is specified in Table 2E.

Paragraph 76. The nanostructural assembly of Paragraph 55, wherein the nanostructural assembly has the form of a 3-dimensional radial array, and wherein the nanostructure node has Dn, tetrahedral, cubeoctahedral, icosahedral or dodecahedral symmetry.

Paragraph 77. The nanostructure assembly with the form of a 3-dimensional radial array of Paragraph 76, wherein the nanostructure node multimeric protein has an amino acid sequence with greater than 80 percent identity to a protein of Table 1.

Paragraph 78. The nanostructural assembly with the form of a 3-dimensional radial array of Paragraph 76, wherein the nanostructural assembly has the form of a 3-dimensional radial array with six arms directed along three mutually perpendicular axes and wherein the nanostructure node has tetrahedral (T23) symmetry.

Paragraph 79. The nanostructural assembly with the form of a 3-dimensional radial array with six arms directed along three mutually perpendicular axes of Paragraph 78, wherein the nanostructure node multimeric protein has an amino acid sequence with greater than 80 percent identity to a T23 symmetry protein of Table 1 (for example, the pdb code: 1pvv protein dodecamer).

Paragraph 80. The nanostructural assembly with the form of a 3-dimensional radial array with six arms directed along three mutually perpendicular axes of Paragraph 78, wherein the nanostructure node is specified in Table 2Q.

Paragraph 81. A nanostructural assembly with the form of a 3-dimensional radial array of Paragraph 76, wherein the nanostructural assembly has the form of a 3-dimensional radial array with 18 arms directed toward the apices of a cuboctahedron and wherein the nanostructure node has cuboctahedral symmetry.

Paragraph 82. The nanostructural assembly with the form of a 3-dimensional radial array with 18 arms directed toward the apices of a cuboctahedron of Paragraph 81, wherein the nanostructure node multimeric protein has an amino acid sequence with greater than 80 percent identity to an O symmetry (cuboctahedral symmetry) 24-subunit protein of Table 1.

Paragraph 83. A nanostructural assembly with the form of a 3-dimensional radial array of Paragraph 76, wherein the nanostructural assembly has the form of a 3-dimensional radial array with 30 arms directed along the dyad axes of a dodecahedron and wherein the nanostructure node has dodecahedral symmetry.

Paragraph 84. The nanostructural assembly with the form of a 3-dimensional radial array with 30 arms directed along the dyad axes of a truncated icosahedron of Paragraph 83, wherein the nanostructure node multimeric protein has an amino acid sequence with greater than 80 percent identity to a truncated icosahedral symmetry protein of Table 1.

Paragraph 85. The nanostructural assembly of Paragraph 55, wherein the nanostructural assembly has the form of a regular 3-dimensional polyhedron.

Paragraph 86. The nanostructural assembly with the form of a regular 3-dimensional polyhedron of Paragraph 85, wherein the nanostructure node multimeric protein has Cn symmetry and has an amino acid sequence with greater than 80 percent identity to a protein of Table 1.

Paragraph 87. The nanostructural assembly with the form of a regular 3-dimensional polyhedron of Paragraph 85, wherein the nanostructural assembly has the form of a regular dodecahedron.

Paragraph 88. The nanostructural assembly with the form of a regular dodecahedron of Paragraph 87, wherein the nanostructure node is the C3-symmetric apex node of Paragraph 40.

Paragraph 89. The nanostructural assembly with the form of a regular 3-dimensional polyhedron of Paragraph 85, wherein the nanostructural assembly has the form of a regular “buckyball” or truncated icosahedron.

Paragraph 90. The nanostructural assembly with the form of a regular “buckyball” or truncated icosahedron of Paragraph 89, wherein the nanostructure node is the C3-symmetric apex node of Paragraph 41.

Paragraph 91. The nanostructural assembly with the form of a regular 3-dimensional polyhedron of Paragraph 85, wherein the nanostructural assembly has the form of a regular icosahedron.

Paragraph 92. The nanostructural assembly with the form of a regular icosahedron of Paragraph 91, wherein the nanostructure node is a C5-symmetric apex node of Paragraph 42.

Paragraph 93. The nanostructural assembly of Paragraph 55, wherein the nanostructural assembly has the form of a three-connected, hexagonal-pattern, 3-dimensional lattice.

Paragraph 94. The nanostructural assembly with the form of a three-connected, hexagonal-pattern, 3-dimensional lattice of Paragraph 93, comprising: a first nanostructure node and a second nanostructure node, wherein the first nanostructure node and the second nanostructure node have D3 symmetry, wherein the nanostructure node multimeric protein of the first nanostructure node and the nanostructure node multimeric protein of the second nanostructure node are the same or different and independently have amino acid sequences with greater than 80 percent identity to a protein of Table 1, wherein the specific binding site of the first nanostructure node binds the nanostructure strut with a first orientation, wherein the specific binding site of the second nanostructure node binds the nanostructure strut with a second orientation, wherein the second orientation is rotated 90 degrees with respect to the first orientation.

Paragraph 95. The nanostructural assembly with the form of a three-connected, hexagonal-pattern, 3-dimensional lattice of Paragraph 94, wherein the nanostructure node multimeric protein of the first nanostructure node and the nanostructure node multimeric protein of the second nanostructure node are the same.

Paragraph 96. The nanostructural assembly with the form of a three-connected, hexagonal-pattern, 3-dimensional lattice of Paragraph 93, wherein the nanostructure node multimeric protein has an amino acid sequence with greater than 80 percent identity to the pdb code: 1hyb hexameric multimer.

Paragraph 97. The nanostructural assembly of Paragraph 55, wherein the nanostructural assembly has the form of a four-connected, cubic-pattern, 3-dimensional lattice.

Paragraph 98. The nanostructural assembly with the form of a four-connected, cubic-pattern, 3-dimensional lattice of Paragraph 97, comprising: a first nanostructure node and a second nanostructure node, wherein the first nanostructure node and the second nanostructure node have D4 symmetry, wherein the nanostructure node multimeric protein of the first nanostructure node and the nanostructure node multimeric protein of the second nanostructure node are the same or different and independently have amino acid sequences with greater than 80 percent identity to a protein of Table 1, wherein the specific binding site of the first nanostructure node binds the nanostructure strut with a first orientation, wherein the specific binding site of the second nanostructure node binds the nanostructure strut with a second orientation, wherein the second orientation is rotated 90 degrees with respect to the first orientation.

Paragraph 99. The nanostructural assembly with the form of a four-connected, cubic-pattern, 3-dimensional lattice of Paragraph 97, wherein the nanostructure node multimeric protein has an amino acid sequence with greater than 80 percent identity to the pdb code: 2h21 octameric multimer.

Paragraph 100. The nanostructural assembly of Paragraph 55, wherein the nanostructural assembly has the form of a six-connected, cubic, 3-dimensional lattice.

Paragraph 101. The nanostructural assembly with the form of a six-connected, cubic, 3-dimensional lattice of Paragraph 100, wherein the nanostructure node has tetrahedral (T23) symmetry.

Paragraph 102. The nanostructural assembly with the form of a six-connected, cubic, 3-dimensional lattice of Paragraph 100, wherein the nanostructure node has tetrahedral (T23) symmetry and wherein the nanostructure node multimeric protein has an amino acid sequence with greater than 80 percent identity to a protein of Table 1.

Paragraph 103. The nanostructural assembly with the form of a six-connected, cubic, 3-dimensional lattice of Paragraph 100, wherein the nanostructure node has tetrahedral (T23) symmetry and wherein the nanostructure node multimeric protein has an amino acid sequence with greater than 80 percent identity to a protein of Table 2Q (for example, the pdb code: 1pvv protein dodecamer).

Composition of Matter: Multimeric Node Protein Architectures

Paragraph 104. A nanostructure node multimeric protein, comprising: at least three polypeptide subunits; at least one specific binding site, comprising a pair of specific amino acid reactive residues, wherein each specific amino acid residue is capable of covalently attaching a biotin group, so that at least one nanostructure strut can be attached to the nanostructure node multimeric protein; and at least one of the following: there are 1, 2, 3, 5, or >5 attachment specific binding sites; there are 4 specific binding sites, but these are not symmetric or these are related by a symmetry other than four-fold rotational symmetry; there are 1, 2, 3, 5, or >5 polypeptide chains that form the at least three subunits; at least one of the specific binding sites comprises a specific amino acid residue other than cysteine; at least one of the specific binding sites comprises is within a ligand binding pocket of the nanostructure node multimeric protein; the C terminus of the polypeptide subunit is not covalently linked to a His6 tag; the subunit is of a thermostable protein or has 80% sequence identity with the sequence of a subunit of a thermostable protein; and/or at least one of the subunits is a genetically engineered protein.

Paragraph 105. The nanostructure node multimeric protein of Paragraph 104, wherein there are 3, 5, or 6 subunits.

Paragraph 106. The nanostructure node multimeric protein of Paragraph 104, wherein the subunits are related by rotational symmetry.

Paragraph 107. The nanostructure node multimeric protein of Paragraph 104, wherein the polypeptide subunits are essentially related by a symmetry selected from the group consisting of tetrahedral symmetry, octahedral symmetry, and icosahedral symmetry.

Paragraph 108. The nanostructure node multimeric protein of Paragraph 104, wherein each subunit comprises one specific binding site.

Paragraph 109. The nanostructure node multimeric protein of Paragraph 104, wherein at least one subunit does not comprise a specific binding site.

Paragraph 110. The nanostructure node multimeric protein of Paragraph 104, wherein there are 4 specific binding sites related by four-fold rotational symmetry.

Paragraph 111. The nanostructure node multimeric protein of Paragraph 104, wherein there are 3, 5, or 6 specific binding sites that lie within the same plane.

Paragraph 112. The nanostructure node multimeric protein of Paragraph 104, wherein there are 3, 5, or 6 specific binding sites that are related by rotational symmetry.

Paragraph 113. The nanostructure node multimeric protein of Paragraph 104, wherein there are 4 specific binding sites and wherein at least one specific binding site does not lie within the same plane as the other specific binding sites.

Paragraph 114. The nanostructure node multimeric protein of Paragraph 104, wherein a first subunit is covalently bonded to a second subunit.

Paragraph 115. The nanostructure node multimeric protein of Paragraph 104, wherein the at least three subunits are formed from one polypeptide chain.

Paragraph 116. The nanostructure node multimeric protein of Paragraph 104, wherein each subunit is thermostable.

Paragraph 117. The nanostructure node multimeric protein of Paragraph 104, wherein the amino acid sequence of at least one subunit is different from the amino acid sequence of another subunit.

Paragraph 118. The nanostructure node multimeric protein of Paragraph 104, wherein each polypeptide subunit comprises an amino acid sequence of at least 50 amino acid residues having at least 80% sequence identity with an amino acid sequence of a protein of a thermophilic organism.

Paragraph 119. The nanostructure node multimeric protein of Paragraph 104, wherein there are three subunits and wherein the amino acid sequence of each polypeptide subunit has at least 80% sequence identity with an amino acid sequence of the uronate isomerase TM0064 from Thermotoga maritima (pdb code: 1j5s).

Paragraph 120. The nanostructure node multimeric protein of Paragraph 104, wherein there are four subunits and wherein the amino acid sequence of each polypeptide subunit has at least 80% amino acid sequence identity with an amino acid sequence of the isopentenyl-diphosphate delta-isomerase (pdbcode: 1vcg) from Thermus thermophilus.

Paragraph 121. The nanostructure node multimeric protein of Paragraph 104, wherein each specific binding site has each specific amino acid residue separated from the other specific amino acid residue by a distance of about 20 Angstroms.

Paragraph 122. The nanostructure node multimeric protein of Paragraph 104, wherein each specific binding site has each separated from the other specific amino acid residue by a distance such that with biotin groups bound to the specific amino acid residues, the biotin groups are positioned to bind with a pair of binding sites on streptavidin.

Paragraph 123. The nanostructure node multimeric protein of Paragraph 104, wherein at least one subunit comprises an amino acid sequence having a designated amino and/or carboxy terminus and further comprising an amino acid (polypeptide) extension of from 5 to 1000 amino acid residues linked with a peptide bond to the designated amino and/or carboxy terminus.

Paragraph 124. The nanostructure node multimeric protein of Paragraph 123, wherein the amino acid extension comprises a binding function for a protein, metallic or other surface.

Paragraph 125. The nanostructure node multimeric protein of Paragraph 123, wherein the amino acid extension comprises an amino acid subsequence that is a substrate for an enzyme.

Paragraph 126. The nanostructure node multimeric protein of Paragraph 123, wherein the amino acid extension comprises a polypeptide subsequence selected from the group consisting of an immunoglobulin polypeptide, a polyhistidine, a streptavidin binding polypeptide, Streptag, an antibody binding polypeptide, staphylococcus Protein A, staphylococcus Protein G, an antigenic polypeptide, and a hapten-binding polypeptide.

Paragraph 127. The nanostructure node multimeric protein of Paragraph 123, wherein the polypeptide extension comprises an antibody binding polypeptide subsequence and further comprising an antibody bound to the antibody binding polypeptide subsequence.

Paragraph 128. A functionalized nanostructure node multimeric protein, comprising: the nanostructure node multimeric protein of Paragraph 104; and a nanostructure strut, wherein the nanostructure strut is bound to the specific binding site.

Paragraph 129. The nanostructure node multimeric protein of Paragraph 104, wherein there are three subunits, wherein two subunits comprise a specific binding site, and wherein one subunit does not comprise a specific binding site.

Paragraph 130. The nanostructure node multimeric protein of Paragraph 104, wherein there are three subunits, wherein one subunit comprises a specific binding site, and wherein two subunits do not comprise a specific binding site.

Paragraph 131. The nanostructure node multimeric protein of Paragraph 104, wherein there are four subunits, wherein three subunits comprise a specific binding site, and wherein one subunit does not comprise a specific binding site.

Paragraph 132. The nanostructure node multimeric protein of Paragraph 104, wherein there are four subunits, wherein two subunits comprise a specific binding site, wherein two subunits do not comprise an attachment locus, wherein the subunits are essentially related by four-fold rotational symmetry, and wherein the subunit that comprises a specific binding site alternates with the subunit that does not comprise a specific binding site in the ordering of subunits about the four-fold rotational symmetry axis.

Paragraph 133. The nanostructure node multimeric protein of Paragraph 104, wherein there are four subunits, wherein two subunits comprise a specific binding site, wherein two subunits do not comprise a specific binding site, wherein the subunits are essentially related by four-fold rotational symmetry, and wherein the two subunits that comprise specific binding sites are consecutive in the ordering of subunits about the four-fold rotational symmetry axis.

Paragraph 134. The nanostructure node multimeric protein of Paragraph 104, wherein there are four subunits, wherein one subunit comprises a specific binding site, and wherein three subunits do not comprise a specific binding site.

Paragraph 135. A protein superstructure, comprising: the nanostructure node multimeric protein of Paragraph 104.

Paragraph 136. The protein superstructure of Paragraph 135, wherein the nanostructure strut comprises streptavidin; wherein biotin groups covalently bound to the specific amino acid residues are bound to biotin sites on the streptavidin.

Paragraph 137. The protein superstructure of Paragraph 136, wherein the streptavidin is immobilized on a surface.

Paragraph 138. The protein superstructure of Paragraph 136, further comprising: a protein comprising an adenosine triphosphate (ATP) binding site (e.g., MJ0577); and a bridge molecule having a biotin group covalently bound to an adenosine triphosphate (ATP) group; wherein the bridge molecule is bound to the biotin binding sites on the streptavidin and the ATP binding site.

Paragraph 139. The protein superstructure comprising: the nanostructure node multimeric protein of Paragraph 104; and a protein comprising an adenosine triphosphate (ATP) binding site (e.g. MJ0577), wherein an adenosine triphosphate (ATP) or a derivative thereof is covalently attached to each specific amino acid residue; wherein the adenosine triphosphate (ATP) group is bound to the ATP binding site.

Paragraph 140. The protein superstructure of Paragraph 135, wherein the second functional group of the linker molecule is adenosine triphosphate (ATP) or a derivative thereof, wherein the complementary protein (e.g., MJ0577) comprises an adenosine triphosphate (ATP) binding site as the complementary binding site, wherein the second functional group of the linker molecule is bound to the adenosine triphosphate (ATP) complementary binding site of the complementary protein, wherein a streptavidin having a biotin binding site is linked to the complementary protein by a second linker molecule, wherein the second linker molecule comprises a thiol-reactive group covalently bonded to a biotin, iminobiotin, or a derivative thereof, wherein the thiol-reactive group of the second linker molecule is covalently bonded to a surface amino acid residue of the complementary protein, and wherein the biotin, iminobiotin, or derivative thereof of the second linker molecule is bound to a biotin binding site of the streptavidin.

Paragraph 141. A kit, comprising: a nanostructure node multimeric protein of Paragraph 23; and a monostructure strut.

Paragraph 142. The kit of Paragraph 141, wherein the nanostructure strut comprises streptavidin.

Paragraph 143. A method of producing a nanostructure node multimeric protein, comprising: culturing a cell host in a culture medium to express the optimal nanostructure node multimeric protein of Paragraph 8, Paragraph 14, or Paragraph 17; and heating the culture medium above a temperature at which the cell host lyses and below the denaturation temperature of the nanostructure node multimeric protein to produce a lysate comprising the nanostructure node multimeric protein.

Paragraph 144. The method of Paragraph 143, further comprising using recombinant DNA technology or site-specific modification techniques to modify a nucleotide sequence of a thermophilic organism for directing the expression of the nanostructure node multimeric protein.

Paragraph 145. The method of Paragraph 143, further comprising using a gene fusion technique to modify a nucleotide sequence of a thermophilic organism for directing the expression of the nanostructure node multimeric protein to have at least two subunits covalently interconnected with a polypeptide linker.

Paragraph 146. The method of Paragraph 143, further comprising inserting the nucleotide sequence of a thermophilic organism or a modified nucleotide sequence of a thermophilic organism in the cell host to direct expression of the nanostructure node multimeric protein by the cell host.

Paragraph 147. The method of Paragraph 143, further comprising isolating the thermostable nanostructure node multimeric protein in substantially pure form from the lysate.

Paragraph 148. A method, comprising: producing bindable subunits of a nanostructure node multimeric protein, the bindable polypeptide subunits comprising a specific binding site for the attachment of a nanostructure strut; producing non-bindable subunits of the nanostructure node multimeric protein, the non-bindable subunits not comprising a specific binding site; combining the bindable polypeptide subunits and the non-bindable polypeptide subunits in a solution; allowing nanostructure node multimeric proteins to form from multimerization of the bindable and non-bindable subunits; and separating the nanostructure node multimeric proteins into fractions according to the number of bindable polypeptide subunits and the number of non-bindable polypeptide subunits in the nanostructure node multimeric protein, wherein the specific binding site comprises at least two specific amino acid reactive residues, and wherein each specific amino acid reactive residue can have a covalently attached biotin group.

Paragraph 149. The method of Paragraph 148, wherein the multisubunit node polypeptides are separated into fractions by chromatography or electrophoresis.

Paragraph 150. The method of Paragraph 148, wherein the nanostructure node multimeric protein is a trimer, wherein the fractions of nanostructure node multimeric proteins comprise trimers having 3 bindable subunits, trimers having 2 bindable subunits and 1 non-bindable subunit, trimers having 1 bindable subunit and 2 non-bindable subunits, and trimers having 3 non-bindable subunits.

Paragraph 151. The method of Paragraph 148, wherein the nanostructure node multimeric protein is a tetramer, wherein the fractions of nanostructure node multimeric proteins comprise tetramers having 4 bindable subunits, tetramers having 3 bindable subunits and 1 non-bindable subunit, tetramers having 2 bindable subunits and 2 non-bindable subunits, tetramers having 1 bindable subunit and 3 non-bindable subunits, and tetramers having 4 non-bindable subunits.

Paragraph 152. The method of Paragraph 151, wherein the subunits of the tetramer are essentially related by four-fold rotational symmetry.

Paragraph 153. The method of Paragraph 151, wherein the subunits of the tetramer are essentially related by four-fold rotational symmetry, wherein the fraction of tetramers having 2 bindable subunits and 2 non-bindable subunits is separated into subfractions of tetramers having the 2 bindable subunits consecutive in the ordering of subunits about the four-fold rotational symmetry axis, and tetramers having the bindable subunit alternate with the non-bindable subunit in the ordering of subunits about the four-fold rotational symmetry axis.

Paragraph 154. The method of Paragraph 151, wherein the subunits of the tetramer are essentially related by D2 or tetrahedral symmetry.

Method of Making a Nanostructure Assembly (Chemical Synthesis)

Paragraph 155. A method of making a protein nanostructure, comprising: providing the nanostructure node multimeric protein of Paragraph 23; providing a nanostructure strut; binding the nanostructure strut binding site of the nanostructure node multimeric protein; and binding the second functional group of the linker molecule to the complementary binding site of the complementary protein.

Paragraph 156. The method of Paragraph 155, wherein binding the nanostructure strut to the specific binding site of the nanostructure node multimeric protein comprises mixing the nanostructure strut with the nanostructure node multimeric protein to form a reaction solution and allowing the nanostructure strut to bind to the specific binding site of the nanostructure node multimeric protein.

Paragraph 157. The method of Paragraph 155, wherein the nanostructure strut comprises streptavidin.

Paragraph 158. The method of Paragraph 155, wherein the specific binding site comprises at least two specific amino acid residues, wherein each specific amino acid reactive residue has a covalently attached iminobiotin group, wherein binding the nanostructure strut to the nanostructure node multimeric protein comprises mixing the linker molecule with the complementary protein to form a reaction solution and increasing the pH of the reaction solution to at least about 7 to induce the iminobiotin group to bind to the streptavidin.

Paragraph 159. The method of Paragraph 155, wherein the specific binding site comprises at least two specific amino acid residues, wherein each specific amino acid residue has a covalently attached photo-activated nucleotide, wherein the nanostructure comprises an adenosine triphosphate binding (ATP) binding site, and wherein binding the nanostructure strut to the nanostructure node multimeric protein comprises mixing the nanostructure strut with the nanostructure node multimeric protein to form a reaction solution and irradiating the reaction solution with light to induce the photo-activated nucleotide to bind to the ATP binding site.

Method of Using a Proteinaceous Nanostructure Assembly as a Pattern or Resist

Paragraph 160. A method of using a proteinaceous nanostructure assembly as a pattern or resist masking material for the fabrication of devices with sub-100 nanometer features.

Paragraph 161. The method of Paragraph 160, using a 2-dimensional proteinaceous nanostructure assembly as a patterning material for the fabrication of devices with sub-100 nanometer features.

Paragraph 162. The method of Paragraph 160, using a 2-dimensional proteinaceous nanostructure assembly as a method of masking a resist material for the fabrication of devices with sub-100 nanometer features.

Paragraph 163. The method of Paragraph 160, using a 3-dimensional proteinaceous nanostructure assembly as a negative patterning material for the fabrication of devices with sub-100 nanometer channels.

Paragraph 164. The method of Paragraph 160, using a 3-dimensional proteinaceous nanostructure assembly as patterning material for the fabrication of devices with sub-100 nanometer features.

Paragraph 165. The method of Paragraph 160, where the device is a nanolithography stamp.

Paragraph 166. The method of Paragraph 160, where the device is a semiconductor device.

Paragraph 167. The method of Paragraph 160, where the device is a zero-mode waveguide.

Paragraph 168. The method of Paragraph 160, where the device is a microelectromechanical system (MEMS).

Paragraph 169. The method of Paragraph 160, where the device is a nanofluidics system.

Method of Making a Device Using a Proteinaceous Nanostructure Assembly as a Pattern or Resist Mask

Paragraph 170. A method of making a proteinaceous nanostructure assembly as a pattern or resist material for the fabrication of devices with sub-100 nanometer features.

Method of Making a Device Using a 2-Dimensional Proteinaceous Nanostructure Assembly as a Pattern

Paragraph 171. The method of Paragraph 170, using a 2-dimensional proteinaceous nanostructure assembly as a patterning material for the fabrication of devices with sub-100 nanometer features comprising the following steps: preparing a substrate surface to introduce specific protein attachment sites; binding a node protein or node protein assembly to the surface at specific attachment sites through chemical linkages; optionally, performing additional assembly steps involving the addition of protein struts or nodes to the surface-immobilized protein assembly to create a 2-dimensional nanostructure assembly; coating the proteinaceous nanostructure assembly with a material, for example, a metal (such as iron), a noble metal (such as gold, platinum, or silver), a glass (such as silicon dioxide), a ceramic, a semiconductor (such as silicon or germanium), a polymer, and/or an organic polymer (such as polytetrafluoroethylene).

Paragraph 172. The method of Paragraph 171, wherein the substrate comprises a metal (such as iron, gold, platinum, or silver), a noble metal (such as gold, platinum, or silver), a glass (such as silicon dioxide), a self-assembling monolayer, plastic, a polymer, an organic polymer (such as polytetrafluoroethylene), an organic material, a ceramic, and/or a semiconductor material (such as silicon or germanium).

Paragraph 173. The method of Paragraph 171, wherein the proteinaceous nanostructure assembly is assembled from node proteins derived from node template structures listed in Table 1, or alternatively, node proteins derived from node template structures listed in Table 1 and streptavidin or streptavidin-incorporating struts.

Method of Making a Device Using a 2-Dimensional Proteinaceous Nanostructure Assembly as a Resist Mask

Paragraph 174. The method of Paragraph 170, using a 2-dimensional proteinaceous nanostructure assembly as a method of patterning a resist material for the fabrication of devices with sub-100 nanometer features comprising the following steps: coating a substrate surface with a continuous resist layer whose chemical properties are altered by irradiation with a suitable wavelength of radiation; preparing the resist layer surface to introduce specific protein attachment sites; placing a node protein or node protein assembly on the resist layer surface at a predetermined location; optionally, performing additional assembly steps involving the addition of protein struts or nodes to the surface-immobilized protein assembly to create a proteinaceous 2-dimensional nanostructure assembly; exposing the surface with the bound proteinaceous 2-dimensional nanostructure assembly to irradiation, wherein the surface of the resist lying underneath the 2-dimensional nanostructure assembly is protected from irradiation; removing the irradiated resist material through chemical action; etching the surface with the bound proteinaceous 2-dimensional nanostructure assembly and underlying resist to form a pattern that is complementary to the structure of the 2-dimensional nanostructure assembly; removing, using chemical or other means, the bound proteinaceous 2-dimensional nanostructure assembly and underlying resist to leave a pattern on the substrate surface that is complementary to the structure of the 2-dimensional nanostructure assembly.

Paragraph 175. The method of Paragraph 174, further comprising binding the node protein or the node protein assembly to the resist layer surface at specific attachment sites through a chemical linkage.

Paragraph 176. The method of Paragraph 174, wherein the substrate comprises a metal (such as iron, gold, platinum, or silver), a noble metal (such as gold, platinum, or silver), a glass (such as silicon dioxide), a self-assembling monolayer, plastic, a polymer, an organic polymer (such as polytetrafluoroethylene), an organic material, a ceramic, and/or a semiconductor material (such as silicon or germanium).

Paragraph 177. The method of Paragraph 174, wherein the proteinaceous nanostructure assembly is assembled from node proteins derived from node template structures listed in Table 1, or alternatively, node proteins derived from node template structures listed in Table 1 and streptavidin or streptavidin-incorporating struts.

Method of Making a Device Using a 3-Dimensional Proteinaceous Nanostructure Assembly as a Negative Pattern

Paragraph 178. The method of Paragraph 170, using a proteinaceous 3-dimensional nanostructure assembly as a patterning material for the fabrication of devices with sub-100 nanometer channels comprising the following steps: preparing a substrate surface to introduce specific protein attachment sites; placing a node protein or node protein assembly on the resist layer surface at a predetermined location; performing additional assembly steps involving the addition of protein struts or nodes to the surface-immobilized protein assembly to create a proteinaceous 3-dimensional nanostructure assembly; coating or embedding the 3-dimensional nanostructure assembly with a matrix material; optionally, treating the assembly with radiation, light, heat, or other chemical treatment to solidify or stabilize the matrix material; treating the assembly with radiation, light, heat, or chemical treatment to remove or ablate the proteinaceous 3-dimensional nanostructure assembly, leaving the matrix material with internal channels presenting a negative impression of the original a proteinaceous 3-dimensional nanostructure assembly.

Paragraph 179. The method of Paragraph 178, further comprising binding the node protein or the node protein assembly to the resist layer surface at specific attachment sites through a chemical linkage.

Paragraph 180. The method of Paragraph 178, wherein the substrate comprises a metal (such as iron, gold, platinum, or silver), a noble metal (such as gold, platinum, or silver), a glass (such as silicon dioxide), a self-assembling monolayer, plastic, a polymer, an organic polymer (such as polytetrafluoroethylene), a ceramic, an organic material, and/or a semiconductor material (such as silicon or germanium).

Paragraph 181. The method of Paragraph 178, wherein the matrix material comprises a metal (such as iron, gold, platinum, or silver), a noble metal (such as gold, platinum, or silver), a glass (such as silicon dioxide), a self-assembling monolayer, plastic, a polymer, an organic polymer (such as polytetrafluoroethylene) a ceramic, an organic material, and/or a semiconductor material (such as silicon or germanium).

Paragraph 182. The method of Paragraph 178, wherein the proteinaceous nanostructure assembly is assembled from node proteins derived from node template structures listed in Table 1, or alternatively, node proteins derived from node template structures listed in Table 1 and streptavidin or streptavidin-incorporating struts.

Method of Making a Device Using a 3-Dimensional Proteinaceous Nanostructure Assembly as a Pattern

Paragraph 183. The method of 170, using a proteinaceous 3-dimensional nanostructure assembly as a patterning material for the fabrication of devices with sub-100 nanometer channels comprising the following steps: preparing a substrate surface to introduce specific protein attachment sites; placing a node protein or node protein assembly on the resist layer surface at a predetermined location; performing additional assembly steps involving the addition of protein struts or nodes to the surface-immobilized protein assembly to create a proteinaceous 3-dimensional nanostructure assembly; coating or embedding the 3-dimensional nanostructure assembly with a first matrix material; optionally, treating the assembly with light, heat, or other chemical treatment to solidify or stabilize the first matrix material; treating the assembly with radiation, light, heat, or chemical treatment to remove or ablate the proteinaceous 3-dimensional nanostructure assembly, leaving the first matrix material with internal channels presenting a negative impression of the original a proteinaceous 3-dimensional nanostructure assembly; coating or chemically treating the negative impression created in the first matrix material to deposit a second matrix material in the negative space originally occupied by the Proteinaceous nanostructure assembly; treating the assembly with radiation, light, heat, or chemical treatment to remove or ablate the first matrix material, leaving a 3-dimensional nanostructure assembly comprising the second matrix material with features of the original proteinaceous 3-dimensional nanostructure assembly.

Paragraph 184. The method of Paragraph 183, further comprising binding the node protein or the node protein assembly to the resist layer surface at specific attachment sites through a chemical linkage.

Paragraph 185. The method of Paragraph 183, wherein the substrate comprises a metal (such as iron, gold, platinum, or silver), a noble metal (such as gold, platinum, or silver), a glass (such as silicon dioxide), a self-assembling monolayer, plastic, a polymer, an organic polymer (such as polytetrafluoroethylene), an organic material, a ceramic, and/or a semiconductor material (such as silicon or germanium).

Paragraph 186. The method of Paragraph 183, wherein the first matrix material comprises a metal (such as iron, gold, platinum, or silver), a noble metal (such as gold, platinum, or silver), a glass (such as silicon dioxide), a self-assembling monolayer, plastic, a polymer, an organic polymer (such as polytetrafluoroethylene), an organic material, a ceramic, and/or a semiconductor material (such as silicon or germanium).

Paragraph 187. The method of Paragraph 183, wherein the second matrix material comprises a metal (such as iron, gold, platinum, or silver), a noble metal (such as gold, platinum, or silver), a glass (such as silicon dioxide), a self-assembling monolayer, plastic, a polymer, an organic polymer (such as polytetrafluoroethylene), an organic material, a ceramic, and/or a semiconductor material (such as silicon or germanium).

Paragraph 188. The method of Paragraph 183, wherein the proteinaceous nanostructure assembly is assembled from node proteins derived from node template structures listed in Table 1, or alternatively, node proteins derived from node template structures listed in Table 1 and streptavidin or streptavidin-incorporating struts.

### Devices

Paragraph 189. The method of Paragraph 170, where the device is a nanolithography stamp.

Paragraph 190. The method of Paragraph 170, where the device is a semiconductor device.

Paragraph 191. The method of Paragraph 170, where the device is a zero-mode waveguide.

Paragraph 192. The method of Paragraph 170, where the device is a microelectromechanical system (MEMS).

Paragraph 193. The method of Paragraph 170, where the device is a nanofluidics system.

Paragraph 194. A device, comprising: a substrate having a surface; a nucleation site on the substrate surface; and a nanostructure node coupled to the nucleation site.

Paragraph 195. The device of Paragraph 194, wherein a plurality of nucleation sites are on the substrate surface and wherein the nucleation sites are arranged in a periodic, quasiperiodic, or nonperiodic pattern.

Paragraph 196. The device of Paragraph 194, wherein the substrate comprises, for example, a metal (such as iron, gold, platinum, or silver), a noble metal (such as gold, platinum, or silver), a glass (such as silicon dioxide), a ceramic, a semiconductor (such as silicon or germanium), a carbon allotrope (such as diamond or graphite), a polymer, an organic polymer (such as tetrafluoroethylene), and/or an organic material and wherein the nucleation site comprises, for example, a metal atom (such as iron, gold, platinum, or silver), a noble metal atom (such as a gold, platinum, silver, or copper), a metal and/or noble metal cluster, a chemically reactive molecule, and/or a patch of chemically reactive molecules.

Paragraph 197. The device of Paragraph 194, wherein the nanostructure node comprises a nanostructure node multimeric protein comprising at least one polypeptide chain, wherein the nanostructure node multimeric protein has a known 3-dimensional structure, wherein the nanostructure node multimeric protein essentially has Cn, Dn, or higher symmetry with a number of subunits, wherein the nanostructure node multimeric protein is stable at a temperature of 70° C. or greater, wherein the nanostructure node multimeric protein has an amino acid sequence not found in nature, wherein the nanostructure node multimeric protein comprises a specific binding site for the attachment of a nanostructure strut with predefined stoichiometry and orientation, wherein the specific binding site comprises at least two specific amino acid reactive residues, and wherein each specific amino acid reactive residue can have a covalently attached biotin group.

Paragraph 198. The device of Paragraph 197, wherein at least one subunit comprises an amino acid sequence having a designated amino and/or carboxy terminus and further comprising an amino acid (polypeptide) extension of from 5 to 1000 amino acid residues linked with a peptide bond to the designated amino and/or carboxy terminus, wherein the amino acid extension comprises a binding function coupled to the nucleation site.

Paragraph 199. The device of Paragraph 197, further comprising a nanostructure strut attached to the specific binding site.

Paragraph 200. A device, comprising: a substrate having a surface with a node-occupied area and a node-unoccupied area; a nanostructure node on the node-occupied area of the surface; and a coating that covers the nanostructure node and covers the surface node-unoccupied area of the surface.

Paragraph 201. The device of Paragraph 200, wherein the coating comprises, for example, a metal (such as iron, gold, platinum, or silver), a noble metal (such as gold, platinum, or silver), a glass (such as silicon dioxide), a ceramic, a semiconductor (such as silicon or germanium), a carbon allotrope (such as diamond or graphite), a polymer, an organic polymer (such as tetrafluoroethylene), and/or an organic material.

Paragraph 202. A device, comprising: a substrate having a surface with a node-occupied area and a node-unoccupied area; the surface coated with a resist layer; and a nanostructure node on the resist layer.

Paragraph 203. The device of Paragraph 202, wherein the node-occupied area of the surface of the substrate is coated with the resist layer, and wherein the node-unoccupied area of the surface of the substrate is not coated with the resist layer.

Paragraph 204. The device of Paragraph 203, wherein the node-unoccupied area of the surface of the substrate is lower than (recessed with respect to) the node-occupied area of the surface of the substrate.

Paragraph 205. A device, comprising a proteinaceous nanostructure assembly comprising a nanostructure node.

Paragraph 206. The device of Paragraph 205, further comprising a substrate having a surface, wherein the proteinaceous nanostructure assembly is coupled to the surface of the substrate.

Paragraph 207. The device of Paragraph 206, further comprising a first matrix, wherein the first matrix interpenetrates the proteinaceous nanostructure assembly.

Paragraph 208. The device of Paragraph 207, wherein the proteinaceous nanostructure assembly has the form of a cubic lattice and wherein the first matrix has the form of a cubic lattice.

Paragraph 209. The device of Paragraph 207, wherein the first matrix comprises a metal (such as iron, gold, platinum, or silver), a noble metal (such as gold, platinum, or silver), a glass (such as silicon dioxide), a ceramic, a semiconductor (such as silicon or germanium), a polymer, an organic polymer (such as tetrafluoroethylene), and/or an organic material.

Paragraph 210. A device, comprising a second matrix material having the same or similar form as a proteinaceous nanostructure assembly.

Paragraph 211. The device of Paragraph 210, wherein the second matrix comprises a metal (such as iron, gold, platinum, or silver), a noble metal (such as gold, platinum, or silver), a glass (such as silicon dioxide), a ceramic, a semiconductor (such as silicon or germanium), a polymer, an organic polymer (such as tetrafluoroethylene), and/or an organic material.

TABLE 1
Symmetry and Applications of Thermostable Protein Nodes

| | Symmetry | Subunit | | Protein Data Bank (PDB) Code |
|---|---|---|---|---|
| Symmetry | Operations | Number | Application | http://www.rcsb.org/pdb/home/home.do |
| C3 | E, C3 | 3 | Planar Node, | 1fsz 1ge8 1isq 1j2v 1kht 1ki9 1kwg |
| | | | 2D lattice, | 1l1s 1ml4 1n2m 1n13 1o5j 1qrf 1thj |
| | | | Dodecahedral | 1ufy 1uku 1v4n 1v8d 1vke 1wvq 1wzn |
| | | | & Polyhedral | 1x25 2b33 2cz4 2dcl 2dhr 2dt4 2hik |
| | | | Node | 2nwl |
| C4 | E, C4, C2 | 4 | Planar Node, | 1bkb 1nc7 1vrd 2cu0 2fk5 2flf |
| | | | 2D lattice, | |
| | | | Polyhedral | |
| | | | Node | |
| C5 | E, C5 | 5 | Planar Node, | 1t0t 1vdh 1w8s 2b99 2bbh 2bbj 2hn1 |
| | | | 2D lattice, | 2hn2 2iub |
| | | | Icosahedral | |
| | | | PolyNode | |
| C6 | E, C6, C3, C2 | 6 | Planar, | 1i8f 1ljo 2a1b 2a18 2di4 2dr3 2ekd |
| | | | 2D lattice | 2ewh |
| | | | Node | |
| C7 | E, C7 | 7 | Planar Node | 1h64 1i4k 1i81 1jbm 1jri 1m5q |
| | | | | 1mgq |
| D2 | E, 3C2 | 4 | Strut | 1a0e 1bxb 1do6 1dof 1gtd 1hyg 1i1g |
| | | | Extenders & | 1ik6 1ixr 1j1y 1j2w 1jg8 1jvb 1knv |
| | | | Adaptors, | 1lk5 1lvw 1m8k 1ma1 1nto 1nvg 1o2a |
| | | | 2D and 3D | 1o54 1r37 1ris 1rtw 1u9y 1udd 1uir |
| | | | Lattice Nodes | 1usy 1uxt 1v6t 1v8o 1v8p 1vc2 1vco |
| | | | | 1vdk 1vjp 1vk8 1vl2 1vlv 1vr6 1w3i |
| | | | | 1wb7 1wb8 1wlu 1ws9 1wyt 1x01 1x1e |
| | | | | 1x10 1xtt 1y56 1z54 2afb 2b5d 2bri |
| | | | | 2cb1 2cd9 2cdc 2cx9 2czc 2d1y 2d8a |
| | | | | 2d29 2df5 2dfa 2drh 2dsl 2e1a 2e9f |
| | | | | 2eba 2ebj 2eo5 2ep5 2gl0 2gm7 2h6e |
| | | | | 2hae 2hmf 2iss 2j4k 2ldb 2p3n 2ph3 |
| | | | | 2yym 3pfk |
| D3 | E, C3, 3C2 | 6 | 2D & 3D | 1aup 1bgv 1bvu 1f9a 1fxk 1gtm 1hyb |
| | | | Nodes & | 1je0 1jku 1kku 1odi 1odk 1pg5 1qw9 |
| | | | Lattices | 1t57 1uan 1ude 1uiy 1v1a 1v1s 1v9l |
| | | | | 1v19 1wkl 1wz8 1wzn 1x0u 2a8y 2afb |
| | | | | 2anu 2bja 2bjk 2cqz 2cz8 2dcn 2ddz |
| | | | | 2dev 2dqb 2dxf 2dya 2eez 2g3m 2i14 |
| | | | | 2ide 2j4j 2j9d 2prd 2q8n |
| D4 | E, C4, 5C2 | 8 | 2D & 3D | 1jpu 1jq5 1m4y 1o4v 1saz 1umg 1vcf |
| | | | Nodes & | 1x9j 2ax3 2cwx 2d69 2h2i 2iel |
| | | | Lattices | |
| D5 | E, C5, 5C2 | 10 | 3D Nodes | 1geh 1w8s 1wm9 1wuq 1wur 1wx0 1znn |
| | | | | 2djw |
| D6 | E, C6, C3, | 12 | 3D Nodes & | 1m4y |
| | 7C2 | | Lattices | |
| D7 | E, D7, 7C2 | 14 | 3D Nodes | 1m5q 1th7 |
| T23 | E, 4C3, 3C2 | 12 | 1) 3D-Radial Nodes | 1pvv 1vlg 1xfo 1y0r 1y0y 1yoy 2clb |
| Tetrahedral | | | 2) Cubic Lattice | 2glf 2v18 2v19 |
| | | | (Diad Strut | |
| | | | Connections) | |
| | | | 2) Tetrahedral | |
| | | | Structures & | |
| | | | Lattices (C3 | |
| | | | Strut Connections) | |
| O (432) | E, 3C4, 4C3, | 24 | 3D-Radial | 1shs 1vlg |
| Cuboctahedral | 9C2 | | Nodes, | |
| | | | Lattices | |
| Dodecahedral | E, 6C5, 10C3, | 60 | 3D-Radial | 1b5s |
| (532) | 15C2 | | Nodes, | |
| | | | Lattices | |

Structures are designated by their Protein Data Bank Codes <http://www.rcsb.org/pdb/home/home.do>. Structures in bold text are illustrated in the application. For a complete description of point group symmetry and symmetry0 operation nomenclature see Vainstein (1994) or: http://www.phys.ncl.ac.uk/staff/njpg/symmetry/index.html and <http://csi.chemie.tu-darmstadt.de/ak/immel/script/redirect.cgi?filename=http://csi.chemie.tu-darmstadt.de/ak/immel/tutorials/symmetry/index.html>

TABLE 2

| | |
|---|---|
| No, | |
| PDB | |
| ID, | |
| Symmetry | Description, Template Sequence, Site-Directed Modifications |

Part 1. Specifications of Thermostable Node Proteins

| | |
|---|---|
| 2.A | Description: Three-fold (C3) Planar |
| lthj | Symmetric Node |
| (C3) | Template Sequence: |
| | MQEITVDEFSNIRENPVTPWNPEPSAPVIDPTAYIDPEASVIGEVTIGANVMVSPMASIRSDEGMPIFVGDRSNVQDGVV |
| | LHALETINEEGEPIEDNIVEVDGKEYAVYIGNNVSLAHQSQVHGPAAVGDDTFIGMQAFVFKSKVGNNCVLEPRSAAIGV |
| | TIPDGRYIPAGMVVTSQAEADKLPEVTDDYAYSHTNEAVVYVNVHLAEGY |
| | KETS |
| | Sequence Modifications: |
| | General: Cys148 to Ala |
| | Specific Biotinylation Sites: |
| | 1. Asp70 to Cys, Tyr200 to Cys |

Part 2. Specifications of Thermostable Node Proteins

| | |
|---|---|
| 2.B | Description: Single-Chain Three-fold (C3) Planar Symmetric Node |
| 1thj | Native template sequence (1thj): |
| (C3) | MQEITVDEFSNIRENPVTPWNPEPSAPVIDPTAYIDPEASVIGEVTIGANVMVSPMASIRSDEGMPIFVGDRSNVQDGVV |
| | LHALETINEEGEPIEDNIVEVDGKEYAVYIGNNVSLAHQSQVHGPAAVGDDTFIGMQAFVFKSKVGNNCVLEPRSAAIGV |
| | TIPDGRYIPAGMVVTSQAEADKLPEVTDDYAYSHTNEAVVYVNVHLAEGYKETS |
| | Single Chain Template Sequences: |
| | Template A: (DEFSNIRENP VTPWNPEPSA PVIDPTAYID PEASVIGEVT IGANVMVSPM ASIRSDEGMP |
| | IFVGDRSNVQ DGVVLHALET INEEGEPIED NIVEVDGKEY AVYIGNNVSL AHQSQVHGPA AVGDDTFIGM |
| | QAFVFKSKVG NNCVLEPRSA AIGVTIPDGR YIPAGMVVTS QAEADKLPEV TDDYAYSHTN EAVVYVNVHL |
| | AEGYKQT) |
| | Template B: (DEFSNIRENP VTPWNPEPSA PVIDPTAYID PEASVIGEVT IGANVMVSPM ASIRSDEGMP |
| | IFVGDRSNVQ DGVVLHALET INEEGEPIED NIVEVDGKEY AVYIGNNVSL AHQSQVHGPA AVGDDTFIGM |
| | QAFVFKSKVG NNCVLEPRSA AIGVTIPDGR YIPAGMVVTS QAEADKLPEV TDDYAYSHTN EAVVYVNVHL |
| | AEGYKQT) |
| | Linker A: (GGGSGGG) |
| | Linker B: (GGGSGGGG) |
| | Sequence Modifications: |
| | General: |
| | Template A: Cys143 to Ala, Delete N-term 6 residues (MQEITV) |
| | Template B: Cys143 to Ala, Delete N-term 6 residues (MQEITV) |
| | Specific Biotinylation Sites: |
| | 1. (Template A) Asp65 to Cys, Tyr195 to Cys |
| | 2. (Template B) None |
| | Single Chain Linked Sequences: |
| | 1. Template A - Linker A - Template A - Linker A - Template A |
| | 2. Template A - Linker B - Template A - Linker B - Template A |
| | 3. Template A - Linker A - Template A - Linker A - Template B |
| | 4. Template A - Linker A - Template B - Linker A - Template B |
| | 5. Template B - Linker A - Template A - Linker A - Template A |
| | 6. Template A - Linker B - Template B - Linker B - Template B |
| | 7. & etc. |

Part 3. Specifications of Thermostable Node Proteins

| | |
|---|---|
| 2.0 | Description: Three-fold (C3) Planar Symmetric Node |
| 1j5s | Template Sequence: |
| (C3) | MGSDKIHHHHHHMFLGEDYLLTNRAAVRLFNEVKDLPIVDPHNHLDAKDIVENKPWNDIWEVEGATDHYVWELMRRCGVS |
| | EEYITGSRSNKEKWLALAKVFPRFVGNPTYEWIHLDLWRRFNIKKVISEETAEEIWEETKKKLPEMTPQKLLRDMKVEIL |
| | CTTDDPVSTLEHHRKAKEAVEGVTILPTWRPDRAMNVDKEGWREYVEKMGERYGEDTSTLDGFLNALWKSHEHFKEHGCV |
| | ASDHALLEPSVYYVDENRARAVHEKAFSGEKLTQDEINDYKAFMMVQFGKMNQETNWVTQLHIGALRDYRDSLFKTLGPD |
| | SGGDISTNFLRIAEGLRYFLNEFDGKLKIVLYVLDPTHLPTISTIARAFPNVYVGAPWWFNDSPFGMEMHLKYLASVDLL |
| | YNLAGMVTDSRKLLSFGSRTEMFRRVLSNVVGEMVEKGQIPIKEARELVKHVSYDGPKALFFG |
| | Sequence Modifications: |
| | General: Cys65 to Ala, Cysl49 to Ser, Cys227 to Ala |
| | Specific Biotinylation Sites: Lys42 to Cys, Ser77 to Cys |
| 2.D | Description: Three-Fold (C3) Polyhedral Node |
| 1v4n | Template Sequence: |
| (C3) | MMIEPKEKASIGIIGGSGLYDPQILTNVKEIKVYTPYGEPSDNIILGELEGRKVAFLPRHGRGHRIPPHKINYRANIWAL |
| | KSLGVKWVIAVSAVGSLRLDYKPGDFVVPNQFIDMTKGRTYTFFDGPTVAHVSMADPFCEHLRSIILDSAKDLGITTHDK |
| | GTYICIEGPRFSTRAESIVWKEVFKADIIGMTLVPEVNLACEAEMCYSVIGMVTDYDVFADIPVTAEEVTKVMAENTAKV |
| | KKLLYEVIRRLPEKPDERKCSCCQALKTALVLEHHHHHHHH |
| | Sequence Modifications: |
| | General: Cys165 to Ala or Ser |
| | Cys139-Cys206(SS) or Cys139 to Ala, Cys 206 to Ala |
| | Cys201-Cys262(SS) or Cys201 to Ala, Cys 262 to Ala |
| | Cys260-Cys263(SS) or Cys260 to Ala, Cys 263 to Ala |
| | Specific Biotinylation Sites: |
| | 1. Dodecahedral Node: Ile24 to Cys, Ile31 to Cys, |
| | 2. Truncated Icosahedral “Bucky” Node: Thr230 to Cys, Lys 267 to Cys |

Part 4. Specifications of Thermostable Node Proteins

| | |
|---|---|
| 2.E | Description: Four Fold (C4) Planar Symmetric Node |
| 1vcg | Template Sequence: |
| (C4) | MNIRERKRKHLEACLEGEVAYQKTTTGLEGFRLRYQALAGLALSEVDLTTPFLGKTLKAPFLIGAMTGGEENGERINLAL |
| | AEAAEALGVGMMLGSGRILLERPEALRSFRVRKVAPKALLIANLGLAQLRRYGRDDLLRLVEMLEADALAFHVNPLQEAV |
| | QRGDTDFRGLVERLAELLPLPFPVMVKEVGHGLSREAALALRDLPLAAVDVAGAGGTSWARVEEWVRFGEVRHPELCEIG |
| | IPTARAILEVREVLPHLPLVASGGVYTGTDGAKALALGADLLAVARPLLRPALEGAERVAAWIGDYLEELRTALFAIGAR |
| | NPKEARGRVERV |
| | Sequence Modifications: |
| | General: Cys14 to Ala, Cys237 to Ser or Ala |
| | Specific Biotinylation Sites: |
| | 1. Ser44 to Cys, Thr49 to Cys, |
| 2.F | Description: Five-fold (C5) Icosahedral Node |
| 1vdh | Template Sequence: |
| (C5) | MERHVPEPTHTLEGWHVLHDFRLLDFARWFSAPLEAREDAWEELKGLVREWRELEEAGQGSYGIYQVVGHKADLLFLNLR |
| | PGLDPLLEAEARLSRSAFARYLGRSYSFYSVVELGSQEKPLDPESPYVKPRLTPRVPKSGYVCFYPMNKRRQGQDNWYML |
| | PAKERASLMKAHGETGRKYQGEVMQVISGAQGLDDWEWGVDLFSEDPVQFKKIVYEMRFDEVSARYGEFGPFFVGKYLDE |
| | EALRAFLGL |
| | Sequence Modifications: |
| | General: Cys 152 to Ala |
| | Specific Biotinylation Sites: |
| | 1. LYS 45 to Cys, Ala 57 to Cys |

Part 5. Specifications of Thermostable Node Proteins

| | |
|---|---|
| 2.G | Description:Biotin Functionalized Streptavidin Derivatives |
| 1stp | Template Sequence: |
| (D2) | DPSKDSKAQVSAAEAGITGTWYNQLGSTFIVTAGADGALTGTYESAVGNAESRYVLTGRYDSAPATDGSGTALGWTVAWK |
| | NNYRNAHSATTWSGQYVGGAEARINTQWLLTSGTTEANAWKSTLVGHDTFTKVKPSAASIDAAKKAGVNNGNPLDAVQQ |
| | Sequence Modifications: |
| | General:None |
| | Specific Biotinylation Sites: |
| | 1. (A)(X-Axis Biotin Binding Blocking Site) Asn49 to Cys |
| | 2. (B)(Y-Dyad Axis) Asn81 to Cys |
| | 3. (C)(Z-Dyad Axis) Asn119 to Cys |
| | 4. A + B + C, 5. A + C, 6. B + C, 7. B + A |

Part 6. Specifications of Thermostable Node Proteins

| | |
|---|---|
| 2.H | Description: D2 Symmetric Node |
| lma1 | Template Sequence: |
| (D2) | MNDLEKKFYELPELPYPYDALEPHISREQLTIHHQKHHQAYVDGANALLRKLDEARESDTDVDIKAALKELSFHVGGYVL |
| | HLFFWGNMGPADECGGEPSGKLAEYIEKDFGSFERFRKEFSQAAISAEGSGWAVLTYCQRTDRLFIMQVEKHNVNVIPHF |
| | RILLVLDVWEHAYYIDYRNVRPDYVEAFWNIVNWKEVEKRFEDIL |
| | Sequence Modifications: |
| | General: Cys93 to Ser or Thr, Cys137 to Ala |
| | Specific Biotinylation Sites: |
| | 1. HX: Ser57 to Cys |
| | 2. VX: Glu127 to Cys |
| | 3. HY: Glu27 to Cys |
| | 4. VY: Tyr176 to Cys |
| | 5. HZ: Gln138 to Cys |
| | 6. VX: Arg 199 to Cys |
| | 7. HX + HY + HZ |
| | 8. VX + VY + VZ |
| | 9. HX + VY + VZ etc. |
| | 10. HX + HY, |
| | 11. HY + HZ |
| | 12. VX + HY etc. |

Part 7. Specifications of Thermostable Node Proteins

| | |
|---|---|
| 2.1 | Description: D2 Symmetric Node |
| into | Template Sequence: |
| (D2) | MRAVRLVEIGKPLSLQEIGVPKPKGPQVLIKVEAAGVCHSDVHMRQGRFGNLRIVEDLGVKLPVTLGHEIAGKIEEVGDE |
| | VVGYSKGDLVAVNPWQGEGNCYYCRIGEEHLCDSPRWLGINFDGAYAEYVIVPHYKYMYKLRRLNAVEAAPLICSGITTY |
| | RAVRKASLDPIKILLVVGAGGGLGTMAVQIAKAVSGATIIGVDVREEAVEAAKRAGADYVINASMQDPLAEIRRITESKG |
| | VDAVIDLNYSEKTLSVYPKALAKQGKYVMVGLFGADLHYHAPLITLSEIQFVGSLVGNQSDFLGIMRLAEAGKVKPMITK |
| | TMKLEEANEAIDNLENFKAIGRQVLIP |
| | Sequence Modifications: |
| | General: Cys38 to Ala, Cys101 to His, Cys112 to His, Cys104 to His, Cys154 to |
| | Ser (Zn Ion binding sites) |
| | Specific Biotinylation Sites: |
| | 1. HX: Glu251 to Cys |
| | 2. VX: Leu283 to Cys |
| | 3. HY: Va181 to Cys |
| | 4. VY: Arg116 to Cys |
| | 5. HZ: Leu99 to Cys |
| | 6. VX: Leu308 to Cys |
| | 7. HX + HY + HZ |
| | 8. VX + VY + VZ |
| | 9. HX + VY + VZ etc. |
| | 10. HX + HY, |
| | 11. HY + HZ 12. VX + HY etc. |

Part 8. Specifications of Thermostable Node Proteins

| | |
|---|---|
| 2.J | Description: D2 Symmetric Node |
| 1rtw | Template Sequence: |
| (D2) | MFSEELIKENENIWRRFLPHKFLIEMAENTIKKENFEKWLVNDYYFVKNALRFMALLMAKAPDDLLPFFAESIYYISKEL |
| | EMFEKKAQELGISLNGEIDWRAKSYVNYLLSVASLGSFLEGFTALYCEEKAYYEAWKWVRENLKERSPYQEFINHWSSQE |
| | FGEYVKRIEKILNSLAEKHGEFEKERAREVFKEVSKFELIFWDIAYGGEGNVLEHHHHHH |
| | Sequence Modifications: |
| | General: Cys 127 to Ala or Ser |
| | Specific Biotinylation Sites: |
| | 1. HX: Lys196 to Cys |
| | 2. VX: Asn42 to Cys |
| | 3. HY: Pro67 to Cys |
| | 4. VY: Ile76 to Cys |
| | 5. HZ: Glu185 to Cys |
| | 6. VZ: Glu177 to Cys |
| | 7. HX + HY + HZ |
| | 8. VX + VY + VZ |
| | 9. HX + VY + VZ etc. |
| | 10. HX + HY, |
| | 11. HY + HZ |
| | 12. VX + HY etc. |
| 2.K | Description: D3 Symmetric Node |
| 1b4b | Template Sequence: |
| (D3) | ALVDVFIKLDGTGNLLVLRILPGNAHAIGVLLDNLDWDEIVGTICGDDICLIICRTPKDAKKVSNQLLSML |
| | Sequence Modifications: |
| | General: Cys123 to Ala, Cys128 to Ala (SS Bridge in Native), Cys132 to Ala |
| | Specific Biotinylation Sites: |
| | 1. HX(+): Leu99 to Cys |
| | 2. HV(+): Asp111 to Cys |
| | 3. HX(−): Val108 to Cys |
| | 4. HV(−): Val81 to Cys |
| | 5. HX(+) + HX(−), |
| | 6. VX(+) + VX(−), |
| | 7. HX(+) + VX(−), |
| | 8. VX(+) + HX(−), |

Part 9. Specifications of Thermostable Node Proteins

| | |
|---|---|
| 2.L | Description: D3 Symmetric Node |
| 1hyb | Template Sequence: |
| (D3) | MMTMRGLLVGRMQPFHRGALQVIKSILEEVDELIICIGSAQLSHSIRDPFTAGERVMMLTKALSENGIPASRYYIIPVQD |
| | IECNALWVGHIKMLIPPFDRVYSGNPLVQRLFSEDGYEVTAPPLFYRDRYSGTEVRRRMLDDGDWRSLLPESVVEVIDEI |
| | NGVERIKHLAKKEVSELGGIS |
| | Sequence Modifications: |
| | General: Cys36 to Ala, Cys83 to Ser or Thr |
| | Specific Biotinylation Sites: |
| | 1. HX(+): Met93 to Cys |
| | 2. VX(+): Ser113 to Cys |
| | 3. HX(−): Glu32 to Cys |
| | 4. VX(−): SER64 to Cys |
| | 5. HX(+) + HX(−), |
| | 6. VX(+) + VX(−), |
| | 7. HX(+) + VX(−), |
| | 8. VX(+) + HX(−), |
| 2.M | Description: D3 Symmetric Node |
| 2prd | Template Sequence: |
| (D3) | ANLKSLPVGDKAPEVVHMVIEVPRGSGNKYEYDPDLGAIKLDRVLPGAQFYPGDYGFIPSTLAEDGDPLDGLVLSTYPLL |
| | PGVVVEVRVVGLLLMEDEKGGDAKVIGVVAEDQRLDHIQDIGDVPEGVKQEIQHFFETYKALEAKKGKWVKVIGWRDRKA |
| | ALEEVRACIARYKG |
| | Sequence Modifications: |
| | General: Cys 168 to Ser or Thr |
| | Specific Biotinylation Sites: |
| | 1.HX(+): Glu126 to Cys |
| | 2.VX(+): Asp120 to Cys |
| | 3.HX(−): Gln133 to Cys |
| | 4.VX(−): Trp149 to Cys |
| | 5. HX(+) + HX(−), |
| | 6. VX(+) + VX(−), |
| | 7. HX(+) + VX(−), |
| | 8. VX(+) + HX(−), |

Part 10. Specifications of Thermostable Node Proteins

| | |
|---|---|
| 2.N | Description: D4 Symmetric Node |
| 104v | Template Sequence: |
| (D4) | MGSDKIHHHHHHVPRVGIIMGSDSDLPVMKQAAEILEEFGIDYEITIVSAHRTPDRMFEYAKNAEERGIEVIIAGAGGAA |
| | HLPGMVASITHLPVIGVPVKISTLNGLDSLFSIVQMPGGVPVATVAINNAKNAGILAASILGIKYPEIARKVKEYKERMK |
| | REVLEKAQRLEQIGYKEYLNQKE |
| | Sequence Modifications: |
| | General: None |
| | Specific Biotinylation Sites: |
| | 1. HX: His79 to Cys |
| | 2. VX: Prot to Cys |
| | 3. HX′: Asn51 to Cys |
| | 4. VX′: Thr41 to Cys |
| | 5. HX + HX′, |
| | 6. HX + VX′, |
| | 7.VX + VX′, |
| | 8. VX + HX′ |
| 2.0 | Description: D4 Symmetric Node |
| 2h2i | Template Sequence: |
| (D4) | MKMKEFLDLLNESRLIVILTGAGISTPSGIPDFRGPNGIYKKYSQNVFDIDFFYSHPEEFYRFAKEGIFPMLQAKPNLAH |
| | VLLAKLEEKGLIEAVITQNIDRLHQRAGSKKVIELHGNVEEYYCVRCEKKYTVEDVIKKLESSDVPLCDDCNSLIRPNIV |
| | FFGENLPQDALREAIGLSSRASLMIVLGSSLVVYPAAELPLITVRSGGKLVIVNLGETPFDDIATLKYNMDVVEFARRVM |
| | EEGGIS |
| | Sequence Modifications: |
| | General: None |
| | (Cys ligate to Zn) or Cys133 to Ala, Cys136 to Ala, Cys156 to |
| | Ala, Cys159 to Ala |
| | Specific Biotinylation Sites: |
| | 1. HX: Cys 124 to Cys |
| | 2. VX: Glu120 to Cys |
| | 3. HX′: Va1145 to Cys |
| | 4. VX′: Asn46 to Cys |

Part 11. Specifications of Thermostable Node Proteins

| | |
|---|---|
| 2.P | Description: D4 Symmetric Node |
| 2iel | Template Sequence: |
| (D4) | MARYLVVAHRTAKSPELAAKLKELLAQDPEARFVLLVPAVPPPGWVYEENEVRRRAEEEAAAAKRALEAQGIPVEEAKAG |
| | DISPLLAIEEELLAHPGAYQGIVLSTLPPGLSRWLRLDVHTQAERFGLPVIHVIAQAA |
| | Sequence Modifications: |
| | General: None |
| | Specific Biotinylation Sites: |
| | 1. HX: Ala94 to Cys |
| | 2. VX: Arg113 to Cys |
| | 3. HX′: Arg53 to Cys |
| | 4. VX′: Va146 to Cys |
| 2.Q | Description: Tetrhedral Node with Diad axes Directed to form Cubic Lattice |
| 1pvv | Template Sequence: |
| (T23) | MVVSLAGRDLLCLQDYTAEEIWTILETAKMFKIWQKIGKPHRLLEGKTLAMIFQKPSTRTRVSFEVAMAHLGGHALYLNA |
| | QDLQLRRGETIADTARVLSRYVDAIMARVYDHKDVEDLAKYATVPVINGLSDFSHPCQALADYMTIWEKKGTIKGVKVVY |
| | VGDGNNVAHSLMIAGTKLGADVVVATPEGYEPDEKVIKWAEQNAAESGGSFELLHDPVKAVKDADVIYTDVWASMGQEAE |
| | AEERRKIFRPFQVNKDLVKHAKPDYMFMHCLPAHRGEEVTDDVIDSPNSVVWDQAENRLHAQKAVLALVMGGIKF |
| | Sequence Modifications: |
| | General: Cys 11 to Thr or Ser, Cys136 to Thr or Ser, Cys269 to Thr or Ser |
| | Specific Biotinylation Sites: |
| | 1. H: Arg7 to Cys |
| | 2. V: Thr122 to Cys |

### Examples of Nanostructures:

Nanotechnology is a broad term covering a number of topics in the physical and materials sciences where novel properties emerge characteristic of structural length-scales of a few nanometers (1nm=10⁻⁹ m=10A). In an embodiment according to the present invention, a molecular “parts box” incorporating symmetrical protein “nodes” with covalently bound biotin groups, can be coupled together using streptavidin “struts”. Structures assembled from the components can be used as substrates for the attachment of sensing components (e.g. antibodies, other proteins with specific binding or signaling functionality, dyes, etc.), ultimately functioning as novel biosensors or biomaterials.

In an embodiment according to the present invention, a flexible set of protein-based molecular components can be used to create a wide range of biomaterials with structural organization on the nanoscale. These components can be used in the context of biomedical research and biomedical devices and can be used to produce new biomaterials serving as substitutes for skin, bone or other tissues, as well as in applications such as biosensors and diagnostic devices.

Components according to an embodiment of the present invention can be used for “research and development of new enabling technologies for the fabrication and use of nanoscale components and systems in diagnostic and therapeutic applications”, that envision the “development of new nanoscale patterning and manipulation systems”. See http://grants.nih.gov/grants/guide/pa-files/PA-09-080.html. For example, nanotechnology and bioengineering technologies can be applied to advanced methods of cancer detection and diagnosis. Nanotechnology-based implantable biomaterials for dental, oral, and craniofacial tissue restoration. The nanocomponents can be applied to the measurement of blood parameters and diagnosis of blood disorders. New technologies based on micro- and nanotechnology can provide sensitive, high throughput, and potentially portable systems capable of measuring environmental exposures and the impact of the exposures on human biology.

Certain engineered proteins and protein assemblies can find application in nanotechnology applications. For example, the thermostable Thermococcus litoralis D-trehalose/D-maltose-binding protein can be used for sugar monitoring (De Stefano et al. 2008) and antibodies can be used for virus detection (Tripp et al. 2007). A biosensor design may incorporate redox proteins for sensing and signaling applications (Gilardi & Fantuzzi 2001). For example, a fusion of cytochrome c and b-amyloid may detect conditions of plaque formation (Baldwin et al. 2006). Membrane proteins may act as nanopores to detect single molecules of DNA (Butler et al. 2008). Motor proteins, capable of turning and/or moving in response to stimuli may be engineered for use in nanotechnology (van den Heuvel & Dekker 2007). The ATP-dependent transport of microtubule shuttles by kinesin motor proteins (Clemmens et al. 2004) may find application. Combinations of biological and synthetic molecules may be used as nanotechnology components. Covalent attachment of protein receptors to photoactive small molecules such as azobenzene (Volgraf et al. 2006) may be useful. Immobilizing engineered antibody domains on semiconductor surfaces may be used to develop field-effect transistor sensors (Eteshola et al. 2008). Integrin arrays may be constructed on synthetic peptide monolayers (Lee et al. 2006). Integral membrane proteins in native (Jones et al. 2008) or engineered (Yoshino et al. 2004) forms may be imbedded in lipid-based nanoparticles. Certain proteins may naturally form organized structures as frameworks that may be useful for the construction of nanodevices. The assembly of S-layer proteins that form regular lattices on the surface of many prokaryotes has been investigated (Sara & Sleytr 2000). Fusion of an engineered S-layer protein to an antibody domain has been investigated in microbead sensor applications (Vollenkle et al. 2004). Virus particles have been investigated as structural substrates for nanotechnology (Brumfield et al. 2004; Young et al. 2008; Steinmetz et al. 2009). The human vault proteins that form ribonucleoparticles have been engineered to carry green fluorescent protein and luciferase (Kickhoefer et al. 2005).

Several key issues may have limited the overall quality and homogeneity of the structures that Ringler-Schulz were able to create, the structures having been generally limited to relatively small 2-D lattices. For example, a factor may have been related to essentially irreversible character of the biotin:streptavidin interaction, that frustrates potential “annealing” reorganizations that lead to the formation of the most stable and symmetric assemblies. Another factor that may have limited the assembly fidelity of the 2-dimensional Ringler-Schulz lattices was a lack of precision in defining the biotin attachment sites on the surface of their C4 aldolase node. The sites chosen could have introduced twist into extended structures, consequently destabilizing the formation of regular planar 2-D lattice structures.

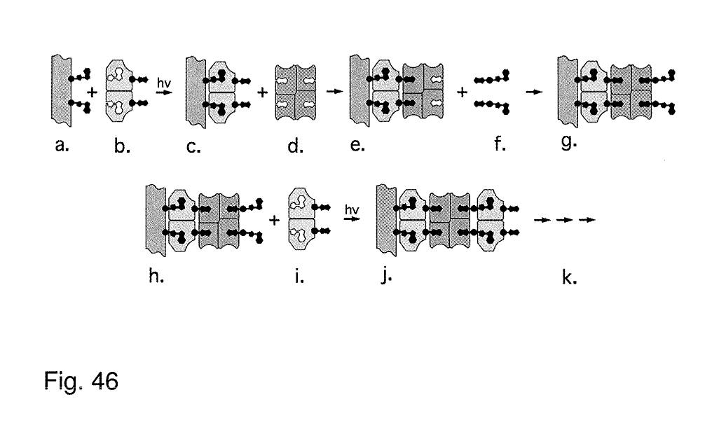

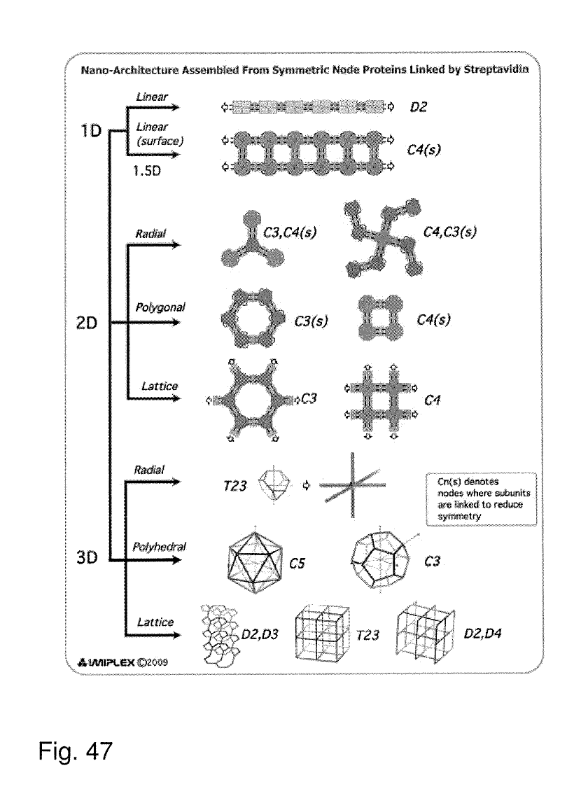

In an embodiment according to the invention, we developed a thiol-reactive iminobiotin reagent. The binding of iminobiotin to streptavididn is pH-dependent (Green 1975; Hoffman et al. 1980), so allowing a strategy for annealing structures formed between iminobiotin-substituted nodes and streptavidin struts. In another embodiment, potentially offering additional flexibility on nanostructure assembly, we developed a “streptavidin macromolecular adaptor protein” or “SAMA” that we have engineered to serve as a reversible protecting group for two of the four biotin binding groups on streptavidin (FIG. 46). Use of the SAMA for macromolecular assembly offers several additional advantages for nanostructure assembly including: 1) geometrical control of reactivity, 2) potential for specific immobilization of a growing molecular assembly, 3) ability to drive reaction equilibria to completion using mass action, and 4) greatly facilitated ability to purify reaction products from reagents. Furthermore, we took great care to develop methods to design cysteine substitution sites in positions that would produce strain-free extended assemblies.

We envision a flexible set of components, enabling the construction of many one, two, and three-dimensional architectures with defined dimensions, that can serve as a substrate for the immobilization of engineered proteins with specific sensing, binding, or other functions. The node:streptavidin arrays are designed to provide a ready framework for the design and assembly of engineered protein components into functional nanodevices. For example, streptavidin has potential as a nanostructural building block (Sligar & Salemme 1992).

In an embodiment according to the present invention, a modular set of protein-based components allow end user construction of a wide variety of nanostructure assemblies. A system of flexible components can enable the construction of highly customizable architectures assembled from “struts”, that are basically linear structural elements, and “nodes”, that have plane or point group symmetry. Struts and nodes can potentially be assembled and functionalized through attachment of other proteins (e.g. antibodies) to create a great variety of precision nanostructures with biomedical applications such as biomaterials, biosensors or diagnostic devices. The struts can incorporate streptavidin tetramers that bind to nodes that have been functionalized through site-specific modification and chemical reaction to incorporate biotin groups that are geometrically complementary to the biotin binding sites on streptavidin.

We have designed engineered proteins corresponding to a C3-symmetric (trimeric) node and a C4-symmetric (tetrameric) node, which, respectively, allow, for example, the assembly of 2-dimensional hexagonal and square lattices. We designed and produced a single-chain version of the C3-symmetric protein where two of three C3 symmetry-related positions on the trimer were modified to allow strut connection, e.g. to allow the formation of planar hexagons. We envision improved methods of production of these molecular components and the design of additional nodes with symmetry properties to expand the variety of architectures that can be assembled. We describe the design of a trimeric node variant that incorporates a Streptococcal protein G antibody-binding domain. We describe potential applications for using our components, for example, in the research biomedical and nanotechnology communities, and in the pharmaceutical diagnostics industry. Our components can find wide application in the biomedical research and device communities and can potentially lead to a host of new biomaterials, biosensors, and diagnostic devices.

We envision a comprehensive platform for the construction of protein-based nanostructures with utility as biosensors, biomaterials, or other devices, where novel properties emerge owing to the ability to control structural or functional organization at the nanoscale. We envision the development of a modular set of building blocks for the assembly of protein-based nanostructures. We envision materials and biosensors based on this platform, for example, for use by the biomedical, biotechnology, and nanofabrication communities for additional research and product development applications. The general availability of a molecular “parts box:” can permit the controlled construction of nanostructures that can facilitate the development of a new generation of functional nanodevices, biosensors, and biomaterials, that in turn can constitute new approaches for disease diagnostics, environmental sensors, and replacement materials for skin, bones, and other tissues.

We have designed, expressed, and characterized C3 and C4 symmetric nodes engineered to be coupled to, and interconnected by streptavidin, capable of forming, for example, hexagonal and square 2-dimensional lattices. The node proteins are based on template proteins derived from thermophiles, so that assembled nanodevices are stable under a variety of manufacturing and storage conditions. We envision developing additional enhancements including a node engineered to incorporate an antibody-binding sequence, manufacturing modular sub-assemblies, and characterizing nodes and node assemblies using a variety of biophysical and structural methods. For example, we envision purifying and scaling the production of first-generation C3- and C4-symmetric engineered nodes. For example, we envision designing and producing (including expressing and purifying) engineered C3 (3-fold symmetric) node structures, for example, single-chain C3 nodes incorporating IgG binding sequences. For example, we envision designing and producing (including expressing and purifying) engineered C4 (4-fold symmetric) nodes, for example, nodes with novel arrangements for strut attachment and single chain C4 nodes with altered valency and geometry. For example, we envision characterizing and developing methods for the characterization of nodes, for example, for the determination of node stability, optimization of conditions for nanostructure assembly, X-ray crystallography of engineered nodes, and electron microscopy of engineered nodes and assemblies.

We envision the development of molecular components, nanostructural modules, arrays, and functional nanodevices. These components can be used, for example, in biomedical and nanostructural research and applications development. We envision developing custom assemblies of our components as products, initially focused on biosensor modules and biomaterials. We envision the development of modular devices, for example, for diagnostic and medical device applications. We envision developing a molecular parts box, including a variety of nodes with symmetry allowing the formation of extended structures with one, two, and three-dimensional geometry, for example, in conjunction with a streptavidin macromolecular adapter (SAMA). Such a parts box can enable the development of a new generation of biosensors and functional biomaterials.

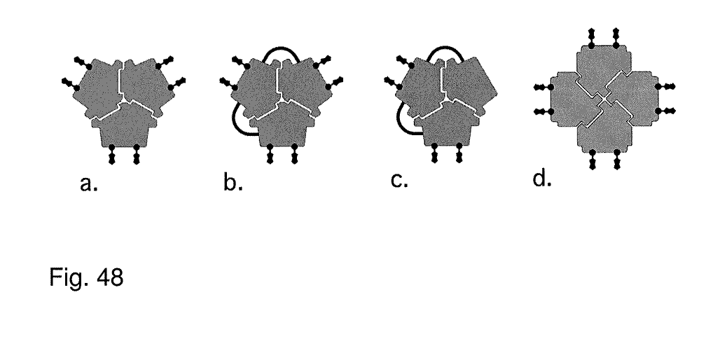

We have developed design methods and designed, expressed, and characterized engineered node proteins designed to be interconnected into nanoassemblies via biotin:streptavidin crosslinks. Nodes of two different symmetries, 3-fold (C3), and 4-fold (C4), were modeled, alone and in complexes with streptavidin (SAV). Two C3 symmetric proteins, Thermotoga maritima uronate isomerase, and g-carbonic anhydrase from the thermophilic archeon Methanosarcina thermophila, were studied as C3 node templates. The Thermus thermophilus type 2 isopentenyl diphosphate isomerase (IPP isomerase) was used as the C4 node template. We developed generalized computational methods for selection of sites on macromolecules to allow assembly of geometrically-defined protein based nanoassemblies. We have selecting node template molecules and determined the locations of site-specific modifications allowing the covalent attachment of biotin in positions (pairwise) complementary to biotin binding sites on streptavidin. We developed expression systems, expressed the proteins, and purified each node. We derivatized nodes using biotinylation reagents and carried out model assembly reactions. Representative proteins of each symmetry were expressed at sufficient levels in E. coli to allow straightforward purification procedures. Engineered node proteins were derivatized using standard biotinylation reagents. Experiments were performed assembling biotin-linked NODE:SAV complexes. Molecules produced are summarized schematically in FIG. 48. Details are described below.

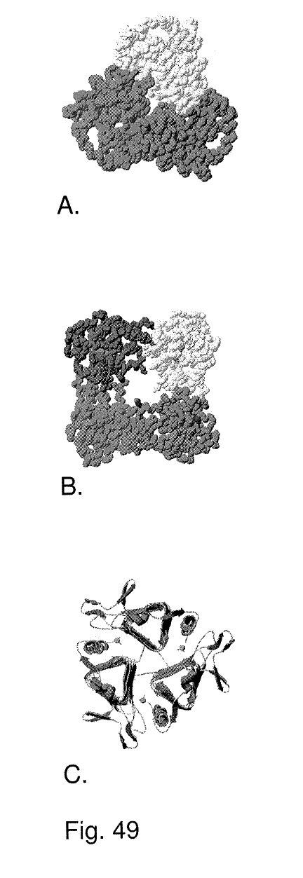

We selected protein structures derived from thermostable organisms with C3 and C4 symmetry suitable as node templates and developed methods to determine optimal biotin attachment sites on each node subunit for complex formation with streptavidin-based struts. The Protein Data Bank (PDB www.rcsb.org) was screened to identify candidates for C3 and C4 symmetric proteins to serve as node templates. We compiled a symmetric node database of crystal structures of proteins from thermophiles containing 30 proteins with C3 symmetry and 7 with C4 symmetry. Uronate isomerase (TM0064) from Thermotoga maritima (Schwarzenbacher et al. 2003, pdb code: 1j5s) was initially selected as a trimeric node template (FIG. 49A) and the type 2 isopentenyl diphosphate isomerase from Thermus thermophilus (Wada et al. 2006; de Ruyck et al. 2005, pdb code: 1vcg) was selected as the candidate 4-fold node template (FIG. 48B). These molecules satisfied the following selection criteria: they possess the desired rotational symmetry, neither has native disulfide bonds (a feature that simplifies engineering and expression), both have only a small number of naturally occurring surface cysteine residues (Nodes are engineered to have only two surface cysteine residues available for reaction with biotin regents in locations that are geometrically complementary to pairs of biotin binding sites on streptavidin.), and both are sufficiently large to offer an extended interaction site with streptavidin. Uronate isomerase did not express well in E. coli. We selected a carbonic anyhdrase from the thermophilic archeon Methanosarcina thermophila (Kisker et al. 1996, pdb code: 1thj) as a second C3 symmetric node (FIG. 49C).

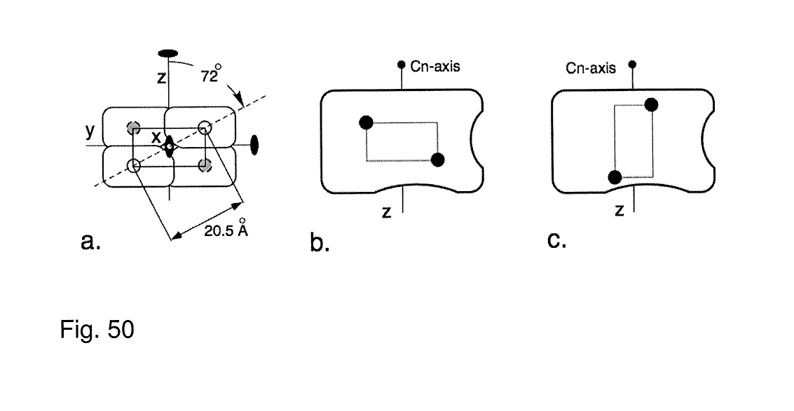

We selected biotin attachment sites. In order to assemble extended structures that do not twist when interconnected by streptavidin, the geometry of site-specific modifications on the node (or more specifically the coordinates of the thiol sulfur atoms of the incorporated cysteine side chains on the node protein) can be complementary to the geometry of the biotin binding sites on streptavidin, and can align the streptavidin z-axis or y-axis (FIG. 50) with the node Cn or z-axis. To achieve this, the modification sites on the nodes can be oriented at an angle (e.g. ˜72 degrees) relative to the Cn rotational (z) axis of the node protein oligomer.

There are generally two orthogonal orientations that streptavidin can take with respect to the major symmetry axes of complexes with Cn symmetry where the y-axis or z-axis of streptavidin is parallel to the node Cn axis (FIG. 50B, 50C). Since the streptavidin tetramer makes an asymmetric interaction with Cn node, there are potentially a large number of possible complementary interactions that are feasible for a Cn-node streptavidin-strut interaction.

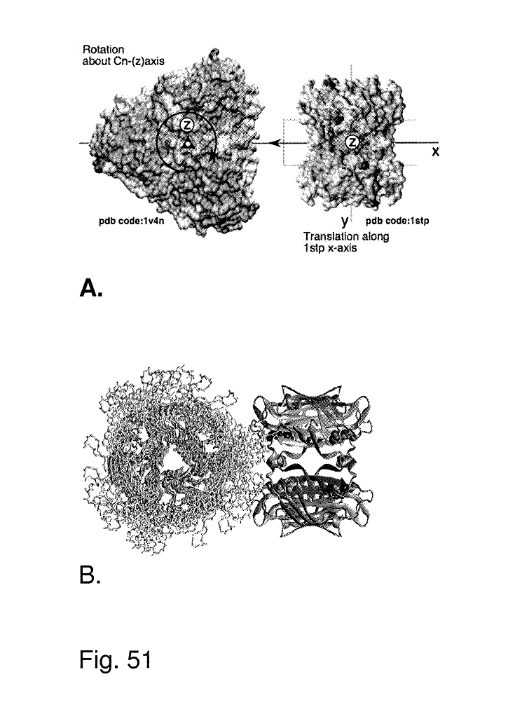

Two alternative computational methods were developed to assist in selecting amino acid positions to serve as biotin attachment sites (Salemme et al. 2009). The first method was developed to find optimal “docking” interactions between streptavidin and nodes with Cn symmetry. This involved performing a constrained geometrical search for favorable interaction complexes. FIG. 51 schematically illustrates the variable search parameters for Cn node structures. The search parameters included a rotation of the Cn node about its z-axis, and a translation of streptavidin along its x-axis in the xy plane of the node (FIG. 51a). The method involved initially orienting the Cn template node and streptavidin so that they a) did not spatially overlap, b) were oriented with the Cn (z-axis) of the node parallel to either the y-axis or z-axis of streptavidin, and c) had similar z coordinate values for their respective centers of mass. The node was incrementally rotated about the Cn axis through an angular range somewhat greater than 360/n degrees. For each angular increment about the Cn axis, the streptavidin tetramer was translated along its dyad x-axis until van der Waals contact or near van der Waals contact was made between the atomic coordinates of the node template and atomic coordinates of streptavidin. Each of the resulting streptavidin:node complexes was then examined using computer graphics (Jones et al. 1990; Humphrey et al. 1996), geometrical or energetic computational methods (Case et al. 2005), or a combination of these methods to determine the quality of overall fit and feasibility of locations of cysteine substitutions on the node template that could provide chemical attachment points for biotin, including the use of coupling reagents with different linker lengths. The process outlined was repeated for small incremental changes in rotation around the template node Cn symmetry axis (typically about 0.1 to 2.0 degrees in rotation), so that interactions of the Cn node surface and streptavidin were extensively sampled, evaluated and compared. The complexes were also inspected using VMD (/www.ks.uiuc.edu/Research/vmd/) and DeepView computer graphics programs (Guex 1996; Guex et al. 1999).

Table 4 lists the substitution residues on each node that were selected based on the computational docking methods described above.

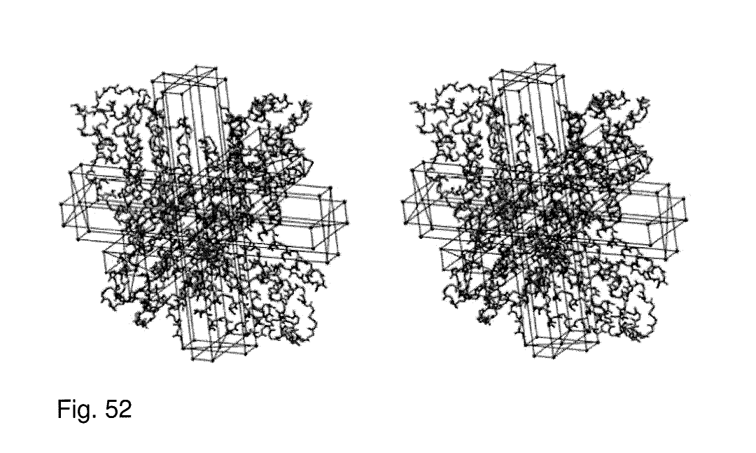

In addition to the constrained geometrical search method described above, we developed a second method for defining cysteine modification sites on node templates with dyad axes of symmetry (e.g. Dn or higher symmetry). This approach relied on developing a geometric representation of streptavidin that is defined by the location of the biotin binding sites (FIG. 52). Basically this involved the superposition of “bounding boxes” (with dimensions of approximately 6.4 Angstroms by 19.5 Angstroms, FIG. 4.5) that represent the projected positions of the potential biotinylation sites (e.g. sites complementary to the biotin bonding sites in each of the 2 possible streptavidin binding orientations) around each dyad axis in a structure. For example, FIG. 52 shows a stereoscopic view of a D2 symmetric node with pairs of bounding boxes embedded along each of the three dyad axes. Using a computer method (e.g. Lee & Richards 1971), a list of the atoms that lie on the surface of a protein was compiled. The list included Ca backbone atoms for Gly residues and Cb sidechain atoms for all other amino acids. A program was written to find the shortest distances between selected side chain atoms in the exposed atom/residue list and the lines defining the bounding box that project the positions of the biotin binding sites. The atoms so identified defined the residues in the node sequence that were to be mutated to Cys residues, and when functionalized by biotinylation, would form sites that are symmetric to streptavidin and align the Cn axis of the node to either the y- or z-axis of streptavidin. With slight modification, this approach is generalizable to nodes of higher symmetries.

Specific Aim 2: Computer modeling of Node:Streptavidin Complexes & 2D Surface Immobilization Linkers. In this specific aim, computational modeling of node: streptavidin complexes was undertaken to 1) guide and refine choices of biotin linkers on the node and to 2) define attachment points for affinity sequences to aid in protein purification and/or orientation of the node on a 2D surface.

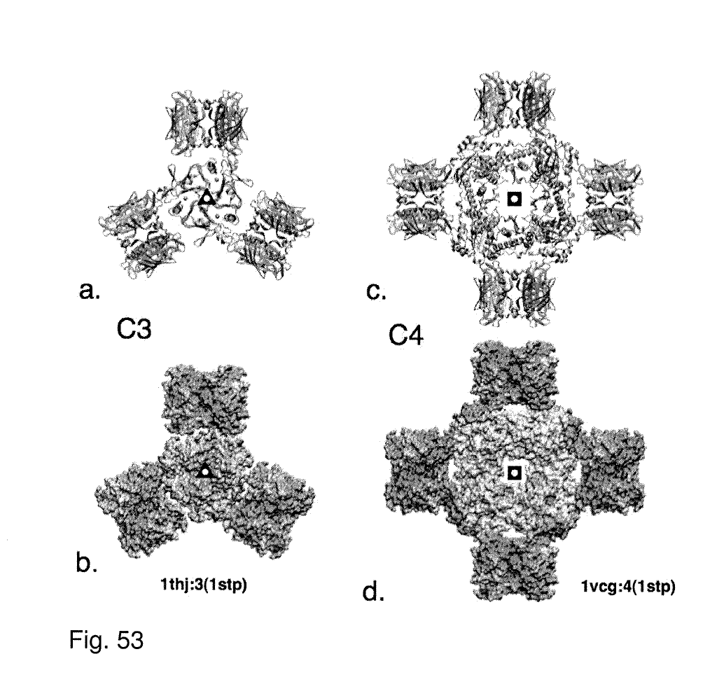

Following the approach outlined above (FIG. 51), computational and computer graphics methods were used to generate and evaluate the Cn node template:streptavidin interaction interfaces. When a suitable interaction was identified, coordinates for the entire set of Cn symmetry-related streptavidin molecules were generated to allow visualization of the complete node:streptavidin complex. FIG. 53 shows examples of optimal docking solutions between uronate isomerase (C3) and IPP isomerase (C4) in complex with a full complement of streptavidin tetramers.

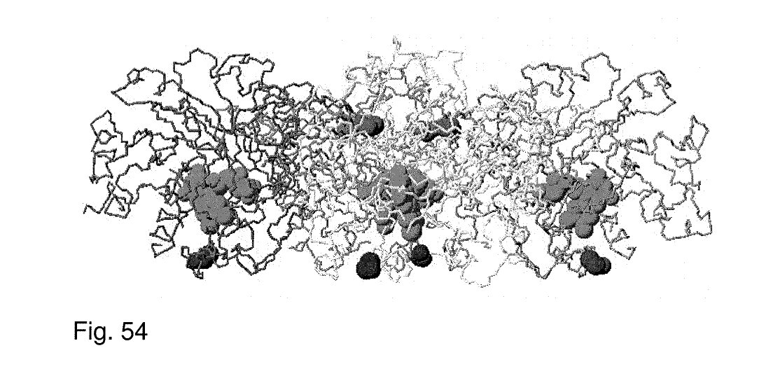

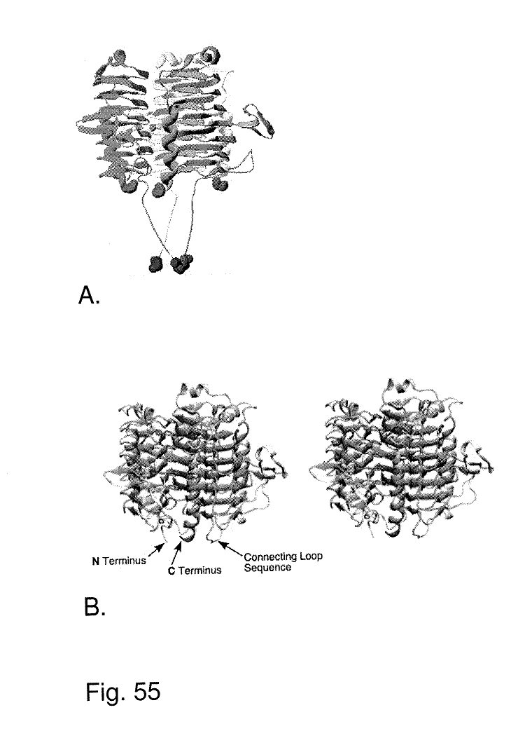

Immobilization of the node protein is an initial step in assembly of 2D surface lattices. Of the many known immobilization strategies, the histidine-tag is among the most versatile for nanotechnology applications because it can be engineered to bind a variety of metal surfaces (e.g. Thess et al. 2002; Cassimjee et al. 2008). For this reason, and to aid in protein purification, His6 tags were added to g-carbonic anhydrase and IPP isomerase polypeptide chain termini. The tag was inserted before the N-terminus of IPP isomerase because the N-termini in the tetramer are located at one surface (FIG. 54). It was known from previous work that T. thermophiles IPP isomerase with an N-terminal His6 tag and Factor Xa cleavage site could be successfully expressed in E. coli (de Ruyck et al. 2005). Attachment of the His-tag on g-carbonic anhydrase was guided by the structure. FIG. 55A shows how the amino termini of the trimer extend from the structure to form a spike that is stabilized by packing interactions between symmetry-related isoleucine (Ile3) and lysine (Lys1) sidechains and hydrogen bonds between the Glu2 carboxylate and the Ile3 backbone amide. Because this packing interaction would place N-terminal tags in close proximity (as would deletion of the N-terminal extension) a feature that might destabilize the structure, His-tags were added to the C-terminus that are well separated in the folded trimer (FIG. 55A).

We designed genes for nodes optimized for expression in E. coli. Synthetic genes were generated that encode IPP isomerase, uronate isomerase, and g-carbonic anhydrase, in both native and their variant forms, by BlueHeron Bio using their high throughput gene synthesis platform. Codon usage was optimized for expression in the E. coli bacterial host strain. Genes synthesized for this project are listed in Table 5.

To increase potential applications of the C3 node, we engineered two single chain variants of g-carbonic anhydrase. In these molecular constructs, the three subunits of the trimer were fused into a single continuous polypeptide chain (FIG. 55B). This was facilitated by the relatively close apposition of N and C termini from successive chains of each monomer in the trimer. The single chain fusion was accomplished by eliminating 5 residues from the N-terminus of two subunits and designing a linker to interconnect each with the C-terminus of the adjacent subunit. The fragment search option of DeepView (Guex 1996; Guex et al. 1999) was used to determine conformationally favorable sequences able to form the interconnecting loop. Formation of a single-chain construct allows the subunits to be non-equivalent. We built genes coding for a single chains with three and two (e.g. with two streptavidin binding sites oriented at 120 degrees) streptavidin binding sites. Specific sequence features are summarized in Table 5.

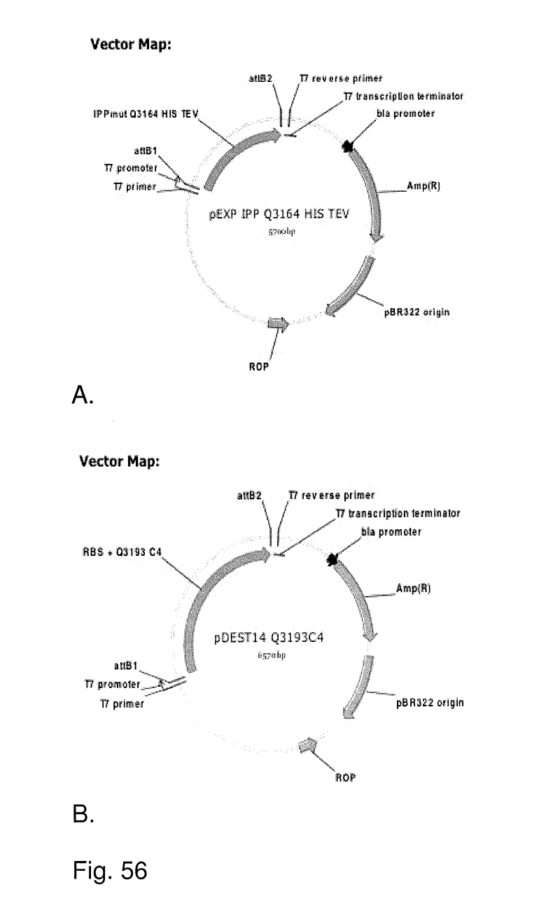

We expressed nodes and purified several milligrams for use in biochemical characterization of node:strut interactions. Similar expression vectors were used for the various constructs (FIG. 56). Sequences of the synthesized genes were verified after transformation into E. coli.

Proteins were expressed in E. coli. Relative expression levels were initially screened using a standard matrix of growth conditions, then 16 L scale batches were fermented under the optimal conditions established in the screen. Growth parameters for 16 L fermentations of native IPP isomerase and the variant (Table 4) are summarized here with data for the variant given in parentheses. E. coli strain BL21 (DE3) pLysS, a bacterial expression strain of Invitrogen, was grown in Terrific Broth culture media supplemented with 100 mg/mL of the antibiotic ampicillin. (Fermentation of the variant was also supplemented with 34 mg/mL chloramphenicol.) An 85 mL (345 mL) initial cell culture was grown overnight and used to inoculate a 16 L flask maintained at 37° C. with the growth chamber flask temperature shifted to 25° C. when the cell culture density reached an OD₆₀₀ of 0.792. (Temperature was not lowered for the variant.) Expression of IPP isomerase was induced by addition of 0.4 mM isopropyl b-D-1-thiogalactopyranoside (IPTG). 200 mg/mL riboflavin were added for the variant. Cells were harvested by centrifugation after 16 hrs of growth (4 hrs), and at that time the cell density OD₆₀₀ was 14.36 (12.15). The yield of cells as a wet paste was 570 g (323 g). Cells were frozen and stored at −80° C. IPP isomerase was identified by LC/MS from gel slices.

Uronate isomerase expressed at low levels in the standard matrix of growth conditions and most protein was present in the insoluble fraction. A second 3-fold node (g-carbonic anhydrase) was selected.

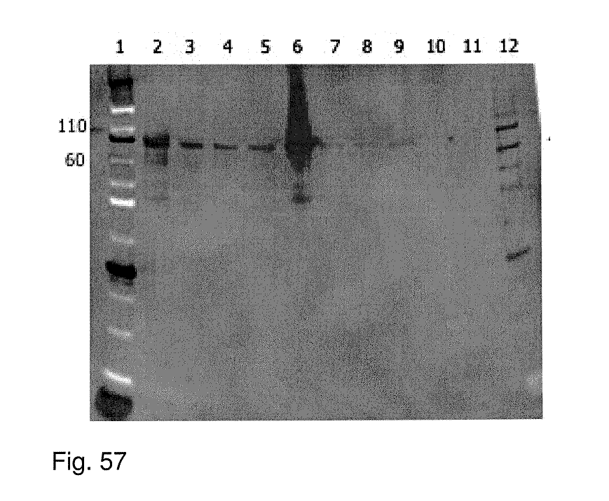

Expression of g-carbonic anhydrase variants (Table 5) in E. coli was screened using a growth condition expression matrix (e.g., FIG. 57). Proteins were detected in soluble lysates, with only the engineered trimer exhibiting a relatively low expression level. 16 L batches were then fermented under the best conditions established in the screens. Growth parameters for 16 L fermentations of trimeric g-carbonic anhydrase and the single chain variant engineered for complexation with 3 streptavidin tetramers are summarized here with data for the variant given in parentheses. E. coli strain BL21 Star (DE3) pLysS was grown in Terrific Broth culture media supplemented with 100 mg/mL of the antibiotic ampicillin and 34 mg/mL chloramphenicol. A 310 mL (375 mL) initial cell culture was grown overnight to OD₆₀₀ of 5.183 (4.276) and used to inoculate a 16 L flask maintained at 37° C. with the growth chamber flask temperature shifted to 25° C. when the cell culture density reached an OD₆₀₀ of 1.004 (1.053). Expression of g-carbonic anhydrase was induced by addition of 0.4 mM IPTG and 0.5 mM zinc sulfate. Cells were harvested by centrifugation after 4 hrs of growth (20 hrs), and at that time the cell density OD₆₀₀ was 1.775 (7.34). The yield of wet cell paste was 71.6 g (182.5 g). Cells were frozen and stored at −80° C.

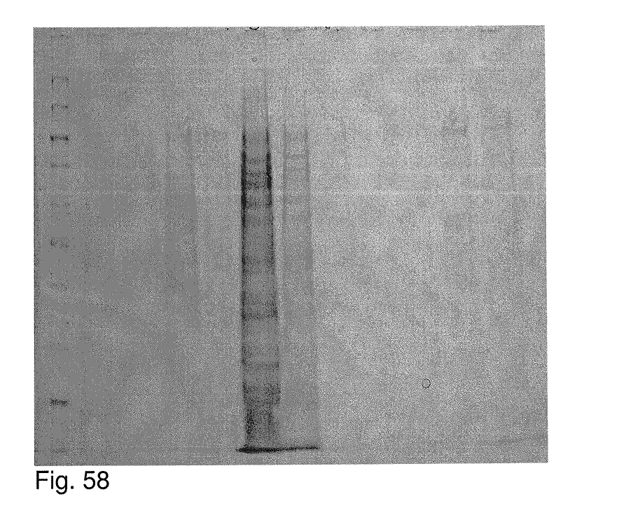

Isolation of both IPP isomerase and g-carbonic anhydrase was achieved by combining His6 affinity steps with other purification protocols. In a typical IPP isomerase preparation adapted from de Ruyck et al. 2005, 4-5 grams of cell paste were disrupted by lysozyme treatment and sonication or by addition of nonionic detergents (B-PER ThermoScientific). The cell suspension was clarified by centrifugation (12 500×g, 15 min) yielding a small pellet and clear supernatant. The supernatant was heated to 60° C. and held at that temperature for 15 minutes. Denatured proteins were removed by centrifugation (12 500×g, 15 min) and the clear supernatant containing IPP isomerase was incubated with 1 mL Ni agarose resin held at 4° C. and gently rocked overnight. Following two washes each of 50 mM sodium phosphate buffer pH 8.0, 300 mM NaCl with 20 and 40 mM imidazole, IPP isomerase was eluted from the Ni agarose using 250 mM imidazole, 300 mM NaCl, 50 mM sodium phosphate buffer pH 8.0. Centrifugal concentrators were used to exchange the buffer to 25 mM sodium phosphate, 0.1M NaCl pH 7.4, then concentrate the bright yellow protein to ˜5 mg/mL. IPP isomerase was >95% pure as judged by SDS-PAGE. Both lysozyme treatment followed by sonication or addition of nonionic detergents (B-PER ThermoScientific) were used to disrupt E. coli cells containing single-chain g-carbonic anhydrase. For lysozyme and sonication treatment, 6-7 grams frozen cell paste was suspended in 30 mL 50 mM potassium phosphate buffer pH 6.8 to which protease inhibitors and DNase I were added. After stirring for 30 minutes in the cold, cells were sonicated with 2 sets of 15 1-second bursts of sonication with 10 seconds of cooling between. Similar ratios of cell paste to solubilization solution were maintained when nonionic detergents were used. Following Alber & Ferry 1996 and Simler et al. 2004, the cell suspension was clarified by centrifugation (20 000×g, 30 min). For experiments with sonication where the cell supernatant contained more total protein, the clear supernatant was chromatographed on Q-sepharose equilibrated with 50 mM potassium phosphate buffer pH 6.8 and developed with the buffer with 1M NaCl added. The pH of g-carbonic anhydrase containing fractions was adjusted to 8.0, and this fraction or the clarified cell supernatant from treatment with nonionic detergents was equilibrated overnight in the cold with Ni agarose resin. The resin was washed, then developed with elution buffers made according to the manufacturer's suggestions (Qiagen). With each buffer, the resin was rocked for 15 minutes then pelleted by centrifugation (1 200×g, 5 min) and the supernatant analyzed (FIG. 58). Carbonic anhydrase activity was determined according to Khalifah (1971), after dialyzing the fractions against 50 mM potassium phosphate pH 6.8, 1 uM zinc sulfate. Active fractions were pooled and concentrated by centrifugation.

A general protocol involved derivatization of the engineered node proteins with biotin, followed by complex formation with streptavidin to form NODE:SAV complexes.

For IPP isomerase biotin was covalently linked to cysteine residues 44 and 49 using the following procedure. For reaction with EZ-Link HPDP (Table 3), protein was equilibrated in 20 mM sodium phosphate buffer pH 6.8 by buffer exchange using centrifugal protein concentrators (PierceNet) to concentrate the protein to about 10 mL followed by adding 2 mL of buffer. Protein was then concentrated to a volume of about 0.1 mL and at least 1 mg/mL. Solutions of biotin-containing reagents were prepared by adding solid reagent to the appropriate buffer. For the sulfur-reactive biotin-linking reagent EZ-Link HPDP, the dissolving solution was dimethyl sulfoxide (DMSO). Linking reagents were added to the protein immediately after dissolution of the solid reagent, at molar concentrations at least 20 times that of protein. The reaction allowed to progress for at least 2 hours. Following derivatization, excess reagent was removed by centrifugation through a size exclusion resin (Zeba Desalting Column, PierceNet).

Streptavidin tetramers were prepared for assembly of complexes with derivatized NODEs. Streptavidin solutions were prepared by dissolving lyophilized Streptomyces avidinii streptavidin (ProZyme) in 50 mM sodium phosphate buffer pH 6.8, supplemented with 0.25M NaCl to minimize higher order streptavidin aggregates (see below). Streptavidin concentration was determined using the 41326 M⁻¹ cm⁻¹ A₂₈₀ extinction coefficient (Suter et al. 1988).

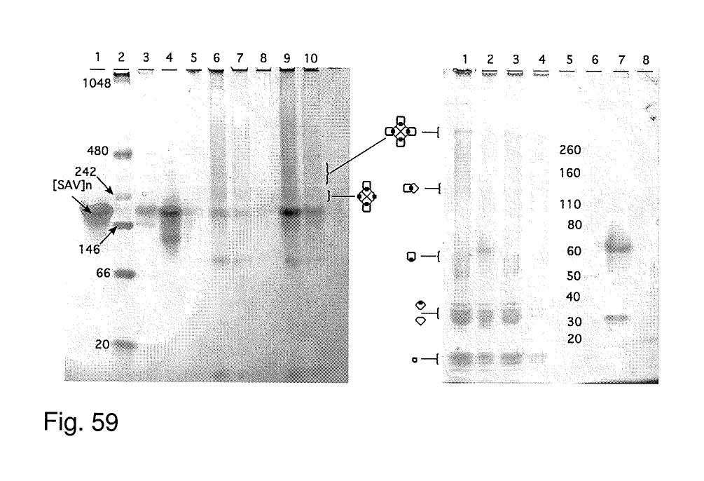

SAV:NODE complexes were formed by mixing the streptavidin and NODE solutions, generally by the addition of more concentrated streptavidin to the derivatized/biotinylated NODE. Based on experience with the streptavidin macromolecular adaptor, a novel streptavidin-capping reagent developed under a separate SBIR, where more discrete complexes tended to form when streptavidin was added in small aliquots until 2- to 3-fold molar excesses were achieved, a reaction volume of 100 mL protein was used to which 2 uL aliquots of streptavidin were added. The reaction equilibrated at room temperature for three days. Under these conditions, discrete SAV:IPP complexes formed along with some higher aggregates (FIG. 59). Complexes were analyzed by a PAGE procedure previously developed. A confounding issue with PAGE analyses of SAV:NODE complexes stemmed from the existence of stable dimeric, trimeric and higher aggregates of streptavidin in most preparations (Sano & Cantor 1990; Kurzban et al. 1991; Waner et al. 2004). We found the aggregates of liganded and unliganded streptavidin stable to heating at 70° C. and the liganded streptavidin aggregates also stable in SDS PAGE running buffers. We exploited these features in a gel assay to help assess complexation in both denaturing and native gels (FIG. 59). This approach complements imaging, light scattering and ESI and MALDI-MS experiments that provide additional data to more accurately quantitate complex formation and help interpret the gel patterns that provide a rapid and inexpensive initial measure of complex formation.

We have developed research approaches and analytical tools. We have physically created 7 synthetic genes, expression vectors, and cell pastes sufficient to purify 3- and 4-fold nodes in quantities sufficient to initiate a variety of studies to investigate a wide range of potential applications. We envision using 3- and 4-fold nodes and NODE: Streptavidin complexes in biosensor, biomedical research, and nanotechnology applications. We envision defining the exact compositions based on current genetic constructions that are best suited for scale up and initial sales.

We envision scaling up production of 4 first-generation C3- and C4-symmetric node proteins developed, designing and producing single-chain C3 nodes incorporating IgG binding domains, designing and producing single-chain C4 nodes, and developing assembly protocols, biophysical methods of analysis, and structural characterization of nodes and assemblies using X-ray crystallography and electron microscopy. We envision substantially expanding the range of accessible nanostructure assemblies.

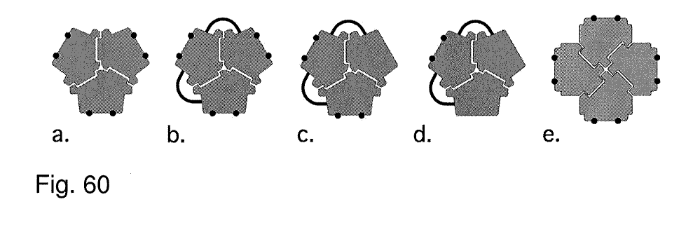

We envision preparing first-generation C3- and C4-symmetric engineered nodes and scaling production. Several first generation C3 and C4-symmetric nodes that have been developed are schematically illustrated in FIG. 60. These include the C3-symmetric node (FIG. 60a), the single-chain C3 nodes with 3 and 2 attachment sites for the streptavidin tetramer (FIGS. 60b, 60c), and the C4 4-fold symmetric node (FIG. 60e). We envision producing a C3 monovalent “capping” node (FIG. 60d). In order to scale production of these nodes for eventual commercialization, we envision optimizing several aspects of their production.

Expression vectors and systems were developed for 3 node variants using heterologous expression in E. coli from 16 liter fermentations that gave expression levels of about 2-4 mgs engineered node protein per gm of wet cell paste after purification. This was efficient for performing gene design, sequence verification, vector production, and protein expression. We envision investigating alternative fermentation strategies, with the objective of obtaining substantially higher yields.

The ability to achieve a substantial purification of the thermally stable nodes from the proteins of the background expression organism can be advantageous. We envision several methods to optimize protein recovery. For example, we envision optimizing recovery during the heating step by determining the melting temperature of each node protein, and then experimenting with heating protocols of the cell lysate within a few degrees of the node Tm to determine conditions of optimum recovery. Structurally intact nodes could be entrained in thermally denatured E. coli proteins during the heating step. We envision strategies for evaluating this possibility, e.g. by using a His-tag antibody to detect node proteins bound to the E. coli protein precipitate obtained after the heating step. As an adjunct to the purification improvements, we envision quantitatively determining the extinction coefficient at A₂₈₀ (or the maximum absorbance wavelength in that region) for each node construct, so that in subsequent reactions we can accurately control reaction stoichiometry. We envision developing an ESI-MS QC procedure for release of protein batches to confirm composition and verify reduced oxidation state of cysteine sulfhydryl groups.

We envision routinely achieving the highest possible extent of derivatization and uniformity for biotin-derivatized nodes. We envision a range of experimental protocols to achieve this objective, including the use of EDTA to prevent metal-promoted oxidation of free cysteine during node isolation, should ESI MS studies show that cysteine oxidation is a problem. Other factors we envision evaluating include variations in stoichiometry between node proteins and linking reagents, as well as optimal solvent conditions for reaction, because several biotinylation reagents have only limited solubility in aqueous solutions. Our results suggest that our engineered node proteins are fully stable in 30% DMSO solutions.

The fidelity of nanostructure assembly can be critically dependent on the purity and homogeneity of the molecular components. It can be important to achieve good separation of unreacted and derivatized nodes. If experiments in PAGE gels run at different pHs show differences in the mobility of fully reacted nodes and partially reacted products, this may be the basis for ion-exchange chromatographic separation to isolate fully derivatized products using the appropriate resin and buffer conditions. Alternatively, analyses of reaction products using ESI MS can be used to show almost exactly which impurities are present and more focused approaches applied. For example, if unreacted cysteine residues are an issue, then these unreacted node products can probably be removed through chromatography using thiol-affinity resins. Alternatively, cysteine residues could potentially be rendered unreactive through oxidation or other unanticipated side reactions. Using the ESI MS data as a guide, potential adduct reaction products can be identified, and undesired products minimized by removal or stoichiometry adjustment of a reactant (e.g. removal of b-mercaptoethanol (BME) can prevent formation of BME adducts). The formation of unreacted sulfinic acids can be avoided by careful elimination of oxygen from reaction mixtures or they can potentially be enzymatically reduced to thiols (Biteau et al. 2003; Woo et al. 2003).

We envision straightforwardly optimizing conditions and isolating 150 mg quantities of C3 and C4 nodes. These amounts are sufficient for applications testing development. We plan to produce nodes with free sulfhydryl groups to provide end-users with maximum flexibility in linking chemistry and to produce biotinylated nodes.

The engineered nodes that we are developing are completely novel molecular constructs providing unique capabilities for controlled nanostructure assembly.

We envision designing and producing single-chain C3 (3-fold symmetric) nodes incorporating IgG binding domains.

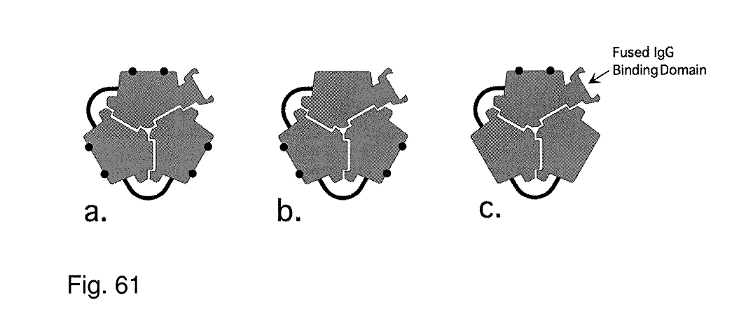

We envision expanding on the successful initial production of the single-chain variants of the g-carbonic anhydrase trimeric nodes (FIGS. 60b, 60c) and producing additional single chain variants that incorporate an IgG binding motif (FIGS. 61a, 61b, 61c), allowing immobilization of intact immunoglobulin molecules.

Antibodies are widely used in diagnostic applications and in biosensor microarrays. These applications are finding increased usage ranging from characterization of complete proteomes to identification of disease markers. However, technical issues limit their effectiveness and utility (Kusnezow & Hoheisel 2002; Winfren & Borrebaeck 2006; Torres et al. 2008). For example, one dominant issue stems from the relatively harsh conditions that are often encountered during antibody immobilization. Array assembly under native binding conditions that avoid harsh solutions for chemical derivatization usually preserves more antigen-binding capacity versus methods that involve IgG derivatization in solution (Kusnezow et al. 2006), owing in part to the reduced handling of IgG molecules, some of which have low stability.

One approach to providing native-like conditions for assembly of antibody-based arrays is to incorporate specific IgG binding domains into the array assembling materials. These small domains such as Streptococcal Protein G (Akerstrom & Bjorck 1986) incorporate ˜60 amino acid residues and bind IgG molecules with high specificity and affinity under native conditions. For example, microbeads incorporating the immunoglobulin binding domain of Protein G showed increases in both IgG binding capacity and antigen capture compared to less specific IgG absorbents (Vollenkle et al. 2003).

Factors leading to losses in antigen selectivity and affinity can be substantially reduced or eliminated using functionalized nanoarrays as specific binding substrates, and node proteins functionalized through incorporation of an immunoglobulin binding domain can be used in diagnostic and sensor applications.

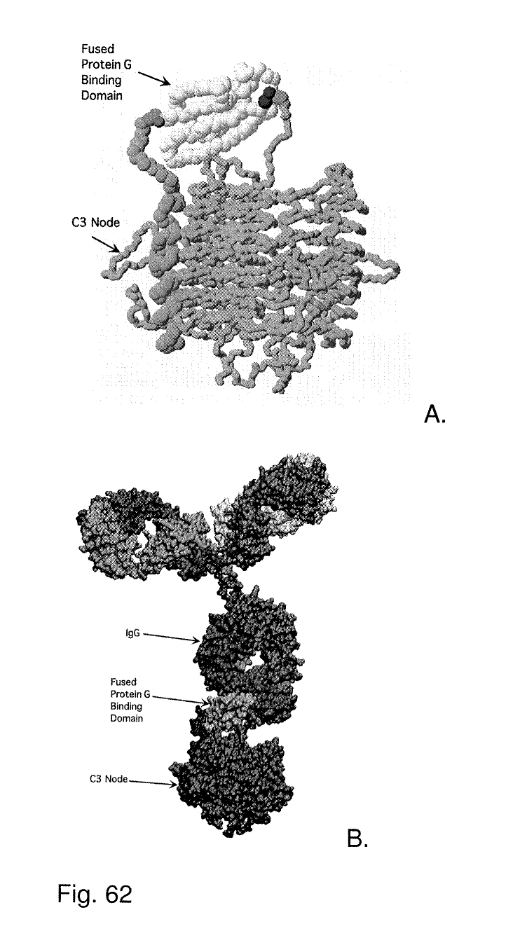

We envision designing a single-chain C3 node incorporating an IgG binding domain. FIG. 62 outlines an initial design concept for a single-chain C3 node based on the g-carbonic anhydrase template that has been developed. As illustrated in FIG. 62A, the arrangement of a C-terminal a-helical extension stemming from a b-sheet makes the molecule well suited for the introduction of a Protein G (or the structurally-related Protein A) IgG binding domain. We envision a more complete structural analysis and model generation process to find sequences of appropriate geometry, length and composition to reconnect the subunit sequences as outlined below. Both “flexible” and more rigid interconnections can be investigated.

We envision expressing and purifying a single-chain C3 node incorporating an IgG binding domain. A single-chain version of the C3 g-carbonic anhydrase node expressed well and retained enzymatic activity. Fusion protein vector construction, protein expression and purification steps can be carried out. While it is difficult to predict the expression characteristics of a new construct, the small size of Protein G relative to the single-chain g-carbonic anhydrase, along with the insertion site in a surface loop near the C-terminus are structural features that can lead to successful expression. We envision expressing three variants of the single-chain C3 IgG binding node based on the previously developed single-chain constructs incorporating 3 (for assembly of hexagonal lattices), 2 (for assembly of hexagons), and 1 (a “cap” with IgG binding functionality) streptavidin binding sites, respectively.

We envision creating several variants of an engineered single-chain C3 node incorporating an integral IgG-binding domain. We expressed a single chain g-carbonic anhydrase construct, and envision successful isolation of the engineered variants, for example, a single-chain structure incorporating the IgG-binding domain. These engineered nodes are completely novel constructs and substantially expand the range of functional nanostructures that can be assembled using our “parts box”.

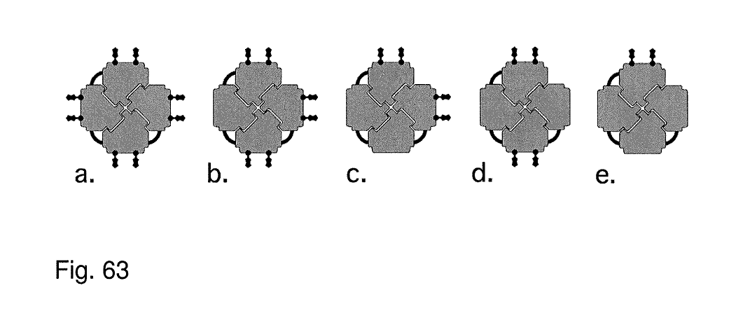

We envision designing and producing 4-fold NODEs with novel arrangements for strut attachment. The objective of this specific aim is to expand on the successful production of the IPP isomerase C4 node and engineer single chain variants able to bind 1-4 streptavidin tetramers with defined geometry (FIG. 63).

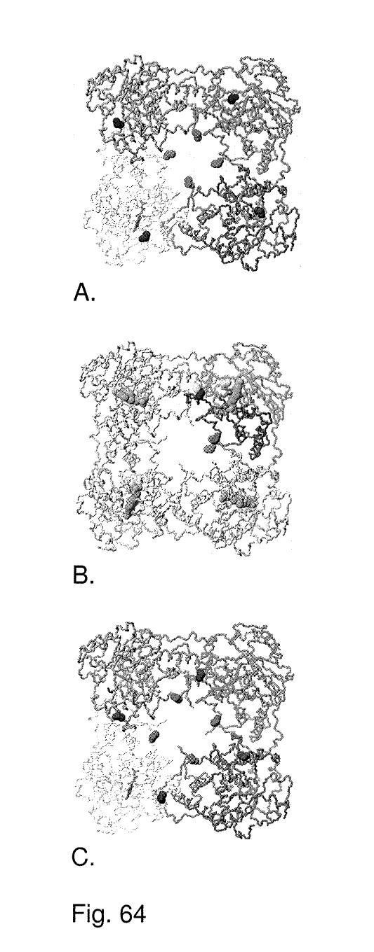

Incorporation of all n subunits of a Cn symmetric node into a single polypeptide chain (for example, in a single-chain C4 node design) allows the formation of nodes where it is possible to control both the geometry and valency of connecting struts, thus greatly expanding the variety of nanostructures that can be assembled. We envision building on experience with the C4 node IPP isomerase to generate the node valencies and geometries as outlined in FIG. 63. Formation of a single chain molecule involves connecting adjacent C- and N-termini for three (of four) subunits. In the 4-fold symmetric IPP isomerase tetramer, the shortest linear distance between C- and N-termini is ˜35 A (FIGS. 56 and 62). In practice, a longer linker is be required to wrap around the protein surface. While linkers of this length are routinely used in production of single chain antibody Fv's (Ladner 2007), more efficient designs can be generated if the individual subunits are first reengineered to introduce new N- and C-termini. Using an approach similar to one used by the PIs for design of interleukin 1b permuteins (Horlick et al. 1992), FIG. 64 shows an initial model of an IPP isomerase variant where new N- and C-termini have been introduced by reconnection of the subunit polypeptide chains. We envision completing the structural analysis and model generation processes to find sequences of appropriate geometry, length, and composition to reconnect the subunit sequences and interconnect the newly generated subunit termini. A combination of computer modeling, computational, and fragment based methods can be used to generate the single-chain node sequence. Such fragment-based approaches, pioneered by the applicants (Finzel et al. 1990; Wendoloski & Salemme 1992), are features of most current crystallographic modeling packages, and have been extensively used by the applicants in drug-discovery protein engineering projects including, for example, the generation of a single chain HCV protease containing the activating portion of NS4A fused to the NS3 protease domain (Taremi et al. 1998, Malcolm et al. U.S. Pat. No. 6,653,127). A model of the IPP isomerase tetramer with engineered termini shows that C- and N-termini in adjacent subunits are in close proximity (˜0.12 A apart) and situated on the same molecular face (FIG. 64). Introduction of a His6 tag into the reconnected single-chain can preserve the orientation established by the His6 tagged native WP isomerase (FIG. 54), an important feature for formation of 2-D assemblies incorporating multiple node types.

With successful design and expression of a single-chain C4 node, we can produce variants designed to bind from 1 to 4 streptavidin tetramers. Cysteine sites for attachment of linked biotin can be taken from the IPP isomerase 4-fold node which has been produced. FIG. 5.4 shows 4-fold node variants that can be produced.

We envision expressing, purifying, and characterizing key single chain 4-fold nodes. A single-chain version of the C3 g-carbonic anhydrase node expressed well and retained enzymatic activity. We can follow the approaches and protocols developed and described to engineer a single-chain C4 node. We can generate the reconnected IPP tetramer with new N- and C-termini (FIG. 64B). Following successful expression, as measured by retention of a thermostable tetramer structure we can engineer the single chain variants.

The reconnections initially modeled are primarily in surface loops on the protein, and there is precedent that such reconnection strategies can work in other engineered proteins. However, several iterations may be necessary (e.g. experimenting with different loop lengths and compositions) before a single-chain that has the desired thermal stability and expresses well in E. coli is produced. We envision developing a computer model of a single-chain C4 node based on an alternative template, where ease of design of a single-chain variant is given highest priority. The stability and high expression level of the C4 node based on the IPP isomerase template makes further pursuit of this template a logical first choice. Development of single-chain C4 nodes can expand the range of structures that can be assembled.

We envision developing technology relevant to node biophysical and structural characterization, as well as methods relating to assembling nanostructures and characterization of their structure. Knowledge of protein thermostability is useful. We have determined that the purified 3-fold trimeric node has a Tm of at least 55° C. (Alber & Ferry 1996) and that the IPP isomerase has a Tm value>65° C. We envision quantitatively measuring the thermal stability of constructs and complexes throughout production and elsewhere, because preservation of thermal stability is an important factor impacting the potential range of nano-assembly applications. For example, an objective is to determine which of the 3-fold constructs that are functionally equivalent (FIGS. 60a, 60b) is more stable, so that higher priority can be given to production of the more stable node. Thermal stabilities can be important in the design and selection of second-generation nodes. Stability of the single-chain C3-protein-G fusions and C4 single-chain variants can be monitored. Analysis of node thermal stabilities as a function of changes in solution conditions can allow refinement of the initial heat step used in node purification and subsequent isolation steps.

Thermal stabilities can be measured using a microplate-compatible format developed by one of the applicants (Pantoliano et al. 2001). The method depends upon the fact that native folded proteins are highly organized structures that melt cooperatively at a specific temperature that is characteristic for each protein and representative of the free energy of stabilization of the protein's folded state. Protein thermal transitions are traditionally measured using a differential scanning calorimeter (DSC). However, the melting effect can be efficiently measured in a microplate format using much less material than required for a DSC experiment by performing a thermal scan while measuring the fluorescence of a dye (typically ANS) that only fluoresces when binding to the melted or “molten globule” state of the protein. In addition, the method can be used to determine effects of solvent environment, chemical modification, or ligand binding on protein thermal stability, which are additionally useful characteristics of the method in the context of the current work. In addition to direct measurements of thermal stability, we can monitor structural integrity and uniformity using dynamic light scattering, alone and in combination with static light scattering. These polymer physics methods provide measures of particle size, anisotropy, and particle molecular weight, and have been implemented in micro formats, making them practical as routine laboratory methods. A relatively high throughput assay for node thermostability can be established using commercial instruments originally developed for quantitative PCR measurements. Instruments operating up to 75° C. are currently available, with instruments operating at higher temperatures in the development stage. For proteins with thermostabilities higher than 75° C., sealed cells in a closed-cell differential scanning calorimeter can be used or alternative approaches and instrumentation such as those applied to the measurement of polymer melting temperatures can be used. The use of ThermoFluor as a monitoring tool for processing and refinement of engineered proteins is innovative.

We envision refining and optimizing conditions for nanostructure assembly. For example, we envision eliminating aggregates of streptavidin under conditions for nanostructure assembly. Several engineered molecules based on avidin and streptavidin have been reported (Sano et al. 1998; Laitinen et al. 2007). Most commercial streptavidin preparations, including a protein from Prozyme, contain stable dimeric, trimeric and higher aggregates of streptavidin tetramers (Sano & Cantor 1990; Kurzban et al. 1991; Waner et al. 2004). We found the aggregates of both liganded and unliganded streptavidin stable to heating at 70° C., and the liganded streptavidin aggregates stable in SDS PAGE running buffers. The aggregates likely present few problems for ELISA or other affinity-based uses of streptavidin. However, in the assembly of the nanostructures envisioned, it can be important to use the pure tetramer form of streptavidin. Based on established principles of protein chemistry and using modern purification methods, we can develop conditions that eliminate aggregates that form on lyophilization using analytical and biophysical methods such as light scattering and PAGE.

We envision preparing single-chain nodes, derivatized nodes, node:streptavidin complexes, and sub-assemblies. One issue concerns polymer formation with single-chain constructs. We were successful in preparing the single-chain C3 g-carbonic anhydrase node. However, the potential exists for single-chain structures to swap domain interactions between different polypeptide chains and form polymers. These polymers may be interesting materials in their own right. However, the formation of domain swapped polymers can frustrate the controlled formation of nanostructures. We envision employing light scattering to closely monitor our process conditions to avoid conditions that promote polymer formation. We envision determining optimal conditions for node derivatization and nanostructure assembly. Table 3 lists a variety of biotinylation reagents potentially useful in complex assembly. We have used several of these effectively to derivatize nodes and make node:streptavidin complexes. We envision investigating additional reagents as well. We have developed a new reagent that allows us to modify cysteine residues with iminobiotin adducts. Although the binding constant for streptavidin for iminobiotin is somewhat reduced relative to biotin (Kd 10⁻¹¹ vs. Kd 10⁻¹⁴), iminobiotin binding is pH-dependent. This can provide important advantages in nanostructure assembly. A difficulty in building structures incorporating biotin:streptavidin linkages can be the essential irreversibility of the interaction. In contrast, most lattice growth mechanisms involve dynamic “annealing” as new molecules are added, so that every molecule in the structure can find an equivalent minimum energy configuration. We envision experimenting with this reagent in the assembly of nanostructures, using dynamic variations in pH to investigate how this can affect the homogeneity of the resulting nanostructures. We envision better monitoring of nanostructure assembly. We have used PAGE to characterize our derivatives and complexes. We envision expanding the range of biophysical and structural tools used to characterize both the assembly process and the final structures of our assemblies. We envision using light scattering as well direct structure determination methods.

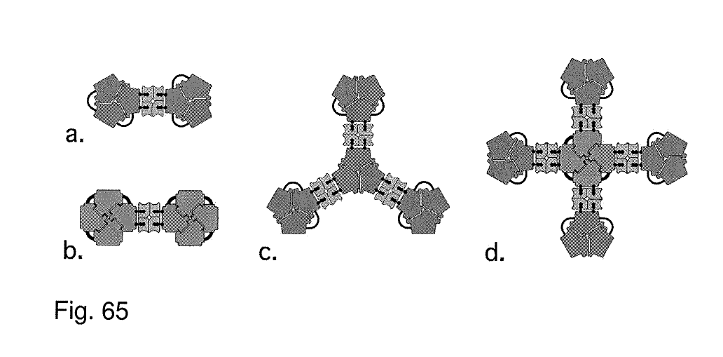

Node proteins can be initially synthesized with poly-His affinity sequences fused to their termini to aid protein purification using metal-chelating resins and to provide specific geometrical orientation of the nodes on 2D surfaces. However, there are instances where it may be useful to remove the affinity tag, for example, to construct subassemblies, such as those shown schematically in FIGS. 65c and 65d. Consequently, envision refining methods to remove the affinity tags and to make both tagged and untagged nodes available to collaborators and customers. We designed node genes with affinity tags that can be cleaved from the node by well-known proteases. We can maintain a database of optimized experimental conditions for efficient removal of affinity tags. The intrinsic thermostability of the node proteins may facilitate use of processing temperatures that are optimal for protease enzymatic activity (Wolf et al. 1995; Nallamsetty et al. 2004).

We envision applying the above-described approaches to both the engineered C3 and C4-symmetric nodes in a step-wise fashion, beginning with the underivatized nodes, and progressing to derivatized nodes, single-chain nodes, and substructure assemblies. Potentially accessible substructures, assembled in solution from streptavidin and biotin- or imino-biotin-linked, monovalent, single-chain “capping” nodes can include structures like those shown in FIGS. 65a and 65b. Immobilization of C3- or C4 symmetric nodes on a Ni-resin through the node terminal His-tags, followed by 1) reaction with streptavidin, 2) reaction with single-chain, single-valence nodes, and 3) proteolytic cleavage of the central node from the Ni-resin substrate, can produce structures such as those shown in FIGS. 65c and 65d. Advantages of the immobilization strategy for nanostructure assembly include 1) ability to prevent interfering interactions between nodes anchored at different sites, 2) ability to completely saturate node valency sites with streptavidin 3) ability to remove any unreacted streptavidin, 4) ability to completely saturate streptavidin valency sites with monovalent “capping” nodes”, and 5) ability to completely remove excess monovalent “capping” nodes prior to release or proteolytic cleavage of the nano-assembly from the Ni-resin substrate.

In addition to preparing substructures concerning appropriate reaction conditions, component stoichiometry requirements, and appropriate purification methods (these can range over chromatography, electrophoresis, isoelectric focusing, or density gradient centrifugation), it is useful to determine x-ray crystal structures of the complexes to verify (and potentially modify) streptavidin:node interaction geometry.

We have engineered Cn nodes with poly-His terminal sequences to aid in isolation and also to geometrically orient the Cn node axis perpendicular to an underlying immobilization surface such as a metal surface or self-assembling-monolayer (FIGS. 54 and 55). This geometry was engineered to be consistent with the formation of geometrically correct interconnections between nodes lying in a plane and bridging streptavidin tetramers, as outlined above (FIG. 50). Consequently, the engineered nodes that we have developed are designed be compatible with the formation of extended 2D hexagonal and square lattices generated using a 2D diffusional self-organization strategy on self-assembled monolayers (SAMs). Ringler & Schulz (2003) incorporated a poly-His tag fused to the C-terminus of each subunit of their C4 aldolase node (although, as noted above, the biotinylation sites they introduced were not oriented optimally for streptavidin interconnections), principally as a means of facilitating 2D lattice assembly through interaction with a SAM incorporating a Ni-chelating lipid (Ni-2-(bis-carboxymethyl-amino)-6-[2-(1,3)-di-O-oleyl-glyceroxy)-acetyl-amino] hexanoic acid or Ni-NTA-DOGA).

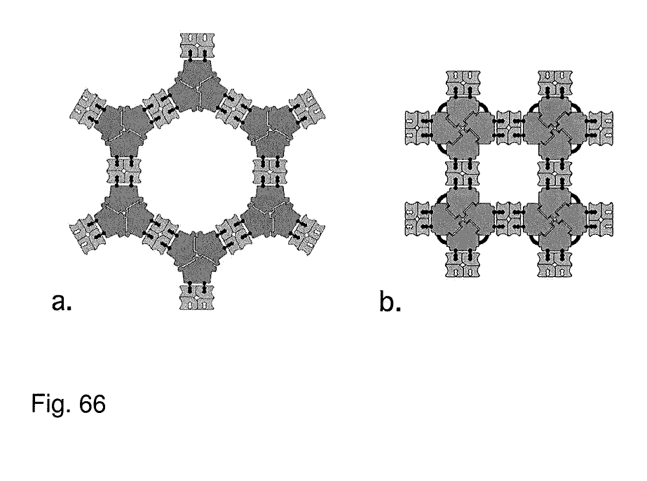

Our nodes can be used to develop both hexagonal and square 2D lattice structures assembled on Ni-NTA-DOGA SAMs. In FIG. 66 we show extended 2D hexagonal (FIG. 66a) and square (FIG. 66b) lattice structures assembled using the C3- and C4-symmetric nodes developed. Many additional structures are also accessible using the variable valency and geometrical control afforded in the single-chain C3 monovalent and divalent nodes (FIGS. 60c, 60d) as well as the variety of C4 single-chain nodes (FIGS. 63b, 63c, 63d, 63e). Using our nodes together with streptavidin, extended nanostructures can be assembled. Precisely assembled nanostructures with controlled architectural features can be produced, as can structures with “local” order extending over a few lattice repeats, which may provide useful and previously unprecedented properties.

Our careful node design process, use of extensive biophysical, optical, and thermal stability measurement methods to carefully monitor the stability and uniformity of both our nodes and streptavidin, as well as development of a pH-dependent iminobiotin cross linker allowing reversible pH-dependent biotin binding and structural annealing, can contribute to successful nanoscale assemblies. We can apply substructure purification methods. The above-discussed techniques enable the formation of continuous lattice structures assembled on SAMs (FIG. 66a, FIG. 5.8a). We envision completely novel protein-based nanostructures providing a framework for the subsequent development of a range of new biomaterials and biosensors.

We envision applying X-ray crystallography techniques to carry out crystal structure determinations of nodes and/or nodes complexed with streptavidin in addition to molecular modeling approaches based on available crystal structures. Examples of target structures include the single chain C3 node structures, including protein G fusions (FIG. 62A), the C4 symmetric node with re-engineered N- and C-termini (FIG. 64B), and the C4 single-chain nodes (FIG. 63). Owing to the overall stability of the C3 g-carbonic anhydrase and C4 IPP isomerase templates (Tm>65° C.), and reasonable levels of expression in E. coli obtained thus far, we expect to be successful in purifying sufficient protein to crystallize the isolated nodes. We expect the structure solutions to progress rapidly using molecular replacement methods applied with X-ray crystallography techniques. We envision completely novel protein-based nanostructures providing a framework for the subsequent development of a range of new biomaterials and biosensors.

In addition to crystallographic work, we envision using electron microscopy to visualize complexes and lattices immobilized on surfaces. A combination of diffraction and imaging methods can be useful in determining the extent of order in nanoassembled arrays. Negative staining EM or potentially, cryo-crystallography image superposition methods can be applied to image substructures and/or 2D lattices. These imaging techniques can be applied to develop completely novel completely materials for manufacturing protein-based nanostructures.

We envision a comprehensive platform allowing the construction of protein-based nanostructures with utility as biosensors, biomaterials, or other devices. The platform incorporates a set of modular components engineered from thermostable proteins that can be used to create structures where novel properties emerge owing to the ability to control structural or functional organization at the nanoscale. Our first-generation building blocks comprise a set of engineered proteins that allow construction of a wide variety of linear, planar and 3-D nanoassemblies. Our components can provide the basis for a new generation of diagnostics, biosensors, biomaterials, and industrial processes that use organized nanostructures as integral components. We envision the sequential development of molecular components, nanostructural modules, arrays, and functional nanodevices. These components can be used in biomedical and nanostructure research and applications development, and to develop biomaterial and biosensor products.

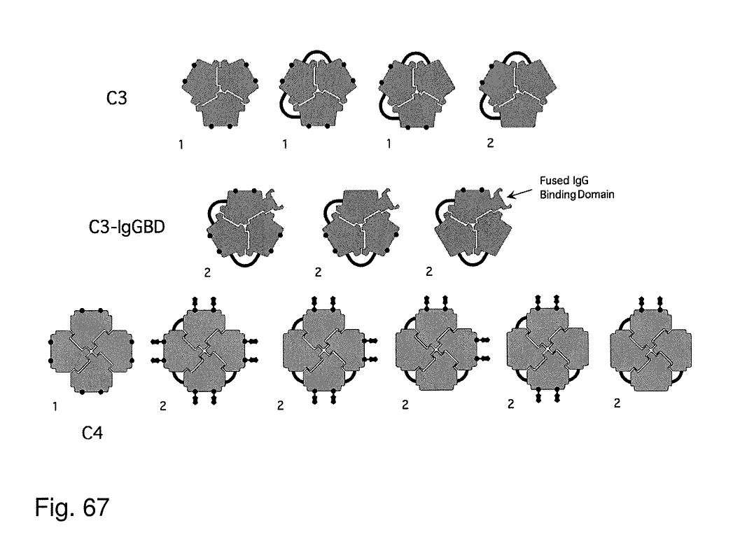

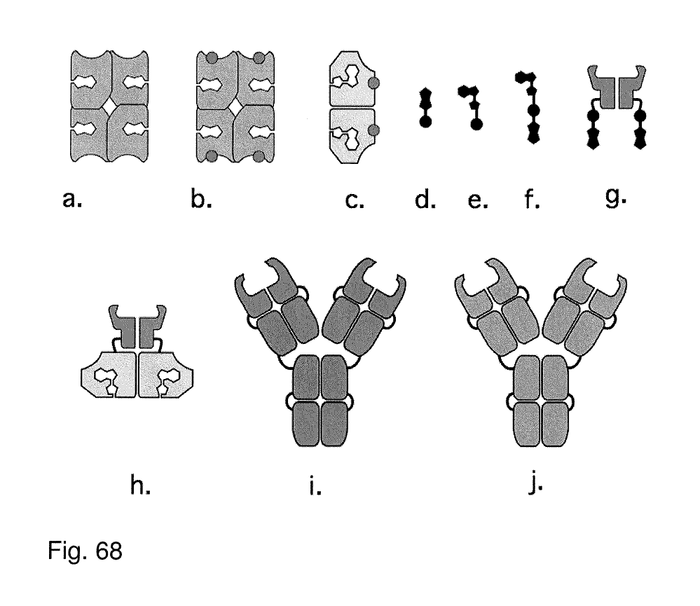

We have developed building blocks that we term “struts” and “nodes” engineered to provide the underlying architecture for devices and materials. Struts are linear structural elements, while nodes are protein structures or assemblies with Cn rotational or 3-dimensional point group symmetry. Our struts can incorporate streptavidin, a tetramer with D2 symmetry that incorporates 4 high-affinity (Kd˜10⁻¹⁴) biotin binding sites oriented approximately as the legs of an “H”. Nodes are site-modified proteins with plane or point group symmetry (typically modified forms of protein multimers) that incorporate covalently bound biotin groups that are pairwise-complementary to the biotin binding sites on streptavidin, and are designed to allow the assembly of 1D, 2D, and ultimately 3D structures with defined geometrical organization. FIG. 67 schematically illustrates the C3 and C4-symmetric node structures developed or being developed. FIG. 68 schematically illustrates additional reagents and engineered proteins useful for nanostructure assembly.

The components shown in FIGS. 67 and 68 embody, for example, three types of functions: 1) node components with different strut-binding geometry and ligation number that control nanostructure architectural features, 2) strut components that interconnect the nodes, and 3) reagents and engineered adaptor proteins that facilitate controlled assembly of the nanostructures or the attachment of additional proteins that can confer functionality on the underlying nanostructure architecture. Multiple functions can be combined in a single molecular component, as, for example, in the single-chain C3 nodes incorporating the integrally fused IgG binding domain (FIG. 67 center row). Controlling nanostructure assembly is important. We have developed two different approaches to achieve this objective. A first approach involved development of a thiol-reactive iminobiotin reagent. Since iminobiotin binding to streptavidin is pH-dependent (dissociating at mildly acid pH), this reagent can be used in many ways to control what may otherwise be spontaneous and irreversible streptavidin:biotin interactions and may be particularly important to allow “annealing” in extended 2D assemblies. A second approach involved the design and engineering of a reversible protecting group for 2 of the binding sites on streptavidin that we termed a “streptavidin macromolecular adapter protein” or “SAMA”. This construct offers a level of controlled nanostructure assembly, as for example illustrated in FIG. 46 for assembling an extended strut. The SAMA is based on a dimeric protein, having 2 ATP binding sites that are geometrically complementary to 2 biotin binding sites on streptavidin, that has been additionally engineered to incorporate two cysteine residues that can be subsequently biotinylated to regenerate streptavidin binding capability while preserving overall strut geometry.

FIG. 68 shows additional reagents and modular components useful in functional nanoassembly fabrication, including a variant dimeric ATP-binding protein (FIG. 68h) incorporating fused, IgG-binding protein-G domains. Biotinylated IgG binding domains that can be directly connected to streptavidin via biotinylation linkers (FIG. 68) are available from research biochemical suppliers (www.piercenet.com) as are additional reagents (www.quantabiodesign.com) and antibodies outlined in FIG. 68.

An application involves the generation of immobilized antibody arrays (in fact, the immobilized species can be IgGs, Fabs, or single-chain Fvs, depending on the application) with controlled geometry.

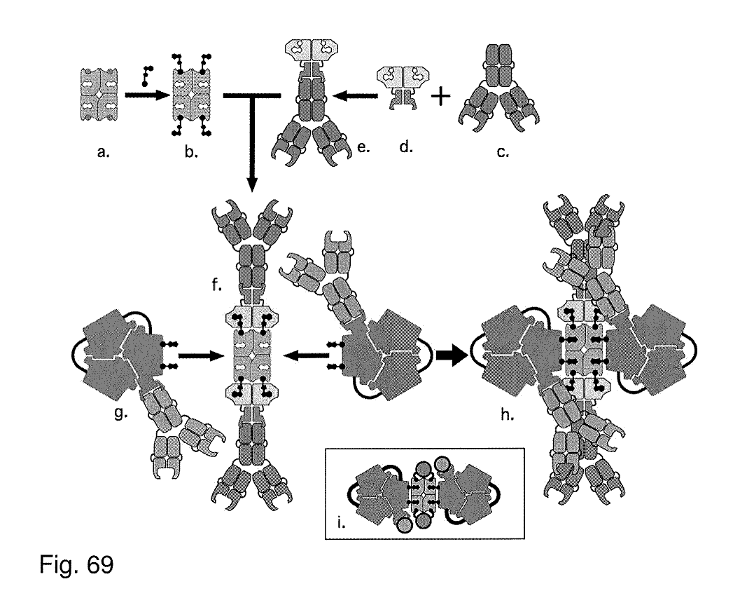

Improvements in detector affinity and specificity are associated with organized, high density immobilization of in IgGs on sensor array surfaces (e.g. Souka et al. 2001). An aspect unique to the constructs and devices that we envision is the ability to control and precisely position the relative orientation of two (or ultimately more) IgG molecules to a substrate surface. e.g. attach to different epitopes of an antigen simultaneously. FIG. 69 illustrates how some of the components described above can be used in a convergent synthesis to assemble a small component incorporating 2 pairs of different antibodies in close proximity. This resulting highly specific capture agent might be developed into a sensor device, since the binding of a hapten would “freeze” the relative orientations of the bound IgGs (whose interdomain connections are otherwise quite flexible), an effect that could be potentially detected using a variety of biophysical methods such as fluorescence resonance energy transfer FRET (http://en.wikipedia.org/wiki/Fluorescence_resonance_energy_transfer) or other methods. Since the single-chain C3 nodes shown in FIG. 69 incorporate terminal orienting His-tags, the structures are readily immobilized on Ni-resins, coated metal surfaces, or self-assembling membrane (SAM) surfaces that incorporate the a Ni-chelating lipid Ni-NTA-DOGA (Ni-2-(bis-carboxymethyl-amino)-6-[2-(1,3)-di-O-oleyl-glyceroxy)-acetyl-amino] hexanoic acid).

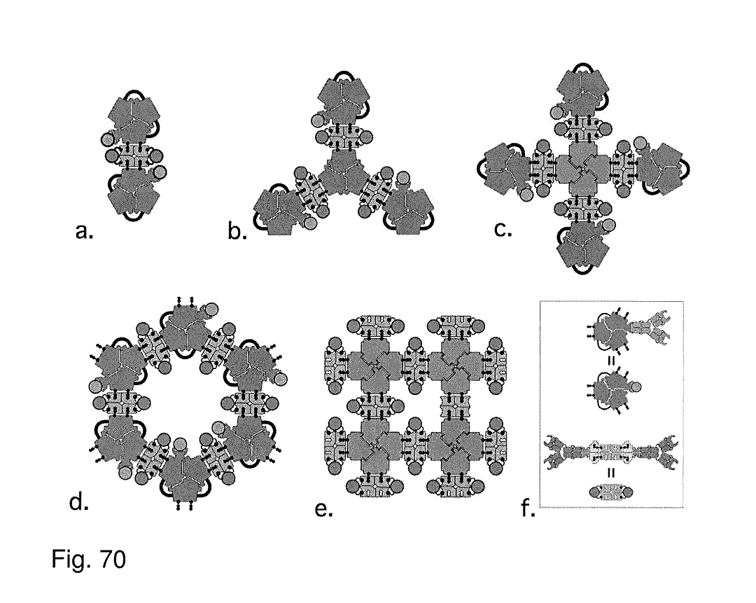

Examples of nanostructures functionalized with either one or two different IgG molecules that can be constructed using the basic component system are illustrated in FIG. 70. FIG. 70a schematically recapitulates the assembly whose construction was outlined in FIG. 69. 70b and 70c show assemblies built on C3 and C4 nodes respectively. Construction of such assemblies could proceed via the initial immobilization of the central C3 or C4 nodes through their terminal His-tags, followed by attachment of Streptavipol:IgG conjugates, and subsequently, attachment of single-chain C3 nodes incorporating IgG binding domains, from which the terminal His-tags had first been removed. FIG. 70d shows a hexagonal structure composed of Streptavipol:IgG conjugates and single-chain C3 nodes incorporating IgG binding domains. FIG. 70e shows a square 2D lattice constructed of Streptavipol:IgG conjugates and C4-symmetric nodes. The assembly of structures such as those shown in FIGS. 70e and 70f might best be approached using “pH-annealable” iminobiotin conjugated nodes, linked through their terminal His-tags to the Ni-chelating lipid Ni-NTA-DOGA, and free to undergo 2-dimensional diffusion on a SAM surface. FIG. 70f shows alternative schematic representations of the Streptavipol:IgG conjugate and C3 single-chain IgG conjugate modules.

A flexible set of nanoassembly components and modules can allow the construction of functionalized lattice structures such as those illustrated in FIG. 70. This can enable a host of new functional applications. Some immediate and important applications of our technology are that our components can facilitate the assembly of diagnostic devices and sensors incorporating multiple molecular detectors whose relative geometry and stoichiometry are precisely controlled. An advantage of such structures is that they potentially offer much greater detection sensitivity and specificity than a detection system incorporating a single antibody (or single-chain Fv, etc.), because two different antibodies can be geometrically constrained, so that they potentially interact with the same antigen simultaneously. The potential improvements in detection sensitivity and specificity to be gained in such applications can be an important application of our components in the protein marker diagnostics market.

Applications for our nano-component products include biomedical diagnostics, proteomics and nanotechnology, including, for example, “personalized” approaches to medicine based on the analysis of specific disease-related protein biomarkers. We believe that our technology can enable numerous advancements in the protein biomarker space, beginning with the basic biosensor applications such as those outlined in FIGS. 70 & 7.5. We envision supplying selected nodes (where the experimenter has to do “chemistry” and purification work to actually make structures) and also supplying modular complexes that can basically “snap” together to form more elaborate functionalized structures like those shown in FIG. 70. We envision using proteins, such as our components, for nanodevice fabrication, for example, protein-based nanostructures for large-scale applications with fundamental public health benefits such as active membranes for water purification.

### REFERENCES

Akerstrom B, Bjorck L “A Physicochemical Study of Protein G, a Molecule with Unique Immunoglobulin G-binding Properties” J Biol Chem (1986) 261:10240-10247.
Alber B E, Ferry J G “Characterization of Heterologously Produced Carbonic Anhydrase from Methanosarcina thermophila” J Bacteriol (1996) 178:3270-3274.
Armstrong J M, Myers D V, Verpoorte J A, Edsall J T “Purification and properties of human erthrocyte carbonic anhydrases” J Biol Chem (1966) 142:5137-5149.
Arrau J, Lauritzen C, Petersen G E, Pedersen J “Current strategies for the use of affinity tags and tag removal for the purification of recombinant proteins” Prot Expr Purif (2006) 48:1-13.
Arnau J, Lauritzen C, Petersen G E, Pedersen J “The use of TAGZyme for the efficient removal of N-terminal His-tags” Methods Mol Biol (2008) 421:229-243.
Avila-Sakar A J, Chiu W “Visualization of b-sheets and side-chain clusters in two-dimensional periodic arrays of streptavidin on phospholipid monolayers by electron crystallography” Biophys J (1996) 70:57-68.
Baldwin A J, Bader R, Christodoulou J, MacPhee C E, Dobson C M, Barker P D “Cytochrome c fused to b-amyloid for plaque detection” J Am Chem Soc (2006) 128:2162-2163.
Biteau B, Labarre J, Toledano M B “ATP-dependent reduction of cysteine-sulfinic acid by S. cerevisiae sulphlredoxin” Nature (2003) 425:980-984.
Brumfield S, Willits D, Tang L, Johnson J E, Douglas T, Young M “Heterologous expression of the modified coat protein of Cowpea chlorotic mottle bromovirus results in the assembly of protein cages with altered architectures and function” J Gen Virol (2004) 85:1049-1053.
Butler T Z, Pavlenok M, Derrington I M, Niederweis M, Gundlach J H “Single-molecule DNA detection with an engineered MspA protein nanopore” Proc Natl Acad Sci USA (2008) 105:20647-20652.
Case D A, Cheatham T E, Darden T, Gohlke H, Luo R, Merz K M, Onufriev A, Simmerling C, Wang B, Woods R “The Amber biomolecular simulation programs” J Computat Chem (2005) 26:1668-1688.
Cassimjee K E, Trummer M, Branneby C, Berglund P “Silica-immobilized His6-tagged enzyme: alanine racemase in hydrophobic solvent” Biotechnol Bioeng (2008) 99:712-716.
Clemmens J, Hess H, Doot R, Matzke C M, Bachand G D, Vogel V “Motor-protein “roundabouts”: microtubules moving on kinesin-coated tracks through engineered networks” Lab Chip (2004) 4:83-86.
de Ruyck J, Rothman S C, Poulter C D, Wouters J “Structure of Thermus thermophilus type 2 isopentenyl diphosphate isomerase inferred from crystallography and molecular dynamics” BBRC (2005) 338:1515-1518.
De Stefano L, Vitale A, Rea I, Staiano M, Rotiroti L, Labella T, Rendina I, Aurilia V, Rossi M, D'Auria S “Enzymes and proteins from extremophiles as hyperstable probes in nanotechnology: the use of D-trehalose/D-maltose-binding protein from the hyperthermophilic archaeon Thermococcus litoralis for sugars monitoring” Extremophiles (2008) 12: 69-73.
Eteshola E, Keener M T, Elias M, Shapiro J, Brillson L J, Bhushan B, Lee S C “Engineered functional protein interfaces for immunologically modified field effect transistor (ImmunoFET) by molecular genetic means” J R Soc Interface (2008) 5:123-127.
Finzel B C, Kimatian S, Ohlendorf D H, Wendoloski J J, Levitt M, Salemme F R “Molecular Modeling with Substructure Libraries Derived from Known Protein Structures” In Crystallographic and Modeling Methods in Molecular Design (S Ealick & C Bugg eds.) Springer Verlag, New York (1990) pp. 175-189.
Frey W, Brink J, Schief W R Jr, Chiu W, Vogel V “Electron crystallographic analysis of two-dimensional streptavidin crystals coordinated to metal-chelated lipid monolayers” Biophys J (1998) 74:2674-2679.
Fu X, Yu L J, Mao-Teng L, Wu C, Yun-Feng M “Evolution of structure in gamma-class carbonic anhydrase and structurally related proteins” Mol Phylogenet Evol (2008) 47:211-220.
Gilardi G, Fantuzzi, A “Manipulating redox systems: application to nanotechnology” Trends Biotechnol (2001) 19:468-476.
Gonzalez M, Bagatolli L A, Echabe I, Arrondo J L R, Argarana C E, Cantor C R, Fidelio G D “Interaction of Biotin with Streptavidin” J Biol Chem (1997) 272:112288-11294.
Guex N “Swiss-PdbViewer: A new fast and easy to use PDB viewer for the Macintosh” Experientia (1996) 52:A26.
Guex N, Diemand A, and M. C. Peitsch M C “Protein Modelling for All” Trends Biochem Sci (1999) 24:364-367.
Green N M “Avidin” Adv Prot Chem (1975) 29:85-133.
Hofmann K, Wood S W, Brinton C C, Montibeller J A, Finn F M “Iminobiotin affinity columns and their application to retrieval of streptavidin” Proc Natl Acad Sci USA (1980) 77:4666-4668.
Horlick R A, George H J, Cooke G M, Tritch R J, Newton R C, Dwivedi A, Lischwe M, Salemme F R, Weber P C, Horuk R “Permuteins of interleukin 1b—a simplified approach for the construction of permutated proteins having new termini” Prot Eng Design Sel (1992) 5:427-431.
Humphrey W, Dalke A, Schulten K “VMD: visual molecular dynamics” J Mol Graph (1996) 14:33-38, 27-28.
Humphrey W, Alber B E, Kisker C, Ferry J G, Rees D C “A Closer Look at the Active Site of gamma-Class Carbonic Anhydrases: High-Resolution Crystallographic Studies of the Carbonic Anhydrase from Methanosarcina thermophila” Biochemistry (2000) 39:9222-9231.
Iverson T M, Dalke A, Schulten K “VMD: visual molecular dynamics” J Mol Graph (1996) 14:33-8 & 27-8.
Jeyakanthan J, Rangarajan S, Mridula P, Kanaujia S P, Shiro Y, Kuramitsu, Yokoyama S, Sekar K “Observation of a calcium-binding site in the gamma-class carbonic anhydrase from Pyrococcus harikoshi” Acta Cryst D Biol Crystallog (2008) 64:1012-1019.
Jones T A, Bergdoll M, Kjeldgaard M “0: A macromolecular modeling environment” In: Crystallographic and Modeling Methods in Molecular Design. C Bugg & S Ealick Eds. Springer-Verlag Press (1990)189-195.
Kenig M, Peternel S, Gaberc-Porekar V, Menart V “Influence of the protein oligomericity on final yield after affinity tag removal in purification of recombinant proteins” J Chromatogr A (2006) 1101:293-306.
Kickhoefer V A, Garcia Y, Mikyas Y, Johansson E, Zhou J C, Ravel-Fernandes S, Minoofar P, Zink J I, Dunn B, Stewart P L, Rome L H “Engineering of vault nanocapsules with enzymatic and fluorescent properties” Proc Natl Acad Sci USA (2005) 102:4348-4352.
Kisker C, Schindelin H, Alber B E, Ferry J G, Rees D C “A left-hand beta-helix revealed by the crystal structure of a carbonic anhydrase from the archaeon Methanosarcina thermophila” EMBO J (1996) 15:2323-2330.
Kurzban G P, Bayer E A, Wilchek M, Horowitz P M “The Quaternary Structure of Streptavidin in Urea” J Biol Chem (1991) 266:14470-14477.
Kusnezow W, Hoheisel J D “Antibody Microarrays: Promises and Problems” Biotechniques (2002) 33:14-23.
Kusnezow W, Syagailo Y V, Ruffer S, Baudenstiel, Gauer C, Hoheisel J D, Wild D, Goychuk I “Optimal Design of Microarray Immunoassays to Compensate for Kinetic Limitations” Mol & Cell Proteom (2006) 5.9:1681-1696.
Ladner R C “Antibodies cut down to size” Nat Biotechnol (2007) 25:875-877.
Laitinen O H, Nordlund H R, Hytonen V P, Kulomaa M S “Genetically engineered avidins and streptavidins” Trends Biotechnol (2007) 25:269-277.
Lee B, Richards F M “The interpretation of protein structures: estimation of static accessibility” J Mol Biol (1971) 55:379-400.
Malcolm B A, Taremi S S, Weber P C, Yao N “Single-chain recombinant complexes of hepatitis C virus NS3 protease and NS4A cofactor peptide” U.S. Pat. No. 6,653,127.
Nallamsetty S, et. al. “Efficient site-specific processing of fusion proteins by tobacco vein mottling virus protease in vivo and in vitro” Prot Expr Purif (2004) 38:108-115.
Padlan E A “Anatomy of the Antibody Molecule” Mol Immunol (1994) 31:169-217.
Pantoliano M W, Petrella E C, Kwasnoski J D, Lobanov V S, Myslik J, Graf E, Carver T, Asel E, Springer B A, Lane P, Salemme F R “High Density Miniaturized Thermal Shift Assay as a General Strategy for Drug Discovery” J Biomol Screening (2001) 6:429-440.
Protein Data Bank. http://www.msb.org/pdb/
Rais-Beghdadi C, Roggero M A, Fasel N, Raymond C D “Purification of recombinant proteins by chemical removal of the affinity tag” Appl Biochem Biotechnol (1998) 74:95-103.
Ringler P, Schulz G “Self-Assembly of Proteins into Designed Networks” Science (2003) 302:106-109.
Salemme F R “Design principles for self-assembling devices from macromolecules” in Bio-nanotechnology V Renugopalakrishan R V Lewis eds. Springer Netherlands (2006) pp. 1-8.
Salemme F R, Rould M A, Weber P C “Computational approaches to define geometrically critical sites in protein-based nanoassemblies” (2009) manuscript in preparation.
Sano T, Cantor C R “Cooperative Biotin Binding by Streptavidin Electrophoretic Behavior and Subunit Association of Streptavidin in the Presence of 6M Urea” J Biol Chem (1990) 265:3369-3373.
Sano T, Vajda S, Cantor C R “Genetic engineering of streptavidin, a versatile affinity tag” J Chromatogr B Biomed Sci Appl (1998) 715:85-91.
Sara M, Sleytr U B “S-Layer Proteins” J Bacteriol (2000) 182:859-868.
Schwarzenbacher R, et. al. “Crystal structure of uronate isomerase (TM0064) from Thermotoga maritima at 2.85 A resolution” (2003) Proteins: Struct Func Gen (2003) 53:142-145.
Simler B R, Doyle B L, Matthews C R “Zinc binding drives the folding and association of the homo-trimeric g-carbonic anhydrase from Methanosarcina thermophila” Prot Eng Des Sel (2004) 17:285-291.
Sligar S G, Salemme F R “Protein engineering for molecular electronics” Curr Opin Biothechnol (1992) 3:388-393.
Soukka T, Harma H, Paukkunen J, Lovgren T “Utilization of Kinetically Enhanced Monovalent Binding Affinity by Immunoassays Based on Multivalent Nanoparticle-Antibody Bioconjugates” Anal Chem (2001) 73:2254-2260.
Steinmetz N F, Lin T, Lomonossoff G P, Johnson J E “Structure-based engineering of an icosahedral virus for nanomedicine and nanotechnology” Curr Top Microbiol Immunol (2009) 327:23-58.
Suter M, Cazin J Jr, Butler J E, Mock D M “Isolation and Characterization of Highly Purified Streptavidin Obtained in a Two-Step Purification Procedure from Streptomyces avidinii Grown in a Synthetic Medium” J Immunol Meth (1988) 113:83-91.
Taremi S S, Beyer B, Maher M, Yao N, Prosise W, Weber P C, Malcolm B A “Construction, expression, and characterization of a novel fully activated recombinant single-chain hepatitis C virus protease” Prot Sci (1998) 7:2143-2149.
Thess A, Hutschenreiter S, Hofmann M, Tampe R, Baumeister W, Guckenberger R “Specific Orientation and Two-dimensional Crystallization of the Proteasome at Metal-chelating Lipid Interfaces” J Biol Chem (2002) 277:36321-36328.
Torres A J, Wu M, Holowka D, Baird B “Nanobiotechnology and Cell Biology: Micro- and Nanofabricated Surfaces to Investigate Receptor-Mediated Signaling” Ann Rev Biophys (2008) 37:265-288.
Tripp R A, Alvarez R, Anderson B, Jones L, Weeks C, Chen W “Bioconjugated nanoparticle detection of respiratory syncytial virus infection” Int J Nanomedicine (2007) 2:117-124.
Unno H, Yamashita S, Ikeda Y, Sekiguchi S, Yoshida N, Yoshimura T, Kusunoki M, Nakayama T, Nishino T, Hemmi H “Flavin's New Role as a General Acid-Base Catalyst with No Redox Function in Type 2 Isopentenyl Diphosphate Isomerase” J Biol Chem (2009) papers in press.

van den Heuvel M G L, Dekker C “Motor Proteins at Work for Nanotechnology” Science (2007) 317:333-336.
Volgraf M, Gorostiza P, Numano R, Kramer R H, Isacoff E Y, Trauner D “Allosteric control of an ionotropic glutamate receptor with an optical switch” Nat Chem Biol (2006) 2:47-52.
Vollenkle C, Weigert S, Ilk N, Egelseer E, Weber V, Loth F, Falkenhagen D, Sleytr U, Sara M “Construction of a Functional S-Layer Fusion Protein Comprising an Immunoglobulin G-Binding Domain for Development of Specific Adsorbents for Extracorporeal Blood Purification” Appl Environ Microbiol (2004) 70:1514-1521.
Wada T, Park S-Y, Tame R H, Kuramitsu S, Yokoyama S “Crystal Structure of IPP isomerase at P43212” PDB entry: 1VCG. Structure deposited, but not described in a publication.
Waner M J, Navrotskaya I, Bain A, Oldham E D, Mascotti D P “Thermal and Sodium Dodecylsulfate Induced Transitions of Streptavidin” Biophys J (2004) 87:2701-2713.
Weber P C, Ganguly A K, Kaler E W U.S. Pat. No. 5,948,688 Method of Determining Monomeric Drug Content
Weber P C, Ohlendorf D H, Wendoloski J J, Salemme F R “Structural Origins of High Affinity Biotin Binding to Streptavidin” Science (1989) 243:85-88.
Weber P C, Wendoloski J J, Pantoliano M W, Salemme F R “Crystallographic and Thermodynamic Comparison of Natural and Synthetic Ligands Bound to Streptavidin” J Amer Chem Soc (1992) 114:3197-3200.
Weber P C, Pantoliano M W, Simons D M, Salemme F R “Structure-Based Design of Synthetic Azobenzene Ligands for Streptavidin” J Amer Chem Soc (1994) 116:2717-2724.
Wendoloski J J, Salemme F R “PROBIT: A Statistical Approach to Modeling Proteins from Partial Coordinate Data Using Substructure Libraries” J Molecular Graphics (1992) 10:124-126.
Williams L, Nguyen T, Li Y, Porter T N, Raushel F M “Uronate isomerase: a nonhydropytic member of the amidohydrolase superfamily with an ambivalent requirement for a divalent metal ion” Biochemistry (2006) 45:7453-7462.
Winfren C, Borrebaeck C A K “Antibody Microarrays: Current Status and Key Technological Advances” (2006) 10:411-427.
Wolf D L, Lin P H, Hollenbach S, Wong A, Phillips D R, Sinha U “Procoagulant activity of reversibly acylated human factor Xa” Blood (1995) 86:4153-4157.
Woo H A, Chae H Z, Hwang S C, Yang K S, Kang S W, Kim K, Rhee S G “Reversing the inactivation of peroxlredoxins caused by cysteine sulfinic acid formation” Science (2003) 300:653-658.
Yoshino T, Takahashi M, Takeyama H, Okamura Y, Kato F, Matsunaga T “Assembly of G Protein-Coupled Receptors onto Nanosized Bacterial Magnetic Particles Using Mms16 as an Anchor Molecule” Appl Environ Microbiol (2004) 70:2880-2885.
Young M, Willits D, Uchida M, Douglas T “Plant viruses as biotemplates for materials and their use in nanotechnology” Ann Rev Phytopathol (2008) 46:361-384.
Adams J J, Anderson B F, Renault J P, Verchere-Beaur C, Morgenstern-Badarau I, Jameson G B (2002) “Structure and properties of the atypical iron superoxide dismutase from Methanobacterium thermoautotrophicum” To be published (2002) (pdb code: 1mai)
Benach J, Edstrom W C, Lee I, Das K, Cooper B, Xiao R, Liu J, Rost B, Acton T B, Montelione G T, Hunt J F. “The 2.35 A structure of the TenA homolog from Pyrococcus furiosus supports an enzymatic function in thiamine metabolism” (2005) Acta Crystallogr., Sect.D 61: 589-598 (pdb code: 1rtw)
Blum A S, Soto C M, Wilson C D, Cole J D, Kim M, Gnade B, Chatterji A, Ochoa W F, Lin T, Johnson J, Ratna B R “Cowpea mosaic virus as a scaffold for 3-D patterning of gold nanoparticles” Nano Lett (2004)4:867.
Blum A S, Soto C M, Wilson C D, Brower T L, Pollack S K, Schull T L, Chatterji A, Lin T, Johnson J E, Amsinck C, Franzon P, Shashidhar R, Ratna B R “An engineered virus as a scaffold for three-dimensional self-assembly on the nanoscale” Small (2005)1:702.
Case, D A Cheatham T E, Darden T, Gohlke H, Luo R, Merz, K M, Onufriev A, Simmerling C, Wang B, Woods R. “The Amber biomolecular simulation programs” (2005) J. Computat. Chem. 26, 1668-1688 (http://amber.scripps.edu/)
Castro G R, Knubovets T “Homogeneous biocatalysis in organic solvents and water-organic mixtures” Crit Rev Biotechnol (2003)23:195-231.
Chatterji A, Ochoa W F, Paine M, Ratna B R, Johnson J E, Lin T “New addresses on an addressable virus nanoblock; uniquely reactive Lys residues on cowpea mosaic virus” Chem Biol (2004)11:855.
Chatterji A, Ochoa W F, Ueno T, Lin T, Johnson J E “A virus-based nanoblock with tunable electrostatic properties” Nano Lett (2005)5:597.
Cosgrove M S, Bever K, Avalos J L, Muhammad S, Zhang X., Wolberger C. “The structural basis of sirtuin substrate affinity” (2006) Biochemistry 45: 7511-7521 (pdb code: 2h21)
Eigler D M, Schweizer E K “Positioning single atoms with a scanning tunnelling microscope” Nature (1990)344:524-526.
Esposito L, Bruno I, Sica F, Raia, C A, Giordano A, Rossi M, Mazzarella L, Zagari A. “Structural study of a single-point mutant of Sulfolobus solfataricus alcohol dehydrogenase with enhanced activity” (2003) Febs Lett. 539: 14-18 (pdb code: 1nto)
Falkner J C, Turner M E, Bosworth J K, Trentler T J, Johnson J E, Lin T, Colvin V L “Virus crystals as nanocomposite scaffolds” J Am Chem Soc (2005)127:5274.
Fitzpatrick P A, Steinmetz A C U, Ringe D, Klibanov A M “Enzyme Crystal Structure in a Neat Organic Solvent” Proc Nat Acad Sci USA (1993)90:8653.
Gupta M N, Roy I “Enzymes in organic media: Forms, functions and applications” Eur J Biochem (2004)271:2575-2583.
Hatzor-de Picciotto A, Wissner-Gross A D, Lavallee G, and Weiss P S “Arrays of Cu²⁺-Complexed Organic Clusters Grown on Gold Nano Dots” (2007) Journal of Experimental Nanoscience, 2: 3-11
Hofmann K, Wood S W, Brinton C C, Montibeller J A, Finn F M “Iminobiotin affinity columns and their application to retrieval of streptavidin” Proc Natl Acad Sci USA (1980)77:4666-4668.
Humphrey W, Dalke A, Schulten K “VMD: visual molecular dynamics” J Mol. Graph. (1996) February; 14(1):33-8-27-8. http://www.ks.uiuc.edu/Research/vmd/
Izard T, Aevarsson A, Allen M D, Westphal A H, Perham R N, de Kok A, Hol W G “Principles of quasi-equivalence and Euclidean geometry govern the assembly of cubic and dodecahedral cores of pyruvate dehydrogenase complexes” (1999) Proc. Natl. Acad. Sci. USA 96: 1240-1245 (pdb code: 1b5s)
Jones T A, Bergdoll M, Kjeldgaard M “0: A macromolecular modeling environment” In: Crystallographic and Modeling Methods in Molecular Design. Eds.: C. Bugg & S. Ealick. Springer-Verlag Press (1990)189-195. (http://xray.bmc.uu.se/˜alwyn/)
Kim K K, Kim R, Kim S H “Crystal structure of a small heat-shock protein” (1998) Nature 394: 595-599 (pdb code: 1shs)
Judy J W, “Microelectromechanical systems (MEMS): fabrication, design and applications” (2001) Smart Mater. Struct. 10 1115-1134
Kisker C, Schindelin, H, Alber B E, Ferry J G, Rees, D C “A left-hand beta-helix revealed by the crystal structure of a carbonic anhydrase from the archaeon Methanosarcina thermophile” (1996) EMBO J. v15 pp. 2323-30 (pdb code: 1thj)
Kitago Y, Yasutake Y, Sakai N, Tsujimura, M, Yao M., Watanabe N., Kawarabayasi, Y., Tanaka, I. (2003) “Crystal structure of 5′-deoxy-5′-methylthioadenosine phosphorylase homologue from Sulfolobus tokodaii” To be Published (pdb code: 1v4n)
Lee B K, Richards F M “The interpretation of protein structures: Estimation of static accessibility” (1971)”J. Mol. Biol. 55, 379-400.
Lee K B, Park S, Mirkin C A, Smith J C, Mrksich M “Protein Nanoarrays Generated By Dip-Pen Nanolithography” Science (2002)295:1702-1705.
Levene M J, Korlach J, Turner S W, Foquet M, Craighead H G, Webb W W “Zero-Mode Waveguides for Single-Molecule Analysis at High Concentrations” (2003) Science 299: 682-686□
Liu G Y, Xu S, Qian Y “Nanofabrication of Self-Assembled Monolayers Using Scanning Probe Lithography” Nanotechnology (1996)7:376-380.
Liu G Y, Amro N A “Positioning protein molecules on surfaces: A nanoengineering approach to supramolecular chemistry” Proc Nat Acad Sci (2002)99:5165-5170.
Massant J, Wouters J, Glansdorff N “Refined structure of Pyrococcus furiosus ornithine carbamoyltransferase at 1.87 A” (2003) Acta Crystallogr., Sect.D 59: 2140-2149 (pdb code: 1pvv)
Medalsy I, Dgany O, Sowwan M, Cohen H, Yukashevska A, Wolf S G, Wolf A Koster A, Almog O, Marton I, Pouny Y, Altman A, Shoseyov O, Porath D “SP1 Protein-Based Nanostructures and Arrays” (2008) Nano Lett., 8 (2), 473-477
Ni J, Sakanyan V, Charlier D, Glansdorff N, Van Duyne G D, “Structure of the arginine repressor from Bacillus stearothermophilus.” (1999) Nat. Struct. Biol. 6: 427-432 (pdb code: 1b4b)
Padilla J E, Colovos C, Yeates T O “Nanohedra: Using symmetry to design self-assembling protein cages, layers, crystals, and filaments” Proc Nat Acad Sci USA (2001)98:2217-2221.
Pearce, P, “Structure in Nature is a Strategy for Design” (1979) MIT Press, Cambridge
Protein Data Bank. http://www.rcsb.org/pdb/
Pugh, A, “Polyhedra a Visual Approach” (1976) University of California Press, Berkeley
Ringler P, Schulz G “Self-Assembly of Proteins into Designed Networks” Science (2003)302:106-109.
Rogers J A. & Nuzzo R G “Recent progress in Soft Lithography” (2005) Materials Today 8:50-56.
Rothemund P W K “Folding DNA to create nanoscale shapes and patterns” Nature (2006)440:297-302.
Rupley J A, Careri G “Protein hydration and function” Adv Protein Chem (1991)41:37-172.
Salemme, F R, Weber, P C “Streptavidin Macromolecular Adaptor And Complexes (2007) US Provisional patent
Saridakis V, Christendat D, Kimber M S, Dharamsi A, Edwards A M, Pai E F “Insights into ligand binding and catalysis of a central step in NAD+ synthesis: structures of Methanobacterium thermoautotrophicum NMN adenylyltransferase complexes.” (2001) J. Biol. Chem. 276: 7225-7232 (pdb code: 1hyb)
Schwarzenbacher R et al. and Wilson I A, “Crystals Structure of Uronate Isomerase (TM0064) From Thermotoga maritima at 2.85 A Resolution” Proteins: Struct, Funct & Bioinform (2003)53:142-145.
Schwarzenbacher R et al. and Wilson I A, “Crystal structure of a phosphoribosylaminoimidazole mutase PurE (TM0446) from Thermotoga maritima at 1.77 A resolution” (2004) Proteins 55: 474-478 (pdb code: 104v)
Seeman N C “From Genes to Machines: DNA Nanomechanical Devices” Trends in Biochemical Sciences (2005a)30:119-235.

Seeman N C “Structural DNA Nanotechnology: An Overview” Methods in Molecular Biology 303: Bionanotechnology Protocols, Editors, Sandra J. Rosenthal and David W. Wright, Humana Press, Totowa, N.J. (2005b) pp. 143-166.
Shih W M, Quispe J D, Joyce G F A “1.7-kilobase single-stranded DNA that folds into a nanoscale octahedron” Nature (2004)427:618-621.
Sleytr U B, Egelseer E M, Ilk N, Pum D, Schuster B. “S Layers as Basic Building Block for a Molecular Construction Kit” (2008) FEBS J. 274:323-334.
Sligar S G, Salemme F R “Protein engineering for molecular electronics” Curr Opin Biotechnol (1992)3:388-393.
Teplyakov, A., Obmolova, G., Wilson, K. S., Ishii, K., Kaji, H., Samejima, T., Kuranova, I. “Crystal structure of inorganic pyrophosphatase from Thermus thermophilus” (1994) Protein Sci. 3: 1098-1107 (pdb code: 2prd)
Weber P C, Ohlendorf D H, Wendoloski J J, Salemme F R “Structural Origins of High Affinity Biotin Binding to Streptavidin” Science (1989)243:85-88. (pdb code: 1stp)
Weber P C, Wendoloski J J, Pantoliano M W, Salemme F R “Crystallographic and Thermodynamic Comparison of Natural and Synthetic Ligands Bound to Streptavidin” J Amer Chem Soc (1992)114:3197-3200.
Weber P C, Wendoloski J J, Pantoliano M W, Salemme F R “Structure-Based Design of Synthetic Azobenzene Ligands for Streptavidin” J Amer Chem Soc (1994)116:2717-2724.
Weber S (1999) http://jcrystal.com/steffenweber/gallery/Fullerenes/Fullerenes.html
Wells A F, “Three Dimensional Nets and Polyhedra” (1977) Wiley, New York
Wells A F, “Further Studies of Three Dimensional Nets” (19779) American Crystallographic Association Monograph No. 8
Widman D, Freidrich H, Mader H, “Technology of Integrated Circuits” (2000) Springer-Verlag New York, LLC (340 pp) ISBN-13: 9783540661993
Williams R, “The Geometrical Foundation of Natural Structure” (1979) Dover, N.Y.
Vainshtein B K, “Modern Crystallography, 1: Fundamentals of Crystals” (1994) Springer, New York,
Whitesides G M, Mathias J P, Seto C T. “Molecular Self Assembly and Nanochemistry: A chemical strategy for the synthesis for the synthesis of nanostructures” (1991) Science 254, 1312-1319
Whitesides G M, Boncheva M “Beyond molecules: Self-assembly of mesoscopic and macroscopic components” Proc Nat Acad Sci USA (2002)99:4769-4774.
Xia Y & Whitesides G “Soft Lithography” (1998) Annu. Rev. Mater. Sci. 28, 153-184.
Yeates T O, Padilla J E, Colovos C, “Self Assembling Proteins” U.S. Pat. No. 6,756,039 (2004)
Zaks A, Klibanov A M “Enzymatic catalysis in nonaqueous solvents” J Biol Chem (1988)263:3194-3201.
Zhu J, Huang J, Stepanyuk G, Chen L, Chang J, Zhao M, Xu H, Liu Z J, Rose J P, Wang B C. “Crystal Structure of Tt0030 from Thermus thermophilus at 1.6 Angstroms Resolution” To be Published 2006 (pdb code: 2iel)

### Outline of the Invention:

Paragraph 1. A fused binding domain node, comprising

a multimeric protein comprising a plurality of subunits;

wherein the subunits are covalently linked through a sequence of amino acid residues;

a binding domain comprising a sequence of amino acid residues;

wherein the binding domain is covalently linked to a subunit.

Paragraph 2. The fused binding domain node of Paragraph 1,

wherein the binding domain is capable of binding an antibody.

Paragraph 3. The fused binding domain node of Paragraph 1,

wherein the binding domain is a Protein G domain.

Paragraph 4. A fused binding domain node—antibody complex, comprising

the fused binding domain node of Paragraph 1; and

and an antibody,

wherein the antibody is bound to the binding domain of the fused binding domain node.

“Linked” and “bound” can refer, for example, to an association of a molecule, a portion of a molecule, a group of atoms, or an atom with another molecule, portion of a molecule, group of atoms, or atom. The “linked” or “bound” state can arise from covalent, ionic, hydrophobic, van der Waals, or other types of interactions or forces.
A “subunit” can refer, for example, to a portion of a molecule, or a portion of a multimeric structure.

TABLE 3
Biotin linking reagents for preparation of NODEs and NODE:streptavidin complexes.
Chemical structures of the maleimide-reactive biotin-linking reagents MAL PEO3 and MAL PEO11, the maleimide-reactive iminobiotin-linking Reagent
MAL PEO3, and the sulfur- reactive biotin-linking reagent, EZ-Link HPDP, are shown along with a schematic representation used elsewhere. The PEO-
containing reagents with ethylene glycol-based chains are more water-soluble than their aliphatic counterparts. The MAL-containing reagents contain a
terminal maleimide that forms a covalent S—C bond on reaction with the cysteine sulfhydryl, while a reversible S—S bond is formed by reaction with
EZ-Link HPDP. Three of the reagents, MAL PEO3 biotin and imino biotin, and EZ-Link HPDP have ~20 atoms to span between the biotin carboxylate and
the NODE cysteine sulfur, while the MAL PEO11 reagent has ~42 intervening atoms.

| Reagent Chemical Structure | | Schematic & Description |
|---|---|---|
| a. | 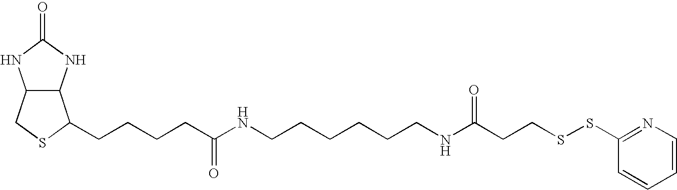 | 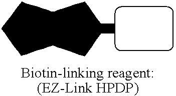 |
| b. | 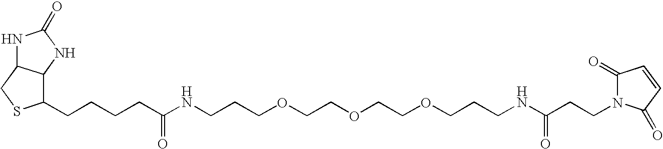 | 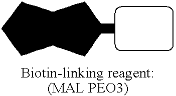 |
| c. | 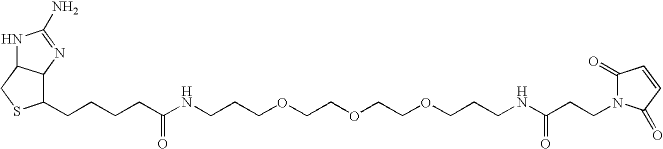 | 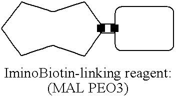 |
| d | 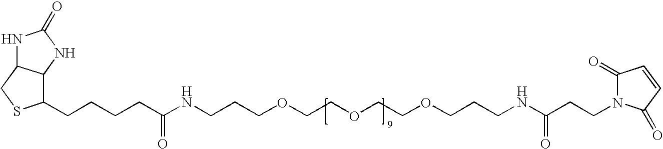 | 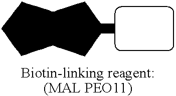 |

TABLE 4
Cysteine Substitution Sites Selected on C3 & C4 Node Template Proteins

| | Thermotoga | Thermus thermophilus type | Methanosarcina |
|---|---|---|---|
| | maritima uronate | 2 isopentenyl diphosphate | thermophila g-carbonic |
| Protein | isomerase | isomerase | anhydrase |
| Node Symmetry | C3 | C4 | C3 |
| Engineered Cysteine | Lys42, Ser77 | Ser44, Thr49 | Asp70, Tyr200 |
| Sites | | | |
| Free Cysteines | Cys65 to Ala, | Cys14 to Ala, | Cys148to Ala* |
| removed from the | Cys149 to Ser, | Cys237 to Ser | |
| native sequence | Cys227 to Ala | | |

*The Cys148 to Ser g-carbonic anhydrase variant has been successfully expressed in E. coli (Simler et al. 2004).

TABLE 5
Genes Synthesized

| | |
|---|---|
| (C4) Uronate Isomerase | Native sequence plus N-terminal His6 tag |
| (C4) IPP Isomerase | Native sequence |
| (C4) IPP Isomerase engineered for | C14A, S44C, T49C, C237S, N-terminal His6 tag with TEV |
| interaction with streptavidin | protease cleavage site |
| C3g-carbonic anhydrase | Native sequence, C-terminal His6 tag with Factor Xa |
| | cleavage site |
| C3g-carbonic anhydrase engineered for | D70C, Y200C, C148A, C-terminal His6 tag with Factor Xa |
| interaction with streptavidin | cleavage site |
| C3Single chaing-carbonic anhydrase | D6 start, D70C, Y200C, C148A, GGSGGG linker (D6 start, |
| engineered for interaction with streptavidin | D70C, Y200C, C148A) GGSGGG linker (D6 start, D70C, |
| at three sites | Y200C, C148A) C-terminal His6 tag with Factor Xa |
| | cleavage site |
| C3Single chaing-carbonic anhydrase | D6 start, D70C, Y200C, C148A, GGSGGG linker (D6 start, |
| engineered for interaction with streptavidin | D70C, Y200C, C148A) GGSGGG linker (D6 start, C148A) |
| at two sites | C-terminal His6 tag with Factor Xa cleavage site |

1. A fused binding domain node, comprising
a multimeric protein comprising a plurality of subunits;
wherein the subunits are covalently linked through a sequence of amino acid residues;
a binding domain comprising a sequence of amino acid residues;
wherein the binding domain is covalently linked to a subunit.

2. The fused binding domain node of claim 1,
wherein the binding domain is capable of binding an antibody.

3. The fused binding domain node of claim 1,
wherein the binding domain is a Protein G domain.

4. A fused binding domain node—antibody complex, comprising
the fused binding domain node of claim 1; and
and an antibody,
wherein the antibody is bound to the binding domain of the fused binding domain node.

5. A nanostructure node, comprising:
a nanostructure node multimeric protein comprising at least one polypeptide chain,
wherein the nanostructure node multimeric protein has a known 3-dimensional structure,
wherein the nanostructure node multimeric protein essentially has Cn, Dn, or higher symmetry with a number of subunits,
wherein the nanostructure node multimeric protein is stable at a temperature of 70° C. or greater,
wherein the nanostructure node multimeric protein has an amino acid sequence not found in nature,
wherein the nanostructure node multimeric protein comprises a specific binding site for the attachment of a nanostructure strut with predefined stoichiometry and orientation,
wherein the specific binding site comprises at least two specific amino acid reactive residues, and
wherein each specific amino acid reactive residue has a covalently attached biotin group.
